(19) United States
(12) Patent Application Publication
(10) Pub. No.: US 2024/0353310 A1
(43) Pub. Date: Oct. 24, 2024

(54) BLOOD CELL ANALYZER, METHOD FOR INDICATING INFECTION STATUS AND USE OF INFECTION MARKER PARAMETER

(71) Applicant: SHENZHEN MINDRAY BIO-MEDICAL ELECTRONICS CO., LTD., Shenzhen (CN)

(72) Inventors: Jin LI, Shenzhen (CN); Shiyao PAN, Shenzhen (CN); Xiaomei ZHANG, Shenzhen (CN); Chuanjian WU, Shenzhen (CN); Wenbo ZHENG, Shenzhen (CN); Bo YE, Shenzhen (CN)

(73) Assignee: SHENZHEN MINDRAY BIO-MEDICAL ELECTRONICS CO., LTD., Shenzhen (CN)

(21) Appl. No.: 18/759,876

(22) Filed: Jun. 29, 2024

Related U.S. Application Data
(63) Continuation of application No. PCT/CN2022/143965, filed on Dec. 30, 2022.

(30) Foreign Application Priority Data
Dec. 31, 2021 (WO) PCT/CN2021/143877

(51) Int. Cl. G01N 15/14; G01N 1/30; G01N 15/10; G01N 21/47; G01N 21/64; G16H 50/20

(52) U.S. Cl. CPC G01N 15/1459; G01N 1/30; G01N 21/6486; G16H 50/20; G01N 2015/1006; G01N 2021/4707; G01N 2021/4726; G01N 2800/26

(57) ABSTRACT

The present invention relates to a blood cell analyzer, a method, and a use of an infection marker parameter. The blood cell analyzer comprises a sample suction device used for aspirating a blood sample to be tested of a subject, a sample preparation device used for preparing a test sample containing a part of a blood sample to be tested, a hemolytic agent, and a staining agent for identifying nucleated red blood cells, an optical detection device used for detecting an test sample to obtain optical information, and a processor. The processor obtains from the optical information at least one leukocyte characteristic parameter of at least one target particle population in a test sample, obtains an infection marker parameter for evaluating an infection status of a subject on the basis of the at least one leukocyte characteristic parameter, and outputs the infection marker parameter.

### CROSS-REFERENCE

This application is a bypass continuation of International Application No. PCT/CN2022/143965, filed Dec. 30, 2022, which claims the benefits of priority of International Application No. PCT/CN2021/143877, entitled “HEMATOLOGY ANALYZER, METHOD FOR INDICATING INFECTION STATUS, AND USE OF INFECTION MARKER PARAMETER” and filed on Dec. 31, 2021. The entire contents of each of the above-referenced applications are expressly incorporated herein by reference.

### TECHNICAL FIELD

The present application relates to the field of in vitro diagnostics, and in particular to a blood cell analyzer, a method for indicating the infection status of a subject, and the use of an infection marker parameter in evaluating the infection status of a subject.

### BACKGROUND

Infectious diseases are common clinical diseases, among which sepsis is a serious infectious disease. The incidence of sepsis is high, with more than 18 million severe sepsis cases worldwide every year. Sepsis is dangerous and has a high case fatality rate, with about 14,000 people dying from its complications worldwide every day. According to foreign epidemiological surveys, the case fatality rate of sepsis has exceeded that of myocardial infarction, and has become the main cause of death for non-heart disease patients in intensive care units. In recent years, despite great advances in anti-infective treatment and organ function support technologies, the case fatality rate of sepsis is still as high as 30% to 70%. The treatment of sepsis is expensive and consumes a lot of medical resources, which seriously affects the quality of human life and has posed a huge threat to human health. Clinicians need to diagnose whether the patient is infected in time and find the pathogen in order to make an effective treatment plan. Therefore, how to quickly and early screen and diagnose infectious diseases has become an urgent problem to be solved in clinical laboratories.

For rapid differential diagnosis of infectious diseases, existing solutions in the industry include: microbial culture, inflammatory markers, such as C-reactive protein (CRP), procalcitonin (PCT), and serum amyloid A (SAA), serum antigen and antibody detection and blood routine test.

Microbial culture is considered to be the most reliable gold standard. It enables directly culture and detection of bacteria in clinical specimens such as body fluid or blood, so as to interpret the type and drug resistance of a bacteria, thereby providing direct guidance for clinical drug use. However, this method has a long turnaround time, the specimen is easily contaminated and the false negative rate is high, which cannot meet the requirements of rapid and accurate clinical results.

Inflammatory factors such as CRP, PCT and SAA are widely used in the auxiliary diagnosis of infectious diseases due to their good sensitivity. However, the specificity of these detections for infectious diseases is weak, and the combined detection of CRP, PCT and SAA is usually required, which increases the economic burden of patients. Moreover, CRP and PCT are interfered by specific diseases, so sometimes they cannot correctly reflect the infection status of patients. For example, CRP is generated in the liver, and infected patients with liver damage have normal CRP levels and will have false negative results in the diagnosis of infectious diseases.

Serum antigen and antibody detection may identify specific virus types, but it has limited effect at situations where the type of pathogen is not clear, and the detection cost is high, which increases the economic burden of patients.

Blood routine test may indicate the occurrence of infection and the identify infection types to a certain extent. However, leukocyte (White Blood Cell, abbreviated as “WBC”) \ neutrophil (Neu) %, etc. in blood routine results are easily affected by many aspects, such as other non-infectious inflammatory responses, and normal physiological fluctuations in the body, and thus cannot accurately and timely reflect the condition of the patient, and has poor diagnostic and therapeutic value for infectious diseases.

### SUMMARY

In order to solve the above-mentioned technical problems, one of the objectives of the disclosure is to provide a solution that can quickly evaluate the infection status of a subject at a low cost, in which novel blood cell morphological parameters are developed using a blood cell analyzer to evaluate the infection status of the subject, including an early prediction of sepsis, diagnosis of sepsis, an identification of a common infection and a severe infection, monitoring of infection, an analysis of sepsis prognosis, an identification of a bacterial infection and a viral infection, an identification of a non-infectious inflammation and an infectious inflammation, or an evaluation of therapeutic effect on sepsis.

In addition, the solution does not require additional testing costs, and can effect the evaluation of infection status while using existing blood cell analyzers for blood routine test.

In order to achieve the above objective of the disclosure, the first aspect of the disclosure provides a blood cell analyzer including:
a sample aspiration device configured to aspirate a blood sample of a subject to be tested;
a sample preparation device configured to prepare a test sample containing a part of the blood sample to be tested, a hemolytic agent, and a staining agent for identifying nucleated red blood cells;
an optical detection device comprising a flow cell, a light source and an optical detector, the flow cell being configured to allow the test sample to pass therethrough, the light source being configured to irradiate with light the test sample passing through the flow cell, and the optical detector being configured to detect optical information generated by the test sample under irradiation when passing through the flow cell; and
a processor configured to:
calculate from the optical information at least one leukocyte characteristic parameter of at least one target particle population in the test sample;
obtain an infection marker parameter for evaluating an infection status of the subject based on the at least one leukocyte characteristic parameter; and
output the infection marker parameter.

In some embodiments, the processor further identifies nucleated red blood cells in the test sample based on the optical information to obtain a nucleated red blood cell count.

In some embodiments, the at least one target particle population is selected from leukocyte population, neutrophil population and lymphocyte population; in some embodiments the at least one target particle population is selected from leukocyte population and neutrophil population.

In some embodiments, in order to calculate from the optical information at least one leukocyte characteristic parameter of at least one target particle population in the test sample, and obtain an infection marker parameter for evaluating an infection status of the subject based on the at least one leukocyte characteristic parameter by the processor,
the processor further obtains one or more leukocyte characteristic parameters from the optical information and obtains the infection marker parameter based on the one or more leukocyte characteristic parameters, the one or more leukocyte characteristic parameters are selected from: a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, and a side fluorescence intensity distribution coefficient of variation of the leukocyte population;
an area of a distribution region of the leukocyte population in a two-dimensional scattergram generated by two light intensities selected from forward scatter intensity, side scatter intensity and side fluorescence intensity, and a volume of a distribution region of the leukocyte population in a three-dimensional scattergram generated by forward scatter intensity, side scatter intensity and side fluorescence intensity;
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, and a side fluorescence intensity distribution coefficient of variation of the neutrophil population;
an area of a distribution region of the neutrophil population in a two-dimensional scattergram generated by two light intensities selected from forward scatter intensity, side scatter intensity and side fluorescence intensity, and a volume of a distribution region of the neutrophil population in a three-dimensional scattergram generated by forward scatter intensity, side scatter intensity and side fluorescence intensity;
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, and a side fluorescence intensity distribution coefficient of variation of the lymphocyte population; and
an area of a distribution region of the lymphocyte population in a two-dimensional scattergram generated by two light intensities selected from forward scatter intensity, side scatter intensity and side fluorescence intensity, and a volume of a distribution region of the lymphocyte population in a three-dimensional scattergram generated by forward scatter intensity, side scatter intensity and side fluorescence intensity.

In some embodiments, in order to calculate from the optical information at least one leukocyte characteristic parameter of at least one target particle population in the test sample, and obtain an infection marker parameter for evaluating an infection status of the subject based on the at least one leukocyte characteristic parameter by the processor, the processor further calculates one or more leukocyte characteristic parameters from the optical information and obtains the infection marker parameter based on the one or more leukocyte characteristic parameters, the one or more leukocyte characteristic parameters are selected from: a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, a side fluorescence intensity distribution coefficient of variation of the leukocyte population, and an area of a distribution region of the leukocyte population in a two-dimensional scattergram generated by two light intensities selected from forward scatter intensity, side scatter intensity and side fluorescence intensity, a volume of a distribution region of the leukocyte population in a three-dimensional scattergram generated by forward scatter intensity, side scatter intensity and side fluorescence intensity;
in some embodiments, the processor further calculates from the optical information one or more of the side fluorescence intensity distribution center of gravity, the forward scatter intensity distribution width, and the side fluorescence intensity distribution width of the leukocyte population, and obtain the infection marker parameter based on the calculation.

In some embodiments, the processor further:
outputs prompt information indicating that the infection marker parameter is abnormal when a value of the infection marker parameter is beyond a preset range.

In some embodiments, the processor further outputs prompt information indicating the infection status of the subject based on the infection marker parameter.

In some embodiments, the infection marker parameter is used for early prediction of sepsis of the subject;
in some embodiments, the processor further obtains from the optical information the forward scatter intensity distribution width of the leukocyte population or the side fluorescence intensity distribution width of the leukocyte population and determines the obtained distribution width as the infection marker parameter; or
in some embodiments, the processor further obtains a combination of the side fluorescence intensity distribution center of gravity of the leukocyte population and the forward scatter intensity distribution width of the leukocyte population from the optical information, and calculates the infection marker parameter based on the combination.

In some embodiments, the processor further: outputs prompt information indicating that the subject is likely to progress to sepsis within a certain period of time starting from when the blood sample to be tested is collected, if the infection marker parameter satisfies a first preset condition; in some embodiments, the certain period of time is not greater than 48 hours, more in some embodiments, the certain period of time is within 24 hours.

In some embodiments, the infection marker parameter is used for diagnosis of sepsis in the subject;
in some embodiments, the processor further obtains the side fluorescence intensity distribution width of the leukocyte population or the side fluorescence intensity distribution width of the neutrophil population from the optical information and determines the obtained distribution width as the infection marker parameter; or
in some embodiments, the processor further obtains from the optical information a combination of the side fluorescence intensity distribution center of gravity of the leukocyte population and the forward scatter intensity distribution width of the leukocyte population, and calculates the infection marker parameter based on the combination.

In some embodiments, the processor further: outputs prompt information indicating that the subject has sepsis, when the infection marker parameter satisfies a second preset condition.

In some embodiments, the infection marker parameter is used for identification between common infection and severe infection in the subject;
in some embodiments, the processor further obtains from the optical information a side fluorescence intensity distribution width of the leukocyte population or an area of the distribution region of the neutrophil population in the two-dimensional scattergram generated by the side scatter intensity and the side fluorescence intensity, and determine the obtained distribution width or area of the distribution region as the infection marker parameter; or
in some embodiments, the processor further obtains from the optical information a combination of a side fluorescence intensity distribution center of gravity of the leukocyte population and a forward scatter intensity distribution width of the leukocyte population, and calculates the infection marker parameter based on the combination.

In some embodiments, the processor further: outputs prompt information indicating that the subject has a severe infection, when the infection marker parameter satisfies a third preset condition.

In some embodiments, the subject is an infected patient, or a patient suffering from a severe infection or sepsis, and the infection marker parameter is used for monitoring the infection status of the subject;
in some embodiments, the processor further determines a side fluorescence intensity distribution width of the leukocyte population as the infection marker parameter; or
in some embodiments, the processor further calculates the infection marker parameter based on a combination of the side fluorescence intensity distribution center of gravity of the leukocyte population and the forward scatter intensity distribution width of the leukocyte population.

In some embodiments, the processor further monitors a progress in the infection status of the subject based on the infection marker parameter;
in some embodiments, the processor further:
obtains multiple values of the infection marker parameter, which are obtained by multiple tests, in particular at least three tests of a blood sample from the subject at different time points; and
determines whether the infection status of the subject is improving or not according to a trend of change in the multiple values of the infection marker parameter obtained by the multiple tests, in some embodiments, outputs prompt information indicating that the infection status of the subject is improving, when the multiple values of the infection marker parameter obtained by the multiple tests gradually tend to decrease.

In some embodiments, the subject is a patient with sepsis who has received a treatment, and the infection marker parameter is used for analysis of sepsis prognosis in the subject, in some embodiments, the processor further: outputs prompt information indicating that the subject is in favorable sepsis prognosis, when the infection marker parameter satisfies a fourth preset condition.

In some embodiments, the infection marker parameter is used for identification between bacterial infection and viral infection in the subject, in some embodiments, the processor further determines whether the subject has the bacterial infection or the viral infection based on the infection marker parameter.

In some embodiments, the infection marker parameter is used for identification between non-infectious inflammation and infectious inflammation of the subject,
in some embodiments, the processor further: outputs prompt information indicating that the subject has an infectious inflammation, when the infection marker parameter satisfies a fifth preset condition.

In some embodiments, the subject is a patient with sepsis who is receiving medication, and the infection marker parameter is used for evaluation of a therapeutic effect on sepsis of the subject.

In some embodiments, the processor further obtains a leukocyte count of the test sample based on the optical information before obtaining from the optical information the at least one leukocyte characteristic parameter of at least one target particle population in the test sample, and outputs a retest instruction to retest the blood sample of the subject when the leukocyte count is less than a preset threshold, wherein a measurement amount of the sample to be retested is greater than a measurement amount of the sample to be tested; and
the processor further obtains at least another leukocyte characteristic parameter of at least another target particle population from the optical information obtained by the retest, and obtains an infection marker parameter for evaluating the infection status of the subject based on the at least another leukocyte characteristic parameter.

In some embodiments, the processor further:
skips outputting a value of the infection marker parameter, or output a value of the infection marker parameter and simultaneously outputs prompt information indicating that the value of the infection marker parameter is unreliable, when a preset characteristic parameter of the target particle population satisfies a sixth preset condition.

In some embodiments, the processor further:
skips outputting a value of the infection marker parameter, or output a value of the infection marker parameter and simultaneously outputs prompt information indicating that the value of the infection marker parameter is unreliable, when a total number of particles of the target particle population is less than a preset threshold and/or when the target particle population overlaps with another particle population.

In some embodiments, the processor further:
skips outputting a value of the infection marker parameter, or output a value of the infection marker parameter and simultaneously outputs prompt information indicating that the value of the infection marker parameter is unreliable, when the subject suffers from a hematological disorder or there are abnormal cells, especially blast cells, in the blood sample to be tested, such as when it is determined based on the optical information that there are abnormal cells, especially blast cells, in the blood sample to be tested.

In some embodiments, in order to calculate at least one leukocyte characteristic parameter of at least one target particle population in the test sample from the optical information, and obtain an infection marker parameter for evaluating an infection status of the subject based on the at least one leukocyte characteristic parameter, the processor further:
calculates a plurality of parameters of the at least one target particle population in the test sample from the optical information;
obtains a plurality of sets of the infection marker parameters for evaluating the infection status of the subject from the plurality of parameters;
assigns a priority for each set of the infection marker parameters of the plurality of sets of the infection marker parameters;
calculates a credibility of each set of the infection marker parameters of the plurality of sets of the infection marker parameters, selects at least one set of the infection marker parameters from the plurality of sets of the infection marker parameters based on the priority and credibility of the plurality of sets of the infection marker parameters so as to obtain the infection marker parameter; or according to the priority of the plurality of sets of the infection marker parameters, successively calculates a credibility of the plurality of sets of the infection marker parameters and determines whether the credibility reaches a corresponding credibility threshold, and when the credibility of a current set of the infection marker parameters reaches the corresponding credibility threshold, obtains the infection marker parameter based on said set of the infection marker parameters and stop calculation and determination;
in some embodiments, the processor further:
calculates a credibility of each set of the infection marker parameters of the plurality of sets of the infection marker parameters, and determines whether the credibility of each set of the infection marker parameters reaches a corresponding credibility threshold;
uses the sets of the infection marker parameters, whose respective credibility reaches the corresponding credibility threshold, among the plurality of sets of the infection marker parameters as candidate sets of the infection marker parameters; and
selects at least one candidate set of the infection marker parameters from the candidate sets of the infection marker parameters according to the priority of the candidate sets of the infection marker parameters, in some embodiments selects a set of the infection marker parameters with a highest priority, so as to obtain the infection marker parameter.

In some embodiments, in order to calculate at least one leukocyte characteristic parameter of at least one target particle population in the test sample from the optical information, and obtain an infection marker parameter for evaluating an infection status of the subject based on the at least one leukocyte characteristic parameter, the processor further:
calculates a plurality of parameters of the at least one target particle population in the test sample from the optical information,
obtains a plurality of sets of the infection marker parameters for evaluating the infection status of the subject from the plurality of parameters,
calculates a credibility of each set of the infection marker parameters of the plurality of sets of the infection marker parameters, selects at least one set of the infection marker parameters from the plurality of sets of the infection marker parameters based on respective credibility of the plurality of sets of the infection marker parameters so as to obtain the infection marker parameter.

In some embodiments, in order to calculate at least one leukocyte characteristic parameter of at least one target particle population in the test sample from the optical information, and obtain an infection marker parameter for evaluating an infection status of the subject based on the at least one leukocyte characteristic parameter, the processor further: determines based on the optical information whether the blood sample to be tested has an abnormality that affects the evaluation of the infection status;
obtains from the optical information the at least one leukocyte characteristic parameter of at least one target particle population unaffected by the abnormality so as to obtain the infection marker parameter, when it is determined that the blood sample to be tested has the abnormality that affects the evaluation of the infection status.

In some embodiments, the processor further selects the at least one leukocyte characteristic parameter and obtain the infection marker parameter based on the at least one leukocyte characteristic parameter such that a diagnostic efficacy of the infection marker parameter is greater than 0.5, in some embodiments greater than 0.6, particularly in some embodiments greater than 0.8.

In order to achieve the above objective of the disclosure, the second aspect of the disclosure provides a method for indicating an infection status of a subject, the method comprising
obtaining a blood sample to be tested from the subject;
preparing a test sample containing a part of the blood sample to be tested, a hemolytic agent, and a staining agent for identifying nucleated red blood cells;
passing particles in the test sample one by one through an optical detection region of the flow cell irradiated with light to obtain optical information generated by the particles in the test sample after being irradiated with light;
calculating at least one leukocyte characteristic parameter of at least one target particle population in the test sample from the optical information;
obtaining an infection marker parameter based on the at least one leukocyte characteristic parameter; and
indicating the infection status of the subject based on the infection marker parameter.

In some embodiments, the calculating at least one leukocyte characteristic parameter of at least one target particle population in the test sample from the optical information and calculating an infection marker parameter based on the at least one leukocyte characteristic parameter comprise:
calculating from the optical information one or more leukocyte characteristic parameter selected from side fluorescence intensity distribution center of gravity, forward scatter intensity distribution width, and side fluorescence intensity distribution width of the leukocyte population, and obtaining the infection marker parameter.

In order to achieve the above purpose of the disclosure, the third aspect of the disclosure further provides a use of an infection marker parameter in evaluating an infection status of a subject, wherein the infection marker parameter is obtained by a method comprising the steps of:
obtaining at least one leukocyte characteristic parameter of at least one target particle population obtained by flow cytometry detection on a test sample containing a blood sample to be tested from the subject, a hemolytic agent and a staining agent for identifying nucleated red blood cells; and
obtaining an infection marker parameter based on the at least one leukocyte characteristic parameter.

In order to achieve the above purpose of the disclosure, the fourth aspect of the disclosure further provides a blood cell analyzer, comprising:
a measurement device configured to mix a blood sample of a subject to be tested, a hemolytic agent and a staining agent to prepare a test sample and perform an optical measurement on the test sample to obtain optical information of the test sample; and
a controller configured to:
receive a mode setting instruction,
when the mode setting instruction indicates that a blood routine test mode is selected, control the measurement device to perform an optical measurement on a test sample at a first measurement amount to obtain optical information of the test sample, and obtain and output blood routine parameters of the test sample based on the optical information,
when the mode setting instruction indicates that a sepsis test mode is selected, control the measurement device to perform an optical measurement on a test sample at a second measurement amount to obtain optical information of the test sample, the second measurement being greater than the first measurement amount, obtain at least one leukocyte characteristic parameter of at least one target particle population in the test sample from the optical information, obtain an infection marker parameter for evaluating an infection status of the subject based on the at least one leukocyte characteristic parameter, and output the infection marker parameter.

In the technical solutions provided in various aspects of the disclosure, leukocyte characteristic parameters including cell characteristic parameters can be obtained from a detection channel for identifying nucleated red blood cells, thereby assisting doctors to predict or diagnose infectious diseases quickly, accurately and efficiently. In particular, prompt information indicating the infection status of the subject can be effectively provided based on the infection marker parameter.

### BRIEF DESCRIPTION OF THE DRAWINGS

FIG. 1 is a schematic diagram of a structure of a blood cell analyzer according to some embodiments of the disclosure.

FIG. 2 is a schematic diagram of a structure of an optical detection device according to some embodiments of the disclosure.

FIG. 3 is an FL-FS two-dimensional scattergram of a test sample according to some embodiments of the disclosure.

FIG. 4 is an SS-FS two-dimensional scattergram of a test sample according to some embodiments of the disclosure.

FIG. 5 is an FL-SS-FS three-dimensional scattergram of a test sample according to some embodiments of the disclosure.

FIG. 6 shows cell characteristic parameters of leukocyte populations in a test sample according to some embodiments of the disclosure.

FIG. 7 is a schematic flowchart for monitoring the progression of the infection status of the patient according to some embodiments of the disclosure.

FIGS. 8-10 are scattergrams showing the presence of abnormalities in a test sample according to some embodiments of the disclosure.

FIG. 11 shows a scattergram before and after logarithmic processing according to some embodiments of the disclosure.

FIG. 12 is a schematic flowchart of a method for indicating the infection status of a subject according to some embodiments of the disclosure.

FIGS. 13-14 are ROC curves in the case of early prediction of sepsis according to some embodiments of the disclosure.

FIGS. 15-16 are ROC curves in the case of severe infection identification according to some embodiments of the disclosure.

FIGS. 17-18 are ROC curves in the case of diagnosis of sepsis according to some embodiments of the disclosure.

FIGS. 19-21 are graphs of numerical variations of infection marker parameters for monitoring the progression of severe infection according to some embodiments of the disclosure.

FIGS. 22 and 23 are graphs of numerical variations of infection marker parameters for monitoring the progression of sepsis condition according to some embodiments of the disclosure.

FIGS. 24A-24D visually show results of detection of efficacy on sepsis using N_WBC_FL_P as a single parameter. FIG. 24A shows N_WBC_FL_P assay values before antibiotic treatment and after 5 days of antibiotic treatment for each patient in the effective and ineffective groups. FIG. 24B shows a box-and-whisker plot of patients in the effective and ineffective groups. FIG. 24C shows a comparison of the mean N_WBC_FL_P assay values before antibiotic treatment and after 5 days of antibiotic treatment in the effective group, and a comparison of the mean N_WBC_FL_P assay value before antibiotic treatment and after 5 days of antibiotic treatment in the ineffective group. FIG. 24D shows the ROC curve of the detection of efficacy on sepsis using N_WBC_FL_P as a single parameter.

FIGS. 25A-25D visually show results of detection of efficacy on sepsis using N_FL_PULWID_MEAN as a single parameter. FIG. 25A shows N_FL_PULWID_MEAN assay values before antibiotic treatment and after 5 days of antibiotic treatment for each patient in the effective and ineffective groups. FIG. 25B shows a box-and-whisker plot of patients in the effective and ineffective groups. FIG. 25C shows a comparison of the mean N_FL_PULWID_MEAN assay values before antibiotic treatment and after 5 days of antibiotic treatment in the effective group, and a comparison of the mean N_FL_PULWID_MEAN assay value before antibiotic treatment and after 5 days of antibiotic treatment in the ineffective group.

FIG. 25D shows the ROC curve of the detection of efficacy on sepsis using N_FL_PULWID_MEAN as a single parameter.

FIGS. 26A-26D visually show results of detection of efficacy on sepsis using N_FS_PULWID_MEAN as a single parameter. FIG. 26A shows N_FS_PULWID_MEAN assay values before antibiotic treatment and after 5 days of antibiotic treatment for each patient in the effective and ineffective groups. FIG. 26B shows a box-and-whisker plot of patients in the effective and ineffective groups. FIG. 26C shows a comparison of the mean N_FS_PULWID_MEAN assay values before antibiotic treatment and after 5 days of antibiotic treatment in the effective group, and a comparison of the mean N_FS_PULWID_MEAN assay value before antibiotic treatment and after 5 days of treatment in the ineffective group. FIG. 26D shows the ROC curve of the detection of efficacy on sepsis using N_FS_PULWID_MEAN as a single parameter.

FIGS. 27A-27D visually show results of detection of efficacy on sepsis using a combination of the two parameters “N_WBC_FL_P” and “N_WBC_FS_W” as the infection marker parameter. FIG. 27A shows the two-parameter combination assay values before antibiotic treatment and after 5 days of antibiotic treatment for each patient in the effective and ineffective groups. FIG. 27B shows a box-and-whisker plot of patients in the effective and ineffective groups. FIG. 27C shows a comparison of the mean values of the two-parameter combination before antibiotic treatment and after 5 days of antibiotic treatment in the effective group, and a comparison of the mean values of the two-parameter combination before antibiotic treatment and after 5 days of antibiotic treatment in the ineffective group. FIG. 27D shows the ROC curve of the detection of efficacy on sepsis using the two-parameter combination.

FIGS. 28A-28D visually show results of detection of efficacy on sepsis using a combination of the two parameters “N_WBC_FL_W” and “N_WBC_FS_P” as the infection marker parameter. FIG. 28A shows the two-parameter combination assay values before antibiotic treatment and after 5 days of antibiotic treatment for each patient in the effective and ineffective groups. FIG. 28B shows a box-and-whisker plot of patients in the effective and ineffective groups. FIG. 28C shows a comparison of the mean values of the two-parameter combination before antibiotic treatment and after 5 days of antibiotic treatment in the effective group, and a comparison of the mean values of the two-parameter combination before antibiotic treatment and after 5 days of antibiotic treatment in the ineffective group. FIG. 28D shows the ROC curve of the detection of efficacy on sepsis using the two-parameter combination.

FIGS. 29A-29D visually show results of detection of efficacy on sepsis using a combination of the two parameters “N_WBC_FL_P” and “N_WBC_FS_CV” as the infection marker parameter. FIG. 29A shows the two-parameter combination assay values before antibiotic treatment and after 5 days of antibiotic treatment for each patient in the effective and ineffective groups. FIG. 29B shows a box-and-whisker plot of patients in the effective and ineffective groups. FIG. 29C shows a comparison of the mean values of the two-parameter combination before antibiotic treatment and after 5 days of antibiotic treatment in the effective group, and a comparison of the mean values of the two-parameter combination before antibiotic treatment and after 5 days of antibiotic treatment in the ineffective group. FIG. 29D shows the ROC curve of the detection of efficacy on sepsis using the two-parameter combination.

FIGS. 30A-30D visually show results of detection of efficacy on sepsis using a combination of the two parameters “N_WBC_FL_W” and “D_Neu_FL_W” as the infection marker parameter. FIG. 30A shows the two-parameter combination assay values before antibiotic treatment and after 5 days of antibiotic treatment for each patient in the effective and ineffective groups. FIG. 30B shows a box-and-whisker plot of patients in the effective and ineffective groups. FIG. 30C shows a comparison of the mean values of the two-parameter combination before antibiotic treatment and after 5 days of antibiotic treatment in the effective group, and a comparison of the mean values of the two-parameter combination before antibiotic treatment and after 5 days of antibiotic treatment in the ineffective group. FIG. 30D shows the ROC curve of the detection of efficacy on sepsis using the two-parameter combination.

FIGS. 31A-31D visually show results of detection of efficacy on sepsis using a combination of the two parameters “N_WBC_FL_W” and “D_Neu_FL_CV” as the infection marker parameter. FIG. 31A shows the two-parameter combination assay values before antibiotic treatment and after 5 days of antibiotic treatment for each patient in the effective and ineffective groups. FIG. 31B shows a box-and-whisker plot of patients in the effective and ineffective groups. FIG. 31C shows a comparison of the mean values of the two-parameter combination before antibiotic treatment and after 5 days of antibiotic treatment in the effective group, and a comparison of the mean values of the two-parameter combination before antibiotic treatment and after 5 days of antibiotic treatment in the ineffective group. FIG. 31D shows the ROC curve of the detection of efficacy on sepsis using the two-parameter combination.

FIG. 32 shows calculation steps of an algorithm of an area parameter D_NEU_FLSS_Area of a neutrophil population according to some embodiments of the disclosure.

FIG. 33 shows ROC curves corresponding to infection marker parameters to some embodiments of the disclosure.

### DETAILED DESCRIPTION

The technical solutions of the embodiments of the disclosure will be described below clearly and comprehensively in conjunction with accompanying drawings of the embodiments of the disclosure. Apparently, the embodiments described are merely some of, rather than all of, the embodiments of the disclosure. Based on the embodiments of the disclosure, all the other embodiments which would have been obtained by those of ordinary skill in the art without any creative efforts shall fall within the scope of protection of the disclosure.

Throughout the specification, unless otherwise specified, the terms used herein should be understood as the meanings commonly used in the art. Therefore, unless otherwise defined, all the technical and scientific terms used herein have the same meaning as commonly understood by those of skill in the art to which the disclosure belongs. In the event of a contradiction, the description in this specification takes precedence.

It should be noted that, in the embodiments of the disclosure, the terms “include”, “including” or any other variation thereof are intended to cover non-exclusive inclusion, so that a method or device including a series of elements includes not only explicitly stated elements, but also other elements not explicitly listed, or elements inherent in implementing the method or device. In the absence of more restrictions, the element defined by the phrase “comprising a/an . . . ” does not exclude the presence of a further related element (for example, steps in the method or units in the apparatus, wherein the unit may be a partial circuit, a partial processor, a partial program, software, or the like) in the method or apparatus that comprises the element.

It should be noted that the term “first/second/third” in the embodiments of the disclosure is only used to distinguish similar objects, and does not represent specific order for the objects. It may be understood that “first/second/third” may be interchanged for specific order or chronological order when allowed. It should be understood that the objects distinguished by “first/second/third” may be interchangeable where appropriate, so that the embodiments of the disclosure described herein can be implemented in an order other than that illustrated or described herein.

It should be noted that the term “at least one” in the embodiment of the disclosure refers to one or more than one under reasonable conditions, for example, two, three, four, five or ten, and the like.

The term “scattergram” referred to in the embodiment of the disclosure is a two-dimensional or three-dimensional diagram generated by a blood cell analyzer, with two-dimensional or three-dimensional feature information about a plurality of particles distributed thereon, wherein an X coordinate axis, a Y coordinate axis and a Z coordinate axis of the scattergram each represent a characteristic of each particle. For example, in a exemplary scattergram, the X coordinate axis represents a forward-scattered light intensity, the Y coordinate axis represents a fluorescence intensity, and the Z coordinate axis represents a side-scattered light intensity. The term “scattergram” used in the disclosure refers not only to a distribution map of at least two sets of data in a rectangular coordinate system in the form of data points, but also to an array of data, that is, not limited by its graphical presentation form.

The term “particle population” or “cell population” referred to in the embodiment of the disclosure is a population of particles formed by a plurality of particles having the identical cell characteristics distributed in a certain region of the scattergram, such as a leukocyte (including all types of leukocytes) population, and a leukocyte subpopulation, such as a neutrophil population, a lymphocyte population, a monocyte population, an cosinophil population, or a basophil population.

The term “ROC curve (receiver operating characteristic curve)” referred to in the embodiment of the disclosure is a receiver operating characteristic curve, which is based on a series of different binary classifications (discrimination thresholds), is plotted with the true positive rate as the ordinate and the false positive rate as the abscissa, and ROC_AUC (area under the curve) represents the area enclosed by the ROC curve and the horizontal coordinate axis.

The principle of plotting the ROC curve is to set a number of different critical values for continuous variables, calculate the corresponding sensitivity and specificity at each critical value, and then plot the curve with sensitivity as the vertical coordinate and 1-specificity as the horizontal coordinate.

Because the ROC curve is composed of multiple critical values representing their respective sensitivity and specificity, the best diagnostic threshold value for a certain diagnostic method can be selected with the help of the ROC curve. The closer the ROC curve is to the upper left corner, the higher the test sensitivity and the lower the misjudgment rate, the better the performance of the diagnosis method. It can be seen that the point on the ROC curve closest to the upper left corner of the ROC curve has the largest sum of sensitivity and specificity, and the value corresponding to this point or its adjacent points is often used as a diagnostic reference value (also known as a diagnostic threshold or a determination threshold or a preset condition or a preset range).

Currently, a blood cell analyzer generally counts and classifies leukocytes through DIFF channels and/or WNB channels. The blood cell analyzer performs a four-part differential of leukocytes via the DIFF channel, and classifies leukocytes into four types of leukocytes: lymphocytes (Lym), monocytes (Mon), neutrophils (Neu), and cosinophils (Eos). The blood cell analyzer can identify the nucleated red blood cells through the WNB channel, and can obtain the nucleated red blood cell count, leukocyte count and basophil count at the same time.

The blood cell analyzer used in the disclosure implements classification and counting of particles in a blood sample through a flow cytometry technique combined with a laser scattering method and a fluorescence staining method. Here, the principle of testing a blood sample by the blood cell analyzer may be, for example: first, aspirating a blood sample, and treating the blood sample with a hemolytic agent and a fluorescent dye, in which red blood cells are destroyed and dissolved by the hemolytic agent, while leukocytes will not be dissolved, but the fluorescent dye can enter a leukocyte nucleus with the help of the hemolytic agent and then is bound with nucleic acid substances of the nucleus; and then, particles in the sample are made to pass through a detection aperture irradiated by a laser beam one by one. When the laser beam irradiates the particles, properties (such as volume, degree of staining, size and content of cell contents, or density of cell nucleus) of the particles themselves may block or change a direction of the laser beam, thereby generating scattered light at various angles that corresponds to the characteristics of the particles, and the scattered light can be received by a signal detector to obtain relevant information about a structure and composition of the particles. Forward scatter (FS) reflects a number and a volume of particles, side scatter (SS) reflects a complexity of a cell internal structure (such as intracellular particles or nucleus), and fluorescence (FL) reflects a content of nucleic acid substances in a cell. The use of the light information can implement differential and counting of the particles in the sample.

FIG. 1 is a schematic diagram of a structure of a blood cell analyzer according to some embodiments of the disclosure. The blood cell analyzer 100 includes a sample suction device 110, a sample preparation device 120, an optical detection device 130, and a processor 140. The blood cell analyzer 100 further has a liquid circuit system for connecting the sample suction device 110, the sample preparation device 120, and the optical detection device 130 for liquid transport between these devices.

The sample suction device 110 is configured to aspirate a blood sample to be tested of a subject.

In some embodiments, the sample suction device 110 has a sampling needle (not shown) for aspirating a blood sample to be tested. In addition, the sample suction device 110 may further include, for example, a driving device configured to drive the sampling needle to quantitatively aspirate a blood sample to be tested through a needle nozzle of the sampling needle. The sample suction device 110 can transport an aspirated blood sample to the sample preparation device 120.

The sample preparation device 120 is configured to prepare a test sample containing a blood sample to be tested, a hemolytic agent, and a staining agent for identifying nucleated red blood cells.

In the embodiment of the disclosure, the hemolytic agent herein is configured to lyse red blood cells in blood to break the red blood cells into fragments, with the morphology of leukocytes substantially unchanged.

In some embodiments, the hemolytic agent may be any one or a combination of a cationic surfactant, a non-ionic surfactant, an anionic surfactant, and an amphiphilic surfactant. In other embodiments, the hemolysis reagent may include at least one of alkyl glycosides, triterpenoid saponins and steroidal saponins. For example, the hemolytic agent may be selected from octyl quinoline bromide, octyl isoquinoline bromide, decyl quinoline bromide, decyl isoquinoline bromide, dodecyl quinoline bromide, dodecyl isoquinoline bromide, tetradecyl quinoline bromide, tetradecyl isoquinoline bromide, octyl trimethyl ammonium chloride, octyl trimethyl ammonium bromide, decyl trimethyl ammonium chloride, decyl trimethyl ammonium bromide, dodecyl trimethyl ammonium chloride, dodecyl trimethyl ammonium bromide, tetradecyl trimethyl ammonium chloride and tetradecyl trimethyl ammonium bromide; dodecyl alcohol polyethylene oxide (23) ether, hexadecyl alcohol polyethylene oxide (25) ether, hexadecyl alcohol polyethylene oxide (30) ether, etc.

In some embodiments, the stain may be a fluorescent dye capable of binding nucleic acid substances in nucleated red blood cells. For example, the following compounds may be used in embodiments of the disclosure.

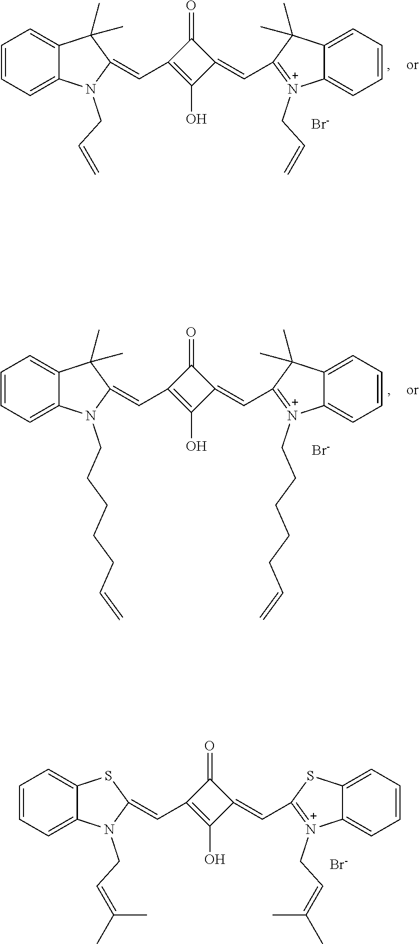 [SMILES: C=CCN1/C(=C/C2=C(O)C(=C/C3=[N+](CC=C)c4ccccc4C3(C)C)/C2=O)C(C)(C)c2ccccc21 ; [Br-] ; C=CCCCCCN1/C(=C/C2=C(O)C(=C/C3=[N+](CCCCCC=C)c4ccccc4C3(C)C)/C2=O)C(C)(C)c2ccccc21 ; [Br-] ; CC(C)=CCN1/C(=C/C2=C(O)C(=C/c3sc4ccccc4[n+]3CC=C(C)C)/C2=O)Sc2ccccc21 ; [Br-]]

In some embodiments, the sample preparation device 120 may comprise at least one reaction cell and a reagent supply device (not shown). The at least one reaction cell is configured to receive the blood sample to be tested aspirated by the sample suction device 110, and the reagent supply device supplies treatment reagents (including the hemolytic reagent, the staining agent, etc.) to the at least one reaction cell, so that the blood sample to be tested aspirated by the sample suction device 110 is mixed, in the reaction cell, with the treatment reagents supplied by the reagent supply device to prepare the test samples.

For example, the at least one reaction cell may include a first reaction cell and a second reaction cell, for instance reagent supply device may include a first reagent supply portion and a second reagent supply portion. The sample suction device 110 is configured to respectively dispense the aspirated blood sample to be tested in part to the first reaction cell and the second reaction cell. The first reagent supply portion is configured to supply the first hemolytic agent and the first staining agent for leukocyte classification to the first reaction cell, so that part of the blood sample to be tested that is dispensed to the first reaction cell is mixed and reacts with the first hemolytic agent and the first staining agent so as to prepare a first test sample. The second reagent supply portion is configured to supply the second hemolytic agent and the second staining agent for identifying nucleated red blood cells to the second reaction cell, so that the part of the test blood sample that is dispensed to the second reaction cell is mixed and reacts with the second hemolytic agent and the second staining agent so as to prepare a second test sample. Reagents currently commercially available for leukocyte four-part differential may be used in the first hemolytic agent and the first staining agent of the disclosure, such as M-60LD and M-6FD. Commercially available reagents for identifying nucleated red blood cells may be used in the second hemolytic agent and the second staining agent of the disclosure, such as M-6LN and M-6FN.

The optical detection device 130 comprises a flow cell, a light source and an optical detector, the flow cell is configured to allow for passage of the test sample, the light source is configured to irradiate the test sample passing through the flow cell with light, and the optical detector is configured to detect optical information generated by the irradiated test sample when passing through the flow cell.

For example, the first test sample and the second test sample pass through the flow cell, respectively, and a light source irradiates the first test sample and the second test sample passing through the flow cell, respectively. The optical detector is used for detecting first optical information and second optical information generated after the first test sample and the second test sample are irradiated by light when they pass through the flow cell, respectively.

It will be understood herein that the first detection channel for leukocyte classification (also referred to as DIFF channel) refers to the detection by the optical detection device 130 of the first test sample prepared by the sample preparation device 120, and the second detection channel for identifying nucleated red blood cells (also referred to as WNB channel) refers to the detection by the optical detection device 130 of the second test sample prepared by the sample preparation device 120.

Herein, the flow cell refers to a cell of focused flow that is suitable for detecting a light scattering signal and a fluorescence signal. When a particle, such as a blood cell, passes through the detection aperture of the flow cell, the particle scatters, to all directions, an incident light beam from the light source directed to the detection aperture. The optical detector may be provided at one or more different angles relative to the incident light beam, to detect light scattered by the particle to obtain a scattered light signal. Since different particles have different light scattering properties, the light scattering signal can be used to distinguish between different particle swarms. Specifically, a light scattering signal detected in the vicinity of the incident beam is often referred to as a forward light scattering signal or a small-angle light scattering signal. In some embodiments, the forward light scattering signal can be detected at an angle of about 1° to about 10° from the incident beam. In some other embodiments, the forward light scattering signal can be detected at an angle of about 2° to about 6° from the incident beam. A light scattering signal detected at about 90° from the incident beam is commonly referred to as a side light scattering signal. In some embodiments, the side light scattering signal can be detected at an angle of about 65° to about 115° from the incident beam. Typically, a fluorescence signal from a blood cell stained with a fluorescent dye is also generally detected at about 90° from the incident beam.

In some embodiments, the optical detector may include a forward scatter detector for detecting a forward scatter signal, a side scatter detector for detecting a side scatter signal, and a fluorescence detector for detecting a fluorescence signal. Accordingly, the optical information may include a forward scatter signal, a side scatter signal, and a fluorescence signal for measuring particles in the sample.

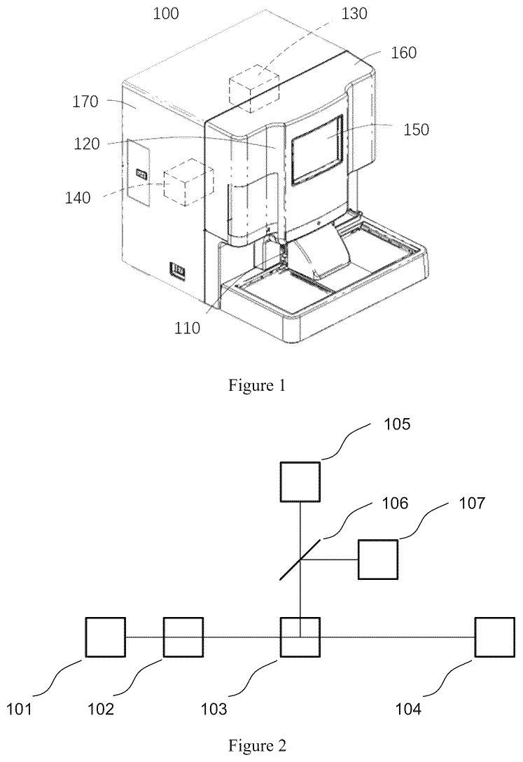

FIG. 2 shows a specific example of the optical detection device 130. The optical test device 130 is provided with a light source 101, a beam shaping assembly 102, a flow cell 103 and a forward scatter detector 104 which are sequentially arranged in a straight line. On one side of the flow cell 103, a dichroscope 106 is arranged at an angle of 45° to the straight line. Part of lateral light emitted by particles in the flow cell 103 is transmitted through the dichroscope 106 and is captured by the fluorescence detector 105 arranged behind the dichroscope 106 at an angle of 45° to the dichroscope 106; and the other part of the lateral light is reflected by the dichroscope 106 and is captured by the side scatter detector 107 arranged in front of the dichroscope 106 at an angle of 45° to the dichroscope 106.

The processor 140 is configured to process and operate data to obtain a required result. For example, a two-dimensional scattergram or a three-dimensional scattergram may be generated based on various collected light signals, and particle analysis can be performed using a method of gating on the scattergram. The processor 140 may also be configured to perform visualization processing on an intermediate operation result or a final operation result, and then display same by a display device 150. In the embodiments of the disclosure, the processor 140 is configured to implement the methods and steps which will be described in detail below.

In embodiments of the disclosure, the processor includes, but is not limited to, a central processing unit (CPU), a micro controller unit (MCU), a field-programmable gate array (FPGA), a digital signal processor (DSP) and other devices for interpreting computer instructions and processing data in computer software. For example, the processor is configured to execute each computer application program in a computer-readable storage medium, so that the blood cell analyzer 100 preforms a corresponding detection process and analyzes, in real time, optical information or optical signals detected by the optical detection device 130.

In addition, the blood cell analyzer 100 may further include a first housing 160 and a second housing 170. The display device 150 may be, for example, a user interface. The optical detection device 130 and the processor 140 are provided inside the second housing 170. The sample preparation device 120 is provided, for example, inside the first housing 160, and the display device 150 is provided, for example, on an outer surface of the first housing 160 and configured to display test results from the blood cell analyzer.

As mentioned in the BACKGROUND, the blood routine test realized by using the blood cell analyzer can indicate the occurrence of infection and the identification of infection types, but the blood routine WBC/Neu % currently used in clinical practice is affected by many aspects and cannot accurately and timely reflect patient condition. Moreover, the sensitivity and specificity of the existing technology in the diagnosis and treatment of bacterial infections and sepsis are poor.

Based on this context, through in-depth study of original signal characteristics of a large number of infected patients' blood samples in the blood routine test, the inventors unexpectedly found that the infection status of the subject can be evaluated with high efficiency using the leukocyte characteristic parameters of WNB channels by such as linear discriminant analysis (LDA). The linear discriminant analysis is an induction of Fisher's linear discriminant method, which uses statistics, pattern recognition, and machine learning methods to characterize or distinguish two types of events (e.g., with or without sepsis, bacterial or viral infection, infectious or non-infectious inflammation, effective or ineffective treatment for sepsis) by finding a linear combination of characteristics of the two types of events and by obtaining one-dimensional data via linearly combining a multi-dimensional data. The coefficient of the linear combination may ensure that the degree of discrimination of the two types of events is maximized. The resulting linear combination can be used to classify subsequent events.

Herein, the embodiment of the disclosure provides a solution that utilizes the leukocyte characteristic parameters of the WNB channel to obtain infection marker parameters for effective infection status evaluation. The solution provided by the embodiment of the disclosure has the advantage that the infection status can be quickly evaluated to realize early prediction of sepsis, differential diagnosis of sepsis, monitoring of infection, prognosis of sepsis, identification of bacterial infection and viral infection, and the like.

In one embodiment, the identification of bacterial infections and viral infections is performed by the method of the disclosure using the blood cell analyzer of the disclosure. Without wishing to be bound by theory, the inventors found that the main active cells involved in bacterial infections are neutrophils and monocytes. These two kinds of cells will undergo morphological changes during bacterial infection, such as increased volume, increased particles, increased number of naive granulocytes, toxic particles, vacuoles, Duller bodies, etc., and dense nuclei. These characteristics can be reflected in the blood cell analyzer of the disclosure by detecting the signal intensity of neutrophil or monocyte particle populations in the direction of SS, FL, and FS. The main active cells in viral infection are lymphocytes. After virus infection, the number of lymphocytes increased significantly, and atypical lymphocytes appeared, which could be reflected in the FL direction of the scattergram.

Therefore, an embodiment of the disclosure first provide a blood cell analyzer, comprising:
a sample suction device 110 configured to aspirate a blood sample to be tested of a subject;
a sample preparation device 120 configured to prepare a test sample containing a part of the blood sample to be tested, a hemolytic agent, and a staining agent for identifying nucleated red blood cells;
an optical detection device 130 comprising a flow cell, a light source and an optical detector, the flow cell being configured to allow for passage of the test sample, the light source being configured to irradiate the test sample passing through the flow cell with light, and the optical detector being configured to detect optical information generated by the irradiated test sample when passing through the flow cell;
a processor 140 configured to:
obtain at least one leukocyte characteristic parameter of at least one target particle population in the test sample from the optical information;
obtain an infection marker parameter for evaluating an infection status of the subject based on the at least one leukocyte characteristic parameter; and
output the infection marker parameter.

It should be understood that the cell characteristic parameters of the target particle population do not include the cell count or classification parameters of the target particle population, but include characteristic parameters reflecting cell characteristics such as the volume, internal granularity, internal nucleic acid content of the cells in the target particle population.

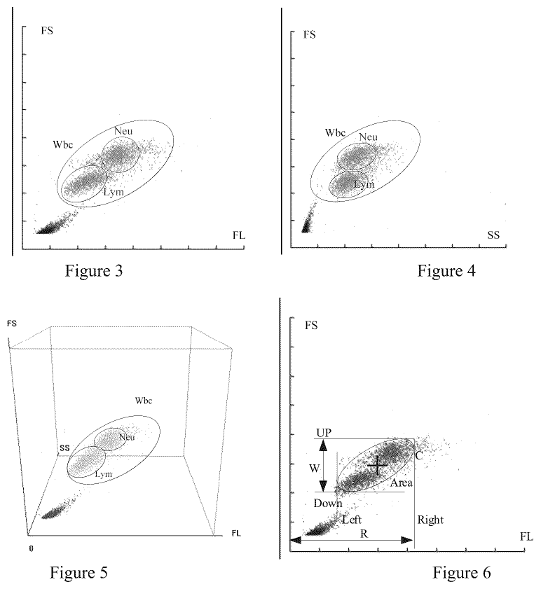

In some embodiments, the leukocyte population Wbc (including all types of leukocytes) in the test sample can be identified based on the forward scatter signal (or forward scatter intensity) FS, the side scatter signal (or side scatter intensity) SS, and the fluorescence signal (or fluorescence intensity) FL in the optical information, while the neutrophil population Neu and the lymphocyte population Lym in the leukocytes in the test sample can be identified, as shown in FIGS. 3 to 5. FIG. 3 is a two-dimensional scattergram generated based on the forward scatter signal FS and the fluorescent signal FL in the optical information, FIG. 4 is a two-dimensional scattergram generated based on the forward scatter signal FS and the side scatter signal SS in the optical information, and FIG. 5 is a three-dimensional scattergram generated based on the forward scatter signal FS, the side scatter signal SS and the fluorescent signal FL in the optical information. Further, the processor 140 is further configured to identify nucleated red blood cells in the test sample based on the optical information to obtain a nucleated red blood cell count.

Accordingly, in some embodiments, the at least one target particle population may comprise at least one cell population among a leukocyte population Wbc, a neutrophil population Neu, and a lymphocyte population Lym in the test sample. For example, the at least one target particle population comprises a lymphocyte population Lym and a leukocyte population Wbc in the test sample, or comprises a neutrophil population Neu and a leukocyte population Wbc in the test sample, or comprises a lymphocyte population Lym and a neutrophil population Neu in the test sample. That is, the at least one leukocyte characteristic parameter may include one or more of the cell characteristic parameters of a lymphocyte population Lym, a neutrophil population Neu, and a leukocyte population Wbc in the sample.

In some embodiments, the at least one target particle population comprises a leukocyte population Wbc and/or a neutrophil population Neu. In the course of studying the original signal of a large number of subject samples in blood routine test, the inventors found that the use of cell characteristic parameters of the leukocyte population Wbc and/or neutrophil population Neu in the test sample is advantageous for the efficient evaluation of infection status. More in some embodiments, the combination of the cellular characteristic parameters of the neutrophil population Neu and the leukocyte population Wbc can give more diagnostically potent infection marker parameters.

In some embodiments, the at least one leukocyte characteristic parameter may comprise one or more parameters of the following cell characteristic parameters: a forward scatter intensity distribution width, a forward scatter intensity distribution center of gravity, a forward scatter intensity distribution coefficient of variation, a side scatter intensity distribution width, a side scatter intensity distribution center of gravity, a side scatter intensity distribution coefficient of variation, a fluorescence intensity distribution width, a fluorescence intensity distribution center of gravity, a fluorescence intensity distribution coefficient of variation of the at least one target particle population (for example, neutrophil population neu and/or leukocyte population Wbc), and an area of a distribution region of the at least one target particle population in a two-dimensional scattergram generated by two light intensities of a forward scatter intensity, a side scatter intensity, and a fluorescence intensity, and a volume of a distribution region of the at least one target particle population in a three-dimensional scattergram generated by a forward scatter intensity, a side scatter intensity, and a fluorescence intensity; for example, the volume of the space occupied by leukocyte population in FIG. 5.

In some specific examples, the at least one leukocyte characteristic parameter may comprise one or more parameters of the following cell characteristic parameters:
a forward scatter intensity distribution center of gravity of the leukocyte population (N_WBC_FS_P), a side scatter intensity distribution center of gravity of the leukocyte population (N_WBC_SS_P), a side fluorescence intensity distribution center of gravity of the leukocyte population (N_WBC_FL_P), a forward scatter intensity distribution width of the leukocyte population (N_WBC_FS_W), a side scatter intensity distribution width of the leukocyte population (N_WBC_SS_W), a side fluorescence intensity distribution width of the leukocyte population (N_WBC_FL_W), a forward scatter intensity distribution coefficient of variation of the leukocyte population (N_WBC_FS_CV), a side scatter intensity distribution coefficient of variation of the leukocyte population (N_WBC_SS_CV), and a side fluorescence intensity distribution coefficient of variation of the leukocyte population (N_WBC_FL_CV); an area of a distribution region of the leukocyte population in a two-dimensional scattergram generated by two light intensities of a forward scatter intensity, a side scatter intensity, and a side fluorescence intensity, a volume of a distribution region of the leukocyte population in a three-dimensional scattergram generated by a forward scatter intensity, a side scatter intensity, and a fluorescence intensity, for example: an area of a distribution region of the leukocyte population in a two-dimensional scattergram generated by a side scatter intensity and a forward scatter intensity (N_WBC_SSFS_Area), an area of a distribution region of the leukocyte population in a two-dimensional scattergram generated by a side fluorescence intensity and a forward scatter intensity (N_WBC_FLFS_Area), and an area of a distribution region of the leukocyte population in a two-dimensional scattergram generated by a side fluorescence intensity and a side scatter intensity (N_WBC_FLSS_Area);
a forward scatter intensity distribution center of gravity of the neutrophil population (N_NEU_FS_P), a side scatter intensity distribution center of gravity of the neutrophil population (N_NEU_SS_P), a side fluorescence intensity distribution center of gravity of the neutrophil population (N_NEU_FL_P), a forward scatter intensity distribution width of the neutrophil population (N_NEU_FS_W), a side scatter intensity distribution width of the neutrophil population (N_NEU_SS_W), a side fluorescence intensity distribution width of the neutrophil population (N_NEU_FL_W), a forward scatter intensity distribution coefficient of variation of the neutrophil population (N_NEU_FS_CV), a side scatter intensity distribution coefficient of variation of the neutrophil population (N_NEU_SS_CV), and a side fluorescence intensity distribution coefficient of variation of the neutrophil population (N_NEU_FL_CV);
an area of a distribution region of the neutrophil population in a two-dimensional scattergram generated by two light intensities of a forward scatter intensity, a side scatter intensity, and a side fluorescence intensity, a volume of a distribution region of the neutrophil population in a three-dimensional scattergram generated by a forward scatter intensity, a side scatter intensity, and a fluorescence intensity, for example: an area of a distribution region of the neutrophil population in a two-dimensional scattergram generated by a side scatter intensity and a forward scatter intensity (N_NEU_SSFS_Area), an area of a distribution region of the neutrophil population in a two-dimensional scattergram generated by a side fluorescence intensity and a forward scatter intensity (N_NEU_FLFS_Area), and an area of a distribution region of the neutrophil population in a two-dimensional scattergram generated by a side fluorescence intensity and a side scatter intensity (N_NEU_FLSS_Area);
a forward scatter intensity distribution center of gravity of the lymphocyte population (N_LYM_FS_P), a side scatter intensity distribution center of gravity of the lymphocyte population (N_LYM_SS_P), a side fluorescence intensity distribution center of gravity of the lymphocyte population (N_LYM_FL_P), a forward scatter intensity distribution width of the lymphocyte population (N_LYM_FS_W), a side scatter intensity distribution width of the lymphocyte population (N_LYM_SS_W), a side fluorescence intensity distribution width of the lymphocyte population (N_LYM_FL_W), a forward scatter intensity distribution coefficient of variation of the lymphocyte population (N_LYM_FS_CV), a side scatter intensity distribution coefficient of variation of the lymphocyte population (N_LYM_SS_CV), and a side fluorescence intensity distribution coefficient of variation of the lymphocyte population (N_LYM_FL_CV); and
an area of a distribution region of the lymphocyte population in a two-dimensional scattergram generated by two light intensities of a forward scatter intensity, a side scatter intensity, and a side fluorescence intensity, a volume of a distribution region of the lymphocyte population in a three-dimensional scattergram generated by a forward scatter intensity, a side scatter intensity, and a fluorescence intensity, for example: an area of a distribution region of the lymphocyte population in a two-dimensional scattergram generated by a side scatter intensity and a forward scatter intensity (N_LYM_SSFS_Area), an area of a distribution region of the lymphocyte population in a two-dimensional scattergram generated by a side fluorescence intensity and a forward scatter intensity (N_LYM_FLFS_Area), and an area of a distribution region of the lymphocyte population in a two-dimensional scattergram generated by a side fluorescence intensity and a side scatter intensity (N_LYM_FLSS_Area).

Those skilled in the art can understand that it is possible to use the overall distribution characteristics of the scattergram of a certain particle swarm, such as the forward scatter intensity distribution width of the entire leukocyte population, or to use the characteristics of the distribution of particles in some areas of a certain particle swarm, such as the distribution region of a portion with a higher density in the middle of a neutrophil population, or an area that is different from the neutrophil or lymphocyte particle swarm of a normal human scattergram.

In some embodiments, the infection marker parameter may be constituted by a single leukocyte characteristic parameter, for example by one of the cell characteristic parameters enumerated above. Alternatively, the infection marker parameter may be a linear function or a nonlinear function of a single leukocyte parameter.

Alternatively, in other embodiments, the infection marker parameter may also be calculated from the combination of the at least one leukocyte characteristic parameter and another leukocyte parameter obtained from the optical information that is different from the leukocyte characteristic parameter, for example, obtained from a combination of a plurality of cell characteristic parameters among the cell characteristic parameters enumerated above, in particular from a combination by a linear function.

For example, in some examples, the processor 140 may be further configured to:
obtain at least one leukocyte characteristic parameter (also referred to as a first leukocyte parameter) of a first leukocyte particle population in the test sample and at least one second leukocyte parameter of a second leukocyte particle population in the test sample from the optical information; and
calculate the infection marker parameter based on the at least one leukocyte characteristic parameter and the at least one second leukocyte parameter.

Herein, the first leukocyte particle population and the second leukocyte particle population are different from each other, for example, the first leukocyte particle population is a leukocyte population and the second leukocyte particle population is a neutrophil population, or conversely, the first leukocyte particle population is a neutrophil population and the second leukocyte particle population is a leukocyte population.

In some embodiments, the at least one second leukocyte parameter comprises a cell characteristic parameter, i.e., the at least one second leukocyte parameter comprises a cell characteristic parameter of a second leukocyte particle population. Thus, an infection marker parameter with further improved diagnostic efficacy can be provided.

Certainly, it is also possible that the second leukocyte parameter includes a classification parameter or a count parameter (e.g., a leukocyte count or a neutrophil count) of the second leukocyte particle population.

In the above embodiments, the processor 140 may be further configured to combine the first leukocyte characteristic parameter and the second leukocyte parameter into an infection marker parameter by a linear function, i.e., to calculate the infection marker parameter by the following formula:

$$Y = A \times X 1 + B \times X 2 + C$$

where Y represents an infection marker parameter, X1 represents a first leukocyte parameter, X2 represents a second leukocyte parameter, and A, B, and C are constants.

Certainly, in other embodiments, the first leukocyte parameter and the second leukocyte parameter may also be combined into an infection marker parameter by a nonlinear function, which is not specifically limited in the disclosure. Those skilled in the art will appreciate that in other embodiments, the first leukocyte parameter and the second leukocyte parameter may be used in combination instead of calculating the two leukocyte parameters by a function, and compared with their respective thresholds to obtain infection marker parameters. For example, diagnostic thresholds are set for the two parameters: threshold 1 and threshold 2, and then the diagnostic efficacy of “parameter 1≥threshold 1 or parameter 2≥threshold 2” is analyzed, and the diagnostic efficacy of “parameter 1≥threshold 1 and parameter 2≥threshold 2” is analyzed.

In some embodiments, cell characteristic parameters of particle populations of WNB channels and DIFF channels may also be used in combination.

In other embodiments, the infection marker parameter may be calculated from the leukocyte parameter and other blood cell parameters, i.e., the infection marker parameter may be calculated from at least one leukocyte parameter and at least one other blood cell parameter. The other blood cell parameters may be classification or counting parameters for platelets (PLTs), nucleated red blood cells (NRBCs), or reticulocytes (RETs).

In other embodiments, the processor 140 may also be further configured to:
obtain at least two leukocyte characteristic parameters of one leukocyte particle population in the test sample from the optical information; and
calculate the infection marker parameter based on the at least two leukocyte characteristic parameters, in particular, by a linear function.

The meanings of the distribution width, the distribution center of gravity, the coefficient of variation, and the area or volume of the distribution region are explained herein with reference to FIG. 6, wherein FIG. 6 shows cell characteristic parameters of the leukocyte population in a test sample according to some embodiments of the disclosure.

As shown in FIG. 6, W (N_WBC_FS_W) represents the forward scatter intensity distribution width of the leukocyte population in the test sample, where N_WBC_FS_W is equal to the difference between the forward scatter intensity distribution upper limit (UP) of the leukocyte population and the forward scatter intensity distribution lower limit (DOWN) of the leukocyte population. N_WBC_FS_P represents the forward scatter intensity distribution center of gravity of the leukocyte population in the test sample, that is, the average position of the leukocytes in the FS direction (at “+” in FIG. 6), where N_WBC_FS_P is calculated by the following formula:

$$N\_WBC \_FS \_P = \frac{{\sum }_{1}^{N} F S ( i )}{N}$$

where FS (i) is the forward scatter intensity of the i-th leukocyte.

N_WBC_FS_CV represents the forward scatter intensity distribution coefficient of variation of the leukocyte population in the test sample, where N_WBC_FS_CV is equal to N_WBC_FS_W divided by N_WBC_FS_P.

In addition, the Area (N_WBC_FLFS_Area) in FIG. 6 represents the area of the distribution region of the leukocyte population in the test sample in the scattergram generated by the forward scatter intensity and the fluorescence intensity.

In some embodiments, as shown in FIG. 6, C represents a contour distribution curve of the leukocyte population, for example, the total number of positions within the contour distribution curve C may be recorded as the area of the leukocyte population. Those skilled in the art can understand that it is easy to obtain the contour distribution curve of the particle swarm by using the classification algorithm of a usual blood analyzer or image processing technology.

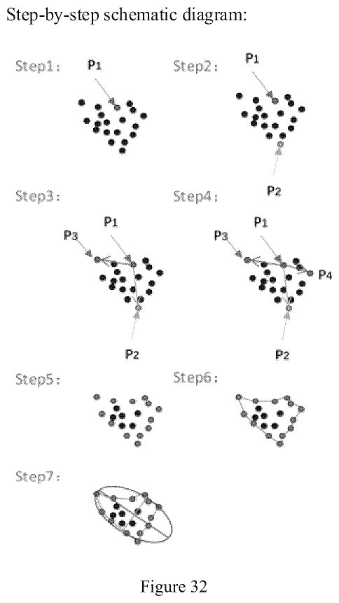

In other embodiments, D_NEU_FLSS_Area may also be implemented by the following algorithmic steps (FIG. 32):
randomly selecting a particle P1 from the neutrophil (NEU) particle population, and finding a particle P2 that is farthest from P1;
constructing a vector V1 (P1-P2), and taking P1 as the starting point of the vector, finding another particle P3 in the neutrophil (NEU) particle population, and constructing a vector V2 (P1-P3) such that the vector V2 (P1-P3) has a maximum angle with the vector V1 (P1-P2);
then, taking P1 as the starting point of the vector, finding another particle P4 in the neutrophil (NEU) particle population, and constructing a vector V3 (P1-P4) such that the vector V3 (P1-P4) has a maximum angle with the vector V1 (P1-P2);
by analogy, obtaining a group of particles P1, P2, P3, P4, . . . . Pn on the outermost side of the neutrophil (NEU) particle population, respectively;
fitting the particle points P1, P2, P3, P4, . . . . Pn by using an ellipse, and obtaining the major axis a and minor axis b of this ellipse;
the D_NEU_FLSS_Area is a product of the major axis a and the minor axis b.

Similarly, the volume parameters of the distribution region of the neutrophil population in the three-dimensional scattergram generated by the forward scatter intensity, the side scatter intensity, and the fluorescence intensity can also be obtained by corresponding calculations.

As will be appreciated herein, definitions of other cell characteristic parameters may be referred in a corresponding manner to the embodiments shown in FIGS. 6 and 32.

In some embodiments, the processor 140 may be further configured to: output prompt information indicating that the infection marker parameter is abnormal when a value of the infection marker parameter is beyond a preset range. For example, when the value of the infection marker parameter is abnormally elevated, an upward pointing arrow may be output to indicate the abnormal elevation.

Alternatively, processor 140 may be further configured to output the preset range.

In some embodiments, the processor 140 may be further configured to: output prompt information indicating the infection status of the subject based on the infection marker parameter. For example, the processor 140 may be configured to output the prompt information to the display device for display. The display device herein may be the display device 150 of the blood cell analyzer 100, or other display devices in communication with the processor 140. For example, the processor 140 may output the prompt information to the display device on the user (doctor) side through the hospital information management system.

Some application scenarios of the infection marker parameters provided in the disclosure are described next, but the disclosure is not limited thereto.

In some embodiments, the infection marker parameter may be used for performing on the subject an early prediction of sepsis, diagnosis of sepsis, an identification of a common infection and a severe infection, monitoring of infection, an analysis of sepsis prognosis, an identification of a bacterial infection and a viral infection, an identification of a non-infectious inflammation and an infectious inflammation, or evaluation of therapeutic effect on sepsis. For example, the processor 140 may be further configured to perform on the subject an early prediction of sepsis, a diagnosis of sepsis, an identification of a common infection and a severe infection, a monitoring of infection, an analysis of sepsis prognosis, an identification of a bacterial infection and a viral infection, an identification of a non-infectious inflammation and an infectious inflammation, or an evaluation of therapeutic effect on sepsis based on the infection marker parameter.

Sepsis is a serious infectious disease with a high incidence and case fatality rate. Every hour of delay in treatment, the mortality rate of patients increases by 7%. Therefore, the early warning of sepsis is particularly important. The early identification and early warning of sepsis can increase the precious diagnosis and treatment time for patients and greatly improve the survival rate.

To this end, in an application scenario of early prediction of sepsis, i.e., the infection marker parameter is used for early prediction of sepsis, the processor 140 may be configured to output prompt information indicating that the subject is likely to progress to sepsis within a certain period of time after the blood sample to be tested is collected, when the infection marker parameter satisfies a first preset condition.

In some embodiments, the certain period of time is not greater than 48 hours, i.e., the embodiment of the disclosure can predict up to two days in advance whether the subject is likely to progress to sepsis. Further, the certain period of time is within 24 hours, that is, the embodiment of the disclosure may predict one day in advance whether the subject is likely to progress to sepsis.

Herein, the first preset condition may be, for example, that the value of the infection marker parameter is greater than a preset threshold. The preset threshold can be determined based on a specific combination of parameters and a blood cell analyzer.

In some embodiments, the infection marker parameter for early prediction of sepsis may be one of the following parameters: N_WBC_FL_W; N_WBC_FS_W; N_WBC_SS_W.

In other embodiments, infection marker parameters are calculated by combining two or more leukocyte characteristic parameters of the disclosure. At the cell type level, for example, both neutrophils and monocytes are the first barrier of the body against infection, and both are valuable in reflecting the degree of infection. Therefore, the combination of neutrophils' characteristic parameters and monocytes' characteristic parameters can improve the predictive, diagnostic, evaluation and/or guiding therapeutic efficacy of the disclosure.

Those skilled in the art can understand that in an embodiment of the disclosure, a leukocyte characteristic parameter is obtained by using a scattergram formed by original optical information and the calculated characteristics of the leukocyte related particle swarm, and an infection marker parameter for evaluating the infection status of the subject is obtained based on the leukocyte characteristic parameter. When the infection marker parameter is obtained based on a single leukocyte characteristic parameter, the single leukocyte characteristic parameter can be regarded as the infection marker parameter directly, or the infection marker parameter can be obtained by calculating the single leukocyte characteristic parameter by a linear or nonlinear function; when the infection marker parameter is obtained based on a plurality of leukocyte characteristic parameters, the plurality of leukocyte characteristic parameters can be used in combination or calculated in combination to obtain the infection marker parameter. In some embodiments, the infection marker parameter is compared with the diagnostic threshold, giving relevant clinical implications.

In some embodiments, infection marker parameters may be calculated by combining the various parameters listed in Table 1 for early prediction of sepsis.

TABLE 1
Parameter combinations for early prediction of sepsis

| No. | Parameter combination |
|---|---|
| 1 | N_WBC_FL_P; N_WBC_FS_W; |
| 2 | N_WBC_SS_W; N_WBC_FL_P; |
| 3 | N_WBC_FS_W; N_NEU_FL_CV; |
| 4 | N_WBC_FS_W; N_NEU_FL_P; |
| 5 | N_WBC_SS_W; N_NEU_FL_P; |
| 6 | N_WBC_SS_W; N_WBC_FL_W; |
| 7 | N_WBC_FL_W; N_WBC_FS_W; |
| 8 | N_WBC_SS_W; N_NEU_FL_CV; |
| 9 | N_NEU_FL_P; N_NEU_FS_W; |
| 10 | N_NEU_FL_P; N_NEU_FS_CV; |
| 11 | N_WBC_FL_P; N_NEU_SS_W; |
| 12 | N_WBC_FL_P; N_NEU_FS_W; |
| 13 | N_WBC_FL_P; N_NEU_FS_CV; |
| 14 | N_NEU_SS_W; N_NEU_FL_P; |
| 15 | N_WBC_SS_W; N_NEU_SS_W; |
| 16 | N_WBC_FL_W; N_NEU_FS_W; |
| 17 | N_WBC_FL_W; N_NEU_SS_W; |
| 18 | N_WBC_SS_W; N_WBC_FS_W; |
| 19 | N_WBC_FL_W; N_NEU_FS_CV; |
| 20 | N_NEU_FL_CV; N_NEU_FS_W; |
| 21 | N_WBC_FL_P; N_WBC_SSFS_Area; |
| 22 | N_WBC_FL_P; N_NEU_SSFS_Area; |
| 23 | N_WBC_FS_W; N_NEU_SS_P; |
| 24 | N_WBC_SS_P; N_WBC_FS_W; |
| 25 | N_WBC_FL_P; N_NEU_SS_CV; |
| 26 | N_WBC_FL_W; N_NEU_SS_CV; |
| 27 | N_WBC_FL_W; N_WBC_FS_P; |
| 28 | N_NEU_FL_P; N_NEU_SSFS_Area; |
| 29 | N_WBC_FL_W; N_NEU_SSFS_Area; |
| 30 | N_WBC_SS_W; N_WBC_FS_P; |
| 31 | N_NEU_SS_W; N_NEU_FL_CV; |
| 32 | N_WBC_FS_W; N_NEU_SS_W; |
| 33 | N_WBC_FS_W; N_NEU_FS_W; |
| 34 | N_WBC_SS_W; N_NEU_FS_P; |
| 35 | N_NEU_SS_CV; N_NEU_FL_P; |
| 36 | N_WBC_FS_W; N_NEU_FS_CV; |
| 37 | N_WBC_SS_P; N_WBC_FL_W; |
| 38 | N_WBC_FL_W; N_NEU_FS_P; |
| 39 | N_WBC_FL_W; N_NEU_SS_P; |
| 40 | N_WBC_FS_W; N_NEU_FL_W; |
| 41 | N_WBC_FS_W; N_WBC_FLFS_Area; |
| 42 | N_WBC_FS_W; N_WBC_FLSS_Area; |
| 43 | N_WBC_FL_W; N_NEU_FLFS_Area; |
| 44 | N_WBC_FL_W; N_NEU_FLSS_Area; |
| 45 | N_WBC_FS_W; N_NEU_SSFS_Area; |
| 46 | N_WBC_FL_P; N_WBC_FLFS_Area; |
| 47 | N_WBC_FL_P; N_NEU_FLSS_Area; |
| 48 | N_NEU_FL_P; N_NEU_FLSS_Area; |
| 49 | N_WBC_SS_W; N_WBC_SSFS_Area; |
| 50 | N_WBC_FL_W; N_NEU_FL_P; |
| 51 | N_WBC_FL_W; N_WBC_SSFS_Area; |
| 52 | N_WBC_FS_W; N_NEU_FLSS_Area; |
| 53 | N_WBC_FL_W; N_NEU_FL_CV; |
| 54 | N_WBC_FLFS_Area; N_NEU_FL_P; |
| 55 | N_WBC_SSFS_Area; N_NEU_FL_P; |
| 56 | N_WBC_SS_W; N_NEU_SS_CV; |
| 57 | N_WBC_FS_W; N_WBC_SSFS_Area; |
| 58 | N_WBC_FL_P; N_WBC_FL_W; |
| 59 | N_WBC_FS_P; N_WBC_FS_W; |
| 60 | N_WBC_FL_W; N_WBC_FLFS_Area; |
| 61 | N_WBC_FL_P; N_WBC_FLSS_Area; |
| 62 | N_WBC_FS_W; N_NEU_FLFS_Area; |
| 63 | N_WBC_FS_W; N_NEU_FS_P; |
| 64 | N_WBC_FL_W; N_NEU_FL_W; |
| 65 | N_WBC_FS_W; N_NEU_SS_CV; |
| 66 | N_NEU_FL_CV; N_NEU_FS_CV; |
| 67 | N_NEU_FL_P; N_NEU_FL_W; |
| 68 | N_NEU_FL_P; N_NEU_FLFS_Area; |
| 69 | N_WBC_FL_P; N_NEU_FL_W; |
| 70 | N_WBC_FL_W; N_WBC_FLSS_Area; |
| 71 | N_NEU_FL_CV; N_NEU_FLSS_Area; |
| 72 | N_WBC_SS_W; N_NEU_SSFS_Area; |
| 73 | N_NEU_FL_W; N_NEU_FL_CV; |
| 74 | N_WBC_FL_P; N_NEU_FLFS_Area; |
| 75 | N_WBC_FLSS_Area; N_NEU_FL_P; |
| 76 | N_WBC_SS_P; N_WBC_SS_W; |
| 77 | N_WBC_SS_W; N_NEU_SS_P; |
| 78 | N_NEU_FL_P; N_NEU_FL_CV; |
| 79 | N_NEU_FL_CV; N_NEU_FLFS_Area; |
| 80 | N_WBC_SS_W; N_NEU_FL_W; |
| 81 | N_WBC_SS_W; N_WBC_FLFS_Area; |

In some embodiments, a combination of N_WBC_FL_P and N_WBC_FS_W, N_WBC_SS_W and N_WBC_FS_W, or N_WBC_FL_and N_NEU_FLSS_Area may be used to calculate infection marker parameters for early prediction of sepsis.

The clinical symptoms in the early stage of sepsis are similar to those of common/severe infections, and patients with sepsis are easily misdiagnosed as common/severe infectious diseases, delaying the timing of treatment. Therefore, the differential diagnosis of sepsis is particularly important.

To this end, in an application scenario of diagnosis of sepsis, i.e., the infection marker parameter is used for sepsis identification, the processor 140 may be configured to output prompt information indicating that the subject has sepsis when the infection marker parameter satisfies a second preset condition. Herein, the second preset condition may likewise be that the value of the infection marker parameter is greater than the preset threshold. The preset threshold can be determined based on a specific combination of parameters and a blood cell analyzer.

In some embodiments, the infection marker parameter for diagnosis of sepsis may be one of the following parameters: N_WBC_FL_W, N_WBC_FL_P, N_NEU_FL_P, N_NEU_FL_W, N_WBC_SS_W, N_NEU_FLFS_Area, N_WBC_FS_W, N_NEU_FS_W, N_NEU_FLSS_Area, N_NEU_SS_W, N_WBC_SS_P, N_NEU_SS_P, N_WBC_FLSS_Area, N_NEU_FS_CV, N_WBC_FLFS_Area, N_WBC_FS_P, N_NEU_SSFS_Area.

In other embodiments, infection marker parameters may be calculated by combining the various parameters listed in Table 2 for diagnosis of sepsis.

TABLE 2
Parameter combinations for diagnosis of sepsis

| No. | Parameter combination |
|---|---|
| 1 | N_WBC_FL_P; N_WBC_FS_W; |
| 2 | N_WBC_FL_W; N_WBC_FS_P; |
| 3 | N_WBC_FL_P; N_WBC_FS_CV; |
| 4 | N_WBC_FL_P; N_NEU_FS_CV; |
| 5 | N_WBC_FL_W; N_NEU_FS_CV; |
| 6 | N_WBC_FL_P; N_NEU_FS_W; |
| 7 | N_WBC_SS_CV; N_WBC_FL_W; |
| 8 | N_WBC_SS_CV; N_WBC_FL_P; |
| 9 | N_WBC_SS_W; N_WBC_FL_P; |
| 10 | N_WBC_SS_W; N_WBC_FL_W; |
| 11 | N_WBC_FL_W; N_NEU_SS_P; |
| 12 | N_WBC_SS_P; N_WBC_FL_W; |
| 13 | N_NEU_FL_P; N_NEU_FS_W; |
| 14 | N_WBC_FL_W; N_NEU_FS_W; |
| 15 | N_NEU_FL_P; N_NEU_FS_CV; |
| 16 | N_WBC_FL_P; N_WBC_FL_CV; |
| 17 | N_WBC_FL_W; N_NEU_SS_CV; |
| 18 | N_WBC_FL_W; N_NEU_SS_W; |
| 19 | N_WBC_FL_W; N_WBC_FLFS_Area; |
| 20 | N_WBC_FS_W; N_NEU_FL_P; |
| 21 | N_WBC_FL_P; N_WBC_FL_W; |
| 22 | N_WBC_FL_W; N_WBC_FL_CV; |
| 23 | N_WBC_FL_W; N_NEU_FS_P; |
| 24 | N_WBC_FL_W; N_NEU_FL_CV; |
| 25 | N_WBC_FL_W; N_WBC_FS_W; |
| 26 | N_WBC_FL_W; N_WBC_FLSS_Area; |
| 27 | N_WBC_FL_W; N_NEU_FL_P; |
| 28 | N_WBC_FL_W; N_WBC_FS_CV; |
| 29 | N_WBC_SS_W; N_NEU_FL_P; |
| 30 | N_WBC_FL_W; N_WBC_SSFS_Area; |
| 31 | N_WBC_FL_W; N_NEU_SSFS_Area; |
| 32 | N_WBC_FL_W; N_NEU_FL_W; |
| 33 | N_WBC_FL_W; N_NEU_FLFS_Area; |
| 34 | N_WBC_FL_W; N_NEU_FLSS_Area; |
| 35 | N_WBC_SS_CV; N_NEU_FL_P; |
| 36 | N_WBC_FL_P; N_NEU_SS_W; |
| 37 | N_WBC_FS_CV; N_NEU_FL_P; |
| 38 | N_NEU_FL_CV; N_NEU_FS_W; |
| 39 | N_NEU_SS_W; N_NEU_FL_P; |
| 40 | N_WBC_FL_P; N_NEU_SSFS_Area; |
| 41 | N_NEU_FL_CV; N_NEU_FS_CV; |
| 42 | N_WBC_FL_P; N_NEU_FL_W; |
| 43 | N_NEU_FL_P; N_NEU_SSFS_Area; |
| 44 | N_WBC_FL_P; N_NEU_SS_CV; |
| 45 | N_WBC_FL_P; N_NEU_FLSS_Area; |
| 46 | N_WBC_FL_P; N_NEU_FL_CV; |
| 47 | N_NEU_FL_P; N_NEU_FL_W; |
| 48 | N_WBC_FL_P; N_NEU_FLFS_Area; |
| 49 | N_NEU_FL_P; N_NEU_FL_CV; |
| 50 | N_WBC_FL_P; N_WBC_SSFS_Area; |
| 51 | N_NEU_FL_P; N_NEU_FLSS_Area; |
| 52 | N_NEU_FL_P; N_NEU_FLFS_Area; |
| 53 | N_WBC_FL_P; N_NEU_SS_P; |
| 54 | N_NEU_FL_W; N_NEU_FL_CV; |
| 55 | N_NEU_SS_CV; N_NEU_FL_P; |
| 56 | N_WBC_FL_CV; N_NEU_FL_P; |
| 57 | N_WBC_FL_P; N_WBC_FLSS_Area; |
| 58 | N_WBC_SS_P; N_WBC_FL_P; |
| 59 | N_WBC_FL_P; N_WBC_FLFS_Area; |
| 60 | N_NEU_SS_P; N_NEU_FL_P; |
| 61 | N_WBC_SS_P; N_NEU_FL_P; |
| 62 | N_WBC_SSFS_Area; N_NEU_FL_P; |
| 63 | N_WBC_FLSS_Area; N_NEU_FL_P; |
| 64 | N_WBC_FL_CV; N_WBC_FS_W; |
| 65 | N_WBC_FS_W; N_NEU_FL_CV; |
| 66 | N_WBC_FLFS_Area; N_NEU_FL_P; |
| 67 | N_WBC_SS_W; N_NEU_FL_CV; |
| 68 | N_WBC_FL_CV; N_WBC_FS_CV; |
| 69 | N_WBC_FS_P; N_NEU_FL_P; |
| 70 | N_WBC_SS_W; N_WBC_FL_CV; |
| 71 | N_WBC_FL_P; N_WBC_FS_P; |
| 72 | N_NEU_SS_W; N_NEU_FL_CV; |
| 73 | N_WBC_FL_P; N_NEU_FS_P; |
| 74 | N_NEU_FL_CV; N_NEU_FLFS_Area; |
| 75 | N_NEU_FL_CV; N_NEU_FLSS_Area; |
| 76 | N_WBC_FL_P; N_NEU_FL_P; |
| 77 | N_WBC_FS_P; N_NEU_FL_W; |
| 78 | N_NEU_SS_P; N_NEU_FL_W; |
| 79 | N_NEU_FL_P; N_NEU_FS_P; |
| 80 | N_WBC_FL_CV; N_NEU_FL_W; |
| 81 | N_WBC_FL_CV; N_NEU_FS_W; |
| 82 | N_WBC_SS_P; N_NEU_FL_W; |
| 83 | N_NEU_FL_CV; N_NEU_SSFS_Area; |
| 84 | N_WBC_SS_W; N_NEU_FL_W; |
| 85 | N_WBC_FS_W; N_NEU_FL_W; |
| 86 | N_WBC_SS_W; N_WBC_FS_P; |
| 87 | N_WBC_SSFS_Area; N_NEU_FL_W; |
| 88 | N_WBC_FL_CV; N_NEU_FS_CV; |
| 89 | N_NEU_FL_W; N_NEU_FS_P; |
| 90 | N_WBC_SS_CV; N_WBC_FS_P; |
| 91 | N_WBC_FL_CV; N_NEU_SS_W; |
| 92 | N_WBC_SS_W; N_WBC_SSFS_Area; |
| 93 | N_WBC_FLFS_Area; N_NEU_FL_W; |
| 94 | N_NEU_SS_CV; N_NEU_FL_W; |
| 95 | N_WBC_SS_CV; N_NEU_FL_W; |
| 96 | N_NEU_FL_W; N_NEU_SSFS_Area; |
| 97 | N_WBC_SS_W; N_NEU_FLFS_Area; |
| 98 | N_NEU_FL_W; N_NEU_FS_W; |
| 99 | N_WBC_FS_CV; N_NEU_FL_W; |
| 100 | N_NEU_SS_W; N_NEU_FL_W; |
| 101 | N_NEU_FL_W; N_NEU_FS_CV; |
| 102 | N_WBC_FS_P; N_WBC_FLSS_Area; |
| 103 | N_NEU_SS_P; N_NEU_FS_CV; |
| 104 | N_NEU_SS_P; N_NEU_FLFS_Area; |
| 105 | N_WBC_SS_CV; N_WBC_FL_CV; |
| 106 | N_WBC_FL_CV; N_NEU_FLFS_Area; |
| 107 | N_NEU_SS_P; N_NEU_FS_W; |
| 108 | N_WBC_SS_P; N_NEU_FS_CV; |
| 109 | N_NEU_FL_W; N_NEU_FLFS_Area; |
| 110 | N_WBC_FS_P; N_NEU_SS_W; |
| 111 | N_WBC_FLSS_Area; N_NEU_FL_W; |
| 112 | N_NEU_FL_W; N_NEU_FLSS_Area; |
| 113 | N_WBC_FS_P; N_NEU_FS_CV; |
| 114 | N_WBC_SS_CV; N_NEU_FL_CV; |
| 115 | N_WBC_SS_P; N_NEU_FS_W; |
| 116 | N_WBC_FS_P; N_NEU_FLFS_Area; |
| 117 | N_WBC_SS_W; N_WBC_FS_W; |
| 118 | N_WBC_SS_P; N_NEU_FLFS_Area; |
| 119 | N_WBC_SS_P; N_WBC_FS_W; |
| 120 | N_WBC_FS_P; N_WBC_FS_CV; |
| 121 | N_WBC_FL_CV; N_NEU_FLSS_Area; |
| 122 | N_WBC_SS_W; N_NEU_FS_W; |
| 123 | N_WBC_SS_W; N_NEU_FLSS_Area; |
| 124 | N_WBC_FS_P; N_WBC_FS_W; |
| 125 | N_WBC_FS_P; N_NEU_FLSS_Area; |
| 126 | N_WBC_FLSS_Area; N_NEU_FL_CV; |
| 127 | N_WBC_FS_P; N_NEU_FS_W; |
| 128 | N_WBC_SSFS_Area; N_NEU_FLFS_Area; |
| 129 | N_WBC_FS_W; N_NEU_SS_P; |
| 130 | N_WBC_SSFS_Area; N_NEU_FLSS_Area; |
| 131 | N_WBC_FS_W; N_WBC_SSFS_Area; |
| 132 | N_WBC_FS_W; N_NEU_FLFS_Area; |
| 133 | N_WBC_SS_W; N_NEU_FS_CV; |
| 134 | N_WBC_FS_W; N_WBC_FS_CV; |
| 135 | N_WBC_FS_W; N_NEU_FLSS_Area; |
| 136 | N_WBC_SS_W; N_NEU_FS_P; |
| 137 | N_WBC_FLSS_Area; N_WBC_SSFS_Area; |
| 138 | N_WBC_SS_P; N_WBC_SS_CV; |
| 139 | N_WBC_FS_P; N_WBC_FLFS_Area; |
| 140 | N_WBC_FL_CV; N_WBC_FLSS_Area; |
| 141 | N_WBC_SS_W; N_WBC_FLSS_Area; |
| 142 | N_NEU_SS_P; N_NEU_FLSS_Area; |
| 143 | N_WBC_SS_W; N_WBC_SS_CV; |
| 144 | N_WBC_SS_W; N_WBC_FLFS_Area; |
| 145 | N_WBC_FS_CV; N_NEU_FL_CV; |
| 146 | N_WBC_FS_W; N_NEU_SS_W; |
| 147 | N_WBC_SS_P; N_WBC_SS_W; |
| 148 | N_WBC_SS_CV; N_NEU_SS_P; |
| 149 | N_WBC_SSFS_Area; N_NEU_FS_W; |
| 150 | N_WBC_SS_W; N_NEU_SS_P; |
| 151 | N_WBC_SS_P; N_NEU_FLSS_Area; |
| 152 | N_WBC_FLFS_Area; N_NEU_FL_CV; |
| 153 | N_WBC_FS_W; N_WBC_FLSS_Area; |
| 154 | N_NEU_SS_P; N_NEU_SS_CV; |
| 155 | N_NEU_SS_W; N_NEU_FLFS_Area; |
| 156 | N_WBC_SSFS_Area; N_NEU_SS_W; |
| 157 | N_WBC_SS_W; N_WBC_FS_CV; |
| 158 | N_NEU_FLFS_Area; N_NEU_SSFS_Area; |
| 159 | N_WBC_SS_W; N_NEU_SS_CV; |
| 160 | N_WBC_SS_CV; N_NEU_FLFS_Area; |
| 161 | N_NEU_FS_P; N_NEU_FS_CV; |
| 162 | N_WBC_FLFS_Area; N_NEU_FLFS_Area; |
| 163 | N_WBC_FS_W; N_NEU_FS_W; |
| 164 | N_WBC_SS_W; N_NEU_SSFS_Area; |
| 165 | N_WBC_FS_W; N_NEU_FS_CV; |
| 166 | N_NEU_FS_P; N_NEU_FLFS_Area; |
| 167 | N_WBC_SS_P; N_WBC_FS_CV; |
| 168 | N_WBC_SS_P; N_NEU_SS_CV; |
| 169 | N_NEU_FS_P; N_NEU_FS_W; |
| 170 | N_NEU_FS_W; N_NEU_FLFS_Area; |
| 171 | N_NEU_SS_P; N_NEU_SS_W; |
| 172 | N_WBC_FLSS_Area; N_NEU_SS_P; |
| 173 | N_WBC_FS_P; N_NEU_SS_CV; |
| 174 | N_WBC_SS_CV; N_WBC_FS_W; |
| 175 | N_WBC_SS_P; N_NEU_SS_W; |
| 176 | N_WBC_FLFS_Area; N_NEU_SS_P; |
| 177 | N_WBC_SS_P; N_WBC_FLSS_Area; |
| 178 | N_WBC_FL_CV; N_WBC_FLFS_Area; |
| 179 | N_WBC_SS_P; N_WBC_FLFS_Area; |
| 180 | N_WBC_SS_W; N_NEU_SS_W; |
| 181 | N_NEU_SS_W; N_NEU_FS_W; |
| 182 | N_NEU_SS_W; N_NEU_SS_CV; |
| 183 | N_WBC_FS_W; N_WBC_FLFS_Area; |
| 184 | N_NEU_FS_W; N_NEU_FLSS_Area; |
| 185 | N_NEU_FLSS_Area; N_NEU_SSFS_Area; |
| 186 | N_NEU_FS_CV; N_NEU_FLFS_Area; |
| 187 | N_NEU_FS_W; N_NEU_FS_CV; |
| 188 | N_WBC_FL_CV; N_NEU_SSFS_Area; |
| 189 | N_WBC_FS_CV; N_NEU_FLFS_Area; |
| 190 | N_WBC_FS_W; N_NEU_SS_CV; |
| 191 | N_NEU_SS_W; N_NEU_FS_P; |
| 192 | N_NEU_FS_P; N_NEU_FLSS_Area; |
| 193 | N_WBC_SS_CV; N_NEU_FLSS_Area; |
| 194 | N_NEU_SS_W; N_NEU_FLSS_Area; |
| 195 | N_WBC_FS_W; N_NEU_FS_P; |
| 196 | N_WBC_FLSS_Area; N_NEU_FS_W; |
| 197 | N_WBC_FS_CV; N_NEU_SS_P; |
| 198 | N_NEU_FS_CV; N_NEU_FLSS_Area; |
| 199 | N_NEU_SS_CV; N_NEU_FLFS_Area; |
| 200 | N_WBC_FLSS_Area; N_NEU_FLFS_Area; |
| 20 | N_WBC_SSFS_Area; N_NEU_SSFS_Area; |
| 202 | N_NEU_FLFS_Area; N_NEU_FLSS_Area; |
| 203 | N_WBC_SS_P; N_NEU_FL_CV; |
| 204 | N_WBC_FS_W; N_NEU_SSFS_Area; |
| 205 | N_WBC_FS_CV; N_NEU_FLSS_Area; |
| 206 | N_WBC_FS_P; N_NEU_SS_P; |
| 207 | N_NEU_SS_W; N_NEU_FS_CV; |
| 208 | N_WBC_FS_CV; N_NEU_FS_W; |
| 209 | N_WBC_FLFS_Area; N_NEU_FS_W; |
| 210 | N_NEU_SS_P; N_NEU_FL_CV; |
| 211 | N_WBC_SS_CV; N_NEU_FS_P; |
| 212 | N_WBC_SS_CV; N_NEU_FS_W; |
| 213 | N_WBC_FL_CV; N_NEU_SS_P; |
| 214 | N_WBC_FLSS_Area; N_NEU_FS_CV; |
| 215 | N_NEU_SS_CV; N_NEU_FLSS_Area; |
| 216 | N_WBC_SS_P; N_WBC_FS_P; |
| 217 | N_NEU_SS_CV; N_NEU_FL_CV; |
| 218 | N_WBC_FLSS_Area; N_NEU_SS_W; |
| 219 | N_WBC_FLSS_Area; N_NEU_FLSS_Area; |
| 220 | N_WBC_FLFS_Area; N_NEU_FLSS_Area; |
| 221 | N_NEU_SS_CV; N_NEU_FS_W; |
| 222 | N_WBC_SS_P; N_WBC_FL_CV; |
| 223 | N_WBC_FLSS_Area; N_NEU_FS_P; |
| 224 | N_WBC_FLFS_Area; N_NEU_SS_W; |
| 225 | N_WBC_FS_P; N_NEU_SSFS_Area; |
| 226 | N_NEU_FS_W; N_NEU_SSFS_Area; |
| 227 | N_WBC_FS_CV; N_NEU_SS_W; |
| 228 | N_WBC_SS_CV; N_NEU_SS_W; |
| 229 | N_NEU_SS_P; N_NEU_SSFS_Area; |
| 230 | N_NEU_SS_W; N_NEU_SSFS_Area; |
| 231 | N_WBC_SS_CV; N_WBC_FLSS_Area; |
| 232 | N_WBC_FLFS_Area; N_NEU_FS_CV; |
| 233 | N_WBC_SS_P; N_NEU_SSFS_Area; |
| 234 | N_WBC_FLFS_Area; N_WBC_SSFS_Area; |
| 235 | N_WBC_SS_P; N_WBC_SSFS_Area; |
| 236 | N_WBC_SS_P; N_NEU_SS_P; |
| 237 | N_WBC_SS_P; N_NEU_FS_P; |
| 238 | N_WBC_SSFS_Area; N_NEU_SS_P; |
| 239 | N_WBC_FS_CV; N_WBC_FLSS_Area; |
| 240 | N_NEU_SS_P; N_NEU_FS_P; |
| 241 | N_WBC_FLSS_Area; N_NEU_SS_CV; |
| 242 | N_WBC_FLFS_Area; N_NEU_FS_P; |
| 243 | N_WBC_SSFS_Area; N_NEU_FS_CV; |
| 244 | N_WBC_FLSS_Area; N_NEU_SSFS_Area; |
| 245 | N_WBC_SS_CV; N_WBC_FLFS_Area; |
| 246 | N_NEU_SS_CV; N_NEU_FS_P; |
| 247 | N_WBC_FLFS_Area; N_WBC_FLSS_Area; |
| 248 | N_WBC_SS_CV; N_NEU_FS_CV; |
| 249 | N_WBC_FS_P; N_WBC_SSFS_Area; |
| 250 | N_NEU_FS_CV; N_NEU_SSFS_Area; |
| 25 | N_WBC_SSFS_Area; N_NEU_FL_CV; |
| 252 | N_WBC_FS_CV; N_NEU_FS_CV; |
| 253 | N_WBC_FL_CV; N_WBC_SSFS_Area; |
| 254 | N_NEU_SS_CV; N_NEU_FS_CV; |
| 255 | N_WBC_FLFS_Area; N_NEU_SS_CV; |
| 256 | N_WBC_FS_CV; N_WBC_FLFS_Area; |
| 257 | N_WBC_FS_CV; N_NEU_FS_P; |
| 258 | N_WBC_FS_P; N_NEU_FL_CV; |
| 259 | N_WBC_FLFS_Area; N_NEU_SSFS_Area; |
| 260 | N_WBC_FL_CV; N_NEU_SS_CV; |
| 261 | N_NEU_FS_P; N_NEU_SSFS_Area; |
| 262 | N_WBC_FS_P; N_NEU_FS_P; |
| 263 | N_WBC_FL_CV; N_WBC_FS_P; |
| 264 | N_WBC_SS_CV; N_NEU_SSFS_Area; |
| 265 | N_WBC_FS_CV; N_NEU_SSFS_Area; |
| 266 | N_NEU_SS_CV; N_NEU_SSFS_Area; |
| 267 | N_WBC_SS_CV; N_WBC_FS_CV; |
| 268 | N_NEU_FL_CV; N_NEU_FS_P; |
| 269 | N_WBC_SSFS_Area; N_NEU_FS_P; |
| 270 | N_WBC_SS_CV; N_NEU_SS_CV; |

In some embodiments, the combination of N_WBC_FL_P and N_WBC_FS_W, the combination of N_WBC_FL_W and N_NEU_FL_P, the combination of N_WBC_FL_W and N_NEU_FLSS_Area, the combination of N_WBC_FL_W and N_NEU_FL_W, or the combination of N_WBC_SS_P and N_WBC_FL_P may be used to calculate the infection marker parameter for diagnosis of sepsis.

Patients with bacterial infection can be divided into common infection and severe infection according to their infection severity and organ function status. The clinical treatment methods and nursing measures of the two infections are different. Therefore, the identification of common infection and severe infection can help doctors identify patients with life-threatening diseases and allocate medical resources more reasonably.

To this end, in an application scenario of identification of a common infection and a severe infection, that is, the infection marker parameter is used to determine whether the subject has a common infection or a severe infection, the processor 140 may be configured to output prompt information indicating that the subject has a severe infection when the infection marker parameter satisfies a third preset condition. Herein, the third preset condition may likewise be that the value of the infection marker parameter is greater than the preset threshold. The preset threshold can be determined based on a specific combination of parameters and a blood cell analyzer.

In some embodiments, the infection marker parameter for identification of a common infection and a severe infection may be one of the following parameters:
N_WBC_FL_W;N_WBC_FL_P;N_NEU_FL_W;N_NEU_FL_P;N_NEU_FLFS_Area;N_WBC_SS_W;N_WBC_FS_W;N_NEU_FLSS_Area;N_NEU_FS_W;N_WBC_FLSS_Area;N_NEU_S S_W;N_WBC_FLFS_Area;N_NEU_FS_CV;N_WBC_SS_P;N_NEU_SS_P;N_WBC_FS_CV; N_NEU_SSFS_Area;N_WBC_FS_P;N_WBC_SS_CV.

In other embodiments, infection marker parameters may be calculated by combining the various parameters listed in Table 3 for identification of a common infection and a severe infection.

TABLE 3
Parameter combinations for identification of
a common infection and a severe infection

| No. | Parameter combination |
|---|---|
| 1 | N_WBC_FL_P; N_WBC_FS_W; |
| 2 | N_WBC_FL_P; N_NEU_FS_W; |
| 3 | N_WBC_FL_W; N_NEU_FS_CV; |
| 4 | N_WBC_FL_W; N_NEU_FS_W; |
| 5 | N_WBC_FL_P; N_NEU_FS_CV; |
| 6 | N_NEU_FL_P; N_NEU_FS_W; |
| 7 | N_WBC_FL_P; N_WBC_FS_CV; |
| 8 | N_WBC_FL_W; N_WBC_FS_P; |
| 9 | N_NEU_FL_P; N_NEU_FS_CV; |
| 10 | N_WBC_SS_W; N_WBC_FL_W; |
| 11 | N_WBC_SS_CV; N_WBC_FL_W; |
| 12 | N_WBC_SS_P; N_WBC_FL_W; |
| 13 | N_WBC_FL_W; N_NEU_SS_P; |
| 14 | N_WBC_FL_W; N_WBC_FS_W; |
| 15 | N_WBC_FS_W; N_NEU_FL_P; |
| 16 | N_WBC_FL_W; N_NEU_SS_W; |
| 17 | N_WBC_SS_W; N_WBC_FL_P; |
| 18 | N_WBC_FL_W; N_NEU_SS_CV; |
| 19 | N_WBC_FL_W; N_NEU_SSFS_Area; |
| 20 | N_WBC_FL_P; N_WBC_FL_CV; |
| 21 | N_WBC_FL_W; N_NEU_FLSS_Area; |
| 22 | N_WBC_FL_W; N_NEU_FLFS_Area; |
| 23 | N_WBC_FL_W; N_NEU_FS_P; |
| 24 | N_WBC_FL_W; N_WBC_FS_CV; |
| 25 | N_WBC_FL_W; N_NEU_FL_CV; |
| 26 | N_WBC_SS_CV; N_WBC_FL_P; |
| 27 | N_WBC_FL_W; N_NEU_FL_W; |
| 28 | N_WBC_FL_P; N_WBC_FL_W; |
| 29 | N_WBC_FL_W; N_WBC_FLFS_Area; |
| 30 | N_WBC_FL_W; N_NEU_FL_P; |
| 31 | N_WBC_FL_W; N_WBC_SSFS_Area; |
| 32 | N_WBC_FL_W; N_WBC_FL_CV; |
| 33 | N_WBC_FL_W; N_WBC_FLSS_Area; |
| 34 | N_WBC_SS_W; N_NEU_FL_P; |
| 35 | N_WBC_FL_P; N_NEU_SS_W; |
| 36 | N_WBC_FL_P; N_NEU_SSFS_Area; |
| 37 | N_WBC_FS_CV; N_NEU_FL_P; |
| 38 | N_WBC_SS_CV; N_NEU_FL_P; |
| 39 | N_NEU_SS_W; N_NEU_FL_P; |
| 40 | N_NEU_FL_CV; N_NEU_FS_W; |
| 41 | N_WBC_FL_P; N_NEU_FL_W; |
| 42 | N_NEU_FL_P; N_NEU_SSFS_Area; |
| 43 | N_WBC_FL_P; N_NEU_FLSS_Area; |
| 44 | N_WBC_FL_P; N_NEU_FLFS_Area; |
| 45 | N_NEU_FL_P; N_NEU_FL_W; |
| 46 | N_WBC_FL_P; N_NEU_FL_CV; |
| 47 | N_NEU_FL_P; N_NEU_FL_CV; |
| 48 | N_NEU_FL_P; N_NEU_FLFS_Area; |
| 49 | N_NEU_FL_P; N_NEU_FLSS_Area; |
| 50 | N_WBC_FL_P; N_WBC_SSFS_Area; |
| 51 | N_NEU_FL_CV; N_NEU_FS_CV; |
| 52 | N_NEU_FL_W; N_NEU_FL_CV; |
| 53 | N_WBC_FL_P; N_NEU_SS_CV; |
| 54 | N_WBC_FL_P; N_WBC_FLSS_Area; |
| 55 | N_WBC_FL_P; N_WBC_FLFS_Area; |
| 56 | N_WBC_FL_P; N_NEU_SS_P; |
| 57 | N_NEU_SS_CV; N_NEU_FL_P; |
| 58 | N_WBC_FL_CV; N_NEU_FL_P; |
| 59 | N_WBC_SS_P; N_WBC_FL_P; |
| 60 | N_WBC_SSFS_Area; N_NEU_FL_P; |
| 61 | N_WBC_FLSS_Area; N_NEU_FL_P; |
| 62 | N_WBC_FL_CV; N_WBC_FS_W; |
| 63 | N_WBC_SS_P; N_NEU_FL_P; |
| 64 | N_NEU_SS_P; N_NEU_FL_P; |
| 65 | N_WBC_FLFS_Area; N_NEU_FL_P; |
| 66 | N_WBC_FL_CV; N_WBC_FS_CV; |
| 67 | N_NEU_FL_CV; N_NEU_FLFS_Area; |
| 68 | N_WBC_FS_W; N_NEU_FL_CV; |
| 69 | N_WBC_FS_P; N_NEU_FL_W; |
| 70 | N_NEU_SS_P; N_NEU_FL_W; |
| 71 | N_NEU_FL_CV; N_NEU_FLSS_Area; |
| 72 | N_WBC_FL_CV; N_NEU_FL_W; |
| 73 | N_WBC_SS_P; N_NEU_FL_W; |
| 74 | N_WBC_SS_W; N_NEU_FL_CV; |
| 75 | N_WBC_FL_CV; N_NEU_FS_W; |
| 76 | N_WBC_FL_P; N_NEU_FS_P; |
| 77 | N_WBC_SS_W; N_WBC_FL_CV; |
| 78 | N_WBC_FS_P; N_NEU_FL_P; |
| 79 | N_WBC_FS_W; N_NEU_FL_W; |
| 80 | N_WBC_FL_P; N_WBC_FS_P; |
| 81 | N_WBC_SS_W; N_NEU_FL_W; |
| 82 | N_NEU_FL_W; N_NEU_FS_P; |
| 83 | N_NEU_SS_W; N_NEU_FL_CV; |
| 84 | N_NEU_FL_P; N_NEU_FS_P; |
| 85 | N_WBC_SSFS_Area; N_NEU_FL_W; |
| 86 | N_NEU_FL_W; N_NEU_FS_W; |
| 87 | N_WBC_FL_P; N_NEU_FL_P; |
| 88 | N_WBC_SS_CV; N_NEU_FL_W; |
| 89 | N_WBC_FS_CV; N_NEU_FL_W; |
| 90 | N_NEU_SS_CV; N_NEU_FL_W; |
| 91 | N_NEU_FL_CV; N_NEU_SSFS_Area; |
| 92 | N_NEU_SS_W; N_NEU_FL_W; |
| 93 | N_NEU_FL_W; N_NEU_FS_CV; |
| 94 | N_NEU_FL_W; N_NEU_SSFS_Area; |
| 95 | N_WBC_FL_CV; N_NEU_FS_CV; |
| 96 | N_WBC_FLFS_Area; N_NEU_FL_W; |
| 97 | N_NEU_FL_W; N_NEU_FLFS_Area; |
| 98 | N_WBC_FLSS_Area; N_NEU_FL_W; |
| 99 | N_WBC_FS_P; N_WBC_FLSS_Area; |
| 100 | N_WBC_FL_CV; N_NEU_FLFS_Area; |
| 101 | N_NEU_FL_W; N_NEU_FLSS_Area; |
| 102 | N_WBC_SS_W; N_NEU_FLFS_Area; |
| 103 | N_NEU_SS_P; N_NEU_FLFS_Area; |
| 104 | N_WBC_SS_W; N_WBC_FS_P; |
| 105 | N_WBC_FS_W; N_NEU_FLFS_Area; |
| 106 | N_WBC_FL_CV; N_NEU_FLSS_Area; |
| 107 | N_WBC_FS_P; N_NEU_FLFS_Area; |
| 108 | N_WBC_SS_P; N_NEU_FLFS_Area; |
| 109 | N_WBC_SS_W; N_WBC_FS_W; |
| 110 | N_WBC_FS_W; N_NEU_FLSS_Area; |
| 111 | N_WBC_FL_CV; N_NEU_SS_W; |
| 112 | N_WBC_SSFS_Area; N_NEU_FLFS_Area; |
| 113 | N_NEU_SS_P; N_NEU_FS_W; |
| 114 | N_WBC_FS_P; N_NEU_FLSS_Area; |
| 115 | N_NEU_SS_P; N_NEU_FS_CV; |
| 116 | N_WBC_SS_CV; N_WBC_FS_P; |
| 117 | N_WBC_SS_P; N_NEU_FS_W; |
| 118 | N_WBC_FS_W; N_WBC_FLSS_Area; |
| 119 | N_WBC_SSFS_Area; N_NEU_FLSS_Area; |
| 120 | N_WBC_SS_P; N_NEU_FS_CV; |
| 121 | N_WBC_FL_CV; N_WBC_FLSS_Area; |
| 122 | N_WBC_SS_W; N_NEU_FLSS_Area; |
| 123 | N_WBC_SS_W; N_NEU_FS_W; |
| 124 | N_WBC_FS_P; N_WBC_FLFS_Area; |
| 125 | N_WBC_SS_W; N_WBC_SSFS_Area; |
| 126 | N_WBC_SS_P; N_WBC_FS_W; |
| 127 | N_WBC_FS_P; N_NEU_FS_CV; |
| 128 | N_WBC_FS_W; N_NEU_SS_P; |
| 129 | N_WBC_FS_W; N_NEU_SS_W; |
| 130 | N_WBC_FLSS_Area; N_WBC_SSFS_Area; |
| 131 | N_WBC_FLSS_Area; N_NEU_FL_CV; |
| 132 | N_NEU_FLFS_Area; N_NEU_SSFS_Area; |
| 133 | N_WBC_SS_W; N_WBC_FLSS_Area; |
| 134 | N_WBC_SS_W; N_WBC_FLFS_Area; |
| 135 | N_WBC_FS_P; N_NEU_FS_W; |
| 136 | N_NEU_SS_W; N_NEU_FLFS_Area; |
| 137 | N_WBC_SS_CV; N_NEU_FLFS_Area; |
| 138 | N_WBC_FS_P; N_WBC_FS_CV; |
| 139 | N_NEU_FS_P; N_NEU_FLFS_Area; |
| 140 | N_WBC_SS_W; N_NEU_FS_CV; |
| 141 | N_WBC_FS_P; N_WBC_FS_W; |
| 142 | N_WBC_FS_P; N_NEU_SS_W; |
| 143 | N_NEU_FS_W; N_NEU_FLFS_Area; |
| 144 | N_WBC_FS_W; N_WBC_SSFS_Area; |
| 145 | N_WBC_FS_W; N_WBC_FS_CV; |
| 146 | N_WBC_FS_W; N_WBC_FLFS_Area; |
| 147 | N_NEU_SS_P; N_NEU_FLSS_Area; |
| 148 | N_WBC_FL_CV; N_WBC_FLFS_Area; |
| 149 | N_WBC_FS_CV; N_NEU_FLFS_Area; |
| 150 | N_WBC_SS_W; N_NEU_FS_P; |
| 151 | N_WBC_FLFS_Area; N_NEU_FL_CV; |
| 152 | N_WBC_SS_CV; N_WBC_FS_W; |
| 153 | N_NEU_FS_CV; N_NEU_FLFS_Area; |
| 154 | N_WBC_FS_CV; N_NEU_FL_CV; |
| 155 | N_WBC_SS_P; N_NEU_FLSS_Area; |
| 156 | N_NEU_FS_W; N_NEU_FLSS_Area; |
| 157 | N_WBC_FLSS_Area; N_NEU_FS_W; |
| 158 | N_NEU_FLSS_Area; N_NEU_SSFS_Area; |
| 159 | N_NEU_SS_CV; N_NEU_FLFS_Area; |
| 160 | N_WBC_FS_W; N_NEU_FS_CV; |
| 161 | N_WBC_FLSS_Area; N_NEU_FLFS_Area; |
| 162 | N_NEU_FLFS_Area; N_NEU_FLSS_Area; |
| 163 | N_WBC_SS_W; N_WBC_FS_CV; |
| 164 | N_WBC_FLFS_Area; N_NEU_FLFS_Area; |
| 165 | N_WBC_FS_W; N_NEU_FS_W; |
| 166 | N_WBC_SS_P; N_WBC_SS_CV; |
| 167 | N_WBC_SS_W; N_WBC_SS_CV; |
| 168 | N_NEU_FS_P; N_NEU_FLSS_Area; |
| 169 | N_WBC_SS_CV; N_NEU_FLSS_Area; |
| 170 | N_WBC_FS_W; N_NEU_SS_CV; |
| 171 | N_WBC_FLFS_Area; N_NEU_SS_P; |
| 172 | N_WBC_SS_CV; N_WBC_FL_CV; |
| 173 | N_NEU_FS_P; N_NEU_FS_CV; |
| 174 | N_WBC_FLSS_Area; N_NEU_SS_P; |
| 175 | N_NEU_SS_W; N_NEU_FS_W; |
| 176 | N_WBC_SS_P; N_WBC_FLFS_Area; |
| 177 | N_WBC_FS_CV; N_NEU_FLSS_Area; |
| 178 | N_NEU_FS_P; N_NEU_FS_W; |
| 179 | N_NEU_FS_CV; N_NEU_FLSS_Area; |
| 180 | N_WBC_SS_P; N_WBC_FLSS_Area; |
| 181 | N_NEU_SS_W; N_NEU_FLSS_Area; |
| 182 | N_WBC_SS_W; N_NEU_SSFS_Area; |
| 183 | N_WBC_SS_P; N_WBC_SS_W; |
| 184 | N_WBC_SS_P; N_WBC_FS_CV; |
| 185 | N_WBC_SS_CV; N_NEU_SS_P; |
| 186 | N_WBC_SS_W; N_NEU_SS_CV; |
| 187 | N_WBC_FLFS_Area; N_NEU_FS_W; |
| 188 | N_WBC_SS_CV; N_NEU_FL_CV; |
| 189 | N_WBC_SSFS_Area; N_NEU_FS_W; |
| 190 | N_WBC_SS_W; N_NEU_SS_P; |
| 191 | N_NEU_FS_W; N_NEU_FS_CV; |
| 192 | N_WBC_SS_W; N_NEU_SS_W; |
| 193 | N_WBC_FLSS_Area; N_NEU_FS_CV; |
| 194 | N_NEU_SS_P; N_NEU_SS_CV; |
| 195 | N_WBC_FS_W; N_NEU_SSFS_Area; |
| 196 | N_WBC_FS_W; N_NEU_FS_P; |
| 197 | N_WBC_FLSS_Area; N_NEU_FS_P; |
| 198 | N_WBC_SSFS_Area; N_NEU_SS_W; |
| 199 | N_WBC_FL_CV; N_NEU_SSFS_Area; |
| 200 | N_WBC_FLSS_Area; N_NEU_SS_W; |
| 201 | N_NEU_SS_CV; N_NEU_FLSS_Area; |
| 202 | N_WBC_SS_CV; N_NEU_FS_W; |
| 203 | N_WBC_FS_CV; N_NEU_SS_P; |
| 204 | N_WBC_SS_P; N_NEU_SS_CV; |
| 205 | N_NEU_SS_W; N_NEU_SS_CV; |
| 206 | N_NEU_SS_W; N_NEU_FS_P; |
| 207 | N_WBC_SS_CV; N_WBC_FLSS_Area; |
| 208 | N_NEU_SS_P; N_NEU_SS_W; |
| 209 | N_WBC_FLFS_Area; N_NEU_FLSS_Area; |
| 210 | N_WBC_FLFS_Area; N_NEU_SS_W; |
| 211 | N_WBC_FLSS_Area; N_NEU_FLSS_Area; |
| 212 | N_NEU_SS_W; N_NEU_FS_CV; |
| 213 | N_WBC_FLFS_Area; N_WBC_SSFS_Area; |
| 214 | N_WBC_SS_P; N_NEU_SS_W; |
| 215 | N_WBC_SS_CV; N_NEU_FS_P; |
| 216 | N_WBC_FLFS_Area; N_NEU_FS_CV; |
| 217 | N_WBC_FS_CV; N_NEU_FS_W; |
| 218 | N_WBC_FS_P; N_NEU_SS_CV; |
| 219 | N_WBC_FS_CV; N_WBC_FLSS_Area; |
| 220 | N_NEU_SS_CV; N_NEU_FS_W; |
| 221 | N_WBC_FS_CV; N_NEU_SS_W; |
| 222 | N_WBC_SS_CV; N_WBC_FLFS_Area; |
| 223 | N_WBC_FLSS_Area; N_NEU_SS_CV; |
| 224 | N_WBC_FLFS_Area; N_NEU_FS_P; |
| 225 | N_NEU_FS_W; N_NEU_SSFS_Area; |
| 226 | N_WBC_FLSS_Area; N_NEU_SSFS_Area; |
| 227 | N_WBC_FLFS_Area; N_WBC_FLSS_Area; |
| 228 | N_WBC_SS_CV; N_NEU_SS_W; |
| 229 | N_NEU_SS_W; N_NEU_SSFS_Area; |
| 230 | N_WBC_FS_P; N_NEU_SS_P; |
| 231 | N_WBC_FS_CV; N_WBC_FLFS_Area; |
| 232 | N_WBC_FLFS_Area; N_NEU_SS_CV; |
| 233 | N_WBC_SS_P; N_NEU_FL_CV; |
| 234 | N_WBC_FS_P; N_NEU_SSFS_Area; |
| 235 | N_WBC_SS_P; N_WBC_FS_P; |
| 236 | N_WBC_SS_CV; N_NEU_FS_CV; |
| 237 | N_WBC_SSFS_Area; N_NEU_SSFS_Area; |
| 238 | N_WBC_FLFS_Area; N_NEU_SSFS_Area; |
| 239 | N_NEU_SS_P; N_NEU_SSFS_Area; |
| 240 | N_WBC_FL_CV; N_NEU_SS_P; |
| 241 | N_WBC_SS_P; N_WBC_FL_CV; |
| 242 | N_NEU_SS_P; N_NEU_FL_CV; |
| 243 | N_WBC_SS_P; N_NEU_SSFS_Area; |
| 244 | N_WBC_FS_CV; N_NEU_FS_CV; |
| 245 | N_NEU_FS_CV; N_NEU_SSFS_Area; |
| 246 | N_NEU_SS_CV; N_NEU_FS_P; |
| 247 | N_WBC_SSFS_Area; N_NEU_FS_CV; |
| 248 | N_WBC_FS_CV; N_NEU_FS_P; |
| 249 | N_NEU_SS_CV; N_NEU_FL_CV; |
| 250 | N_NEU_SS_CV; N_NEU_FS_CV; |
| 251 | N_WBC_SS_P; N_NEU_FS_P; |
| 252 | N_WBC_SS_P; N_NEU_SS_P; |
| 253 | N_WBC_SS_P; N_WBC_SSFS_Area; |
| 254 | N_WBC_FL_CV; N_WBC_SSFS_Area; |
| 255 | N_WBC_FS_P; N_WBC_SSFS_Area; |
| 256 | N_NEU_SS_P; N_NEU_FS_P; |
| 257 | N_WBC_SSFS_Area; N_NEU_SS_P; |
| 258 | N_WBC_SSFS_Area; N_NEU_FL_CV; |
| 259 | N_WBC_SS_CV; N_NEU_SSFS_Area; |
| 260 | N_WBC_SS_CV; N_WBC_FS_CV; |
| 261 | N_WBC_FS_CV; N_NEU_SSFS_Area; |
| 262 | N_NEU_FS_P; N_NEU_SSFS_Area; |
| 263 | N_WBC_FS_CV; N_NEU_SS_CV; |
| 264 | N_WBC_FL_CV; N_NEU_SS_CV; |
| 265 | N_WBC_FS_P; N_NEU_FL_CV; |
| 266 | N_NEU_SS_CV; N_NEU_SSFS_Area; |
| 267 | N_WBC_FS_CV; N_WBC_SSFS_Area; |
| 268 | N_WBC_SS_CV; N_NEU_SS_CV; |
| 269 | N_WBC_FL_CV; N_WBC_FS_P; |
| 270 | N_WBC_SS_CV; N_WBC_SSFS_Area; |
| 271 | N_WBC_FS_P; N_NEU_FS_P; |
| 272 | N_WBC_SSFS_Area; N_NEU_FS_P |

In the application scenario of infection monitoring, the subject is an infected patient (that is, a patient with infectious inflammation), especially a patient with severe infection or sepsis, for example, the subject is from a patient with severe infection or sepsis in an intensive care unit. Sepsis is a serious infectious disease with a high incidence and case fatality rate. The condition of patients with sepsis fluctuates greatly and requires daily monitoring to prevent patients from deterioration that might go untreated in a timely manner. Therefore, it is very important to determine the progress and treatment effect of sepsis patients with clinical symptoms combined with laboratory test results.

To this end, the processor 140 may be configured to monitor the progression of the infection of the subject based on infection marker parameters.

In some embodiments, the processor 140 may be further configured to monitor the progression of the infection of the subject by:
obtaining multiple values of the infection marker parameter, which are obtained by multiple tests, in particular at least three tests of a blood sample from the subject at different time points; and
determining whether the infection status of the patient has improved or not according to the trend of changes in the values of the infection marker parameter obtained through the multiple tests.

In specific examples, the processor 140 may be further configured to: when the value of the infection marker parameter obtained by the multiple tests gradually tends to decrease, output prompt information indicating that the condition of the subject is improving; and when the value of the infection marker parameter obtained by the multiple tests gradually increases, output prompt information indicating that the condition of the subject is aggravated. The multiple tests herein can be continuous detections every day, or they can be regularly spaced multiple tests.

For example, the values of the infection marker parameter of a patient are obtained for several consecutive days, such as 7 consecutive days, after the diagnosis of sepsis. When these values of the infection marker parameter show a downward trend, the condition of the patient is considered to be improving, and a prompt of improvement is given.

In other embodiments, the processor 140 may also be further configured to prompt the progression of the condition of the subject by:
obtaining a current value of the infection marker parameter obtained by a current detection of a current blood sample from a subject, and obtaining a prior value of the infection marker parameter obtained by a previous detection of a previous blood sample from the subject; and
monitoring the progression of the condition of the subject based on a comparison of the prior value of the infection marker parameter with a first threshold and a comparison of the prior value of the infection marker parameter with the current value of the infection marker parameter.

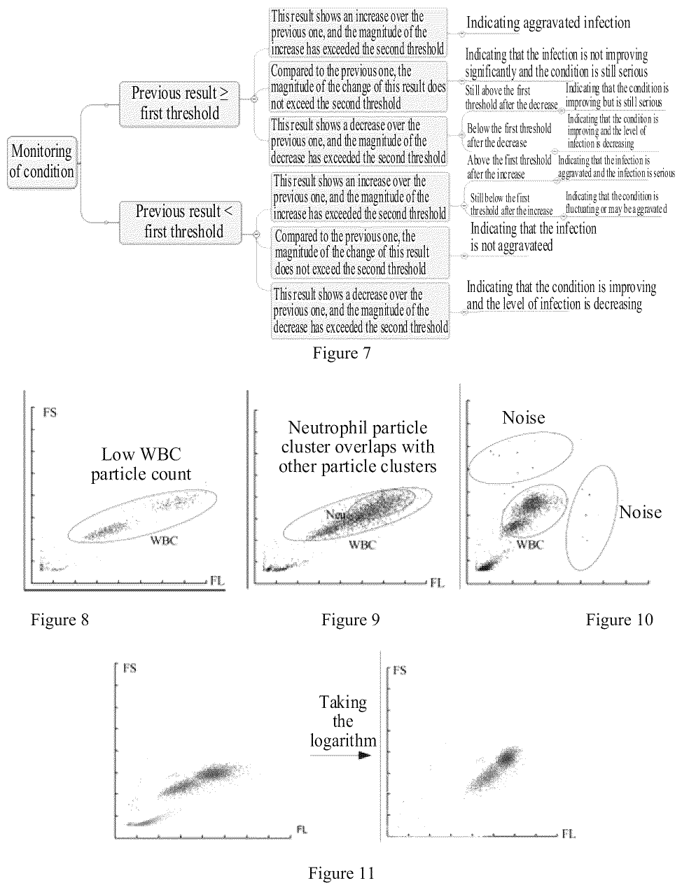

FIG. 7 is a schematic flowchart for monitoring the progression of the infection status of the patient according to some embodiments of the disclosure.

As shown in FIG. 7, the processor 140 may be further configured to, when the prior value of the infection marker parameter is greater than or equal to the first threshold:
if the current value of the infection marker parameter (i.e., the current result in FIG. 7) is greater than the prior value of the infection marker parameter (i.e., the previous result in FIG. 7) and the difference between the two is greater than a second threshold, output prompt information indicating that the condition of the subject is aggravated;
if the current value of the infection marker parameter is less than the prior value of the infection marker parameter and the difference between the two is greater than the second threshold, and the current value of the infection marker parameter is less than the first threshold, output prompt information indicating that the condition of the subject is improving and the degree of infection is decreasing;
if the current value of the infection marker parameter is less than the prior value of the infection marker parameter and the difference between the two is greater than the second threshold, but the current value of the infection marker parameter is greater than or equal to the first threshold, output prompt information indicating that the condition of the subject is improving but the infection is still heavy or skip outputting any prompt information; and
if the difference between the current value of the infection marker parameter and the prior value of the infection marker parameter is not greater than the second threshold, output prompt information indicating that the condition of the subject has not improved significantly and the infection is still heavy or skip outputting any prompt information.

Further, as shown in FIG. 7, the processor 140 may be configured to: when the prior value of the infection marker parameter is less than the first threshold:
if the current value of the infection marker parameter is less than the prior value of the infection marker parameter and the difference between the two is greater than the second threshold, output prompt information indicating that the condition of the subject is improving and the degree of infection is decreasing;
if the current value of the infection marker parameter is greater than the prior value of the infection marker parameter and the difference between the two is greater than the second threshold, and the current value of the infection marker parameter is greater than the first threshold, output prompt information indicating that the condition of the subject is aggravated and the infection is relatively serious;
if the current value of the infection marker parameter is greater than the prior value of the infection marker parameter and the difference between the two is greater than the second threshold, but the current value of the infection marker parameter is less than the first threshold, output prompt information indicating fluctuations in the condition of the subject or possible aggravation of the infection or skip outputting any prompt information; and
if the difference between the current value of the infection marker parameter and the prior value of the infection marker parameter is not greater than the second threshold, output prompt information indicating that the infection of the subject is not aggravated or skip outputting any prompt information.

In the embodiment shown in FIG. 7, when the infection marker parameter is used to monitor the progression of the condition of a patient with a severe infection, the first threshold may be a preset threshold for determining whether the subject has a severe infection. When the infection marker parameter is used to monitor the progression of the condition of a patient with sepsis, the first threshold may be a preset threshold for determining whether the subject has sepsis.

In some embodiments, the infection marker parameter for infection monitoring may be one of the following parameters:
N_WBC_SS_P, N_WBC_SS_W, N_WBC_SS_CV, N_WBC_FL_P, N_WBC_FL_W, N_WBC_FL_CV, N_WBC_FS_P, N_WBC_FS_W, N_WBC_FS_CV, N_WBC_FLFS_Area, N_WBC_FLSS_Area, N_WBC_SSFS_Area.

In other embodiments, an infection marker parameter may be calculated using a combination of N_WBC_FL_P and N_WBC_FS_W for infection monitoring.

In the application scenario of analysis of sepsis prognosis, the subject is a sepsis patient who has received treatment, and the infection marker parameter is used to determine whether the sepsis prognosis of the subject is good. In this regard, the processor 140 may be further configured to determine whether the sepsis prognosis of the subject is good based on the infection marker parameter. For example, when the infection marker parameter satisfies the fourth preset condition, prompt information indicating that the sepsis prognosis of the subject is good is output.

In some embodiments, the infection marker parameter for analysis of sepsis prognosis may be one of the following parameters: N_WBC_FL_W, N_WBC_FS_W, N_WBC_FLSS_Area, N_WBC_FS_CV, N_WBC_FLFS_Area, N_WBC_SS_W, N_WBC_FL_P, N_WBC_SS_CV, N_WBC_SSFS_Area, N_WBC_SS_P, N_WBC_FS_P, N_WBC_FL_CV.

In other embodiments, infection marker parameters may be calculated by combining the various parameters listed in Table 4 for analysis of sepsis prognosis.

TABLE 4
Parameter combinations for analysis of sepsis prognosis

| No. | Parameter combination |
|---|---|
| 1 | N_WBC_FL_P; N_WBC_FS_CV |
| 2 | N_WBC_FL_W; N_WBC_FS_W |
| 3 | N_WBC_SS_W; N_WBC_FL_W |
| 4 | N_WBC_FL_P; N_WBC_FL_CV |
| 5 | N_WBC_SS_P; N_WBC_FL_W |
| 6 | N_WBC_SS_CV; N_WBC_FL_W |
| 7 | N_WBC_FL_W; N_WBC_FS_CV |
| 8 | N_WBC_FL_P; N_WBC_FS_W |
| 9 | N_WBC_FL_W; N_WBC_FS_P |
| 10 | N_WBC_FL_W; N_WBC_SSFS_Area |
| 11 | N_WBC_FL_W; N_WBC_FLSS_Area |
| 12 | N_WBC_FL_P; N_WBC_FL_W |
| 13 | N_WBC_FL_W; N_WBC_FL_CV |
| 14 | N_WBC_FL_W; N_WBC_FLFS_Area |
| 15 | N_WBC_SS_W; N_WBC_FL_P |
| 16 | N_WBC_SS_CV; N_WBC_FL_P |
| 17 | N_WBC_FL_CV; N_WBC_FS_CV |
| 18 | N_WBC_FL_P; N_WBC_SSFS_Area |
| 19 | N_WBC_FL_P; N_WBC_FLSS_Area |
| 20 | N_WBC_FL_P; N_WBC_FLFS_Area |
| 21 | N_WBC_FL_CV; N_WBC_FS_W |
| 22 | N_WBC_FS_W; N_WBC_FLSS_Area |
| 23 | N_WBC_SS_W; N_WBC_FS_W |
| 24 | N_WBC_SS_CV; N_WBC_FS_W |
| 25 | N_WBC_SS_P; N_WBC_FS_W |
| 26 | N_WBC_FS_W; N_WBC_FLFS_Area |
| 27 | N_WBC_SS_P; N_WBC_FS_CV |
| 28 | N_WBC_FS_W; N_WBC_SSFS_Area |
| 29 | N_WBC_FS_P; N_WBC_FS_CV |
| 30 | N_WBC_FS_W; N_WBC_FS_CV |
| 31 | N_WBC_FS_P; N_WBC_FS_W |
| 32 | N_WBC_SS_W; N_WBC_FLSS_Area |
| 33 | N_WBC_SS_P; N_WBC_FL_P |
| 34 | N_WBC_SS_W; N_WBC_FLFS_Area |
| 35 | N_WBC_FS_P; N_WBC_FLSS_Area |
| 36 | N_WBC_SS_P; N_WBC_FLFS_Area |
| 37 | N_WBC_FS_CV; N_WBC_FLSS_Area |
| 38 | N_WBC_SS_P; N_WBC_FLSS_Area |
| 39 | N_WBC_SS_W; N_WBC_FS_CV |
| 40 | N_WBC_FLSS_Area; N_WBC_SSFS_Area |
| 41 | N_WBC_FS_CV; N_WBC_FLFS_Area |
| 42 | N_WBC_SS_CV; N_WBC_FLSS_Area |
| 43 | N_WBC_SS_W; N_WBC_FL_CV |
| 44 | N_WBC_SS_CV; N_WBC_FLFS_Area |
| 45 | N_WBC_FS_P; N_WBC_FLFS_Area |
| 46 | N_WBC_FL_CV; N_WBC_FLSS_Area |
| 47 | N_WBC_SS_W; N_WBC_FS_P |
| 48 | N_WBC_SS_CV; N_WBC_FS_P |
| 49 | N_WBC_FL_CV; N_WBC_FLFS_Area |
| 50 | N_WBC_FLFS_Area; N_WBC_FLSS_Area |
| 51 | N_WBC_SS_CV; N_WBC_FS_CV |
| 52 | N_WBC_FLFS_Area; N_WBC_SSFS_Area |
| 53 | N_WBC_FS_CV; N_WBC_SSFS_Area |
| 54 | N_WBC_SS_P; N_WBC_SS_CV |
| 55 | N_WBC_SS_P; N_WBC_SS_W |
| 56 | N_WBC_SS_W; N_WBC_SS_CV |
| 57 | N_WBC_SS_CV; N_WBC_FL_CV |
| 58 | N_WBC_SS_W; N_WBC_SSFS_Area |
| 59 | N_WBC_FL_P; N_WBC_FS_P |
| 60 | N_WBC_SS_P; N_WBC_SSFS_Area |

Infectious diseases can be divided into different types of infection such as bacterial infection, viral infection, and fungal infection, among which bacterial infection and viral infection are the most common. While the clinical symptoms of the two infections are roughly the same, the treatments are completely different, so it is helpful to make clear the type of infection to choose the correct treatment method. To this end, the infection marker parameter is used for the identification of a bacterial infection and a viral infection, and the processor 140 may be further configured to determine whether the subject's infection type is a viral infection or a bacterial infection based on the infection marker parameter.

In some embodiments, the infection marker parameter for the identification of a bacterial infection and a viral infection may be one of the following parameters: N_WBC_FS_P, N_WBC_FL_P, N_WBC_FS_W, N_WBC_FL_W, N_WBC_FLFS_Area, N_WBC_FLSS_Area, N_WBC_SS_P, N_WBC_SS_W, N_WBC_FL_CV, N_WBC_FS_CV, N_WBC_SSFS_Area, N_WBC_SS_CV.

In other embodiments, infection marker parameters may be calculated by combining the various parameters listed in Table 5 for the identification of a bacterial infection and a viral infection.

TABLE 5
Parameter combinations for identification of
a bacterial infection and a viral infection

| No. | Parameter combination |
|---|---|
| 1 | N_WBC_FL_CV; N_WBC_FS_W |
| 2 | N_WBC_FS_P; N_WBC_FLFS_Area |
| 3 | N_WBC_FS_P; N_WBC_FLSS_Area |
| 4 | N_WBC_FL_P; N_WBC_FS_W |
| 5 | N_WBC_FS_P; N_WBC_FS_W |
| 6 | N_WBC_FL_W; N_WBC_FS_P |
| 7 | N_WBC_FS_P; N_WBC_FS_CV |
| 8 | N_WBC_FS_W; N_WBC_FS_CV |
| 9 | N_WBC_FL_P; N_WBC_FS_P |
| 10 | N_WBC_FL_P; N_WBC_SSFS_Area |
| 11 | N_WBC_FL_P; N_WBC_FLFS_Area |
| 12 | N_WBC_FL_P; N_WBC_FS_CV |
| 13 | N_WBC_FL_CV; N_WBC_FS_CV |
| 14 | N_WBC_FL_P; N_WBC_FLSS_Area |
| 15 | N_WBC_FS_P; N_WBC_SSFS_Area |
| 16 | N_WBC_SS_CV; N_WBC_FS_P |
| 17 | N_WBC_SS_W; N_WBC_FS_P |
| 18 | N_WBC_FL_W; N_WBC_FS_W |
| 19 | N_WBC_FL_CV; N_WBC_FLFS_Area |
| 20 | N_WBC_SS_W; N_WBC_FL_P |
| 21 | N_WBC_SS_P; N_WBC_FS_P |
| 22 | N_WBC_FL_CV; N_WBC_FLSS_Area |
| 23 | N_WBC_FL_CV; N_WBC_FS_P |
| 24 | N_WBC_SS_P; N_WBC_FL_P |
| 25 | N_WBC_SS_CV; N_WBC_FL_P |
| 26 | N_WBC_FS_W; N_WBC_FLFS_Area |
| 27 | N_WBC_FL_P; N_WBC_FL_CV |
| 28 | N_WBC_FL_P; N_WBC_FL_W |
| 29 | N_WBC_SS_CV; N_WBC_FS_W |
| 30 | N_WBC_FS_W; N_WBC_FLSS_Area |
| 31 | N_WBC_FL_W; N_WBC_FL_CV |
| 32 | N_WBC_SS_P; N_WBC_FS_W |
| 33 | N_WBC_FL_CV; N_WBC_SSFS_Area |
| 34 | N_WBC_FL_W; N_WBC_FLFS_Area |
| 35 | N_WBC_FL_W; N_WBC_FLSS_Area |
| 36 | N_WBC_SS_P; N_WBC_FL_W |
| 37 | N_WBC_FS_W; N_WBC_SSFS_Area |
| 38 | N_WBC_SS_W; N_WBC_FS_W |
| 39 | N_WBC_FL_W; N_WBC_SSFS_Area |
| 40 | N_WBC_SS_P; N_WBC_FLFS_Area |
| 41 | N_WBC_SS_W; N_WBC_FL_CV |
| 42 | N_WBC_FL_W; N_WBC_FS_CV |
| 43 | N_WBC_SS_W; N_WBC_FL_W |
| 44 | N_WBC_FLFS_Area; N_WBC_SSFS_Area |
| 45 | N_WBC_SS_CV; N_WBC_FL_W |
| 46 | N_WBC_FLSS_Area; N_WBC_SSFS_Area |
| 47 | N_WBC_SS_P; N_WBC_FLSS_Area |
| 48 | N_WBC_SS_P; N_WBC_FL_CV |
| 49 | N_WBC_SS_W; N_WBC_FLFS_Area |
| 50 | N_WBC_SS_CV; N_WBC_FLFS_Area |
| 51 | N_WBC_SS_P; N_WBC_FS_CV |
| 52 | N_WBC_FS_CV; N_WBC_FLFS_Area |
| 53 | N_WBC_FLFS_Area; N_WBC_FLSS_Area |
| 54 | N_WBC_SS_CV; N_WBC_FL_CV |
| 55 | N_WBC_SS_CV; N_WBC_FLSS_Area |
| 56 | N_WBC_SS_W; N_WBC_FLSS_Area |
| 57 | N_WBC_FS_CV; N_WBC_FLSS_Area |
| 58 | N_WBC_SS_P; N_WBC_SSFS_Area |
| 59 | N_WBC_SS_P; N_WBC_SS_CV |
| 60 | N_WBC_SS_W; N_WBC_SS_CV |
| 61 | N_WBC_SS_P; N_WBC_SS_W |
| 62 | N_WBC_SS_W; N_WBC_SSFS_Area |
| 63 | N_WBC_SS_W; N_WBC_FS_CV |
| 64 | N_WBC_SS_CV; N_WBC_FS_CV |
| 65 | N_WBC_FS_CV; N_WBC_SSFS_Area |
| 66 | N_WBC_SS_CV; N_WBC_SSFS_Area |

In addition, inflammation is divided into infectious inflammation caused by pathogenic microbial infection, and non-infectious inflammation caused by physical factors, chemical factors or tissue necrosis. The clinical symptoms of the two types of inflammation are roughly the same, and symptoms such as redness and fever will appear, but the treatment methods of the two types of inflammation are not exactly the same, so it is helpful for symptomatic treatment to clarify what factors cause the patient's inflammatory response.

To this end, the infection marker parameter is used for the identification of a non-infectious inflammation and an infectious inflammation, and the processor 140 may be further configured to determine whether the subject has an infectious inflammation or a non-infectious inflammation based on the infection marker parameter. For example, when the infection marker parameter satisfies the fifth preset condition, prompt information indicating that the subject has an infectious inflammation is output.

In some embodiments, the infection marker parameter for the identification of an infectious inflammation and a non-infectious inflammation may be one of the following parameters: N_WBC_FL_W, N_WBC_FL_P, N_WBC_SS_W, N_WBC_FS_W, N_WBC_SS_P, N_WBC_FS_P, N_WBC_FS_CV, N_WBC_SS_CV, N_WBC_FL_CV.

In other embodiments, infection marker parameters may be calculated by combining the various parameters listed in Table 6 for identification of an infectious inflammation and a non-infectious inflammation.

TABLE 6
Parameter combinations for identification of an infectious
inflammation and a non-infectious inflammation

| No. | Parameter combination |
|---|---|
| 1 | N_WBC_FL_P; N_WBC_FS_W |
| 2 | N_WBC_FL_P; N_WBC_FS_CV |
| 3 | N_WBC_SS_W; N_WBC_FL_P |
| 4 | N_WBC_FL_W; N_WBC_FS_W |
| 5 | N_WBC_SS_CV; N_WBC_FL_P |
| 6 | N_WBC_SS_W; N_WBC_FL_W |
| 7 | N_WBC_FL_W; N_WBC_FS_P |
| 8 | N_WBC_SS_P; N_WBC_FL_W |
| 9 | N_WBC_SS_CV; N_WBC_FL_W |
| 10 | N_WBC_FL_W; N_WBC_FS_CV |
| 11 | N_WBC_FL_CV; N_WBC_FS_W |
| 12 | N_WBC_FL_P; N_WBC_FL_CV |
| 13 | N_WBC_FL_P; N_WBC_FL_W |
| 14 | N_WBC_FL_W; N_WBC_FL_CV |
| 15 | N_WBC_FL_CV; N_WBC_FS_CV |
| 16 | N_WBC_SS_P; N_WBC_FL_P |
| 17 | N_WBC_SS_W; N_WBC_FL_CV |
| 18 | N_WBC_FL_P; N_WBC_FS_P |
| 19 | N_WBC_SS_W; N_WBC_FS_P |
| 20 | N_WBC_SS_CV; N_WBC_FS_P |
| 21 | N_WBC_SS_W; N_WBC_FS_W |
| 22 | N_WBC_SS_P; N_WBC_FS_W |
| 23 | N_WBC_FS_P; N_WBC_FS_CV |
| 24 | N_WBC_FS_P; N_WBC_FS_W |
| 25 | N_WBC_FS_W; N_WBC_FS_CV |
| 26 | N_WBC_SS_P; N_WBC_SS_CV |
| 27 | N_WBC_SS_W; N_WBC_SS_CV |
| 28 | N_WBC_SS_CV; N_WBC_FS_W |
| 29 | N_WBC_SS_P; N_WBC_SS_W |
| 30 | N_WBC_SS_P; N_WBC_FS_CV |
| 31 | N_WBC_SS_W; N_WBC_FS_CV |
| 32 | N_WBC_SS_CV; N_WBC_FL_CV |
| 33 | N_WBC_SS_P; N_WBC_FS_P |
| 34 | N_WBC_SS_P; N_WBC_FL_CV |
| 35 | N_WBC_FL_CV; N_WBC_FS_P |
| 36 | N_WBC_SS_CV; N_WBC_FS_CV |

After the doctor conducts consultation and physical examination on the patient, there is usually one or several preliminary disease diagnoses. Then differential diagnoses or definitive diagnosis of the disease is carried out through laboratory tests, imaging examinations, and other means. Therefore, it can be said that the doctor goes to make the laboratory checklist with the purpose. In other words, when going to make the laboratory checklist, the doctor has already clarified which scenario the parameters should be applied to. Here's an example: a fever patient in a general outpatient clinic without symptoms of organ damage sees a doctor. The doctor initially determines that it is a common infection, not a severe infection or sepsis. However, for the specific drugs to be prescribed, it needs to be clear whether it is a viral infection or a bacterial infection, so a blood routine test is prescribed. When the results come out, attention will be paid to whether the parameters are greater than the threshold of “bacterial infection VS viral infection” rather than the threshold of “diagnosis of sepsis”. Therefore, the infection marker parameters output in the disclosure are clinically used as a reference for doctors, and are not for diagnostic purposes.

Some embodiments for further ensuring the reliability of diagnosis or prompt based on infection marker parameters will be described next, although it will be understood that embodiments of the disclosure are not limited thereto.

In order to avoid the leukocyte characteristic parameter for calculating the infection marker parameter itself interfering with the reliability of diagnosis or prompt, in some embodiments, the processor 140 may be further configured to either skip outputting the value of the infection marker parameter (i.e., mask the value of the infection marker parameter) or output the value of the infection marker parameter and simultaneously output prompt information indicating that the value of the infection marker parameter is unreliable when the preset characteristic parameter of the target particle population satisfies a sixth preset condition.

When the processor 140 is further configured to output the prompt information indicating the infection status of the subject based on the infection marker parameter, if the preset characteristic parameter of the target particle population satisfies a sixth preset condition, the processor 140 does not output prompt information indicating the infection status of the subject, or outputs prompt information indicating the infection status of the subject and outputs additional information indicating that the prompt information is unreliable.

In some specific examples, the processor 140 may be configured to, when the total number of particles of the target particle population is less than a preset threshold, skip outputting the value of the infection marker parameter, or output the value of the infection marker parameter and simultaneously output prompt information indicating that the value of the infection marker parameter is unreliable.

That is to say, when the total number of particles in the target particle population is less than the preset threshold, that is, the number of particles in the target particle population is small, and the amount of information characterized by the particles is limited, the calculation results of infection marker parameters may not be reliable. For example, as shown in FIG. 8, the total number of particles of the leukocyte population in the test sample is too low, which may cause the infection marker parameter calculated from the leukocyte characteristic parameter of the leukocyte population to be unreliable.

Herein, for example, it is possible to determine whether the preset characteristic parameters of the target particle population are abnormal, for example, whether the total number of particles of the target particle population is lower than the preset threshold value, based on the optical information.

In other examples, the processor 140 may be configured to, when the target particle population overlaps with other particle populations, skip outputting prompt information indicating the infection status of the subject, skip outputting the value of the infection marker parameter, or output the value of the infection marker parameter and simultaneously output prompt information indicating that the value of the infection marker parameter is unreliable.

For example, as shown in FIG. 9, the neutrophil population in the test sample overlaps with other particles, which may cause the infection marker parameter calculated from the leukocyte characteristic parameter of the neutrophil population to be unreliable. Herein, for example, it is possible to determine whether the target particle population overlaps with other particle populations based on the optical information.

Similarly, when the processor 140 is further configured to output prompt information indicating the infection status of the subject based on the infection marker parameter, if the total number of particles of the target particle population is less than a preset threshold, and/or if the target particle population overlaps with other particle populations, the processor 140 does not output prompt information indicating the infection status of the subject, or outputs prompt information indicating the infection status of the subject and outputs additional information indicating that the prompt information is unreliable.

In addition, the disease status of the subject, as well as the abnormal cells (e.g., blast cells, abnormal lymphocytes, naïve granulocytes) in the blood of the subject, may also affect the diagnosis or prompt effectiveness of the infection marker parameters. To this end, processor 140 may be further configured to: determine the reliability of infection marker parameters based on whether the subject has a specific disease and/or based on the presence of predefined types of abnormal cells (e.g., blast cells, abnormal lymphocytes, and naïve granulocytes) in the blood sample to be tested.

In some specific examples, the processor 140 may be configured to: when the subject suffers from a hematological disorder or there are abnormal cells, especially blast cells, in the blood sample to be tested, skip outputting the value of the infection marker parameter, or output the value of the infection marker parameter and simultaneously output prompt information indicating that the value of the infection marker parameter is unreliable. It will be appreciated that an abnormal hemogram of a subject with a hematological disorder results in an unreliable prompt based on the infection marker parameter.

Processor 140 may, for example, obtain whether the subject suffers from a hematological disorder based on the subject's identity information.

In some embodiments, processor 140 may be configured to determine whether abnormal cells, in particular blast cells, are present in the blood sample to be tested based on the optical information.

In some embodiments, the processor 140 may further be configured to perform data processing, such as de-noising (as shown in FIG. 10) or logarithmic processing (as shown in FIG. 11) on the leukocyte characteristic parameters prior to calculating the infection marker parameters, in order to more accurately calculate the infection marker parameters, e.g. to avoid signal variations caused by different instruments, or different reagents.

The manner in which the processor 140 assigns a priority for each set of infection marker parameters will be described below in conjunction with some of the following embodiments.

In some embodiments, the processor 140 may be further configured to: assign a priority for each set of infection marker parameters based on at least one of infection diagnostic efficacy, parametric stability, and parametric limitations.

In some embodiments herein, the processor 140 may be further configured to: assign a priority for each set of infection marker parameters based at least on the infection diagnostic efficacy. For example, the processor 140 may assign a priority for each set of infection marker parameters based only on infection diagnostic efficacy. For still another example, the processor 140 may assign a priority for each set of infection marker parameters based on infection diagnostic efficacy and parametric stability; For yet another example, the processor 140 may assign a priority for each set of infection marker parameters based on infection diagnostic efficacy, parametric stability, and parametric limitations.

In some embodiments, the set of infection marker parameters of the disclosure may be used for evaluation of a variety of infection statuses, for example, performing on the subject an early prediction of sepsis, a diagnosis of sepsis, an identification of a common infection and a severe infection, a monitoring of infection, an analysis of sepsis prognosis, an identification of a bacterial infection and a viral infection, an evaluation of therapeutic effect on sepsis, or an identification of a non-infectious inflammation and an infectious inflammation based on the infection marker parameter. Correspondingly, taking the identification scenario of a common infection and a severe infection as an example, the diagnostic efficacy on the infection includes a diagnostic efficacy for the identification of a common infection and a severe infection. For example, when the set of infection marker parameters of the disclosure is set only for evaluation of one infection status, for example, only for severe infection identification, each set of infection marker parameters may be assigned a priority based on diagnostic efficacy for the evaluation of infection status, for example, severe infection identification.

As some implementations, the processor 140 may be further configured to: assign a priority for each set of infection marker parameters according to the area ROC_AUC enclosed by the ROC curve of each set of infection marker parameters and the horizontal coordinate axis, wherein the larger the ROC_AUC, the higher the priority of the corresponding set of infection marker parameters. In this case, the ROC curve is a receiver operating characteristic curve drawn with the true positive rate as the ordinate and the false positive rate as the abscissa. The ROC_AUC of each set of infection marker parameters may reflect the infection diagnostic efficacy of the set of infection marker parameters.

In some embodiments, the parametric stability includes at least one of numerical repeatability, aging stability, temperature stability, and inter-machine consistency. Among them, numerical repeatability refers to the consistency of the values of the set of infection marker parameters used when the same instrument is configured to perform multiple repeated tests on the same blood sample to be tested in a short period of time in the same environment; aging stability refers to the numerical stability of the set of infection marker parameters used when the same instrument is configured to test the same blood sample to be tested at different time points in the same environment; temperature stability refers to the numerical stability of the set of infection marker parameters used when the same instrument is configured to test the same blood sample to be tested under different temperature environments; and inter-machine consistency refers to the consistency of the values of the set of infection marker parameters used when different instruments are configured to test the same blood sample to be tested in the same environment.

In some examples, if the same instrument is configured to perform multiple repeated tests on the same blood sample to be tested in a short period of time in the same environment, the higher the consistency of the values of the set of infection marker parameters used, that is, the higher the numerical repeatability, the higher the priority of the set of infection marker parameters.

Alternatively or additionally, if the same instrument is configured to perform a test on the same blood sample to be tested at different time points in the same environment, the higher the stability of the value of the set of infection marker parameters used (that is, the smaller the fluctuation degree of the value), that is, the higher the aging stability, the higher the priority of the set of infection marker parameters.

Alternatively or additionally, if the same instrument is configured to perform a test on the same blood sample to be tested in different temperature environments, the higher the stability of the value of the set of infection marker parameters used (that is, the smaller the fluctuation degree of the value), that is, the higher the temperature stability, the higher the priority of the set of infection marker parameters.

Alternatively or additionally, when different instruments are configured to perform tests on the same blood sample to be tested in the same environment, the higher the consistency of the values of the set of infection marker parameters used, that is, the higher the inter-machine consistency, the higher the priority of the set of infection marker parameters.

In some embodiments, the parametric limitation refers to the range of subjects to which the infection marker parameter s applicable. In some examples, if the range of subjects to which the set of infection marker parameters is applicable is larger, it means that the parametric limitation of the set of infection marker parameters is smaller, and correspondingly, the priority of the set of infection marker parameters is higher.

In some embodiments, the priorities of the plurality of sets of infection marker parameters obtained by the processor 140 are preset, for example, based on at least one of infection diagnostic efficacy, parametric stability, and parametric limitations. Here, the processor 140 may assign a priority for each set of infection marker parameters based on the preset. For example, the priorities of the plurality of sets of infection marker parameters may be stored in a memory in advance, and the processor 140 may invoke the priorities of the plurality of sets of infection marker parameters from the memory.

Next, the manner in which the processor 140 calculates the credibility of the set of infection marker parameters will be further described in conjunction with some of the following embodiments.

The inventors of the disclosure have found through research that there may be abnormal classification results and/or abnormal cells in the blood samples of the subjects, resulting in unreliable sets of infection marker parameters used. Accordingly, the blood analyzer provided in the disclosure can calculate the credibility for the obtained plurality of sets of infection marker parameters in order to screen out a more reliable set of infection marker parameters from the plurality of sets of infection marker parameters based on the priority and credibility of each set of infection marker parameters.

In some embodiments, the processor 140 may be configured to calculate a credibility for each set of infection marker parameters as follows:

the credibility of the set of infection marker parameters is calculated from the classification result of at least one target particle population used to obtain the set of infection marker parameters and/or from the abnormal cells in the blood sample to be tested.

In some embodiments, the classification result may include at least one of a count value of the target particle population, a count value percentage of the target particle population to another particle population, and a degree of overlap (also referred to as a degree of adhesion) between the target particle population and its adjacent particle population. For example, the degree of overlap between the target particle population and its adjacent particle population may be determined by the distance between the center of gravity of the target particle population and the center of gravity of its adjacent particle population. For example, if the total number of particles of the target particle population, that is, the count value, is less than the preset threshold, that is, the particles of the target particle population are few, and the amount of information characterized by the particles is limited, at this time, the set of infection marker parameters obtained through the relevant parameters of the target particle population may be unreliable, so the credibility of the set of infection marker parameters is low.

Next, the manner in which the processor 140 screens the set of infection marker parameters will be further described in conjunction with some embodiments.

In an embodiment of the disclosure, the processor 140 may be configured to calculate credibility for all of the sets of infection marker parameters in the plurality of sets of infection marker parameters at a time, and then select at least one set of infection marker parameters from all of the sets of infection marker parameters based on the priority and credibility of all of the sets of infection marker parameters and output their parameter values.

In other embodiments, the processor 140 may be configured to perform the following steps to screen the set of infection marker parameters and output its parameter values:
obtaining a plurality of parameters of at least one target particle population in the test sample from optical information;
obtaining a plurality of sets of infection marker parameters for evaluating an infection status of the subject from the plurality of parameters;
according to the priority of the plurality of sets of infection marker parameters, successively calculating the credibility of the plurality of sets of infection marker parameters and determining whether the credibility reaches the corresponding credibility threshold;
when the credibility of the current set of infection marker parameters reaches the corresponding credibility threshold, outputting the parameter value of the set of infection marker parameters and stopping the calculation and determination.

In some embodiments, the processor 140 may be further configured to: when the parameter value of the selected set of infection marker parameters is greater than the infection positive threshold, output an alarm prompt.

Herein, for example, each set of infection marker parameters may be normalized to ensure that the infection positivity thresholds of each of the infection marker parameters are consistent.

In other embodiments, the processor 140 may be further configured to obtain a plurality of parameters of at least one target particle population in the test sample from the optical information,
obtain a plurality of sets of infection marker parameters for evaluating the infection status of the subject from the plurality of parameters,
calculate the credibility of each set of infection marker parameters of the plurality of sets of infection marker parameters, select at least one set of infection marker parameters from the plurality of sets of infection marker parameters based on the credibility of the plurality of sets of infection marker parameters and output their parameter values.

In some embodiments, the processor may be further configured to:
for each set of infection marker parameters, calculate the credibility of the set of infection marker parameters based on a classification result of at least one target particle population used to obtain the set of infection marker parameters and/or based on abnormal cells in the blood sample to be tested.

The classification result may include, for example, at least one of a count value of the target particle population, a count value percentage of the target particle population to another particle population, and a degree of overlap between the target particle population and its adjacent particle population.

Further, the processor is further configured to:
when the parameter value of the selected set of infection marker parameters is greater than the infection positive threshold, output an alarm prompt.

In other embodiments, the processor 140 may be further configured to determine, based on the optical information, whether the blood sample to be tested has abnormalities that affect the evaluation of infection status; when it is determined that there is an abnormality in the blood sample to be tested that affects the evaluation of infection status, obtain an infection marker parameter matching (i.e. unaffected by) the abnormality and used to evaluate the infection status of the subject from the optical information.

In one example, if it is determined that there is an abnormal classification result affecting the evaluation of infection status in the blood sample to be tested, for example, there is an overlap between the monocyte population and the neutrophil population in the blood sample to be tested, a plurality of parameters of other cell populations (such as lymphocyte populations) other than the monocyte population and the neutrophil population can be obtained from the optical information, and infection marker parameters for evaluating the infection status of the subject can be obtained from the plurality of parameters of the other cell populations.

In another example, if it is determined that there are abnormal cells, such as blast cells, affecting the evaluation of infection status in the blood sample to be tested, a plurality of parameters of other cell populations other than the cell populations affected by the blast cells can be obtained from the optical information, and infection marker parameters for evaluating the infection status of the subject can be obtained from the plurality of parameters of the other cell populations.

Next, the manner in which the processor 140 controls the retest will be further described in conjunction with some embodiments.

In some embodiments, the processor may be further configured to:
obtain a leukocyte count of the test sample based on the optical information before obtaining at least one leukocyte characteristic parameter of at least one target particle population in the test sample from the optical information, and output a retest instruction to retest the blood sample of the subject when the leukocyte count is less than a preset threshold, wherein the measurement amount of sample for the measurement based on the retest instruction is greater than the measurement amount of sample for the measurement to obtain the optical information; and
the processor is further configured to obtain at least another leukocyte characteristic parameter of at least another target particle population from the optical information measured based on the retest instruction, and to obtain an infection marker parameter for evaluating the infection status of the subject based on the at least another leukocyte characteristic parameter; herein, another target particle used in the retest could be the same as that used in the test, in some embodiments different from that used in the test.

The disclosure further provides yet another blood analyzer comprising a measurement device and a controller, wherein
the measurement device is configured to mix a blood sample to be tested of a subject, a hemolytic agent and a staining agent to prepare a test sample and perform optical measurement on the test sample to obtain optical information of the test sample;
the controller is configured to: receive a mode setting instruction,
when the mode setting instruction indicates that a blood routine test mode is selected, control the measurement device to optically measure a test sample at a first measurement amount to obtain optical information of the test sample, and obtain and output blood routine parameters of the test sample based on the optical information,
when the mode setting instruction indicates that a sepsis test mode is selected, control the measurement device to optically measure a test sample at a second measurement amount greater than the first measurement amount to obtain optical information of the test sample, obtain at least one leukocyte characteristic parameter of at least one target particle population in the test sample from the optical information, obtain an infection marker parameter for evaluating the infection status of the subject based on the at least one leukocyte characteristic parameter, and output the infection marker parameter.

To this end, it is possible to control the sample analyzer to perform a retest action when the leukocyte count in the sample is less than a preset threshold, resulting in unreliable test parameter results, so as to obtain more accurate infection marker parameters for evaluating the infection status of the subject.

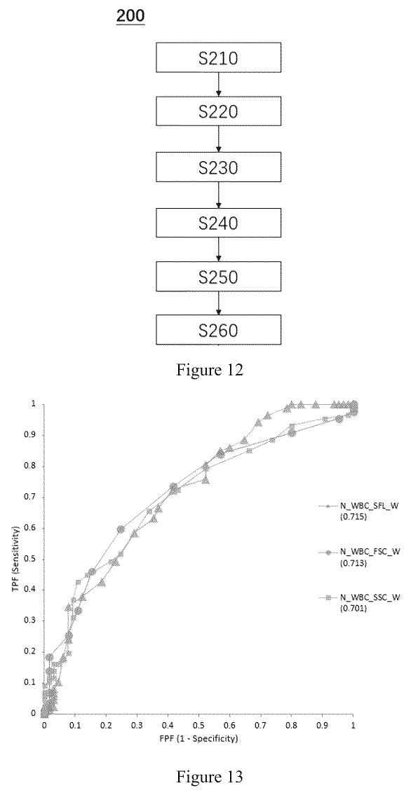

Embodiments of the disclosure also provide a method for indicating the infection status of a subject. As shown in FIG. 12, the method 200 comprises the steps of:
S210: obtaining a blood sample to be tested collected from the subject;
S220: preparing a test sample containing the blood sample to be tested, a hemolytic agent, and a staining agent for identifying nucleated red blood cells;
S230: passing the particles in the test sample through the optical detection region irradiated by light one by one to obtain optical information generated by the particles in the test sample after being irradiated by light;
S240: obtaining at least one leukocyte characteristic parameter of at least one target particle population in the test sample from the optical information;
S250: calculating an infection marker parameter based on the at least one leukocyte characteristic parameter; and
S260: evaluating the infection status of the subject based on the infection marker parameter and optionally outputting prompt information indicating the infection status of the subject.

The method 200 provided in the embodiment of the disclosure is implemented, in particular, by the blood cell analyzer 100 described above in the embodiment of the disclosure.

In some embodiments, the method may further comprise: identifying nucleated red blood cells in the test sample based on the optical information to obtain a nucleated red blood cell count.

In some embodiments, the at least one target particle population may be selected from one or more of a leukocyte population, a neutrophil population, a lymphocyte population; in some embodiments the at least one target particle population comprises a leukocyte population and/or a neutrophil population.

In some embodiments, the infection marker parameter may be selected from one of the following cell characteristic parameters or may be obtained from a combination of a plurality of cell characteristic parameters of the following cell characteristic parameters, in particular from a combination by a linear function:
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, a side fluorescence intensity distribution coefficient of variation of the leukocyte population;
an area of a distribution region of the leukocyte population in a two-dimensional scattergram generated by two light intensities of a forward scatter intensity, a side scatter intensity, and a side fluorescence intensity, a volume of a distribution region of the leukocyte population in a three-dimensional scattergram generated by a forward scatter intensity, a side scatter intensity, and a fluorescence intensity;
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, a side fluorescence intensity distribution coefficient of variation of the neutrophil population;
an area of a distribution region of the neutrophil population in a two-dimensional scattergram generated by two light intensities of a forward scatter intensity, a side scatter intensity, and a side fluorescence intensity, a volume of a distribution region of the neutrophil population in a three-dimensional scattergram generated by a forward scatter intensity, a side scatter intensity, and a fluorescence intensity;
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, a side fluorescence intensity distribution coefficient of variation of the lymphocyte population; and
an area of a distribution region of the lymphocyte population in a two-dimensional scattergram generated by two light intensities of a forward scatter intensity, a side scatter intensity, and a side fluorescence intensity, a volume of a distribution region of the lymphocyte population in a three-dimensional scattergram generated by a forward scatter intensity, a side scatter intensity, and a fluorescence intensity.

In some embodiments, evaluating the infection status of the subject based on the infection marker parameters may comprise: performing an early prediction of sepsis, a diagnosis of sepsis, an identification of a common infection and a severe infection, a monitoring of infection, an analysis of sepsis prognosis, an identification of a bacterial infection and a viral infection, or an identification of a non-infectious inflammation and an infectious inflammation based on the infection marker parameters.

In some embodiments, step S260 may comprise: when the infection marker parameter satisfies the first preset condition, outputting prompt information indicating that the subject is likely to progress to sepsis within a certain period of time after the blood sample to be tested is collected; in some embodiments, the certain period of time is not greater than 48 hours, especially within 24 hours.

In some embodiments, step S260 may comprise: when the infection marker parameter satisfies a second preset condition, outputting prompt information indicating that the subject has sepsis.

In some embodiments, step S260 may comprise: when the infection marker parameter satisfies a third preset condition, outputting prompt information indicating that the subject has a severe infection.

In some embodiments, the subject is an infected patient, in particular a patient with a severe infection or a patient with sepsis. Correspondingly, step S260 may comprise: monitoring the progression of the infection of the subject based on the infection marker parameter.

In some specific examples, monitoring the progression of the infection of the subject based on the infection marker parameters comprises:
obtaining values of the infection marker parameter obtained by consecutive multiple tests of blood samples from subjects at different time points;
determining whether the infection status of the patient is improving or not according to the trend of changes in the values of the infection marker parameter obtained through the consecutive multiple tests, in some embodiments, when the value of the infection marker parameter obtained by the consecutive multiple tests gradually tends to decrease, output prompt information indicating that the condition of the subject is improving.

In other examples, monitoring the progression of the infection of the subject based on the infection marker parameter comprises:
obtaining a current value of the infection marker parameter obtained by a current detection of a current blood sample from a subject, and obtaining a prior value of the infection marker parameter obtained by a previous detection of a previous blood sample from the subject, such as a prior value obtained in a blood routine test on the previous day; and
monitoring the progression of the infection status of the patient based on a comparison of the prior value of the infection marker parameter with a first threshold and a comparison of the prior value of the infection marker parameter with the current value of the infection marker parameter.

In addition, the subject may be a treated septic patient. Correspondingly, step S260 may comprise: determining whether the sepsis prognosis of the subject is good or not based on the infection marker parameter. For example, when the infection marker parameter satisfies the fourth preset condition, output prompt information indicating that the sepsis prognosis of the subject is good.

In some embodiments, step S260 may comprise: determining whether the infection type of the subject is a viral infection or a bacterial infection based on the infection marker parameter.

In some embodiments, step S260 may comprise: determining whether the subject has an infectious inflammation or a non-infectious inflammation based on the infection marker parameter. For example, when the infection marker parameter satisfies the fifth preset condition, prompt information indicating that the subject has an infectious inflammation is output.

In some embodiments, the method may further comprise: when a preset characteristic parameter of a target particle population satisfies a sixth preset condition, such as when the total number of particles of the target particle population is less than a preset threshold and/or when the target particle population overlaps with other particle populations, skipping outputting the value of the infection marker parameter, or outputting the value of the infection marker parameter and simultaneously outputting prompt information indicating that the value of the infection marker parameter is unreliable.

Alternatively or additionally, the method may further comprise: when the subject suffers from a hematological disorder or there are abnormal cells, especially blast cells, in the blood sample to be tested, such as when it is determined that there are abnormal cells, especially blast cells, in the blood sample to be tested based on the optical information, skip outputting a value of the infection marker parameter, or output a value of the infection marker parameter and simultaneously output prompt information indicating that the value of the infection marker parameter is unreliable.

Further embodiments and advantages of the method 200 provided by the embodiment of the disclosure may be referred to in the above description of the blood cell analyzer 100 provided by the embodiment of the disclosure, in particular the description of the method and steps performed by the processor 140, and will not be described here in detail.

Embodiments of the disclosure also provide a use of an infection marker parameter in evaluating the infection status of a subject, wherein the infection marker parameter is obtained by:
obtaining at least one leukocyte characteristic parameter of at least one target particle population obtained by flow cytometry detection of a test sample containing a blood sample to be tested from the subject, a hemolytic agent, and a staining agent for identifying nucleated red blood cells; and
calculating an infection marker parameter based on the at least one leukocyte characteristic parameter.

Further embodiments and advantages of the use of the infection marker parameters provided by the embodiments of the disclosure in evaluating the infection status of a subject may be referred to in the above description of the blood cell analyzer 100 provided by the embodiments of the disclosure, and in particular the description of the methods and steps performed by the processor 140, and will not be repeated herein.

Next, the disclosure and its advantages will be further explained with some specific examples.

The true positive rate %, false positive rate %, true negative rate %, and false negative rate % of the embodiment of the disclosure are calculated by the following formulas:

$$True positive rate \% = T P / ( T P + F N ) \times 100 \% ;$$

$$True negative rate \% = T N / ( F P + T N ) \times 100 \% ;$$

$$False positive rate \% = 1 - true negative rate \% ; and$$

$$False negative rate \% = 1 - true positive rate \% ;$$

wherein TP is the number of true positive individuals, FP is the number of false positive individuals, TN is the number of true negative individuals, and FN is the number of false negative individuals.

### Example 1 Early Prediction of Sepsis

Using the BC-6800 Plus blood cell analyzer produced by SHENZHEN MINDRAY BIO-MEDICAL ELECTRONICS CO., LTD., using the supporting hemolytic agents M-60LD and M-6LN and staining agents M-6FD and M-6FN of SHENZHEN MINDRAY BIO-MEDICAL ELECTRONICS CO., LTD., the blood samples from 152 donors were tested by blood routine test, and the scattergrams of WNB channels and DIFF channels were obtained, and early prediction of sepsis was performed according to the method provided in the embodiment of the disclosure. The next day, among these samples, 87 blood samples were clinically diagnosed as positive samples for sepsis and 65 blood samples were negative samples (without progressing to sepsis).

Inclusion criteria for these 152 donors: adult ICU patients with or without acute infection. Exclusion criteria: pregnant people, myelosuppressed people on chemotherapy, people on immunosuppressant treatment, patients with hematologic diseases.

For the donors of the sepsis samples: they have a suspicious or definite infection site, a positive laboratory culture result, and organ failure; they have suspicious or confirmed acute infection, and SOFA score ≥2, where the suspected infection has any of the following (1)-(3) and has no deterministic results for (4); or has any one of the following (1)-(3) and (5).
(1) Acute (within 72 hours) fever or hypothermia;
(2) Increased or decreased total number of leukocytes;
(3) Increased CRP and IL-6;
(4) Increased PCT, SAA and HBP;
(5) Presence of suspicious infection sites.

The SOFA scoring criteria are shown in the Table A below:

TABLE A
SOFA score calculation method

| Organ | Variable | Score 0 | Score 1 | Score 2 | Score 3 | Score 4 |
|---|---|---|---|---|---|---|
| Respiratory system | [symbol] | [symbol] | [symbol] | [symbol] | [symbol] | [symbol] |
| Blood system | [symbol] | [symbol] | [symbol] | [symbol] | [symbol] | [symbol] |
| Liver | Bilirubin [symbol] | [symbol] | [symbol] | [symbol] | [symbol] | [symbol] |
| Central nervous system | [symbol] Score | [symbol] | [symbol] | [symbol] | [symbol] | [symbol] |
| Kidney | Creatinine [symbol] | [symbol] | [symbol] | [symbol] | [symbol] | [symbol] |
| | Urine volume [symbol] | [symbol] | [symbol] | [symbol] | [symbol] | [symbol] |
| Circulation | Mean arterial pressure [symbol] | [symbol] | [symbol] | [symbol] | [symbol] | [symbol] |
| | Dopamine [symbol] | [symbol] | [symbol] | [symbol] | [symbol] | [symbol] |
| | Dobutamine | Any dose | | | | |
| | Epinephrine [symbol] | [symbol] | [symbol] | [symbol] | [symbol] | [symbol] |
| | Norepinephrine [symbol] | [symbol] | [symbol] | [symbol] | [symbol] | [symbol] |

Note [symbol] [symbol]
indicates data missing or illegible when filed

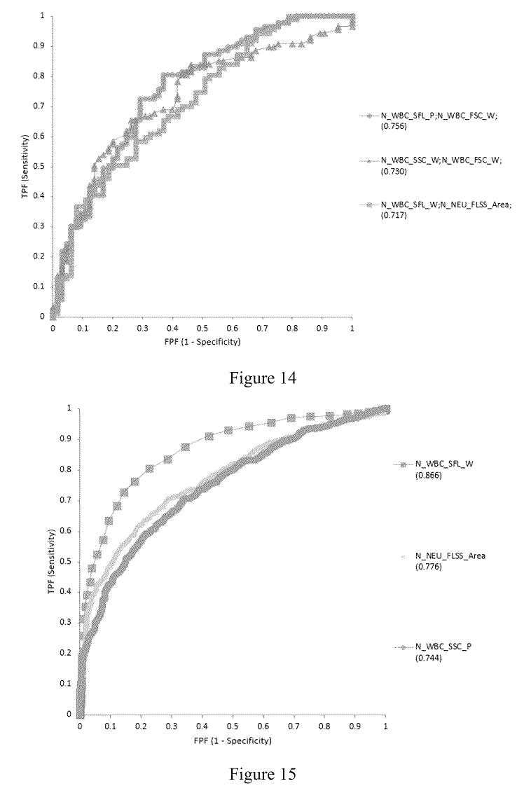

Table 7 shows the infection marker parameters used and their corresponding diagnostic efficacy, and FIGS. 13 and 14 show ROC curves corresponding to the infection marker parameters in Table 7. In Table 7:

$$Combination parameter 1 = 0.00174639 \times N\_WBC \_FL \_P + 0.00788 \; 254 \times N\_WBC \_FS \_W - 10.4569 ;$$

$$Combination parameter 2 = 0.00160514 \times N\_WBC \_SS \_W + 0.00480 \; 886 \times N\_WBC \_FS \_W - 6.62685 ;$$

$$Combination parameter 3 = 0.00278754 \times N\_WBC \_FL \_W + 0.00010 \; 201 \times N\_NEU \_FLSS \_Area .$$

TABLE 7
Efficacy of different infection marker parameters for early prediction of sepsis risk

| Infection | | | False | True | True | False |
|---|---|---|---|---|---|---|
| marker | | Determination | positive | positive | negative | negative |
| parameter | ROC_AUC | threshold | rate | rate | rate | rate |
| N_WBC_FL_W | 0.7148 | >1936 | 36.90% | 66.70% | 63.10% | 33.30% |
| N_WBC_FS_W | 0.7131 | >976 | 24.60% | 59.80% | 75.40% | 40.20% |
| N_WBC_SS_W | 0.7014 | >1328 | 33.80% | 65.50% | 66.20% | 34.50% |
| Combination | 0.7556 | >0.2094 | 29.20% | 72.40% | 70.8% | 27.6% |
| parameter 1 | | | | | | |
| Combination | 0.73 | >0.1726 | 26.20% | 65.50% | 73.8% | 34.5% |
| parameter 2 | | | | | | |
| Combination | 0.7169 | >0.3718 | 27.70% | 58.60% | 72.3% | 41.4% |
| parameter 3 | | | | | | |

In addition, Tables 8-1 to 8-4 show the efficacy of using other parameter combinations for early prediction of sepsis risk in this example, wherein infection marker parameters are calculated by the function Y=A×X1+B×X2+C based on the parameter combinations in the table, where Y represents an infection marker parameter, X1 represents the first leukocyte parameter, X2 represents the second leukocyte parameter, and A, B, and C are constants.

TABLE 8-1
Efficacy of parameter combinations containing N_WBC_SS_W for early prediction of sepsis risk

| | | | False | True | True | False | | | |
|---|---|---|---|---|---|---|---|---|---|
| Parameter | ROC_{—} | Determination | positive | positive | negative | negative | | | |
| combination | AUC | threshold | rate % | rate % | rate % | rate % | A | B | C |
| N_WBC_SS_W; | 0.7537 | >−0.0125 | 33.8 | 77 | 66.2 | 23 | 0.003131 | 0.001778 | −7.16465 |
| N_WBC_FL_P; | | | | | | | | | |
| N_WBC_SS_W; | 0.7507 | >0.2234 | 26.2 | 65.5 | 73.8 | 34.5 | 0.003018 | 0.001656 | −7.04123 |
| N_NEU_FL_P; | | | | | | | | | |
| N_WBC_SS_W; | 0.7424 | >0.3728 | 23.1 | 62.1 | 76.9 | 37.9 | 0.001917 | 0.002552 | −7.32822 |
| N_WBC_FL_W; | | | | | | | | | |
| N_WBC_SS_W; | 0.7415 | >0.0522 | 36.9 | 75.9 | 63.1 | 24.1 | 0.003743 | −3.98225 | −1.85707 |
| N_NEU_FL_CV; | | | | | | | | | |
| N_WBC_SS_W; | 0.7335 | >0.1643 | 26.2 | 66.7 | 73.8 | 33.3 | 0.008768 | −0.00609 | −4.3112 |
| N_NEU_SS_W; | | | | | | | | | |
| N_WBC_SS_W; | 0.73 | >0.1726 | 26.2 | 65.5 | 73.8 | 34.5 | 0.001605 | 0.004809 | −6.62685 |
| N_WBC_FS_W; | | | | | | | | | |
| N_WBC_SS_W; | 0.7215 | >0.0414 | 38.5 | 78.2 | 61.5 | 21.8 | 0.002676 | 0.004245 | −8.93893 |
| N_WBC_FS_P; | | | | | | | | | |
| N_WBC_SS_W; | 0.7202 | >0.2036 | 27.7 | 65.5 | 72.3 | 34.5 | 0.002546 | 0.003443 | −8.17848 |
| N_NEU_FS_P; | | | | | | | | | |
| N_WBC_SS_W; | 0.7154 | >0.0226 | 36.9 | 71.3 | 63.1 | 28.7 | 0.003798 | −0.00018 | −3.35203 |
| N_WBC_SSFS_{—} | | | | | | | | | |
| Area; | | | | | | | | | |
| N_WBC_SS_W; | 0.7143 | >0.1463 | 30.8 | 70.1 | 69.2 | 29.9 | 0.004076 | −2.37943 | −2.87791 |
| N_NEU_SS_CV; | | | | | | | | | |
| N_WBC_SS_W; | 0.7099 | >0.0832 | 30.8 | 67.8 | 69.2 | 32.2 | 0.003508 | −0.00015 | −3.52294 |
| N_NEU_SSFS_{—} | | | | | | | | | |
| Area; | | | | | | | | | |
| N_WBC_SS_P; | 0.7059 | >0.1788 | 27.7 | 63.2 | 72.3 | 36.8 | 0.000725 | 0.002613 | −4.1775 |
| N_WBC_SS_W; | | | | | | | | | |
| N_WBC_SS_W; | 0.7054 | >0.0886 | 36.9 | 73.6 | 63.1 | 26.4 | 0.00242 | 0.001401 | −4.72465 |
| N_NEU_SS_P; | | | | | | | | | |
| N_WBC_SS_W; | 0.7019 | >0.2261 | 23.1 | 58.6 | 76.9 | 41.4 | 0.002208 | 0.00134 | −4.65695 |
| N_NEU_FL_W; | | | | | | | | | |
| N_WBC_SS_W; | 0.7012 | >0.2122 | 26.2 | 62.1 | 73.8 | 37.9 | 0.002254 | 0.000137 | −4.17901 |
| N_WBC_FLFS_{—} | | | | | | | | | |
| Area; | | | | | | | | | |
| N_WBC_SS_W; | 0.6998 | >0.1905 | 26.2 | 59.8 | 73.8 | 40.2 | 0.002385 | 0.001727 | −4.15841 |
| N_NEU_FS_W; | | | | | | | | | |
| N_WBC_SS_W; | 0.6993 | >0.1424 | 30.8 | 66.7 | 69.2 | 33.3 | 0.002271 | 6.77E−05 | −3.77935 |
| N_WBC_FLSS_{—} | | | | | | | | | |
| Area; | | | | | | | | | |
| N_WBC_SS_W; | 0.6985 | >0.3004 | 20 | 57.5 | 80 | 42.5 | 0.002302 | 0.000158 | −4.10915 |
| N_NEU_FLFS_{—} | | | | | | | | | |
| Area; | | | | | | | | | |
| N_WBC_SS_W; | 0.6966 | >0.1819 | 29.2 | 60.9 | 70.8 | 39.1 | 0.002399 | 6.12E−05 | −3.72422 |
| N_NEU_FLSS_{—} | | | | | | | | | |
| Area; | | | | | | | | | |
| N_WBC_SS_W; | 0.6945 | >0.2437 | 21.5 | 55.2 | 78.5 | 44.8 | 0.002481 | 2.321197 | −4.21839 |
| N_NEU_FS_CV; | | | | | | | | | |

TABLE 8-2
Efficacy of parameter combinations containing N_WBC_FL_W for early prediction of sepsis risk

| | | | False | True | True | False | | | |
|---|---|---|---|---|---|---|---|---|---|
| Parameter | ROC_{—} | Determination | positive | positive | negative | negative | | | |
| combination | AUC | threshold | rate % | rate % | rate % | rate % | A | B | C |
| N_WBC_SS_W; | 0.7424 | >0.3728 | 23.1 | 62.1 | 76.9 | 37.9 | 0.001917 | 0.002552 | −7.32822 |
| N_WBC_FL_W; | | | | | | | | | |
| N_WBC_FL_W; | 0.7422 | >0.1961 | 32.3 | 70.1 | 67.7 | 29.9 | 0.002365 | 0.00454 | −8.73389 |
| N_WBC_FS_W; | | | | | | | | | |
| N_WBC_FL_W; | 0.7309 | >0.4337 | 26.2 | 59.8 | 73.8 | 40.2 | 0.002872 | 0.001769 | −6.45903 |
| N_NEU_FS_W; | | | | | | | | | |
| N_WBC_FL_W; | 0.7308 | >0.3632 | 27.7 | 62.1 | 72.3 | 37.9 | 0.002753 | 0.001552 | −6.98478 |
| N_NEU_SS_W; | | | | | | | | | |
| N_WBC_FL_W; | 0.7283 | >0.4442 | 27.7 | 58.6 | 72.3 | 41.4 | 0.002945 | 2.347587 | −6.51489 |
| N_NEU_FS_CV; | | | | | | | | | |
| N_WBC_FL_W; | 0.7231 | >0.5284 | 21.5 | 56.3 | 78.5 | 43.7 | 0.003006 | 2.145841 | −7.79154 |
| N_NEU_SS_CV; | | | | | | | | | |
| N_WBC_FL_W; | 0.7222 | >0.0659 | 40 | 77 | 60 | 23 | 0.003122 | 0.004524 | −11.6565 |
| N_WBC_FS_P; | | | | | | | | | |
| N_WBC_FL_W; | 0.7217 | >0.3594 | 27.7 | 62.1 | 72.3 | 37.9 | 0.002922 | 0.000164 | −6.54216 |
| N_NEU_SSFS_{—} | | | | | | | | | |
| Area; | | | | | | | | | |
| N_WBC_SS_P; | 0.7188 | >0.3229 | 29.2 | 63.2 | 70.8 | 36.8 | 0.002863 | 0.002823 | −8.52992 |
| N_WBC_FL_W; | | | | | | | | | |
| N_WBC_FL_W; | 0.7187 | >0.3181 | 32.3 | 66.7 | 67.7 | 33.3 | 0.002999 | 0.004475 | −11.9545 |
| N_NEU_FS_P; | | | | | | | | | |
| N_WBC_FL_W; | 0.7185 | >0.2007 | 35.4 | 71.3 | 64.6 | 28.7 | 0.002771 | 0.002889 | −8.51787 |
| N_NEU_SS_P; | | | | | | | | | |
| N_WBC_FL_W; | 0.7174 | >0.4201 | 27.7 | 60.9 | 72.3 | 39.1 | 0.002819 | 0.000144 | −6.28811 |
| N_NEU_FLFS_{—} | | | | | | | | | |
| Area; | | | | | | | | | |
| N_WBC_FL_W; | 0.7169 | >0.3718 | 27.7 | 58.6 | 72.3 | 41.4 | 0.002788 | 0.000102 | −6.27365 |
| N_NEU_FLSS_{—} | | | | | | | | | |
| Area; | | | | | | | | | |
| N_WBC_FL_W; | 0.7148 | >0.1976 | 38.5 | 72.4 | 61.5 | 27.6 | 0.003453 | −0.00021 | −6.01757 |
| N_NEU_FL_P; | | | | | | | | | |
| N_WBC_FL_W; | 0.7147 | >0.2599 | 35.4 | 69 | 64.6 | 31 | 0.002953 | 0.000119 | −6.52304 |
| N_WBC_SSFS_{—} | | | | | | | | | |
| Area; | | | | | | | | | |
| N_WBC_FL_W; | 0.7146 | >0.1977 | 40 | 72.4 | 60 | 27.6 | 0.00325 | 0.172788 | −6.15783 |
| N_NEU_FL_CV; | | | | | | | | | |
| N_WBC_FL_P; | 0.7141 | >0.2035 | 38.5 | 72.4 | 61.5 | 27.6 | −7.8E−05 | 0.003294 | −5.97341 |
| N_WBC_FL_W; | | | | | | | | | |
| N_WBC_FL_W; | 0.7134 | >0.1929 | 36.9 | 70.1 | 63.1 | 29.9 | 0.00281 | 0.000108 | −6.23476 |
| N_WBC_FLFS_{—} | | | | | | | | | |
| Area; | | | | | | | | | |
| N_WBC_FL_W; | 0.7124 | >0.4656 | 26.2 | 58.6 | 73.8 | 41.4 | 0.002819 | 0.000925 | −6.48923 |
| N_NEU_FL_W; | | | | | | | | | |
| N_WBC_FL_W; | 0.7103 | >0.2249 | 36.9 | 67.8 | 63.1 | 32.2 | 0.002758 | 7.56E−05 | −6.09636 |
| N_WBC_FLSS_{—} | | | | | | | | | |
| Area; | | | | | | | | | |

TABLE 8-3
Efficacy of parameter combinations containing N_WBC_FS_W for early prediction of sepsis risk

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_WBC_FL_P; | 0.7556 | >0.2094 | 29.2 | 72.4 | 70.8 | 27.6 | 0.001746 | 0.007883 | −10.4569 |
| N_WBC_FS_W; | | | | | | | | | |
| N_WBC_FS_W; | 0.7517 | >0.3833 | 21.5 | 67.8 | 78.5 | 32.2 | 0.010195 | −4.6007 | −6.13933 |
| N_NEU_FL_CV; | | | | | | | | | |
| N_WBC_FS_W; | 0.7515 | >0.2796 | 27.7 | 70.1 | 72.3 | 29.9 | 0.007732 | 0.001665 | −10.416 |
| N_NEU_FL_P; | | | | | | | | | |
| N_WBC_FL_W; | 0.7422 | >0.1961 | 32.3 | 70.1 | 67.7 | 29.9 | 0.002365 | 0.00454 | −8.73389 |
| N_WBC_FS_W; | | | | | | | | | |
| N_WBC_SS_W; | 0.73 | >0.1726 | 26.2 | 65.5 | 73.8 | 34.5 | 0.001605 | 0.004809 | −6.62685 |
| N_WBC_FS_W; | | | | | | | | | |
| N_WBC_FS_W; | 0.7241 | >0.1496 | 32.3 | 71.3 | 67.7 | 28.7 | 0.005984 | 0.002454 | −8.45397 |
| N_NEU_SS_P; | | | | | | | | | |
| N_WBC_SS_P; | 0.7238 | >0.2018 | 29.2 | 69 | 70.8 | 31 | 0.002133 | 0.006189 | −8.23148 |
| N_WBC_FS_W; | | | | | | | | | |
| N_WBC_FS_W; | 0.7205 | >0.2076 | 27.7 | 63.2 | 72.3 | 36.8 | 0.00578 | 0.000976 | −6.55936 |
| N_NEU_SS_W; | | | | | | | | | |
| N_WBC_FS_W; | 0.7204 | >0.2231 | 24.6 | 63.2 | 75.4 | 36.8 | 0.008262 | −0.00088 | −7.18548 |
| N_NEU_FS_W; | | | | | | | | | |
| N_WBC_FS_W; | 0.72 | >0.2139 | 24.6 | 63.2 | 75.4 | 36.8 | 0.007844 | −0.87663 | −6.95753 |
| N_NEU_FS_CV; | | | | | | | | | |
| N_WBC_FS_W; | 0.7185 | >0.2323 | 26.2 | 64.4 | 73.8 | 35.6 | 0.006506 | 0.000529 | −6.79983 |
| N_NEU_FL_W; | | | | | | | | | |
| N_WBC_FS_W; | 0.7179 | >0.2189 | 29.2 | 65.5 | 70.8 | 34.5 | 0.00632 | 7.62E−05 | −6.62271 |
| N_WBC_FLFS_Area; | | | | | | | | | |
| N_WBC_FS_W; | 0.7178 | >0.202 | 27.7 | 66.7 | 72.3 | 33.3 | 0.006171 | 5.37E−05 | −6.46005 |
| N_WBC_FLSS_Area; | | | | | | | | | |
| N_WBC_FS_W; | 0.7164 | >0.2297 | 26.2 | 64.4 | 73.8 | 35.6 | 0.008425 | −0.00011 | −7.13369 |
| N_NEU_SSFS_Area; | | | | | | | | | |
| N_WBC_FS_W; | 0.7146 | >0.2084 | 26.2 | 64.4 | 73.8 | 35.6 | 0.006676 | 3.12E−05 | −6.57266 |
| N_NEU_FLSS_Area; | | | | | | | | | |
| N_WBC_FS_W; | 0.7141 | >0.1445 | 33.8 | 69 | 66.2 | 31 | 0.008703 | −0.00012 | −7.11313 |
| N_WBC_SSFS_Area; | | | | | | | | | |
| N_WBC_FS_P; | 0.7139 | >0.2272 | 26.2 | 64.4 | 73.8 | 35.6 | 0.001759 | 0.006869 | −8.69703 |
| N_WBC_FS_W; | | | | | | | | | |
| N_WBC_FS_W; | 0.7129 | >0.1677 | 33.8 | 70.1 | 66.2 | 29.9 | 0.006802 | 3.99E−05 | −6.65063 |
| N_NEU_FLFS_Area; | | | | | | | | | |
| N_WBC_FS_W; | 0.7126 | >0.1867 | 24.6 | 63.2 | 75.4 | 36.8 | 0.007298 | −0.00032 | −6.37387 |
| N_NEU_FS_P; | | | | | | | | | |
| N_WBC_FS_W; | 0.7124 | >0.3501 | 21.5 | 59.8 | 78.5 | 40.2 | 0.006528 | 0.913928 | −7.03193 |
| N_NEU_SS_CV; | | | | | | | | | |

TABLE 8-4
Efficacy of the remaining parameter combinations for early prediction of sepsis risk

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_NEU_FL_P; | 0.7388 | >0.2465 | 32.3 | 70.1 | 67.7 | 29.9 | 0.001614 | 0.003473 | −5.07104 |
| N_NEU_FS_W; | | | | | | | | | |
| N_NEU_FL_P; | 0.7363 | >0.2603 | 32.3 | 69 | 67.7 | 31 | 0.001611 | 4.665652 | −4.91734 |
| N_NEU_FS_CV; | | | | | | | | | |
| N_WBC_FL_P; | 0.736 | >0.1707 | 32.3 | 69 | 67.7 | 31 | 0.001848 | 0.002828 | −6.45434 |
| N_NEU_SS_W; | | | | | | | | | |
| N_WBC_FL_P; | 0.736 | >0.3229 | 26.2 | 64.4 | 73.8 | 35.6 | 0.00165 | 0.003499 | −4.90998 |
| N_NEU_FS_W; | | | | | | | | | |
| N_WBC_FL_P; | 0.7355 | >0.1829 | 36.9 | 74.7 | 63.1 | 25.3 | 0.001652 | 4.727271 | −4.77736 |
| N_NEU_FS_CV; | | | | | | | | | |
| N_NEU_SS_W; | 0.7344 | >0.2809 | 24.6 | 62.1 | 75.4 | 37.9 | 0.002733 | 0.001743 | −6.41208 |
| N_NEU_FL_P; | | | | | | | | | |
| N_NEU_FL_CV; | 0.7267 | >0.2428 | 32.3 | 71.3 | 67.7 | 28.7 | −3.26098 | 0.00429 | −0.05185 |
| N_NEU_FS_W; | | | | | | | | | |
| N_WBC_FL_P; | 0.7243 | >0.4112 | 20 | 58.6 | 80 | 41.4 | 0.002091 | 0.000391 | −6.90332 |
| N_WBC_SSFS_Area; | | | | | | | | | |
| N_WBC_FL_P; | 0.7243 | >0.3839 | 21.5 | 60.9 | 78.5 | 39.1 | 0.001973 | 0.000435 | −6.22265 |
| N_NEU_SSFS_Area; | | | | | | | | | |
| N_WBC_FL_P; | 0.7231 | >0.353 | 24.6 | 63.2 | 75.4 | 36.8 | 0.002029 | 4.146446 | −7.59235 |
| N_NEU_SS_CV; | | | | | | | | | |
| N_NEU_FL_P; | 0.7222 | >0.2542 | 32.3 | 65.5 | 67.7 | 34.5 | 0.001881 | 0.000423 | −6.25673 |
| N_NEU_SSFS_Area; | | | | | | | | | |
| N_NEU_SS_W; | 0.7206 | >0.1167 | 36.9 | 69 | 63.1 | 31 | 0.003532 | −4.25774 | −0.83821 |
| N_NEU_FL_CV; | | | | | | | | | |
| N_NEU_SS_CV; | 0.7201 | >0.3197 | 27.7 | 62.1 | 72.3 | 37.9 | 4.004842 | 0.001922 | −7.54159 |
| N_NEU_FL_P; | | | | | | | | | |
| N_WBC_FL_P; | 0.7162 | >0.1379 | 40 | 74.7 | 60 | 25.3 | 0.001638 | 0.000335 | −5.87053 |
| N_WBC_FLFS_Area; | | | | | | | | | |
| N_WBC_FL_P; | 0.7162 | >0.4325 | 24.6 | 56.3 | 75.4 | 43.7 | 0.00173 | 0.00025 | −5.55311 |
| N_NEU_FLSS_Area; | | | | | | | | | |
| N_NEU_FL_P; | 0.7162 | >0.2467 | 36.9 | 66.7 | 63.1 | 33.3 | 0.001658 | 0.000244 | −5.60632 |
| N_NEU_FLSS_Area; | | | | | | | | | |
| N_WBC_FLFS_Area; | 0.7144 | >0.1178 | 41.5 | 72.4 | 58.5 | 27.6 | 0.000312 | 0.001501 | −5.62125 |
| N_NEU_FL_P; | | | | | | | | | |
| N_WBC_SSFS_Area; | 0.7144 | >0.3249 | 29.2 | 62.1 | 70.8 | 37.9 | 0.000361 | 0.001907 | −6.59162 |
| N_NEU_FL_P; | | | | | | | | | |
| N_WBC_FL_P; | 0.713 | >0.085 | 43.1 | 74.7 | 56.9 | 25.3 | 0.001668 | 0.000215 | −5.55784 |
| N_WBC_FLSS_Area; | | | | | | | | | |
| N_NEU_FL_CV; | 0.7118 | >0.2911 | 29.2 | 65.5 | 70.8 | 34.5 | −3.01293 | 5.637929 | −0.00259 |
| N_NEU_FS_CV; | | | | | | | | | |
| N_NEU_FL_P; | 0.7116 | >0.3444 | 30.8 | 64.4 | 69.2 | 35.6 | 0.00148 | 0.00308 | −6.85343 |
| N_NEU_FL_W; | | | | | | | | | |
| N_NEU_FL_P; | 0.7111 | >0.1248 | 38.5 | 74.7 | 61.5 | 25.3 | 0.001571 | 0.000388 | −5.66478 |
| N_NEU_FLFS_Area; | | | | | | | | | |
| N_WBC_FL_P; | 0.7103 | >0.4014 | 26.2 | 63.2 | 73.8 | 36.8 | 0.001545 | 0.003174 | −6.88047 |
| N_NEU_FL_W; | | | | | | | | | |
| N_NEU_FL_CV; | 0.71 | >0.0249 | 43.1 | 80.5 | 56.9 | 19.5 | −4.62372 | 0.000359 | −0.21684 |
| N_NEU_FLSS_Area; | | | | | | | | | |

TABLE 8-5
Efficacy of PCT (procalcitonin) of prior art and the parameters
of the DIFF channel for early prediction of sepsis risk;

| | | | False | True | True | False |
|---|---|---|---|---|---|---|
| Infection marker | | Determination | positive | positive | negative | negative |
| parameter | ROC_AUC | threshold | rate | rate | rate | rate |
| PCT | 0.634 | >2 | 14.0% | 39.7% | 86.0% | 60.3% |
| (procalcitonin); | | | | | | |
| D_Neu_SS_W | 0.613 | >253 | 47.7% | 67.8% | 52.3% | 32.2% |
| D_Neu_FL_W | 0.633 | >205 | 47.7% | 72.4% | 52.3% | 27.6% |
| D_Neu_FS_W | 0.543 | >559 | 32.3% | 48.3% | 67.7% | 51.7% |

From the comparison between Table 8-5 and Table 8-1, 8-2, 8-3, 8-4, it can be seen that parameters of WNB channel have better diagnostic performance than the parameters of DIFF channel and PCT for sepsis prediction. D_Neu_SS_W in the table refers to the side scatter intensity distribution width of the neutrophil population in the DIFF channel scattergram; D_Neu_FL_W refers to the fluorescence intensity distribution width of the neutrophil population in the DIFF channel scattergram; D_Neu_FS_W refers to the forward scatter intensity distribution width of the neutrophil population in the DIFF channel scattergram.

TABLE 8-6
Illustration of the statistical methods and testing methods
used in this example by taking 3 parameters as examples

| Infection | | | | |
|---|---|---|---|---|
| marker | Positive sample | Negative sample | | |
| parameter | Mean ± SD | Mean ± SD | F value | P value |
| N_WBC_FL_W | 2035.9 ± 220.6 | 1851.6 ± 262.4 | 22.06 | <0.0001. |
| N_WBC_FS_W | 1010.0 ± 109.7 | 941.3 ± 78.6 | 18.43 | <0.0001. |
| N_WBC_SS_W | 1467.2 ± 279.5 | 1307.1 ± 169.8 | 16.71 | <0.0001. |

As can be seen from Table 8-6, this parameter is analyzed by Welch test, and there is a significant statistical difference between the two groups (p<0.0001.)

As can be seen from Tables 7 and 8-1 to 8-6, the infection marker parameter provided in the disclosure can be used to predict the risk of sepsis effectively one day in advance, and can predict that the patient will progress to sepsis one day in advance when the patient does not have the symptoms of sepsis. The diagnostic and therapeutic performance is better than that of the existing PCT standard, and surprisingly, the characteristics of WNB channel scattergram based on blood routine test have better diagnostic and therapeutic performance compared to the characteristics of DIFF channel scattergram. It is generally believed that the function of the DIFF channel is the four-part differential of leukocytes, can more accurately distinguish various leukocyte subsets, and can more easily finds infection-related features in the scattergram data, while in the WNB channel, the hemolysis intensity is relatively weak, and the distinction among different types of leukocyte subsets is not as good as that of DIFF channel, so it is not easy to find infection-related features. However, the inventors accidentally discovered through in-depth research that the WNB channel can find better features than the DIFF channel to predict the progression of sepsis. Although not wishing to be bound by theory, the inventors speculate that after the cells are treated with the reagents of the WNB channel, the infection-related monocytes, immature granulocytes, and atypical lymphocytes are all distributed in positions in the scattergram where the fluorescence signal is stronger and the side scatter signal is stronger. After the patient was infected, the number and position of these cells in the scattergram would change significantly, while other cells unrelated to infection would not change significantly, so the changes in the scattergram of the WNB channel after infection would be more significant, and were easier to be captured by detection devices.

### Example 2 Identification of a Common Infection and a Severe Infection

Blood samples from 1548 donors were subjected to blood routine test using the BC-6800 Plus blood cell analyzer produced by SHENZHEN MINDRAY BIO-MEDICAL ELECTRONICS CO., LTD. in accordance with the steps similar to example 1 of the disclosure, and the aforementioned method was adopted for identification of a severe infection based on the scattergram. Among them, there were 756 severe infection samples, that is, positive samples, and 792 non-severe infection samples, that is, negative samples.

Inclusion criteria for 1548 donors in this example: adult ICU patients with or without acute infection. Exclusion criteria: pregnant people, myelosuppressed people on chemotherapy, people on immunosuppressant treatment, patients with hematologic diseases.

For the donor of the severe infection samples: they have a suspicious or definite infection site, a positive laboratory culture result, and organ failure, which met any one or more of the following:
(1) Presence of evidence of systemic, extensive, and coelomic disseminated infection
(2) Presence of life-threatening special site infections
(3) Abnormal organ function index caused by at least one infection

Others were non-severe infection samples.

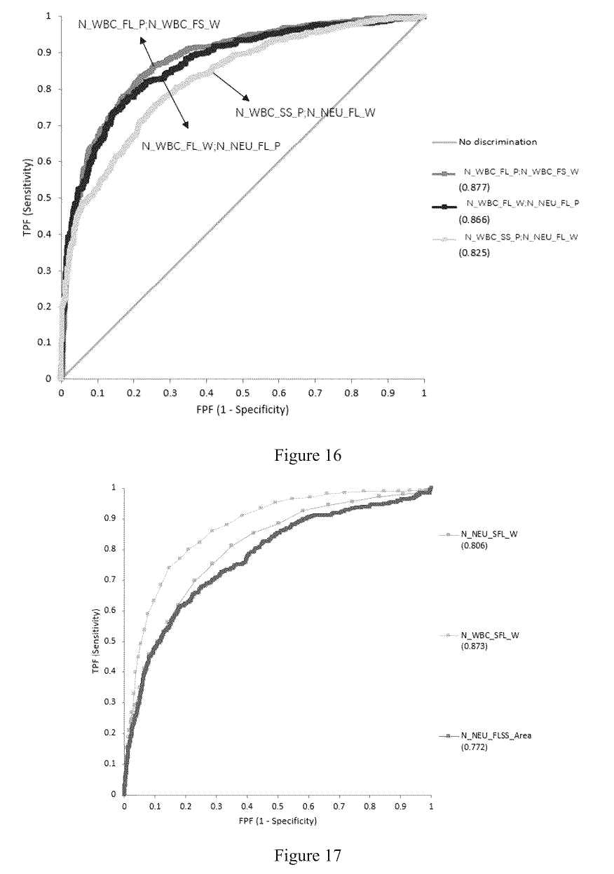

Table 9 shows the infection marker parameters used and their corresponding diagnostic efficacy, and FIGS. 15 and 16 show ROC curves corresponding to the infection marker parameters in Table 9. In Table 9:

$$Combination parameter 1 = 0.003755 \times N\_WBC \_FL \_P + 0.009 \; 192 \times N\_WBC \_FS \_W - 15.0 \; 973 ;$$

$$Combination parameter 2 = 0.005945 \times N\_WBC \_FL \_W + 0.000 \; 248 \times N\_NEU \_FL \_P - 6.62 \; 685 ;$$

$$Combination parameter 3 = 0.005249 \times N\_WBC \_SS \_P + 0.005 \; 132 \times N\_NEU \_FL \_W - 13.216 .$$

TABLE 9
Efficacy of different infection marker parameters for identification
of a common infection and a severe infection

| | | | False | True | True | False |
|---|---|---|---|---|---|---|
| Infection marker | | Determination | positive | positive | negative | negative |
| parameter | ROC_AUC | threshold | rate | rate | rate | rate |
| N_WBC_FL_W | 0.866 | >1840 | 17.9% | 76.5% | 82.1% | 23.5% |
| N_NEU_FLSS_Area | 0.776 | >10583.04 | 28.5% | 70.8% | 71.5% | 29.2% |
| N_WBC_SS_P | 0.744 | >1138.981 | 33.8% | 70.3% | 66.2% | 29.7% |
| Combination | 0.877 | >−0.3362 | 20.6% | 81.7% | 79.4% | 18.3% |
| parameter 1 | | | | | | |
| Combination | 0.866 | >−0.2372 | 18.6% | 77.6% | 81.4% | 22.4% |
| parameter 2 | | | | | | |
| Combination | 0.825 | >−0.291 | 25.9% | 75.8% | 74.1% | 24.2% |
| parameter 3 | | | | | | |

True positive means that the prompt results obtained in this example indicate severe infection, which are consistent with the patient's clinical condition; False positive means that the prompt results obtained in this example indicate severe infection, but the actual condition of the patient is common infection; True negative means that the prompt results obtained in this example indicate common infection, which are consistent with the patient's clinical condition; False negativity means that the prompt results obtained in this example indicate common infection, but the actual condition of the patient is severe infection.

In addition, Table 10 shows the efficacy of using other single leukocyte characteristic parameters as infection marker parameters for identification of a common infection and a severe infection in this example, and Tables 11-1 to 11-4 show the efficacy of using other combination parameters as infection marker parameters for identification of a common infection and a severe infection in this example, wherein infection marker parameters are calculated by the function Y=A×X1+B×X2+C based on the parameter combinations in the Table 11, where Y represents an infection marker parameter, X1 represents the first leukocyte parameter, X2 represents the second leukocyte parameter, and A, B, and C are constants.

TABLE 10
Efficacy of other single leukocyte characteristic parameters for
identification of a common infection and a severe infection

| | | | False | True | True | False |
|---|---|---|---|---|---|---|
| Single | | Determination | positive | positive | negative | negative |
| parameter | ROC_AUC | threshold | rate % | rate % | rate % | rate % |
| N_WBC_FL_P | 0.8106 | >1599.2285 | 23.9 | 74 | 76.1 | 26 |
| N_NEU_FL_W | 0.8079 | >1360 | 25 | 71.3 | 75 | 28.7 |
| N_NEU_FL_P | 0.8013 | >1715.2215 | 28.4 | 76.8 | 71.6 | 23.2 |
| N_NEU_FLFS_Area | 0.7859 | >7459.84 | 21.1 | 66.7 | 78.9 | 33.3 |
| N_WBC_SS_W | 0.7821 | >1328 | 23.6 | 70.1 | 76.4 | 29.9 |
| N_WBC_FS_W | 0.7786 | >944 | 30.8 | 72.8 | 69.2 | 27.2 |
| N_NEU_FS_W | 0.7705 | >624 | 30.3 | 68.1 | 69.7 | 31.9 |
| N_WBC_FLSS_Area | 0.7651 | >12835.84 | 28.8 | 69.2 | 71.2 | 30.8 |
| N_NEU_SS_W | 0.7618 | >1168 | 28.4 | 70.5 | 71.6 | 29.5 |
| N_WBC_FLFS_Area | 0.7555 | >9620.48 | 24.6 | 65.3 | 75.4 | 34.7 |
| N_NEU_FS_CV | 0.7495 | >0.4405 | 28 | 65.6 | 72 | 34.4 |
| N_NEU_SS_P | 0.7406 | >1162.0325 | 33.2 | 68.9 | 66.8 | 31.1 |
| N_WBC_FS_CV | 0.7152 | >0.7445 | 29.4 | 62.1 | 70.6 | 37.9 |
| N_NEU_SSFS_Area | 0.7148 | >6814.72 | 29.8 | 62.2 | 70.2 | 37.8 |
| N_WBC_FS_P | 0.7125 | >1284.492 | 33.8 | 66.4 | 66.2 | 33.6 |
| N_WBC_SS_CV | 0.7108 | >1.1665 | 25.9 | 61.3 | 74.1 | 38.7 |

TABLE 11-1
Efficacy of parameter combinations containing N_WBC_FL_W
for identification of a common infection and a severe infection

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_WBC_FL_W; | 0.8763 | >−0.1915 | 17.1 | 78.3 | 82.9 | 21.7 | 0.005593 | 4.762605 | −12.5899 |
| N_NEU_FS_CV; | | | | | | | | | |
| N_WBC_FL_W; | 0.876 | >−0.1588 | 17.2 | 78.2 | 82.8 | 21.8 | 0.004938 | 0.002903 | −11.0845 |
| N_NEU_FS_W; | | | | | | | | | |
| N_WBC_FL_W; | 0.8747 | >−0.1703 | 18.4 | 79.9 | 81.6 | 20.1 | 0.005758 | 0.007376 | −20.2999 |
| N_WBC_FS_P; | | | | | | | | | |
| N_WBC_FL_W; | 0.874 | >−0.3137 | 21.8 | 81 | 78.2 | 19 | 0.00588 | −0.00037 | −9.71223 |
| N_LYM_FLSS_Area; | | | | | | | | | |
| N_WBC_SS_W; | 0.8725 | >−0.3262 | 21.5 | 80.8 | 78.5 | 19.2 | 0.001093 | 0.005008 | −10.8573 |
| N_WBC_FL_W; | | | | | | | | | |
| N_WBC_FL_W; | 0.8724 | >−0.2289 | 20.2 | 79.5 | 79.8 | 20.5 | 0.006008 | −0.00058 | −9.69179 |
| N_LYM_FLFS_Area; | | | | | | | | | |
| N_WBC_SS_CV; | 0.8715 | >−0.2721 | 19.2 | 78.7 | 80.8 | 21.3 | 1.564145 | 0.005821 | −12.7279 |
| N_WBC_FL_W; | | | | | | | | | |
| N_WBC_SS_P; | 0.8715 | >−0.2556 | 19.2 | 78.8 | 80.8 | 21.2 | 0.003811 | 0.005522 | −14.7533 |
| N_WBC_FL_W; | | | | | | | | | |
| N_WBC_FL_W; | 0.8714 | >−0.28 | 19.6 | 78.9 | 80.4 | 21.1 | 0.005557 | 0.003259 | −14.2543 |
| N_NEU_SS_P; | | | | | | | | | |
| N_WBC_FL_W; | 0.8714 | >−0.1876 | 17.9 | 78.6 | 82.1 | 21.4 | 0.00519 | 0.001474 | −11.1159 |
| N_WBC_FS_W; | | | | | | | | | |
| N_WBC_FL_W; | 0.8707 | >−0.2771 | 21.7 | 80.6 | 78.3 | 19.4 | 0.005817 | −0.00068 | −9.11971 |
| N_LYM_SSFS_Area; | | | | | | | | | |
| N_WBC_FL_W; | 0.8706 | >−0.3111 | 21.5 | 80.4 | 78.5 | 19.6 | 0.005113 | 0.000994 | −10.7654 |
| N_NEU_SS_W; | | | | | | | | | |
| N_WBC_FL_W; | 0.8702 | >−0.3045 | 18.4 | 78.3 | 81.6 | 21.7 | 0.006667 | −0.00013 | −12.3557 |
| N_LYM_SS_P; | | | | | | | | | |
| N_WBC_FL_W; | 0.8699 | >−0.3069 | 19.6 | 79.1 | 80.4 | 20.9 | 0.00673 | −0.00013 | −12.4439 |
| N_LYM_FL_P; | | | | | | | | | |
| N_WBC_FL_W; | 0.8697 | >−0.3098 | 18.2 | 77.5 | 81.8 | 22.5 | 0.006667 | −0.00013 | −12.4695 |
| N_LYM_FS_CV; | | | | | | | | | |
| N_WBC_FL_W; | 0.8697 | >−0.2469 | 19 | 78 | 81 | 22 | 0.005924 | 1.337057 | −12.453 |
| N_NEU_SS_CV; | | | | | | | | | |
| N_WBC_FL_W; | 0.8693 | >−0.2597 | 20.2 | 79.8 | 79.8 | 20.2 | 0.005277 | 0.000119 | −10.6778 |
| N_NEU_SSFS_Area; | | | | | | | | | |
| N_WBC_FL_W; | 0.8692 | >−0.3066 | 18.2 | 77.9 | 81.8 | 22.1 | 0.006663 | −0.00013 | −12.3364 |
| N_LYM_FS_P; | | | | | | | | | |
| N_WBC_FL_W; | 0.8691 | >−0.1439 | 16.8 | 78.3 | 83.2 | 21.7 | 0.005058 | 0.00116 | −10.3538 |
| N_LYM_FL_W; | | | | | | | | | |
| N_WBC_FL_W; | 0.8685 | >−0.1114 | 16.5 | 76.6 | 83.5 | 23.4 | 0.005004 | 9.9E−05 | −10.4221 |
| N_NEU_FLSS_Area; | | | | | | | | | |
| N_WBC_FL_W; | 0.8683 | >−0.1155 | 16.2 | 76.7 | 83.8 | 23.3 | 0.004999 | 0.000165 | −10.5541 |
| N_NEU_FLFS_Area; | | | | | | | | | |
| N_WBC_FL_W; | 0.8681 | >−0.2141 | 19 | 77.9 | 81 | 22.1 | 0.005953 | 0.003091 | −15.5542 |
| N_NEU_FS_P; | | | | | | | | | |
| N_WBC_FL_W; | 0.8679 | >−0.2761 | 19 | 78.9 | 81 | 21.1 | 0.006067 | 0.726425 | −11.8816 |
| N_WBC_FS_CV; | | | | | | | | | |
| N_WBC_FL_W; | 0.8676 | >−0.2354 | 19.3 | 78.7 | 80.7 | 21.3 | 0.00552 | 0.000674 | −10.7217 |
| N_LYM_SS_W; | | | | | | | | | |
| N_WBC_FL_W; | 0.867 | >−0.2277 | 18.2 | 77.2 | 81.8 | 22.8 | 0.006213 | 0.380365 | −11.9104 |
| N_NEU_FL_CV; | | | | | | | | | |
| N_WBC_FL_W; | 0.8666 | >−0.23 | 20.1 | 79.4 | 79.9 | 20.6 | 0.004921 | 0.001226 | −10.8734 |
| N_NEU_FL_W; | | | | | | | | | |
| N_WBC_FL_W; | 0.8666 | >−0.2032 | 18 | 76.8 | 82 | 23.2 | 0.006666 | −0.00013 | −12.4694 |
| N_LYM_SS_CV; | | | | | | | | | |
| N_WBC_FL_W; | 0.8663 | >−0.2032 | 18 | 76.8 | 82 | 23.2 | 0.006666 | −0.00013 | −12.4694 |
| N_LYM_FL_CV; | | | | | | | | | |
| N_WBC_FL_P; | 0.8662 | >−0.2245 | 19 | 78.1 | 81 | 21.9 | 0.00083 | 0.005485 | −11.608 |
| N_WBC_FL_W; | | | | | | | | | |
| N_WBC_FL_W; | 0.8661 | >−0.2316 | 19.2 | 77.8 | 80.8 | 22.2 | 0.005615 | −7.6E−06 | −10.4052 |
| N_WBC_FLFS_Area; | | | | | | | | | |
| N_WBC_FL_W; | 0.8657 | >−0.2372 | 18.6 | 77.6 | 81.4 | 22.4 | 0.005945 | 0.000248 | −11.5513 |
| N_NEU_FL_P; | | | | | | | | | |
| N_WBC_FL_W; | 0.8655 | >−0.2363 | 20.2 | 78.7 | 79.8 | 21.3 | 0.005627 | −1.5E−05 | −10.3599 |
| N_WBC_SSFS_Area; | | | | | | | | | |
| N_WBC_FL_W; | 0.8653 | >−0.2112 | 18.7 | 77.9 | 81.3 | 22.1 | 0.006206 | −1.39982 | −9.97502 |
| N_WBC_FL_CV | | | | | | | | | |
| N_WBC_FL_W; | 0.8653 | >−0.1459 | 17.7 | 76.6 | 82.3 | 23.4 | 0.005417 | 2.31E−05 | −10.4141 |
| N_WBC_FLSS_Area; | | | | | | | | | |
| N_WBC_FL_W; | 0.8618 | >−0.1998 | 20.7 | 78.6 | 79.3 | 21.4 | 0.006013 | −0.00768 | −8.8051 |
| N_LYM_FS_W; | | | | | | | | | |

TABLE 11-2
Efficacy of parameter combinations containing N_NEU_FLSS_Area
for identification of a common infection and a severe infection

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_WBC_FL_W; | 0.8685 | >−0.1114 | 16.5 | 76.6 | 83.5 | 23.4 | 0.005004 | 9.9E−05 | −10.4221 |
| N_NEU_FLSS_Area; | | | | | | | | | |
| N_WBC_FL_P; | 0.8544 | >−0.2072 | 20.6 | 78.1 | 79.4 | 21.9 | 0.003446 | 0.000334 | −9.32385 |
| N_NEU_FLSS_Area; | | | | | | | | | |
| N_NEU_FL_P; | 0.8507 | >−0.2262 | 20.8 | 77.6 | 79.2 | 22.4 | 0.003157 | 0.000334 | −9.31683 |
| N_NEU_FLSS_Area; | | | | | | | | | |
| N_LYM_FL_W; | 0.8487 | >−0.3419 | 23.7 | 77.1 | 76.3 | 22.9 | 0.004525 | 0.000329 | −7.18737 |
| N_NEU_FLSS_Area; | | | | | | | | | |
| N_NEU_FL_CV; | 0.8277 | >−0.1351 | 23.3 | 75.1 | 76.7 | 24.9 | −5.41616 | 0.000494 | −1.15362 |
| N_NEU_FLSS_Area; | | | | | | | | | |
| N_LYM_FLSS_Area; | 0.8088 | >0.0788 | 21.2 | 68 | 78.8 | 32 | −0.00057 | 0.00048 | −3.33026 |
| N_NEU_FLSS_Area; | | | | | | | | | |
| N_LYM_SSFS_Area; | 0.8087 | >−0.064 | 27.8 | 73.3 | 72.2 | 26.7 | −0.00109 | 0.000472 | −2.41758 |
| N_NEU_FLSS_Area; | | | | | | | | | |
| N_NEU_FL_W; | 0.8078 | >−0.2062 | 26.9 | 73.5 | 73.1 | 26.5 | 0.005139 | 5.91E−05 | −7.75577 |
| N_NEU_FLSS_Area; | | | | | | | | | |
| N_WBC_FL_CV; | 0.8033 | >−0.0671 | 23.5 | 71.5 | 76.5 | 28.5 | −3.85824 | 0.000442 | −0.4091 |
| N_NEU_FLSS_Area; | | | | | | | | | |
| N_WBC_FS_W; | 0.8005 | >−0.1616 | 24 | 70.6 | 76 | 29.4 | 0.004381 | 0.000235 | −6.84949 |
| N_NEU_FLSS_Area; | | | | | | | | | |
| N_WBC_FS_P; | 0.7986 | >−0.1045 | 24.9 | 68.7 | 75.1 | 31.3 | 0.008353 | 0.000316 | −14.3048 |
| N_NEU_FLSS_Area; | | | | | | | | | |
| N_WBC_SSFS_Area; | 0.797 | >−0.1323 | 28.4 | 72.6 | 71.6 | 27.4 | −0.00064 | 0.000742 | −2.38417 |
| N_NEU_FLSS_Area; | | | | | | | | | |
| N_WBC_SS_W; | 0.7968 | >−0.2067 | 24.2 | 71 | 75.8 | 29 | 0.001983 | 0.00023 | −5.29195 |
| N_NEU_FLSS_Area; | | | | | | | | | |
| N_LYM_FLFS_Area; | 0.7925 | >0.0321 | 23.7 | 67.1 | 76.3 | 32.9 | −0.00077 | 0.000459 | −3.02896 |
| N_NEU_FLSS_Area; | | | | | | | | | |
| N_NEU_SS_P; | 0.7899 | >−0.2309 | 26.8 | 70.5 | 73.2 | 29.5 | 0.005087 | 0.000259 | −8.90703 |
| N_NEU_FLSS_Area; | | | | | | | | | |
| N_LYM_SS_W; | 0.7879 | >−0.0849 | 23.2 | 67.2 | 76.8 | 32.8 | 0.002521 | 0.000353 | −5.47052 |
| N_NEU_FLSS_Area; | | | | | | | | | |
| N_WBC_SS_P; | 0.787 | >−0.2101 | 27 | 70.3 | 73 | 29.7 | 0.005354 | 0.00025 | −9.00394 |
| N_NEU_FLSS_Area; | | | | | | | | | |
| N_NEU_FS_W; | 0.787 | >−0.1676 | 26.9 | 69.4 | 73.1 | 30.6 | 0.004115 | 0.000229 | −5.18863 |
| N_NEU_FLSS_Area; | | | | | | | | | |
| N_NEU_FLSS_Area; | 0.7864 | >−0.1003 | 24.6 | 69.2 | 75.4 | 30.8 | 0.000701 | −0.00061 | −3.4506 |
| N_NEU_SSFS_Area; | | | | | | | | | |
| N_NEU_FLFS_Area; | 0.7858 | >−0.066 | 22.7 | 67.2 | 77.3 | 32.8 | 0.000483 | 0.000103 | −4.75943 |
| N_NEU_FLSS_Area; | | | | | | | | | |
| N_NEU_FS_P; | 0.785 | >−0.1442 | 25.8 | 69.1 | 74.2 | 30.9 | 0.004288 | 0.000337 | −9.86805 |
| N_NEU_FLSS_Area; | | | | | | | | | |
| N_WBC_SS_CV; | 0.7846 | >−0.1384 | 23.3 | 69 | 76.7 | 31 | 2.274314 | 0.00031 | −6.11909 |
| N_NEU_FLSS_Area; | | | | | | | | | |
| N_WBC_FS_CV; | 0.7825 | >−0.1648 | 25.6 | 69.3 | 74.4 | 30.7 | 3.736974 | 0.000313 | −6.26565 |
| N_NEU_FLSS_Area; | | | | | | | | | |
| N_NEU_FS_CV; | 0.7824 | >−0.2784 | 31.6 | 73.5 | 68.4 | 26.5 | 5.590812 | 0.000269 | −5.49494 |
| N_NEU_FLSS_Area; | | | | | | | | | |
| N_NEU_SS_W; | 0.7819 | >−0.1778 | 26 | 70 | 74 | 30 | 0.001291 | 0.000274 | −4.62602 |
| N_NEU_FLSS_Area; | | | | | | | | | |
| N_LYM_SS_P; | 0.7789 | >−0.1722 | 27.6 | 70.3 | 72.4 | 29.7 | −6.8E−05 | 0.000392 | −4.28046 |
| N_NEU_FLSS_Area; | | | | | | | | | |
| N_LYM_FS_P; | 0.7788 | >−0.1923 | 28.5 | 71.1 | 71.5 | 28.9 | −6.8E−05 | 0.000392 | −4.27279 |
| N_NEU_FLSS_Area; | | | | | | | | | |
| N_LYM_FS_CV; | 0.7788 | >−0.1906 | 28.6 | 71.1 | 71.4 | 28.9 | −6.8E−05 | 0.000392 | −4.33911 |
| N_NEU_FLSS_Area; | | | | | | | | | |
| N_LYM_SS_CV; | 0.7787 | >−0.1906 | 28.6 | 71.1 | 71.4 | 28.9 | −6.8E−05 | 0.000392 | −4.33907 |
| N_NEU_FLSS_Area; | | | | | | | | | |
| N_LYM_FL_CV; | 0.7786 | >−0.1906 | 28.6 | 71.1 | 71.4 | 28.9 | −6.8E−05 | 0.000392 | −4.33907 |
| N_NEU_FLSS_Area; | | | | | | | | | |
| N_LYM_FL_P; | 0.7767 | >−0.1852 | 28.5 | 70.7 | 71.5 | 29.3 | −5.4E−05 | 0.000389 | −4.24041 |
| N_NEU_FLSS_Area; | | | | | | | | | |
| N_NEU_SS_CV; | 0.7764 | >−0.1003 | 23.8 | 67.8 | 76.2 | 32.2 | 0.514821 | 0.000358 | −4.49283 |
| N_NEU_FLSS_Area; | | | | | | | | | |
| N_LYM_FS_W; | 0.7754 | >−0.0906 | 24.5 | 67.3 | 75.5 | 32.7 | −0.00027 | 0.000366 | −3.95933 |
| N_NEU_FLSS_Area; | | | | | | | | | |
| N_WBC_FLFS_Area; | 0.7749 | >−0.108 | 25.8 | 67.6 | 74.2 | 32.4 | −4.7E−05 | 0.000394 | −3.89478 |
| N_NEU_FLSS_Area; | | | | | | | | | |
| N_WBC_FLSS_Area; | 0.7747 | >−0.0938 | 24.9 | 67.5 | 75.1 | 32.5 | −6E−05 | 0.00043 | −3.94398 |
| N_NEU_FLSS_Area; | | | | | | | | | |

TABLE 11-3
Efficacy of parameter combinations containing N_WBC_SS_P
for identification of a common infection and a severe infection

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_WBC_SS_P; | 0.8715 | >−0.2556 | 19.2 | 78.8 | 80.8 | 21.2 | 0.003811 | 0.005522 | −14.7533 |
| N_WBC_FL_W; | | | | | | | | | |
| N_WBC_SS_P; | 0.8399 | >−0.3027 | 24.4 | 79.3 | 75.6 | 20.7 | 0.007509 | 0.00325 | −14.0837 |
| N_WBC_FL_P; | | | | | | | | | |
| N_WBC_SS_P; | 0.8348 | >−0.2796 | 24.3 | 78.5 | 75.7 | 21.5 | 0.00763 | 0.002986 | −14.2335 |
| N_NEU_FL_P; | | | | | | | | | |
| N_WBC_SS_P; | 0.828 | >−0.3761 | 24.4 | 76.6 | 75.6 | 23.4 | 0.007971 | 0.004407 | −12.7802 |
| N_LYM_FL_W; | | | | | | | | | |
| N_WBC_SS_P; | 0.8245 | >−0.291 | 25.9 | 75.8 | 74.1 | 24.2 | 0.005249 | 0.005132 | −13.216 |
| N_NEU_FL_W; | | | | | | | | | |
| N_WBC_SS_P; | 0.8018 | >−0.1786 | 25.3 | 71 | 74.7 | 29 | 0.005759 | 0.000487 | −10.3611 |
| N_NEU_FLFS_Area; | | | | | | | | | |
| N_WBC_SS_P; | 0.7977 | >−0.2226 | 26.7 | 72.2 | 73.3 | 27.8 | 0.006483 | 0.00596 | −11.4177 |
| N_NEU_FS_W; | | | | | | | | | |
| N_WBC_SS_P; | 0.797 | >−0.1873 | 26.1 | 70.9 | 73.9 | 29.1 | 0.007481 | 8.962429 | −12.7608 |
| N_NEU_FS_CV; | | | | | | | | | |
| N_WBC_SS_P; | 0.7963 | >−0.1632 | 22.5 | 70.2 | 77.5 | 29.8 | 0.005737 | 0.006394 | −12.9218 |
| N_WBC_FS_W; | | | | | | | | | |
| N_WBC_SS_P; | 0.787 | >−0.2101 | 27 | 70.3 | 73 | 29.7 | 0.005354 | 0.00025 | −9.00394 |
| N_NEU_FLSS_Area; | | | | | | | | | |
| N_WBC_SS_P; | 0.7851 | >−0.2933 | 28.2 | 72.5 | 71.8 | 27.5 | 0.007893 | 3.467251 | −13.3231 |
| N_WBC_SS_CV; | | | | | | | | | |
| N_WBC_SS_P; | 0.7829 | >−0.1119 | 22.8 | 65.8 | 77.2 | 34.2 | 0.006853 | 0.000316 | −11.0627 |
| N_WBC_FLFS_Area; | | | | | | | | | |
| N_WBC_SS_P; | 0.7822 | >−0.1513 | 24.9 | 67.7 | 75.1 | 32.3 | 0.00616 | 0.000195 | −9.78463 |
| N_WBC_FLSS_Area; | | | | | | | | | |
| N_WBC_SS_P; | 0.7813 | >−0.3604 | 32 | 75.6 | 68 | 24.4 | 0.005101 | 0.002415 | −9.31351 |
| N_WBC_SS_W; | | | | | | | | | |
| N_WBC_SS_P; | 0.7812 | >−0.1762 | 24.1 | 69.7 | 75.9 | 30.3 | 0.007843 | 6.839149 | −14.2931 |
| N_WBC_FS_CV; | | | | | | | | | |
| N_WBC_SS_P; | 0.7758 | >−0.2858 | 30.3 | 73 | 69.7 | 27 | 0.008472 | 2.750157 | −12.7386 |
| N_NEU_SS_CV; | | | | | | | | | |
| N_WBC_SS_P; | 0.7736 | >−0.2929 | 30.6 | 72.6 | 69.4 | 27.4 | 0.00633 | 0.001958 | −9.81609 |
| N_NEU_SS_W; | | | | | | | | | |
| N_WBC_SS_P; | 0.7707 | >−0.1716 | 28.1 | 69.7 | 71.9 | 30.3 | 0.010147 | −0.00062 | −10.2594 |
| N_LYM_SSFS_Area; | | | | | | | | | |
| N_WBC_SS_P; | 0.765 | >−0.2614 | 31.8 | 72.6 | 68.2 | 27.4 | 0.009958 | −0.00028 | −10.6721 |
| N_LYM_FLSS_Area; | | | | | | | | | |
| N_WBC_SS_P; | 0.7592 | >−0.2125 | 27.8 | 68.9 | 72.2 | 31.1 | 0.009869 | −2.16942 | −9.80203 |
| N_NEU_FL_CV; | | | | | | | | | |
| N_WBC_SS_P; | 0.7584 | >−0.2239 | 30 | 69.3 | 70 | 30.7 | 0.007596 | 0.006005 | −16.6431 |
| N_WBC_FS_P; | | | | | | | | | |
| N_WBC_SS_P; | 0.7546 | >−0.1764 | 27 | 67.2 | 73 | 32.8 | 0.009846 | −1.77831 | −9.43562 |
| N_WBC_FL_CV; | | | | | | | | | |
| N_WBC_SS_P; | 0.7534 | >−0.1188 | 25.4 | 64 | 74.6 | 36 | 0.007912 | 0.000163 | −10.3801 |
| N_NEU_SSFS_Area; | | | | | | | | | |
| N_WBC_SS_P; | 0.7511 | >−0.2129 | 30.6 | 68.5 | 69.4 | 31.5 | 0.009821 | −0.00024 | −10.824 |
| N_LYM_FLFS_Area; | | | | | | | | | |
| N_WBC_SS_P; | 0.7481 | >−0.1905 | 28.7 | 65.8 | 71.3 | 34.2 | 0.009161 | 0.001401 | −11.5759 |
| N_LYM_SS_W; | | | | | | | | | |
| N_WBC_SS_P; | 0.7454 | >−0.2744 | 33.8 | 70.5 | 66.2 | 29.5 | 0.009583 | −2.3E−05 | −11.1703 |
| N_LYM_SS_P; | | | | | | | | | |
| N_WBC_SS_P; | 0.7453 | >−0.2742 | 33.8 | 70.5 | 66.2 | 29.5 | 0.00958 | −2.2E−05 | −11.1651 |
| N_LYM_FS_P; | | | | | | | | | |
| N_WBC_SS_P; | 0.7452 | >−0.2755 | 33.8 | 70.5 | 66.2 | 29.5 | 0.009577 | −2.1E−05 | −11.1837 |
| N_LYM_SS_CV; | | | | | | | | | |
| N_WBC_SS_P; | 0.7452 | >−0.2755 | 33.8 | 70.5 | 66.2 | 29.5 | 0.009577 | −2.1E−05 | −11.1837 |
| N_LYM_FL_CV; | | | | | | | | | |
| N_WBC_SS_P; | 0.7452 | >−0.2755 | 33.8 | 70.5 | 66.2 | 29.5 | 0.009577 | −2.1E−05 | −11.1837 |
| N_LYM_FS_CV; | | | | | | | | | |
| N_WBC_SS_P; | 0.7448 | >−0.2342 | 32.4 | 68.7 | 67.6 | 31.3 | 0.009716 | −0.00155 | −10.8548 |
| N_LYM_FS_W; | | | | | | | | | |
| N_WBC_SS_P; | 0.7443 | >−0.2125 | 30 | 66.8 | 70 | 33.2 | 0.009112 | 0.00131 | −12.5169 |
| N_NEU_FS_P; | | | | | | | | | |
| N_WBC_SS_P; | 0.7442 | >−0.2235 | 30.8 | 66.9 | 69.2 | 33.1 | 0.00783 | 0.001644 | −11.0996 |
| N_NEU_SS_P; | | | | | | | | | |
| N_WBC_SS_P; | 0.744 | >−0.2704 | 33.7 | 69.8 | 66.3 | 30.2 | 0.009577 | −9.9E−06 | −11.1712 |
| N_LYM_FL_P; | | | | | | | | | |
| N_WBC_SS_P; | 0.7439 | >−0.1982 | 29.6 | 66 | 70.4 | 34 | 0.009612 | −3.5E−06 | −11.1894 |
| N_WBC_SSFS_Area; | | | | | | | | | |

TABLE 11-4
Efficacy of the remaining parameter combinations for identification of a common infection and a severe infection

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_WBC_FL_P; | 0.877 | >−0.1173 | 17.7 | 77.7 | 82.3 | 22.3 | 0.003887 | 0.008809 | −12.0598 |
| N_NEU_FS_W; | | | | | | | | | |
| N_WBC_FL_P; | 0.8752 | >−0.2016 | 20 | 79.9 | 80 | 20.1 | 0.003983 | 13.15541 | −12.4447 |
| N_NEU_FS_CV; | | | | | | | | | |
| N_NEU_FL_P; | 0.8749 | >−0.1686 | 18 | 78.4 | 82 | 21.6 | 0.003601 | 0.008839 | −12.1455 |
| N_NEU_FS_W; | | | | | | | | | |
| N_WBC_FL_P; | 0.8748 | >−0.3413 | 20.3 | 81.5 | 79.7 | 18.5 | 0.004121 | 12.26878 | −15.974 |
| N_WBC_FS_CV; | | | | | | | | | |
| N_NEU_FL_P; | 0.8726 | >−0.1261 | 17.3 | 77.5 | 82.7 | 22.5 | 0.003686 | 13.16114 | −12.5081 |
| N_NEU_FS_CV; | | | | | | | | | |
| N_WBC_FS_W; | 0.8709 | >−0.2086 | 18.1 | 78.1 | 81.9 | 21.9 | 0.008974 | 0.003395 | −14.7995 |
| N_NEU_FL_P; | | | | | | | | | |
| N_WBC_SS_W; | 0.8699 | >−0.3676 | 21.4 | 81.7 | 78.6 | 18.3 | 0.003818 | 0.003719 | −11.3922 |
| N_WBC_FL_P; | | | | | | | | | |
| N_WBC_FL_P; | 0.8685 | >−0.3703 | 20.4 | 80.7 | 79.6 | 19.3 | 0.006494 | 8.995866 | −21.0577 |
| N_WBC_FL_CV; | | | | | | | | | |
| N_WBC_SS_CV; | 0.867 | >−0.3509 | 21 | 80 | 79 | 20 | 6.368643 | 0.00417 | −14.3858 |
| N_WBC_FL_P; | | | | | | | | | |
| N_WBC_SS_W; | 0.8641 | >−0.3207 | 20.3 | 79.1 | 79.7 | 20.9 | 0.003714 | 0.003355 | −11.1513 |
| N_NEU_FL_P; | | | | | | | | | |
| N_LYM_FL_W; | 0.8631 | >−0.273 | 19.1 | 77.1 | 80.9 | 22.9 | 0.004898 | 0.007767 | −8.88144 |
| N_NEU_FS_W; | | | | | | | | | |
| N_LYM_FL_W; | 0.8627 | >−0.2374 | 17.8 | 75.2 | 82.2 | 24.8 | 0.005421 | 12.77322 | −10.0356 |
| N_NEU_FS_CV; | | | | | | | | | |
| N_WBC_FL_P; | 0.8618 | >−0.3247 | 21.3 | 79.7 | 78.7 | 20.3 | 0.003819 | 0.00374 | −10.8877 |
| N_NEU_SS_W; | | | | | | | | | |
| N_WBC_FL_P; | 0.8604 | >−0.1931 | 20.9 | 77.7 | 79.1 | 22.3 | 0.004145 | 0.000583 | −10.8647 |
| N_NEU_SSFS_Area; | | | | | | | | | |
| N_WBC_FS_CV; | 0.8597 | >−0.1363 | 17.5 | 75.8 | 82.5 | 24.2 | 11.04951 | 0.003596 | −14.7346 |
| N_NEU_FL_P; | | | | | | | | | |
| N_WBC_SS_CV; | 0.8577 | >−0.2697 | 19.6 | 77.6 | 80.4 | 22.4 | 6.008779 | 0.003736 | −13.8036 |
| N_NEU_FL_P; | | | | | | | | | |
| N_LYM_FL_W; | 0.8576 | >−0.1088 | 16.4 | 74.6 | 83.6 | 25.4 | 0.004254 | 0.00516 | −10.48 |
| N_NEU_FL_W; | | | | | | | | | |
| N_NEU_SS_W; | 0.8569 | >−0.4039 | 23.8 | 79.9 | 76.2 | 20.1 | 0.003663 | 0.003477 | −10.7469 |
| N_NEU_FL_P; | | | | | | | | | |
| N_NEU_FL_CV; | 0.8568 | >−0.2384 | 23 | 80 | 77 | 20 | −7.8833 | 0.013671 | −2.59829 |
| N_NEU_FS_W; | | | | | | | | | |
| N_WBC_FL_P; | 0.8563 | >−0.2147 | 21.4 | 78.3 | 78.6 | 21.7 | 0.003211 | 0.005538 | −12.9157 |
| N_NEU_FL_W; | | | | | | | | | |
| N_WBC_FS_W; | 0.8561 | >−0.33 | 21.6 | 80 | 78.4 | 20 | 0.006831 | 0.004347 | −10.0777 |
| N_LYM_FL_W; | | | | | | | | | |
| N_NEU_FL_P; | 0.8546 | >−0.1488 | 20.3 | 76.2 | 79.7 | 23.8 | 0.003786 | 0.000568 | −10.7345 |
| N_NEU_SSFS_Area; | | | | | | | | | |
| N_WBC_SS_W; | 0.8541 | >−0.4648 | 23.4 | 81 | 76.6 | 19 | 0.003237 | 0.004474 | −8.02466 |
| N_LYM_FL_W; | | | | | | | | | |
| N_WBC_FL_P; | 0.8541 | >−0.0986 | 19.1 | 75.1 | 80.9 | 24.9 | 0.003401 | 0.000598 | −10.0457 |
| N_NEU_FLFS_Area; | | | | | | | | | |
| N_NEU_FL_P; | 0.8531 | >−0.0311 | 15.9 | 74.3 | 84.1 | 25.7 | 0.002917 | 0.005534 | −12.8608 |
| N_NEU_FL_W; | | | | | | | | | |
| N_WBC_FL_P; | 0.8528 | >−0.1715 | 21.3 | 77.4 | 78.7 | 22.6 | 0.006549 | 8.685591 | −17.6179 |
| N_NEU_FL_CV; | | | | | | | | | |
| N_NEU_FL_P; | 0.8522 | >−0.1474 | 18.4 | 76 | 81.6 | 24 | 0.006428 | 9.360302 | −18.9035 |
| N_NEU_FL_CV; | | | | | | | | | |
| N_WBC_FS_CV; | 0.8512 | >−0.3058 | 21.3 | 78.3 | 78.7 | 21.7 | 10.31765 | 0.005173 | −11.8525 |
| N_LYM_FL_W; | | | | | | | | | |
| N_NEU_FL_P; | 0.851 | >−0.1584 | 20.3 | 76 | 79.7 | 24 | 0.003114 | 0.000599 | −10.0401 |
| N_NEU_FLFS_Area; | | | | | | | | | |
| N_WBC_FL_P; | 0.8506 | >−0.2014 | 20.8 | 77 | 79.2 | 23 | 0.004345 | 0.000466 | −11.3567 |
| N_WBC_SSFS_Area; | | | | | | | | | |
| N_NEU_FL_CV; | 0.8498 | >−0.2003 | 22.8 | 78.1 | 77.2 | 21.9 | −8.30848 | 21.17501 | −2.95579 |
| N_NEU_FS_CV; | | | | | | | | | |
| N_LYM_FL_W; | 0.8497 | >−0.23 | 20.2 | 75.1 | 79.8 | 24.9 | 0.004423 | 0.000578 | −7.80865 |
| N_NEU_FLFS_Area; | | | | | | | | | |
| N_NEU_FL_W; | 0.8479 | >−0.1276 | 19.6 | 76.2 | 80.4 | 23.8 | 0.008878 | −7.10272 | −6.6473 |
| N_NEU_FL_CV; | | | | | | | | | |
| N_WBC_FL_P; | 0.8475 | >−0.2848 | 22.7 | 78.1 | 77.3 | 21.9 | 0.004189 | 5.483054 | −12.5536 |
| N_NEU_SS_CV; | | | | | | | | | |
| N_WBC_FL_P; | 0.8474 | >−0.2031 | 20.8 | 77 | 79.2 | 23 | 0.003458 | 0.000275 | −9.34104 |
| N_WBC_FLSS_Area; | | | | | | | | | |
| N_LYM_FL_W; | 0.8471 | >−0.3658 | 20.6 | 76.5 | 79.4 | 23.5 | 0.004713 | 0.003215 | −7.69923 |
| N_NEU_SS_W; | | | | | | | | | |
| N_WBC_FL_P; | 0.845 | >−0.1389 | 19 | 75 | 81 | 25 | 0.003555 | 0.000454 | −10.2312 |
| N_WBC_FLFS_Area; | | | | | | | | | |
| N_WBC_FLSS_Area; | 0.8445 | >−0.3473 | 23.4 | 78 | 76.6 | 22 | 0.000278 | 0.00465 | −7.37143 |
| N_LYM_FL_W; | | | | | | | | | |
| N_WBC_FL_P; | 0.8424 | >−0.2239 | 22.3 | 76.6 | 77.7 | 23.4 | 0.003283 | 0.006941 | −13.6389 |
| N_NEU_SS_P; | | | | | | | | | |
| N_NEU_SS_CV; | 0.8424 | >−0.3906 | 23.9 | 79.6 | 76.1 | 20.4 | 5.371518 | 0.003846 | −12.4468 |
| N_NEU_FL_P; | | | | | | | | | |
| N_WBC_SS_CV; | 0.8422 | >−0.4239 | 22.5 | 78.8 | 77.5 | 21.2 | 5.214553 | 0.005164 | −10.2747 |
| N_LYM_FL_W; | | | | | | | | | |
| N_LYM_FL_W; | 0.841 | >−0.2811 | 20.5 | 75.8 | 79.5 | 24.2 | 0.005083 | 0.000475 | −7.33095 |
| N_NEU_SSFS_Area; | | | | | | | | | |
| N_WBC_FL_CV; | 0.8409 | >−0.128 | 20.4 | 74.3 | 79.6 | 25.7 | 6.499233 | 0.005155 | −16.7444 |
| N_NEU_FL_P; | | | | | | | | | |
| N_WBC_SS_P; | 0.8399 | >−0.3027 | 24.4 | 79.3 | 75.6 | 20.7 | 0.007509 | 0.00325 | −14.0837 |
| N_WBC_FL_P; | | | | | | | | | |
| N_WBC_FLFS_Area; | 0.8376 | >−0.2991 | 23.1 | 75.9 | 76.9 | 24.1 | 0.000422 | 0.004606 | −7.73354 |
| N_LYM_FL_W; | | | | | | | | | |
| N_WBC_FLSS_Area; | 0.8369 | >−0.1316 | 25 | 76.1 | 75 | 23.9 | 0.000536 | −0.00088 | −4.10513 |
| N_LYM_FLSS_Area; | | | | | | | | | |
| N_WBC_SSFS_Area; | 0.8368 | >−0.2752 | 23.4 | 77.2 | 76.6 | 22.8 | 0.000416 | 0.003831 | −10.6321 |
| N_NEU_FL_P; | | | | | | | | | |
| N_WBC_FLSS_Area; | 0.8361 | >−0.1494 | 20.3 | 76 | 79.7 | 24 | 0.000258 | 0.003051 | −8.89749 |
| N_NEU_FL_P; | | | | | | | | | |
| N_WBC_FL_CV; | 0.8357 | >−0.1823 | 21.5 | 75 | 78.5 | 25 | −6.69002 | 0.013505 | −5.41146 |
| N_WBC_FS_W; | | | | | | | | | |
| N_LYM_FLSS_Area; | 0.8349 | >−0.2053 | 28.3 | 77.9 | 71.7 | 22.1 | −0.00053 | 0.007059 | −7.91454 |
| N_NEU_FL_W; | | | | | | | | | |
| N_WBC_SS_P; | 0.8348 | >−0.2796 | 24.3 | 78.5 | 75.7 | 21.5 | 0.00763 | 0.002986 | −14.2335 |
| N_NEU_FL_P; | | | | | | | | | |
| N_NEU_SS_P; | 0.8346 | >−0.2721 | 23.7 | 77.2 | 76.3 | 22.8 | 0.006883 | 0.00298 | −13.5163 |
| N_NEU_FL_P; | | | | | | | | | |
| N_WBC_FLFS_Area; | 0.8344 | >−0.1925 | 21.9 | 75.9 | 78.1 | 24.1 | 0.000424 | 0.003146 | −9.73597 |
| N_NEU_FL_P; | | | | | | | | | |
| N_WBC_FLSS_Area; | 0.8328 | >−0.0858 | 25.4 | 76.1 | 74.6 | 23.9 | 0.00051 | −0.00162 | −2.66451 |
| N_LYM_SSFS_Area; | | | | | | | | | |
| N_WBC_FL_CV; | 0.8315 | >−0.2336 | 25.3 | 78 | 74.7 | 22 | −9.1743 | 21.54822 | −5.58919 |
| N_WBC_FS_CV; | | | | | | | | | |
| N_NEU_FL_CV; | 0.8309 | >−0.1681 | 23.9 | 76.3 | 76.1 | 23.7 | −5.4707 | 0.000885 | −2.29357 |
| N_NEU_FLFS_Area; | | | | | | | | | |
| N_LYM_SSFS_Area; | 0.8305 | >−0.0354 | 24.9 | 74.1 | 75.1 | 25.9 | −0.00103 | 0.006981 | −7.01457 |
| N_NEU_FL_W; | | | | | | | | | |
| N_WBC_FS_W; | 0.8303 | >−0.1734 | 21.5 | 74.3 | 78.5 | 25.7 | 0.012106 | −5.51072 | −7.4299 |
| N_NEU_FL_CV; | | | | | | | | | |
| N_WBC_FS_P; | 0.8302 | >−0.2185 | 25.4 | 75.9 | 74.6 | 24.1 | 0.008425 | 0.005718 | −18.816 |
| N_NEU_FL_W; | | | | | | | | | |
| N_WBC_FLFS_Area; | 0.8283 | >−0.1792 | 26.1 | 75.9 | 73.9 | 24.1 | 0.00084 | −0.00084 | −5.2576 |
| N_LYM_FLSS_Area; | | | | | | | | | |
| N_LYM_FL_W; | 0.8282 | >−0.3514 | 22.8 | 75.6 | 77.2 | 24.4 | 0.004445 | 0.007318 | −12.2232 |
| N_NEU_SS_P; | | | | | | | | | |
| N_WBC_SS_P; | 0.828 | >−0.3761 | 24.4 | 76.6 | 75.6 | 23.4 | 0.007971 | 0.004407 | −12.7802 |
| N_LYM_FL_W; | | | | | | | | | |
| N_NEU_SS_P; | 0.8278 | >−0.255 | 23.7 | 74.3 | 76.3 | 25.7 | 0.005117 | 0.005202 | −13.2792 |
| N_NEU_FL_W; | | | | | | | | | |
| N_WBC_FL_CV; | 0.8277 | >−0.0105 | 20.3 | 72.5 | 79.7 | 27.5 | −5.0466 | 0.007783 | −4.92579 |
| N_NEU_FL_W; | | | | | | | | | |
| N_WBC_SSFS_Area; | 0.8258 | >−0.188 | 19.4 | 73.4 | 80.6 | 26.6 | 0.000334 | 0.005231 | −7.17242 |
| N_LYM_FL_W; | | | | | | | | | |
| N_WBC_FLFS_Area; | 0.8253 | >0.0064 | 23.6 | 72.1 | 76.4 | 27.9 | 0.00083 | −0.00163 | −3.89007 |
| N_LYM_SSFS_Area; | | | | | | | | | |
| N_LYM_FL_W; | 0.825 | >−0.3775 | 22.3 | 75.2 | 77.7 | 24.8 | 0.005395 | 4.621483 | −9.0677 |
| N_NEU_SS_CV; | | | | | | | | | |
| N_WBC_SS_W; | 0.8238 | >−0.3277 | 23.2 | 74.7 | 76.8 | 25.3 | 0.004926 | −4.96632 | −2.91854 |
| N_NEU_FL_CV; | | | | | | | | | |
| N_WBC_FL_CV; | 0.8227 | >−0.0244 | 22.5 | 71.3 | 77.5 | 28.7 | −5.91964 | 0.011792 | −0.78341 |
| N_NEU_FS_W; | | | | | | | | | |
| N_LYM_FLFS_Area; | 0.8225 | >−0.0475 | 24.7 | 72.8 | 75.3 | 27.2 | −0.00077 | 0.006954 | −7.594 |
| N_NEU_FL_W; | | | | | | | | | |
| N_WBC_FL_P; | 0.8222 | >−0.264 | 24.9 | 77.6 | 75.1 | 22.4 | 0.003602 | 0.009204 | −19.1153 |
| N_NEU_FS_P; | | | | | | | | | |
| N_WBC_SS_W; | 0.8221 | >−0.2409 | 20.8 | 72.1 | 79.2 | 27.9 | 0.005176 | −5.35641 | −1.00027 |
| N_WBC_FL_CV; | | | | | | | | | |
| N_WBC_FS_P; | 0.822 | >−0.1815 | 23.9 | 75.8 | 76.1 | 24.2 | 0.010154 | 0.003172 | −18.824 |
| N_NEU_FL_P; | | | | | | | | | |
| N_WBC_FS_W; | 0.8217 | >−0.2299 | 26.5 | 75.3 | 73.5 | 24.7 | 0.002776 | 0.00467 | −9.15479 |
| N_NEU_FL_W; | | | | | | | | | |
| N_WBC_FL_P; | 0.8205 | >−0.1375 | 22.8 | 74.6 | 77.2 | 25.4 | 0.003334 | 0.008533 | −16.5249 |
| N_WBC_FS_P; | | | | | | | | | |
| N_LYM_SS_W; | 0.8193 | >−0.0197 | 19.1 | 70.1 | 80.9 | 29.9 | 0.002089 | 0.005629 | −9.09241 |
| N_NEU_FL_W; | | | | | | | | | |
| N_WBC_SS_W; | 0.8193 | >−0.35 | 29.4 | 78.6 | 70.6 | 21.4 | 0.001407 | 0.004581 | −8.28464 |
| N_NEU_FL_W; | | | | | | | | | |
| N_NEU_FL_W; | 0.8192 | >−0.1293 | 22.8 | 71.5 | 77.2 | 28.5 | 0.005917 | 0.004628 | −14.8261 |
| N_NEU_FS_P; | | | | | | | | | |
| N_WBC_FL_P; | 0.8188 | >−0.2776 | 24.8 | 77 | 75.2 | 23 | 0.002856 | 0.001961 | −6.25204 |
| N_LYM_FL_W; | | | | | | | | | |
| N_NEU_SS_W; | 0.8188 | >−0.391 | 27.6 | 78.5 | 72.4 | 21.5 | 0.005073 | −5.61184 | −1.8486 |
| N_NEU_FL_CV; | | | | | | | | | |
| N_NEU_FL_P; | 0.8162 | >−0.2352 | 25.8 | 76.4 | 74.2 | 23.6 | 0.003329 | 0.009433 | −19.4902 |
| N_NEU_FS_P; | | | | | | | | | |
| N_LYM_FL_W; | 0.8149 | >−0.2576 | 24.7 | 75.4 | 75.3 | 24.6 | 0.002393 | 0.002468 | −6.32258 |
| N_NEU_FL_P; | | | | | | | | | |
| N_WBC_FL_P; | 0.8147 | >−0.1951 | 24.3 | 75.1 | 75.7 | 24.9 | 0.003639 | 0.001517 | −6.93882 |
| N_LYM_SS_W; | | | | | | | | | |
| N_WBC_FLSS_Area; | 0.8144 | >−0.0757 | 24 | 72.1 | 76 | 27.9 | 0.000507 | −0.00133 | −3.28386 |
| N_LYM_FLFS_Area; | | | | | | | | | |
| N_WBC_SSFS_Area; | 0.8143 | >−0.2019 | 28.2 | 76.1 | 71.8 | 23.9 | −0.00025 | 0.007404 | −8.00332 |
| N_NEU_FL_W; | | | | | | | | | |
| N_LYM_FLSS_Area; | 0.8128 | >−0.065 | 26.5 | 73.9 | 73.5 | 26.1 | −0.00054 | 0.000826 | −4.33042 |
| N_NEU_FLFS_Area; | | | | | | | | | |
| N_WBC_SS_W; | 0.8126 | >−0.2867 | 27.3 | 75.8 | 72.7 | 24.2 | 0.004719 | −0.00105 | −3.87457 |
| N_LYM_SSFS_Area; | | | | | | | | | |
| N_WBC_FL_P; | 0.812 | >−0.1716 | 23.7 | 74 | 76.3 | 26 | 0.003779 | −3E−05 | −6.18667 |
| N_LYM_FL_P; | | | | | | | | | |
| N_WBC_FL_P; | 0.8118 | >−0.1723 | 23.8 | 74 | 76.2 | 26 | 0.003758 | −2.6E−05 | −6.16164 |
| N_LYM_SS_P; | | | | | | | | | |
| N_WBC_FL_P; | 0.8118 | >−0.1764 | 23.9 | 74.2 | 76.1 | 25.8 | 0.003758 | −2.6E−05 | −6.15924 |
| N_LYM_FS_P; | | | | | | | | | |
| N_WBC_FL_P; | 0.8117 | >−0.1751 | 23.9 | 74.2 | 76.1 | 25.8 | 0.003757 | −2.6E−05 | −6.18351 |
| N_LYM_SS_CV; | | | | | | | | | |
| N_WBC_FL_P; | 0.8117 | >−0.1751 | 23.9 | 74.2 | 76.1 | 25.8 | 0.003757 | −2.6E−05 | −6.18351 |
| N_LYM_FL_CV; | | | | | | | | | |
| N_WBC_FL_P; | 0.8117 | >−0.1751 | 23.9 | 74.2 | 76.1 | 25.8 | 0.003757 | −2.6E−05 | −6.18352 |
| N_LYM_FS_CV; | | | | | | | | | |
| N_WBC_FS_W; | 0.8114 | >−0.2009 | 26.9 | 73 | 73.1 | 27 | 0.010017 | −0.00098 | −7.24541 |
| N_LYM_SSFS_Area; | | | | | | | | | |
| N_NEU_FL_W; | 0.8114 | >−0.154 | 24.5 | 72 | 75.5 | 28 | 0.004919 | 0.001941 | −8.0548 |
| N_NEU_FS_W; | | | | | | | | | |
| N_WBC_FL_P; | 0.8114 | >−0.2255 | 25.2 | 75.6 | 74.8 | 24.4 | 0.00374 | 0.00189 | −6.74709 |
| N_LYM_FS_W; | | | | | | | | | |
| N_LYM_SS_P; | 0.8112 | >−0.2798 | 26.2 | 73.4 | 73.8 | 26.6 | −9.5E−05 | 0.006797 | −9.33398 |
| N_NEU_FL_W; | | | | | | | | | |
| N_LYM_SSFS_Area; | 0.8111 | >−0.0512 | 26.6 | 73.9 | 73.4 | 26.1 | −0.00104 | 0.000817 | −3.45424 |
| N_NEU_FLFS_Area; | | | | | | | | | |
| N_WBC_FL_P; | 0.811 | >−0.1804 | 24.1 | 74.2 | 75.9 | 25.8 | 0.004759 | −0.00098 | −6.07355 |
| N_NEU_FL_P; | | | | | | | | | |
| N_WBC_FL_P; | 0.8109 | >−0.2689 | 26.1 | 76.6 | 73.9 | 23.4 | 0.003956 | 0.000237 | −7.11025 |
| N_LYM_SSFS_Area; | | | | | | | | | |
| N_WBC_FL_CV; | 0.8107 | >−0.2885 | 24.1 | 74.6 | 75.9 | 25.4 | 2.309984 | 0.005789 | −7.27298 |
| N_LYM_FL_W; | | | | | | | | | |
| N_LYM_FS_P; | 0.8107 | >−0.2851 | 28.7 | 76.2 | 71.3 | 23.8 | −9.4E−05 | 0.006794 | −9.32201 |
| N_NEU_FL_W; | | | | | | | | | |
| N_WBC_SS_CV; | 0.8107 | >−0.2481 | 27.1 | 74.8 | 72.9 | 25.2 | 0.888278 | 0.00598 | −9.33162 |
| N_NEU_FL_W; | | | | | | | | | |
| N_WBC_FL_P; | 0.8106 | >1599.2285 | 23.9 | 74 | 76.1 | 26 | 1 | 0 | 0 |
| N_WBC_FL_P; | 0.8104 | >−0.2381 | 25.9 | 75.9 | 74.1 | 24.1 | 0.003821 | 5.16E−05 | −6.46286 |
| N_LYM_FLSS_Area; | | | | | | | | | |
| N_WBC_FS_CV; | 0.8103 | >−0.2751 | 28.2 | 75.8 | 71.8 | 24.2 | 1.16291 | 0.006095 | −9.3118 |
| N_NEU_FL_W; | | | | | | | | | |
| N_NEU_SS_CV; | 0.8103 | >−0.1953 | 26.3 | 73.4 | 73.7 | 26.6 | −1.32828 | 0.007079 | −8.42144 |
| N_NEU_FL_W; | | | | | | | | | |
| N_NEU_FL_CV; | 0.8102 | >−0.0796 | 22.7 | 70.9 | 77.3 | 29.1 | −6.63149 | 0.000806 | −0.39229 |
| N_NEU_SSFS_Area; | | | | | | | | | |
| N_WBC_FL_P; | 0.8101 | >−0.2313 | 24.9 | 75.2 | 75.1 | 24.8 | 0.003876 | 0.000254 | −7.03921 |
| N_LYM_FLFS_Area; | | | | | | | | | |
| N_WBC_FLFS_Area; | 0.8101 | >−0.1548 | 27.9 | 74.5 | 72.1 | 25.5 | 0.000838 | −0.00137 | −4.53676 |
| N_LYM_FLFS_Area; | | | | | | | | | |
| N_NEU_SS_W; | 0.8099 | >−0.1315 | 24.4 | 71.7 | 75.6 | 28.3 | 0.000628 | 0.005232 | −8.01082 |
| N_NEU_FL_W; | | | | | | | | | |
| N_NEU_FL_W; | 0.8096 | >−0.2013 | 25.7 | 72.8 | 74.3 | 27.2 | 0.00595 | 1.502886 | −8.91316 |
| N_NEU_FS_CV; | | | | | | | | | |
| N_NEU_FL_W; | 0.8095 | >−0.2075 | 28.2 | 75.4 | 71.8 | 24.6 | 0.006454 | −0.00011 | −8.16828 |
| N_NEU_SSFS_Area; | | | | | | | | | |
| N_LYM_FS_CV; | 0.8093 | >−0.2838 | 25.4 | 72.2 | 74.6 | 27.8 | −9.5E−05 | 0.006795 | −9.4157 |
| N_NEU_FL_W; | | | | | | | | | |
| N_WBC_FL_CV; | 0.8093 | >−0.111 | 28 | 74.3 | 72 | 25.7 | −6.00257 | 17.61594 | −0.99563 |
| N_NEU_FS_CV; | | | | | | | | | |
| N_WBC_FLFS_Area; | 0.8087 | >−0.1442 | 25.1 | 72.5 | 74.9 | 27.5 | −8E−05 | 0.006409 | −8.08351 |
| N_NEU_FL_W; | | | | | | | | | |
| N_WBC_FS_P; | 0.8087 | >−0.4004 | 28.2 | 78 | 71.8 | 22 | 0.008486 | 0.004368 | −14.456 |
| N_LYM_FL_W; | | | | | | | | | |
| N_NEU_FL_W; | 0.8086 | >−0.2025 | 26.6 | 74.5 | 73.4 | 25.5 | 0.005019 | 0.000118 | −7.8207 |
| N_NEU_FLFS_Area; | | | | | | | | | |
| N_LYM_SS_W; | 0.8084 | >−0.1038 | 22.2 | 71.7 | 77.8 | 28.3 | 0.002045 | 0.003342 | −7.279 |
| N_NEU_FL_P; | | | | | | | | | |
| N_WBC_FLSS_Area; | 0.8084 | >−0.2282 | 28.4 | 75.3 | 71.6 | 24.7 | 1.36E−05 | 0.005639 | −7.97765 |
| N_NEU_FL_W; | | | | | | | | | |
| N_WBC_FS_P; | 0.8082 | >−0.2157 | 28.2 | 73.6 | 71.8 | 26.4 | 0.010412 | 0.000279 | −17.2117 |
| N_WBC_FLSS_Area; | | | | | | | | | |
| N_NEU_FL_W; | 0.8079 | >1360 | 25 | 71.3 | 75 | 28.7 | 1 | 0 | 0 |
| N_WBC_FL_CV; | 0.8079 | >−0.036 | 22.4 | 70.9 | 77.6 | 29.1 | −3.93911 | 0.000796 | −1.40767 |
| N_NEU_FLFS_Area; | | | | | | | | | |
| N_LYM_FL_W; | 0.8073 | >−0.2587 | 21.3 | 72.3 | 78.7 | 27.7 | 0.006034 | 2.579018 | −6.835 |
| N_NEU_FL_CV; | | | | | | | | | |
| N_WBC_SS_W; | 0.8066 | >−0.1629 | 22.1 | 70.6 | 77.9 | 29.4 | 0.002031 | 0.000439 | −6.10693 |
| N_NEU_FLFS_Area; | | | | | | | | | |
| N_LYM_FS_W; | 0.8066 | >−0.1398 | 25 | 71.3 | 75 | 28.7 | −0.00036 | 0.005817 | −7.92982 |
| N_NEU_FL_W; | | | | | | | | | |
| N_LYM_FL_W; | 0.8059 | >−0.2957 | 25.5 | 73.3 | 74.5 | 26.7 | 0.005906 | −0.00852 | −2.01041 |
| N_LYM_FS_W; | | | | | | | | | |
| N_LYM_FL_W; | 0.8057 | >−0.326 | 22.8 | 73.9 | 77.2 | 26.1 | 0.005376 | −6.8E−05 | −4.21674 |
| N_LYM_FS_P; | | | | | | | | | |
| N_LYM_SS_CV; | 0.8057 | >−0.2838 | 25.1 | 71.8 | 74.9 | 28.2 | −9.5E−05 | 0.006795 | −9.41564 |
| N_NEU_FL_W; | | | | | | | | | |
| N_LYM_FL_W; | 0.8056 | >−0.294 | 23 | 74.8 | 77 | 25.2 | 0.004756 | 0.008209 | −15.5493 |
| N_NEU_FS_P; | | | | | | | | | |
| N_WBC_SS_W; | 0.8056 | >−0.335 | 27.8 | 75.9 | 72.2 | 24.1 | 0.004528 | −0.00048 | −4.63232 |
| N_LYM_FLSS_Area; | | | | | | | | | |
| N_NEU_SS_P; | 0.805 | >−0.1629 | 24.7 | 71.1 | 75.3 | 28.9 | 0.005485 | 0.000501 | −10.2792 |
| N_NEU_FLFS_Area; | | | | | | | | | |
| N_LYM_SS_P; | 0.8048 | >−0.3257 | 23.3 | 73.8 | 76.7 | 26.2 | −6.9E−05 | 0.005377 | −4.22385 |
| N_LYM_FL_W; | | | | | | | | | |
| N_LYM_FL_CV; | 0.8047 | >−0.2838 | 25.3 | 72.2 | 74.7 | 27.8 | −9.5E−05 | 0.006795 | −9.41564 |
| N_NEU_FL_W; | | | | | | | | | |
| N_LYM_FL_P; | 0.8039 | >−0.2575 | 25.3 | 72.1 | 74.7 | 27.9 | −8.2E−05 | 0.006743 | −9.2522 |
| N_NEU_FL_W; | | | | | | | | | |
| N_WBC_SS_W; | 0.8037 | >−0.3303 | 27.2 | 75.9 | 72.8 | 24.1 | 0.003314 | 0.009236 | −16.5689 |
| N_WBC_FS_P; | | | | | | | | | |
| N_WBC_FS_W; | 0.8035 | >−0.0824 | 21.6 | 69.2 | 78.4 | 30.8 | 0.003879 | 0.00044 | −7.07005 |
| N_NEU_FLFS_Area; | | | | | | | | | |
| N_LYM_FL_P; | 0.8029 | >−0.274 | 28.2 | 76.8 | 71.8 | 23.2 | −3.4E−05 | 0.003495 | −6.23459 |
| N_NEU_FL_P; | | | | | | | | | |
| N_WBC_FS_P; | 0.8028 | >−0.1406 | 28.1 | 72.6 | 71.9 | 27.4 | 0.007693 | 0.000572 | −14.2468 |
| N_NEU_FLFS_Area; | | | | | | | | | |
| N_LYM_FLSS_Area; | 0.8028 | >−0.2193 | 26.7 | 74.7 | 73.3 | 25.3 | −7.7E−05 | 0.003407 | −5.84793 |
| N_NEU_FL_P; | | | | | | | | | |
| N_LYM_SS_P; | 0.8027 | >−0.2771 | 28.4 | 77 | 71.6 | 23 | −3.1E−05 | 0.003472 | −6.20395 |
| N_NEU_FL_P; | | | | | | | | | |
| N_LYM_FS_P; | 0.8027 | >−0.2766 | 28.4 | 77 | 71.6 | 23 | −3E−05 | 0.003471 | −6.2006 |
| N_NEU_FL_P; | | | | | | | | | |
| N_LYM_SS_CV; | 0.8026 | >−0.2764 | 28.4 | 77 | 71.6 | 23 | −3E−05 | 0.003471 | −6.22972 |
| N_NEU_FL_P; | | | | | | | | | |
| N_LYM_FL_CV; | 0.8026 | >−0.2764 | 28.4 | 77 | 71.6 | 23 | −3E−05 | 0.003471 | −6.22971 |
| N_NEU_FL_P; | | | | | | | | | |
| N_LYM_FS_CV; | 0.8026 | >−0.2764 | 28.4 | 77 | 71.6 | 23 | −3E−05 | 0.003471 | −6.22973 |
| N_NEU_FL_P; | | | | | | | | | |
| N_WBC_FS_W; | 0.8024 | >−0.2129 | 28.4 | 73.5 | 71.6 | 26.5 | 0.009456 | −0.00043 | −7.7322 |
| N_LYM_FLSS_Area; | | | | | | | | | |
| N_WBC_SS_P; | 0.8018 | >−0.1786 | 25.3 | 71 | 74.7 | 29 | 0.005759 | 0.000487 | −10.3611 |
| N_NEU_FLFS_Area; | | | | | | | | | |
| N_LYM_SSFS_Area; | 0.8015 | >−0.2754 | 28.6 | 77 | 71.4 | 23 | −2.7E−05 | 0.003445 | −6.10961 |
| N_NEU_FL_P; | | | | | | | | | |
| N_LYM_FS_W; | 0.8015 | >−0.2078 | 26.6 | 74.7 | 73.4 | 25.3 | 0.000861 | 0.003449 | −6.45814 |
| N_NEU_FL_P; | | | | | | | | | |
| N_WBC_SS_W; | 0.8014 | >−0.2165 | 23.1 | 72.8 | 76.9 | 27.2 | 0.002189 | 0.00465 | −7.56978 |
| N_WBC_FS_W; | | | | | | | | | |
| N_NEU_FL_P; | 0.8013 | >1715.2215 | 28.4 | 76.8 | 71.6 | 23.2 | 1 | 0 | 0 |
| N_LYM_FL_W; | 0.8012 | >−0.3268 | 25.2 | 74.4 | 74.8 | 25.6 | 0.005368 | −6.8E−05 | −4.27749 |
| N_LYM_FS_CV; | | | | | | | | | |
| N_LYM_FLFS_Area; | 0.8009 | >−0.2272 | 26.8 | 74.7 | 73.2 | 25.3 | 3.11E−05 | 0.003464 | −6.29476 |
| N_NEU_FL_P; | | | | | | | | | |
| N_LYM_FL_W; | 0.8009 | >−0.3603 | 27.1 | 76.7 | 72.9 | 23.3 | 0.004917 | −0.00018 | −3.46068 |
| N_LYM_SSFS_Area; | | | | | | | | | |
| N_WBC_FL_CV; | 0.8002 | >−0.2825 | 26.8 | 74.2 | 73.2 | 25.8 | −4.93435 | 0.004849 | −0.30807 |
| N_NEU_SS_W; | | | | | | | | | |
| N_WBC_SSFS_Area; | 0.8001 | >−0.1022 | 26.4 | 72.1 | 73.6 | 27.9 | −0.0005 | 0.001142 | −4.02688 |
| N_NEU_FLFS_Area; | | | | | | | | | |

TABLE 11-5
Efficacy of using PCT (procalcitonin) of prior art, and the parameters of the
DIFF channel for identification of a common infection and a severe infection

| | | | False | True | True | False |
|---|---|---|---|---|---|---|
| Infection marker | | Determination | positive | positive | negative | negative |
| parameter | ROC_AUC | threshold | rate | rate | rate | rate |
| PCT | 0.806 | >0.46 | 31.8% | 80.5% | 68.2% | 19.5% |
| D_Neu_SS_W | 0.664 | >259.324 | 39.3% | 633.3% | 60.7% | 36.7% |
| D_Neu_FL_W | 0.758 | >220.767 | 13.6% | 54.3% | 86.4% | 45.7% |
| D_Neu_FS_W | 0.542 | >572.274 | 34.3% | 41.9% | 65.7% | 58.1% |

It has been reported in the prior art (Crouser E. Parrillo J. Seymour C et al. Improved Early Detection of Sepsis in the ED With a Novel Monocyte Distribution Width Biomarker. CHEST. 2017; 152 (3): 518-526) that, from the blood routine test scattergram of the DIFF channel of BCI blood analyzer, the distribution width of neutrophils was used to identify a common infection and a severe infection, and the ROC_AUC was 0.79, the determination threshold was >20.5, the false positive rate was 27%, the true positive rate was 77.0%, the true negative rate was 73%, and the false negative rate was 23%. From the reported data, it was similar to MINDRAY's DIFF channel for identifying a common infection and a severe infection.

From the comparison between Table 11-5 and Table 9, 10, 11-1, 11-2, 11-3, 11-4, it can be seen that the parameters of the WNB channel have similar diagnostic efficacy to PCT or even better diagnostic efficacy than PCT in the differential diagnosis of severe infection, are possible to replace PCT markers, and realize the use of blood routine test data to give prompt for identification of a common infection and a severe infection without additional cost; In addition, the parameters of the WNB channel have better diagnostic performance than the parameters of the DIFF channel in the differential diagnosis of severe infection.

TABLE 11-6
Illustration of the statistical methods and testing methods
used in this example by taking three parameters as examples

| Infection | | | | |
|---|---|---|---|---|
| marker | Positive sample | Negative sample | | |
| parameter | Mean ± SD | Mean ± SD | F value | P value |
| N_WBC_FL_W | 2031.5 ± 287.5 | 1683.7 ± 207.1 | 740.08 | <0.0001 |
| N_NEU_FLSS_Area | 14534.6371 ± 3651.0351 | 11908.1115 ± 2034.0094 | 301.83 | <0.0001 |
| N_WBC_SS_P | 1206.8579 ± 117.4999 | 1118.5766 ± 69.5627 | 319.26 | <0.0001 |

As can be seen from Table 11-6, this parameter is analyzed by Welch test, and there is a significant statistical difference between the two groups (p<0.0001)

As can be seen from Tables 9, 10, and 11-1 to 11-6, the infection marker parameters provided in the disclosure can be used to effectively determine whether a subject has a severe infection. For the same reasons as example 1, the disclosure accidentally discovered through in-depth investigation that a more useful feature can be found from the WNB channel than from the DIFF channel to identify a severe infection and a common infection.

### Example 3 Diagnosis of Sepsis

1748 blood samples were subjected to blood routine test using the BC-6800 Plus blood cell analyzer produced by SHENZHEN MINDRAY BIO-MEDICAL ELECTRONICS CO., LTD. in accordance with the steps similar to example 1 of the disclosure, and the aforementioned method was adopted for diagnosis of sepsis based on the scattergram. Among them, there were 506 sepsis samples, that is, positive samples, and 1,242 non-sepsis samples, that is, negative samples.

Inclusion criteria for these 1748 cases: adult ICU patients with or without acute infection. Exclusion criteria: pregnant people, myelosuppressed people on chemotherapy, people on immunosuppressant treatment, patients with hematologic diseases.

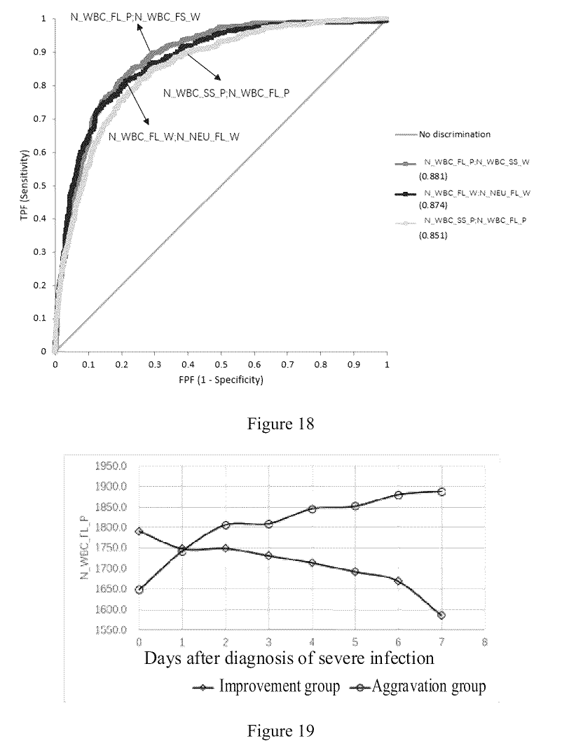

Table 12 shows the infection marker parameters used and their corresponding diagnostic efficacy, and FIGS. 17 and 18 show ROC curves corresponding to the infection marker parameters in Table 12. In Table 12:

$$Combination parameter 1 = 0.004088 \times N\_WBC \_FL \_P + 0.009 \; 059 \times N\_WBC \_FS \_W ;$$

$$Combination parameter 2 = 0.006086 \times N\_WBC \_FL \_W - 0.00 \; 017 \times N\_NEU \_FL \_W ;$$

$$Combination parameter 3 = 0.007722 \times N\_WBC \_SS \_P + 0.003 \; 547 \times N\_WBC \_FL \_P .$$

TABLE 12
Efficacy of different infection marker parameters for diagnosis of sepsis

| | | | False | True | True | False |
|---|---|---|---|---|---|---|
| Infection marker | | Determination | positive | positive | negative | negative |
| parameter | ROC_AUC | threshold | rate | rate | rate | rate |
| N_WBC_FL_W | 0.873 | >1872 | 20.8% | 80% | 79.2% | 20% |
| N_NEU_FL_W | 0.806 | >1360 | 28.7% | 75.3% | 71.3% | 24.7% |
| N_NEU_FLSS_Area | 0.772 | >10951.68 | 25.2% | 67.8% | 74.8% | 32.2% |
| Combination | 0.881 | >−1.0161 | 19.4% | 81.2% | 80.6% | 18.8% |
| parameter 1 | | | | | | |
| Combination | 0.874 | >−1.1218 | 21.3% | 81.2% | 78.7% | 18.8% |
| parameter 2 | | | | | | |
| Combination | 0.851 | >−1.1751 | 25.5% | 82% | 74.5% | 18% |
| parameter 3 | | | | | | |

In addition, Table 13 shows the efficacy of using other single leukocyte characteristic parameters as infection marker parameters for diagnosis of sepsis in this example, and Tables 14 show the efficacy of using other parameter combinations as infection marker parameters for diagnosis of sepsis in this example, wherein infection marker parameters are calculated by the function Y=A×X1+B×X2+C based on the parameter combinations in the Table 14, where Y represents an infection marker parameter. X1 represents the first leukocyte parameter. X2 represents the second leukocyte parameter, and A, B, and C are constants.

TABLE 13
Efficacy of other single parameters for diagnosis of sepsis

| | | | False | True | True | False |
|---|---|---|---|---|---|---|
| | | Determination | positive | positive | negative | negative |
| Parameter | ROC_AUC | threshold | rate % | rate % | rate % | rate % |
| N_WBC_FL_P | 0.832 | >1638.1165 | 23.3 | 76.2 | 76.7 | 23.8 |
| N_NEU_FL_P | 0.8246 | >1812.7065 | 21.5 | 73.5 | 78.5 | 26.5 |
| N_WBC_SS_W | 0.7869 | >1328 | 27.7 | 75.5 | 72.3 | 24.5 |
| N_NEU_FLFS_Area | 0.78 | >7429.12 | 26.2 | 70.8 | 73.8 | 29.2 |
| N_WBC_FS_W | 0.7782 | >976 | 21.6 | 67.6 | 78.4 | 32.4 |
| N_NEU_FS_W | 0.7738 | >624 | 31.4 | 72.7 | 68.6 | 27.3 |
| N_NEU_SS_W | 0.7641 | >1168 | 31.5 | 74.3 | 68.5 | 25.7 |
| N_WBC_SS_P | 0.7595 | >1145.5385 | 30.6 | 71.3 | 69.4 | 28.7 |
| N_NEU_SS_P | 0.7578 | >1162.0325 | 33.2 | 74.3 | 66.8 | 25.7 |
| N_WBC_FLSS_Area | 0.7543 | >12876.8 | 30.3 | 71.1 | 69.7 | 28.9 |
| N_NEU_FS_CV | 0.7515 | >0.4405 | 30.5 | 68.3 | 69.5 | 31.7 |

TABLE 14-1
Efficacy of parameter combinations containing N_WBC_FL_W for diagnosis of sepsis

| | | | False | True | True | False | | | |
|---|---|---|---|---|---|---|---|---|---|
| Parameter | | Determination | positive | positive | negative | positive | | | |
| combination | ROC_AUC | threshold | rate % | rate % | rate % | rate % | A | B | C |
| N_WBC_FL_W; | 0.8804 | >−0.9208 | 18.3 | 78.8 | 81.7 | 21.2 | 0.006093 | 0.005877 | −20.071 |
| N_WBC_FS_P; | | | | | | | | | |
| N_WBC_FL_W; | 0.8793 | >−0.9156 | 17.2 | 78.8 | 82.8 | 21.2 | 0.006184 | 3.089254 | −14.0448 |
| N_NEU_FS_CV; | | | | | | | | | |
| N_WBC_SS_CV; | 0.8787 | >−1.0489 | 19.4 | 80.2 | 80.6 | 19.8 | 2.07475 | 0.006185 | −15.135 |
| N_WBC_FL_W; | | | | | | | | | |
| N_WBC_SS_W; | 0.8785 | >−0.9117 | 17.5 | 78.7 | 82.5 | 21.3 | 0.001368 | 0.005296 | −12.8668 |
| N_WBC_FL_W; | | | | | | | | | |
| N_WBC_FL_W; | 0.878 | >−1.0008 | 18.1 | 80.2 | 81.9 | 19.8 | 0.005834 | 0.003859 | −16.5927 |
| N_NEU_SS_P; | | | | | | | | | |
| N_WBC_SS_P; | 0.8775 | >−0.9698 | 18 | 79.4 | 82 | 20.6 | 0.003896 | 0.005861 | −16.5875 |
| N_WBC_FL_W; | | | | | | | | | |
| N_WBC_FL_W; | 0.8772 | >−0.8696 | 16.9 | 78.1 | 83.1 | 21.9 | 0.005601 | 0.001618 | −12.5692 |
| N_NEU_FS_W; | | | | | | | | | |
| N_WBC_FL_W; | 0.8761 | >−0.8641 | 16.8 | 77.4 | 83.2 | 22.6 | 0.006372 | 1.220541 | −14.2735 |
| N_NEU_SS_CV; | | | | | | | | | |
| N_WBC_FL_W; | 0.876 | >−0.9252 | 18 | 78.3 | 82 | 21.7 | 0.005496 | 0.00105 | −12.6327 |
| N_NEU_SS_W; | | | | | | | | | |
| N_WBC_FL_W; | 0.8759 | >−0.8418 | 18 | 78.5 | 82 | 21.5 | 0.006575 | −0.00013 | −12.0489 |
| N_WBC_FLFS_Area; | | | | | | | | | |
| N_WBC_FL_P; | 0.8756 | >−1.0231 | 20.8 | 80.2 | 79.2 | 19.8 | 0.001069 | 0.005643 | −13.3866 |
| N_WBC_FL_W; | | | | | | | | | |
| N_WBC_FL_W; | 0.8751 | >−0.9962 | 20.6 | 80.2 | 79.4 | 19.8 | 0.006543 | −1.54258 | −11.5245 |
| N_WBC_FL_CV; | | | | | | | | | |
| N_WBC_FL_W; | 0.875 | >−1.1053 | 20.7 | 80.4 | 79.3 | 19.6 | 0.006541 | 0.000684 | −14.2997 |
| N_NEU_FS_P; | | | | | | | | | |
| N_WBC_FL_W; | 0.8749 | >−1.0909 | 20.9 | 80.8 | 79.1 | 19.2 | 0.006545 | −0.55401 | −12.8881 |
| N_NEU_FL_CV; | | | | | | | | | |
| N_WBC_FL_W; | 0.8749 | >−0.9635 | 19.3 | 79.4 | 80.7 | 20.6 | 0.005845 | 0.000568 | −12.5343 |
| N_WBC_FS_W; | | | | | | | | | |
| N_WBC_FL_W; | 0.8748 | >−0.8735 | 18.2 | 77.9 | 81.8 | 22.1 | 0.006263 | −3.7E−05 | −12.2701 |
| N_WBC_FLSS_Area; | | | | | | | | | |
| N_WBC_FL_W; | 0.8746 | >−1.1319 | 21.9 | 81.4 | 78.1 | 18.6 | 0.006091 | 0.000525 | −13.4157 |
| N_NEU_FL_P; | | | | | | | | | |
| N_WBC_FL_W; | 0.8743 | >−0.9725 | 20.6 | 80.2 | 79.4 | 19.8 | 0.006605 | −0.04159 | −13.4086 |
| N_WBC_FS_CV; | | | | | | | | | |
| N_WBC_FL_W; | 0.8739 | >−0.8312 | 18 | 77.5 | 82 | 22.5 | 0.006228 | −8.8E−05 | −11.8941 |
| N_WBC_SSFS_Area; | | | | | | | | | |
| N_WBC_FL_W; | 0.8738 | >−0.9321 | 18.7 | 78.7 | 81.3 | 21.3 | 0.005839 | 5.62E−05 | −12.362 |
| N_NEU_SSFS_Area; | | | | | | | | | |
| N_WBC_FL_W; | 0.8735 | >−1.1218 | 21.3 | 81.2 | 78.7 | 18.8 | 0.006086 | −0.00017 | −12.2035 |
| N_NEU_FL_W; | | | | | | | | | |
| N_WBC_FL_W; | 0.8731 | >−0.9481 | 19 | 78.9 | 81 | 21.1 | 0.005809 | 4.77E−05 | −12.2679 |
| N_NEU_FLFS_Area; | | | | | | | | | |
| N_WBC_FL_W; | 0.8731 | >−0.9238 | 18.7 | 78.7 | 81.3 | 21.3 | 0.005704 | 4.54E−05 | −12.2148 |
| N_NEU_FLSS_Area; | | | | | | | | | |

TABLE 14-2
Efficacy of parameter combinations containing N_NEU_FL_W for diagnosis of sepsis

| | | | False | True | True | False | | | |
|---|---|---|---|---|---|---|---|---|---|
| Parameter | | Determination | positive | positive | negative | positive | | | |
| combination | ROC_AUC | threshold | rate % | rate % | rate % | rate % | A | B | C |
| N_WBC_FL_W; | 0.8735 | >−1.1218 | 21.3 | 81.2 | 78.7 | 18.8 | 0.006086 | −0.00017 | −12.2035 |
| N_NEU_FL_W; | | | | | | | | | |
| N_WBC_FL_P; | 0.861 | >−1.0286 | 23.3 | 81 | 76.7 | 19 | 0.003432 | 0.004769 | −13.2418 |
| N_NEU_FL_W; | | | | | | | | | |
| N_NEU_FL_P; | 0.8581 | >−0.9716 | 21.5 | 79.8 | 78.5 | 20.2 | 0.003219 | 0.004848 | −13.4874 |
| N_NEU_FL_W; | | | | | | | | | |
| N_NEU_FL_W; | 0.8541 | >−0.8535 | 20.8 | 78.4 | 79.2 | 21.6 | 0.008541 | −7.71388 | −6.71761 |
| N_NEU_FL_CV; | | | | | | | | | |
| N_WBC_FS_P; | 0.8305 | >−0.9702 | 24.8 | 75.4 | 75.2 | 24.6 | 0.008409 | 0.005411 | −19.3267 |
| N_NEU_FL_W; | | | | | | | | | |
| N_NEU_SS_P; | 0.8299 | >−1.2579 | 27.5 | 78.8 | 72.5 | 21.2 | 0.006538 | 0.004769 | −15.3746 |
| N_NEU_FL_W; | | | | | | | | | |
| N_WBC_FL_CV; | 0.8295 | >−0.8478 | 23.3 | 75.6 | 76.7 | 24.4 | −4.69578 | 0.007167 | −5.42342 |
| N_NEU_FL_W; | | | | | | | | | |
| N_WBC_SS_P; | 0.8256 | >−1.1738 | 26.9 | 77.6 | 73.1 | 22.4 | 0.006437 | 0.004703 | −14.9938 |
| N_NEU_FL_W; | | | | | | | | | |
| N_WBC_SS_W; | 0.8213 | >−1.1754 | 26.7 | 76.1 | 73.3 | 23.9 | 0.002205 | 0.003995 | −9.57136 |
| N_NEU_FL_W; | | | | | | | | | |
| N_WBC_FS_W; | 0.8189 | >−1.1615 | 28.7 | 78.1 | 71.3 | 21.9 | 0.002825 | 0.004719 | −10.2468 |
| N_NEU_FL_W; | | | | | | | | | |
| N_WBC_SSFS_Area; | 0.8178 | >−1.0562 | 29.3 | 78.1 | 70.7 | 21.9 | −0.00028 | 0.007498 | −8.72203 |
| N_NEU_FL_W; | | | | | | | | | |
| N_NEU_FL_W; | 0.8155 | >−1.0683 | 26.6 | 76 | 73.4 | 24 | 0.005928 | 0.00392 | −14.7912 |
| N_NEU_FS_P; | | | | | | | | | |
| N_WBC_FLFS_Area; | 0.8094 | >−1.0062 | 26.4 | 74.3 | 73.6 | 25.7 | −0.00014 | 0.006835 | −9.04692 |
| N_NEU_FL_W; | | | | | | | | | |
| N_NEU_SS_CV; | 0.8093 | >−1.1275 | 28.6 | 75.8 | 71.4 | 24.2 | −0.93584 | 0.006829 | −9.44505 |
| N_NEU_FL_W; | | | | | | | | | |
| N_WBC_SS_CV; | 0.8091 | >−1.0973 | 26.5 | 74.3 | 73.5 | 25.7 | 1.811711 | 0.005657 | −10.9655 |
| N_NEU_FL_W; | | | | | | | | | |
| N_NEU_FL_W; | 0.8086 | >−1.076 | 28.3 | 75.9 | 71.7 | 24.1 | 0.006506 | −0.00012 | −9.1259 |
| N_NEU_SSFS_Area; | | | | | | | | | |
| N_NEU_FL_W; | 0.8085 | >−1.0826 | 27.5 | 75.5 | 72.5 | 24.5 | 0.005121 | 0.00153 | −9.02662 |
| N_NEU_FS_W; | | | | | | | | | |
| N_WBC_FS_CV; | 0.8084 | >−1.1121 | 28.2 | 75.8 | 71.8 | 24.2 | 0.960148 | 0.006173 | −10.2395 |
| N_NEU_FL_W; | | | | | | | | | |
| N_NEU_SS_W; | 0.8083 | >−1.0495 | 26.2 | 73.3 | 73.8 | 26.7 | 0.001259 | 0.004721 | −9.05092 |
| N_NEU_FL_W; | | | | | | | | | |
| N_NEU_FL_W; | 0.808 | >−1.0345 | 25.6 | 73.3 | 74.4 | 26.7 | 0.006073 | 1.068831 | −9.85554 |
| N_NEU_FS_CV; | | | | | | | | | |
| N_NEU_FL_W; | 0.8053 | >−1.0453 | 26.9 | 74.5 | 73.1 | 25.5 | 0.005061 | 0.000113 | −8.79843 |
| N_NEU_FLFS_Area; | | | | | | | | | |
| N_WBC_FLSS_Area; | 0.8051 | >−1.0103 | 28 | 75.3 | 72 | 24.7 | 7.98E−06 | 0.005726 | −8.97855 |
| N_NEU_FL_W; | | | | | | | | | |
| N_NEU_FL_W; | 0.8049 | >−1.0505 | 27.3 | 74.3 | 72.7 | 25.7 | 0.004987 | 7.36E−05 | −8.66016 |
| N_NEU_FLSS_Area; | | | | | | | | | |

TABLE 14-3
Efficacy of parameter combinations containing N_NEU_FLSS_Area for diagnosis of sepsis

| | | | False | True | True | False | | | |
|---|---|---|---|---|---|---|---|---|---|
| Parameter | | Determination | positive | positive | negative | positive | | | |
| combination | ROC_AUC | threshold | rate % | rate % | rate % | rate % | A | B | C |
| N_WBC_FL_W; | 0.8731 | >−0.9238 | 18.7 | 78.7 | 81.3 | 21.3 | 0.005704 | 4.54E−05 | −12.2148 |
| N_NEU_FLSS_Area; | | | | | | | | | |
| N_WBC_FL_P; | 0.8586 | >−1.0972 | 24.3 | 81.6 | 75.7 | 18.4 | 0.003701 | 0.000299 | −10.3634 |
| N_NEU_FLSS_Area; | | | | | | | | | |
| N_NEU_FL_P; | 0.8552 | >−1.1393 | 24.5 | 82.2 | 75.5 | 17.8 | 0.003488 | 0.000305 | −10.6116 |
| N_NEU_FLSS_Area; | | | | | | | | | |
| N_NEU_FL_CV; | 0.8325 | >−0.9255 | 23.1 | 75.2 | 76.9 | 24.8 | −6.50152 | 0.000513 | −1.47551 |
| N_NEU_FLSS_Area; | | | | | | | | | |
| N_NEU_FL_W; | 0.8049 | >−1.0505 | 27.3 | 74.3 | 72.7 | 25.7 | 0.004987 | 7.36E−05 | −8.66016 |
| N_NEU_FLSS_Area; | | | | | | | | | |
| N_WBC_FL_CV; | 0.8019 | >−0.9149 | 25.9 | 72.7 | 74.1 | 27.3 | −4.1213 | 0.000439 | −0.98817 |
| N_NEU_FLSS_Area; | | | | | | | | | |
| N_WBC_SS_W; | 0.8011 | >−1.1392 | 24.6 | 74.5 | 75.4 | 25.5 | 0.002793 | 0.000198 | −7.04498 |
| N_NEU_FLSS_Area; | | | | | | | | | |
| N_WBC_FS_P; | 0.8005 | >−0.9853 | 26.6 | 72.5 | 73.4 | 27.5 | 0.008906 | 0.000312 | −15.892 |
| N_NEU_FLSS_Area; | | | | | | | | | |
| N_WBC_SSFS_Area; | 0.8002 | >−1.0045 | 30.9 | 75.1 | 69.1 | 24.9 | −0.00068 | 0.000751 | −3.04041 |
| N_NEU_FLSS_Area; | | | | | | | | | |
| N_WBC_FS_W; | 0.7973 | >−1.0833 | 26.2 | 72.1 | 73.8 | 27.9 | 0.004219 | 0.00026 | −7.94057 |
| N_NEU_FLSS_Area; | | | | | | | | | |
| N_NEU_SS_P; | 0.7952 | >−1.1264 | 26.5 | 72.9 | 73.5 | 27.1 | 0.006898 | 0.000232 | −11.7334 |
| N_NEU_FLSS_Area; | | | | | | | | | |
| N_WBC_SS_P; | 0.7913 | >−1.1408 | 27.7 | 73.5 | 72.3 | 26.5 | 0.007071 | 0.000221 | −11.6417 |
| N_NEU_FLSS_Area; | | | | | | | | | |
| N_NEU_FS_W; | 0.7839 | >−1.0955 | 28.7 | 72.3 | 71.3 | 27.7 | 0.003575 | 0.000262 | −6.14334 |
| N_NEU_FLSS_Area; | | | | | | | | | |
| N_NEU_FLSS_Area; | 0.7839 | >−1.0247 | 28.1 | 71.9 | 71.9 | 28.1 | 0.000742 | −0.00068 | −4.38426 |
| N_NEU_SSFS_Area; | | | | | | | | | |
| N_NEU_FS_P; | 0.7819 | >−0.8879 | 21.5 | 66.3 | 78.5 | 33.7 | 0.003774 | 0.000358 | −10.2954 |
| N_NEU_FLSS_Area; | | | | | | | | | |
| N_WBC_SS_CV; | 0.7818 | >−1.0871 | 25.5 | 72.1 | 74.5 | 27.9 | 2.951766 | 0.000317 | −7.97043 |
| N_NEU_FLSS_Area; | | | | | | | | | |
| N_NEU_SS_W; | 0.7817 | >−1.0373 | 24.9 | 69.8 | 75.1 | 30.2 | 0.001895 | 0.000253 | −6.08439 |
| N_NEU_FLSS_Area; | | | | | | | | | |
| N_NEU_FS_CV; | 0.7807 | >−1.2248 | 33.4 | 76.8 | 66.6 | 23.2 | 5.079869 | 0.000297 | −6.50925 |
| N_NEU_FLSS_Area; | | | | | | | | | |
| N_NEU_FLFS_Area; | 0.7798 | >−1.0015 | 26.6 | 70.8 | 73.4 | 29.2 | 0.000416 | 0.000155 | −5.7575 |
| N_NEU_FLSS_Area; | | | | | | | | | |
| N_WBC_FS_CV; | 0.7777 | >−1.1267 | 28.7 | 72.5 | 71.3 | 27.5 | 2.843449 | 0.000352 | −6.98464 |
| N_NEU_FLSS_Area; | | | | | | | | | |
| N_NEU_SS_CV; | 0.7734 | >−1.0216 | 26.9 | 70.3 | 73.1 | 29.7 | 0.680228 | 0.000373 | −5.76496 |
| N_NEU_FLSS_Area; | | | | | | | | | |
| N_WBC_FLSS_Area; | 0.7724 | >−1.0158 | 28.4 | 69.6 | 71.6 | 30.4 | −0.00013 | 0.000518 | −4.91898 |
| N_NEU_FLSS_Area; | | | | | | | | | |
| N_WBC_FLFS_Area; | 0.7716 | >−0.9705 | 26.1 | 68 | 73.9 | 32 | −0.00019 | 0.000496 | −4.54333 |
| N_NEU_FLSS_Area; | | | | | | | | | |

TABLE 14-4
Efficacy of parameter combinations containing N_WBC_FL_P for diagnosis of sepsis

| | | | False | True | True | False | | | |
|---|---|---|---|---|---|---|---|---|---|
| Parameter | | Determination | positive | positive | negative | positive | | | |
| combination | ROC_AUC | threshold | rate % | rate % | rate % | rate % | A | B | C |
| N_WBC_FL_P; | 0.881 | >−1.0161 | 19.4 | 81.2 | 80.6 | 18.8 | 0.004088 | 0.009059 | −16.6003 |
| N_WBC_FS_W; | | | | | | | | | |
| N_WBC_FL_P; | 0.88 | >−1.1129 | 21 | 82.4 | 79 | 17.6 | 0.004626 | 12.43796 | −18.0312 |
| N_WBC_FS_CV; | | | | | | | | | |
| N_WBC_FL_P; | 0.8795 | >−0.8932 | 19.1 | 80.8 | 80.9 | 19.2 | 0.004164 | 11.89733 | −13.2122 |
| N_NEU_FS_CV; | | | | | | | | | |
| N_WBC_FL_P; | 0.8791 | >−0.9409 | 19.8 | 81 | 80.2 | 19 | 0.004042 | 0.007792 | −12.6941 |
| N_NEU_FS_W; | | | | | | | | | |
| N_WBC_SS_CV; | 0.8787 | >−1.2006 | 21.2 | 82.8 | 78.8 | 17.2 | 7.261505 | 0.004653 | −17.3391 |
| N_WBC_FL_P; | | | | | | | | | |
| N_WBC_SS_W; | 0.8786 | >−1.25 | 22.1 | 83 | 77.9 | 17 | 0.004255 | 0.004042 | −13.617 |
| N_WBC_FL_P; | | | | | | | | | |
| N_WBC_FL_P; | 0.8764 | >−0.98 | 18.9 | 78.4 | 81.1 | 21.6 | 0.00719 | 9.406125 | −23.7851 |
| N_WBC_FL_CV; | | | | | | | | | |
| N_WBC_FL_P; | 0.8756 | >−1.0231 | 20.8 | 80.2 | 79.2 | 19.8 | 0.001069 | 0.005643 | −13.3866 |
| N_WBC_FL_W; | | | | | | | | | |
| N_WBC_FL_P; | 0.8699 | >−1.157 | 22 | 80.8 | 78 | 19.2 | 0.004109 | 0.003952 | −12.6813 |
| N_NEU_SS_W; | | | | | | | | | |
| N_WBC_FL_P; | 0.8646 | >−0.964 | 21.3 | 78.8 | 78.7 | 21.2 | 0.004349 | 0.000544 | −11.952 |
| N_NEU_SSFS_Area; | | | | | | | | | |
| N_WBC_FL_P; | 0.861 | >−1.0286 | 23.3 | 81 | 76.7 | 19 | 0.003432 | 0.004769 | −13.2418 |
| N_NEU_FL_W; | | | | | | | | | |
| N_WBC_FL_P; | 0.859 | >−1.0381 | 22.2 | 79.2 | 77.8 | 20.8 | 0.004528 | 5.618989 | −14.2856 |
| N_NEU_SS_CV; | | | | | | | | | |
| N_WBC_FL_P; | 0.8586 | >−1.0972 | 24.3 | 81.6 | 75.7 | 18.4 | 0.003701 | 0.000299 | −10.3634 |
| N_NEU_FLSS_Area; | | | | | | | | | |
| N_WBC_FL_P; | 0.8586 | >−1.0879 | 24.7 | 81 | 75.3 | 19 | 0.006394 | 7.373055 | −17.329 |
| N_NEU_FL_CV; | | | | | | | | | |
| N_WBC_FL_P; | 0.8577 | >−0.9089 | 20.7 | 77.4 | 79.3 | 22.6 | 0.003662 | 0.000523 | −10.928 |
| N_NEU_FLFS_Area; | | | | | | | | | |
| N_WBC_FL_P; | 0.8563 | >−1.2069 | 25.2 | 81.8 | 74.8 | 18.2 | 0.004682 | 0.000441 | −12.7205 |
| N_WBC_SSFS_Area; | | | | | | | | | |
| N_WBC_FL_P; | 0.8543 | >−1.0014 | 20.1 | 78 | 79.9 | 22 | 0.003595 | 0.007638 | −16.0002 |
| N_NEU_SS_P; | | | | | | | | | |
| N_WBC_FL_P; | 0.8534 | >−0.9231 | 20.6 | 76.6 | 79.4 | 23.4 | 0.0038 | 0.000251 | −10.5982 |
| N_WBC_FLSS_Area; | | | | | | | | | |
| N_WBC_SS_P; | 0.8507 | >−1.1751 | 25.5 | 82 | 74.5 | 18 | 0.007722 | 0.003547 | −15.8201 |
| N_WBC_FL_P; | | | | | | | | | |
| N_WBC_FL_P; | 0.8498 | >−0.9339 | 21 | 76.4 | 79 | 23.6 | 0.003918 | 0.000402 | −11.3441 |
| N_WBC_FLFS_Area; | | | | | | | | | |
| N_WBC_FL_P; | 0.8367 | >−0.9304 | 22.9 | 76.4 | 77.1 | 23.6 | 0.003721 | 0.007322 | −16.5442 |
| N_WBC_FS_P; | | | | | | | | | |
| N_WBC_FL_P; | 0.8346 | >−0.8695 | 21.3 | 74.3 | 78.7 | 25.7 | 0.00401 | 0.007524 | −18.3424 |
| N_NEU_FS_P; | | | | | | | | | |
| N_WBC_FL_P; | 0.8323 | >−0.9135 | 21.6 | 74.5 | 78.4 | 25.5 | 0.004959 | −0.00069 | −7.87546 |
| N_NEU_FL_P; | | | | | | | | | |

TABLE 14-5
Efficacy of the remaining parameter combinations for diagnosis of sepsis

| | | | False | True | True | False | | | |
|---|---|---|---|---|---|---|---|---|---|
| Parameter | | Determination | positive | positive | negative | positive | | | |
| combination | ROC_AUC | threshold | rate % | rate % | rate % | rate % | A | B | C |
| N_NEU_FL_P; | 0.8774 | >−1.0027 | 20.3 | 82.2 | 79.7 | 17.8 | 0.00385 | 0.007984 | −13.0857 |
| N_NEU_FS_W; | | | | | | | | | |
| N_NEU_FL_P; | 0.8771 | >−0.9418 | 19 | 81.4 | 81 | 18.6 | 0.003966 | 12.16253 | −13.6053 |
| N_NEU_FS_CV; | | | | | | | | | |
| N_WBC_FS_W; | 0.8759 | >−0.9353 | 17.7 | 78.4 | 82.3 | 21.6 | 0.008902 | 0.003809 | −16.5624 |
| N_NEU_FL_P; | | | | | | | | | |
| N_WBC_SS_W; | 0.874 | >−1.3306 | 23.1 | 83.2 | 76.9 | 16.8 | 0.004186 | 0.003761 | −13.6248 |
| N_NEU_FL_P; | | | | | | | | | |
| N_WBC_SS_CV; | 0.8713 | >−1.1145 | 19.8 | 79.2 | 80.2 | 20.8 | 6.895398 | 0.004289 | −16.9472 |
| N_NEU_FL_P; | | | | | | | | | |
| N_WBC_FS_CV; | 0.8673 | >−0.8932 | 17.5 | 76.6 | 82.5 | 23.4 | 11.04644 | 0.00417 | −16.8512 |
| N_NEU_FL_P; | | | | | | | | | |
| N_NEU_FL_CV; | 0.8667 | >−0.9658 | 21.5 | 81.8 | 78.5 | 18.2 | −8.89142 | 0.013838 | −2.91538 |
| N_NEU_FS_W; | | | | | | | | | |
| N_NEU_SS_W; | 0.8657 | >−1.1328 | 21.1 | 78.6 | 78.9 | 21.4 | 0.00393 | 0.003857 | −12.8227 |
| N_NEU_FL_P; | | | | | | | | | |
| N_NEU_FL_CV; | 0.8641 | >−0.9934 | 22.4 | 81.6 | 77.6 | 18.4 | −9.56057 | 22.0269 | −3.35479 |
| N_NEU_FS_CV; | | | | | | | | | |
| N_NEU_FL_P; | 0.8599 | >−1.0643 | 22.7 | 80.2 | 77.3 | 19.8 | 0.004103 | 0.000543 | −12.1594 |
| N_NEU_SSFS_Area; | | | | | | | | | |
| N_NEU_FL_P; | 0.8581 | >−0.9716 | 21.5 | 79.8 | 78.5 | 20.2 | 0.003219 | 0.004848 | −13.4874 |
| N_NEU_FL_W; | | | | | | | | | |
| N_NEU_FL_P; | 0.8571 | >−1.0911 | 23.1 | 80.2 | 76.9 | 19.8 | 0.006463 | 8.324475 | −19.1775 |
| N_NEU_FL_CV; | | | | | | | | | |
| N_NEU_FL_P; | 0.8552 | >−1.1393 | 24.5 | 82.2 | 75.5 | 17.8 | 0.003488 | 0.000305 | −10.6116 |
| N_NEU_FLSS_Area; | | | | | | | | | |
| N_NEU_FL_P; | 0.8545 | >−0.9166 | 20.7 | 77.2 | 79.3 | 22.8 | 0.003451 | 0.000535 | −11.1883 |
| N_NEU_FLFS_Area; | | | | | | | | | |
| N_NEU_FL_W; | 0.8541 | >−0.8535 | 20.8 | 78.4 | 79.2 | 21.6 | 0.008541 | −7.71388 | −6.71761 |
| N_NEU_FL_CV; | | | | | | | | | |
| N_NEU_SS_CV; | 0.8541 | >−1.099 | 22.4 | 77.6 | 77.6 | 22.4 | 5.603823 | 0.004281 | −14.5091 |
| N_NEU_FL_P; | | | | | | | | | |
| N_WBC_FL_CV; | 0.8538 | >−1.0969 | 22.4 | 79.2 | 77.6 | 20.8 | 6.609103 | 0.005833 | −19.1609 |
| N_NEU_FL_P; | | | | | | | | | |
| N_NEU_SS_P; | 0.8491 | >−1.0742 | 22.4 | 78 | 77.6 | 22 | 0.007665 | 0.003355 | −16.1445 |
| N_NEU_FL_P; | | | | | | | | | |
| N_WBC_SS_P; | 0.8475 | >−1.0445 | 22 | 77.2 | 78 | 22.8 | 0.007977 | 0.003342 | −16.2863 |
| N_NEU_FL_P; | | | | | | | | | |
| N_WBC_SSFS_Area; | 0.8455 | >−1.0905 | 24 | 78.8 | 76 | 21.2 | 0.000395 | 0.004285 | −12.2871 |
| N_NEU_FL_P; | | | | | | | | | |
| N_WBC_FLSS_Area; | 0.8447 | >−1.0065 | 22.8 | 77.6 | 77.2 | 22.4 | 0.000237 | 0.003498 | −10.4296 |
| N_NEU_FL_P; | | | | | | | | | |
| N_WBC_FL_CV; | 0.843 | >−1.1721 | 26.1 | 81.2 | 73.9 | 18.8 | −7.30651 | 0.013998 | −6.17109 |
| N_WBC_FS_W; | | | | | | | | | |
| N_WBC_FS_W; | 0.8429 | >−1.229 | 26.6 | 82 | 73.4 | 18 | 0.013279 | −7.06111 | −8.3981 |
| N_NEU_FL_CV; | | | | | | | | | |
| N_WBC_FLFS_Area; | 0.8413 | >−1.0561 | 23.5 | 77.8 | 76.5 | 22.2 | 0.000376 | 0.003615 | −11.1295 |
| N_NEU_FL_P; | | | | | | | | | |
| N_WBC_SS_W; | 0.841 | >−1.2903 | 23.5 | 79.2 | 76.5 | 20.8 | 0.006031 | −6.89323 | −3.97758 |
| N_NEU_FL_CV; | | | | | | | | | |
| N_WBC_FL_CV; | 0.8391 | >−1.0002 | 22.9 | 77.2 | 77.1 | 22.8 | −10.5231 | 23.08598 | −6.19496 |
| N_WBC_FS_CV; | | | | | | | | | |
| N_WBC_FS_P; | 0.8376 | >−1.0451 | 26 | 79 | 74 | 21 | 0.009216 | 0.003536 | −19.2245 |
| N_NEU_FL_P; | | | | | | | | | |
| N_WBC_SS_W; | 0.8372 | >−1.1709 | 22.9 | 76.8 | 77.1 | 23.2 | 0.006 | −6.41215 | −1.90749 |
| N_WBC_FL_CV; | | | | | | | | | |
| N_NEU_SS_W; | 0.8348 | >−1.1744 | 21.9 | 77.6 | 78.1 | 22.4 | 0.006036 | −7.46951 | −2.58522 |
| N_NEU_FL_CV; | | | | | | | | | |
| N_NEU_FL_CV; | 0.8343 | >−0.849 | 21.2 | 74.1 | 78.8 | 25.9 | −6.50016 | 0.000902 | −2.5804 |
| N_NEU_FLFS_Area; | | | | | | | | | |
| N_NEU_FL_CV; | 0.8325 | >−0.9255 | 23.1 | 75.2 | 76.9 | 24.8 | −6.50152 | 0.000513 | −1.47551 |
| N_NEU_FLSS_Area; | | | | | | | | | |
| N_WBC_FS_P; | 0.8305 | >−0.9702 | 24.8 | 75.4 | 75.2 | 24.6 | 0.008409 | 0.005411 | −19.3267 |
| N_NEU_FL_W; | | | | | | | | | |
| N_NEU_SS_P; | 0.8299 | >−1.2579 | 27.5 | 78.8 | 72.5 | 21.2 | 0.006538 | 0.004769 | −15.3746 |
| N_NEU_FL_W; | | | | | | | | | |
| N_NEU_FL_P; | 0.8298 | >−0.9906 | 24.4 | 76.2 | 75.6 | 23.8 | 0.003785 | 0.00797 | −19.1846 |
| N_NEU_FS_P; | | | | | | | | | |
| N_WBC_FL_CV; | 0.8295 | >−0.8478 | 23.3 | 75.6 | 76.7 | 24.4 | −4.69578 | 0.007167 | −5.42342 |
| N_NEU_FL_W; | | | | | | | | | |
| N_WBC_FL_CV; | 0.8277 | >−0.7779 | 21.1 | 71.7 | 78.9 | 28.3 | −5.8471 | 0.01119 | −1.39602 |
| N_NEU_FS_W; | | | | | | | | | |
| N_WBC_SS_P; | 0.8256 | >−1.1738 | 26.9 | 77.6 | 73.1 | 22.4 | 0.006437 | 0.004703 | −14.9938 |
| N_NEU_FL_W; | | | | | | | | | |
| N_NEU_FL_P; | 0.8246 | >1812.7065 | 21.5 | 73.5 | 78.5 | 26.5 | 1 | 0 | 0 |
| N_NEU_FL_CV; | 0.8218 | >−1.073 | 27.6 | 78 | 72.4 | 22 | −8.21225 | 0.000891 | −0.69765 |
| N_NEU_SSFS_Area; | | | | | | | | | |
| N_WBC_SS_W; | 0.8213 | >−1.1754 | 26.7 | 76.1 | 73.3 | 23.9 | 0.002205 | 0.003995 | −9.57136 |
| N_NEU_FL_W; | | | | | | | | | |
| N_WBC_FS_W; | 0.8189 | >−1.1615 | 28.7 | 78.1 | 71.3 | 21.9 | 0.002825 | 0.004719 | −10.2468 |
| N_NEU_FL_W; | | | | | | | | | |
| N_WBC_SS_W; | 0.8188 | >−1.1789 | 25 | 76.4 | 75 | 23.6 | 0.003863 | 0.009845 | −19.0978 |
| N_WBC_FS_P; | | | | | | | | | |
| N_WBC_SSFS_Area; | 0.8178 | >−1.0562 | 29.3 | 78.1 | 70.7 | 21.9 | −0.00028 | 0.007498 | −8.72203 |
| N_NEU_FL_W; | | | | | | | | | |
| N_WBC_FL_CV; | 0.8168 | >−0.9404 | 28.4 | 75.4 | 71.6 | 24.6 | −6.11202 | 17.09984 | −1.5466 |
| N_NEU_FS_CV; | | | | | | | | | |
| N_NEU_FL_W; | 0.8155 | >−1.0683 | 26.6 | 76 | 73.4 | 24 | 0.005928 | 0.00392 | −14.7912 |
| N_NEU_FS_P; | | | | | | | | | |
| N_WBC_SS_CV; | 0.8146 | >−1.0467 | 23.9 | 74.3 | 76.1 | 25.7 | 6.072386 | 0.014308 | −26.7066 |
| N_WBC_FS_P; | | | | | | | | | |
| N_WBC_FL_CV; | 0.8126 | >−1.277 | 30.6 | 80.4 | 69.4 | 19.6 | −5.79591 | 0.005439 | −0.98134 |
| N_NEU_SS_W; | | | | | | | | | |
| N_WBC_SS_W; | 0.8103 | >−1.3592 | 27.4 | 76.1 | 72.6 | 23.9 | 0.006964 | −0.00048 | −6.33179 |
| N_WBC_SSFS_Area; | | | | | | | | | |
| N_WBC_FLFS_Area; | 0.8094 | >−1.0062 | 26.4 | 74.3 | 73.6 | 25.7 | −0.00014 | 0.006835 | −9.04692 |
| N_NEU_FL_W; | | | | | | | | | |
| N_NEU_SS_CV; | 0.8093 | >−1.1275 | 28.6 | 75.8 | 71.4 | 24.2 | −0.93584 | 0.006829 | −9.44505 |
| N_NEU_FL_W; | | | | | | | | | |
| N_WBC_SS_CV; | 0.8091 | >−1.0973 | 26.5 | 74.3 | 73.5 | 25.7 | 1.811711 | 0.005657 | −10.9655 |
| N_NEU_FL_W; | | | | | | | | | |
| N_NEU_FL_W; | 0.8086 | >−1.076 | 28.3 | 75.9 | 71.7 | 24.1 | 0.006506 | −0.00012 | −9.1259 |
| N_NEU_SSFS_Area; | | | | | | | | | |
| N_WBC_SS_W; | 0.8086 | >−1.1535 | 25 | 74.7 | 75 | 25.3 | 0.002788 | 0.000385 | −7.73843 |
| N_NEU_FLFS_Area; | | | | | | | | | |
| N_NEU_FL_W; | 0.8085 | >−1.0826 | 27.5 | 75.5 | 72.5 | 24.5 | 0.005121 | 0.00153 | −9.02662 |
| N_NEU_FS_W; | | | | | | | | | |
| N_WBC_FS_CV; | 0.8084 | >−1.1121 | 28.2 | 75.8 | 71.8 | 24.2 | 0.960148 | 0.006173 | −10.2395 |
| N_NEU_FL_W; | | | | | | | | | |
| N_NEU_SS_W; | 0.8083 | >−1.0495 | 26.2 | 73.3 | 73.8 | 26.7 | 0.001259 | 0.004721 | −9.05092 |
| N_NEU_FL_W; | | | | | | | | | |
| N_NEU_FL_W; | 0.808 | >−1.0345 | 25.6 | 73.3 | 74.4 | 26.7 | 0.006073 | 1.068831 | −9.85554 |
| N_NEU_FS_CV; | | | | | | | | | |
| N_WBC_FS_P; | 0.8074 | >−1.0389 | 27.4 | 72.7 | 72.6 | 27.3 | 0.011077 | 0.000274 | −18.9368 |
| N_NWBC_FLSS_Area; | | | | | | | | | |
| N_NEU_SS_P; | 0.8071 | >−0.9908 | 22.3 | 70.5 | 77.7 | 29.5 | 0.008499 | 8.682447 | −15.0131 |
| N_NEU_FS_CV; | | | | | | | | | |
| N_NEU_SS_P; | 0.8071 | >−0.9957 | 22.3 | 70.3 | 77.7 | 29.7 | 0.007113 | 0.000456 | −12.8551 |
| N_NEU_FLFS_Area; | | | | | | | | | |
| N_WBC_SS_CV; | 0.8071 | >−1.273 | 29.3 | 75.6 | 70.7 | 24.4 | 10.12011 | −7.87491 | −3.87777 |
| N_WBC_FL_CV; | | | | | | | | | |
| N_WBC_FL_CV; | 0.8063 | >−0.8175 | 22.5 | 71.3 | 77.5 | 28.7 | −4.14429 | 0.000776 | −1.93617 |
| N_NEU_FLFS_Area; | | | | | | | | | |
| N_NEU_SS_P; | 0.806 | >−1.1398 | 26.2 | 73.3 | 73.8 | 26.7 | 0.007735 | 0.0055 | −13.7478 |
| N_NEU_FS_W; | | | | | | | | | |
| N_NEU_FL_W; | 0.806 | >−1360 | 28.7 | 75.3 | 71.3 | 24.7 | 1 | 0 | 0 |
| N_WBC_SS_P; | 0.8055 | >−1.0424 | 25.1 | 72.1 | 74.9 | 27.9 | 0.008817 | 8.304781 | −14.9931 |
| N_NEU_FS_CV; | | | | | | | | | |
| N_NEU_FL_W; | 0.8053 | >−1.0453 | 26.9 | 74.5 | 73.1 | 25.5 | 0.005061 | 0.000113 | −8.79843 |
| N_NEU_FLFS_Area; | | | | | | | | | |
| N_WBC_FS_P; | 0.8052 | >−1.0692 | 25 | 73.9 | 75 | 26.1 | 0.010497 | 0.003454 | −18.8226 |
| N_NEU_SS_W; | | | | | | | | | |
| N_WBC_FLSS_Area; | 0.8051 | >−1.0103 | 28 | 75.3 | 72 | 24.7 | 7.98E−06 | 0.005726 | −8.97855 |
| N_NEU_FL_W; | | | | | | | | | |
| N_NEU_FL_W; | 0.8049 | >−1.0505 | 27.3 | 74.3 | 72.7 | 25.7 | 0.004987 | 7.36E−05 | −8.66016 |
| N_NEU_FLSS_Area; | | | | | | | | | |
| N_WBC_FS_P; | 0.8048 | >−0.948 | 26.9 | 73.5 | 73.1 | 26.5 | 0.011489 | 10.38377 | −20.483 |
| N_NEU_FS_CV; | | | | | | | | | |
| N_WBC_SS_CV; | 0.8045 | >−1.2676 | 29 | 77 | 71 | 23 | 9.550306 | −7.66297 | −6.31609 |
| N_NEU_FL_CV; | | | | | | | | | |
| N_WBC_SS_P; | 0.8041 | >−0.9504 | 22.3 | 69.5 | 77.7 | 30.5 | 0.008016 | 0.005302 | −13.7463 |
| N_NEU_FS_W; | | | | | | | | | |
| N_WBC_FS_P; | 0.8036 | >−0.9728 | 27.3 | 73.1 | 72.7 | 26.9 | 0.00837 | 0.000551 | −15.8949 |
| N_NEU_FLFS_Area; | | | | | | | | | |
| N_WBC_SS_W; | 0.8032 | >−1.2705 | 26.9 | 78.7 | 73.1 | 21.3 | 0.003193 | 0.003511 | −8.85574 |
| N_WBC_FS_W; | | | | | | | | | |
| N_WBC_SS_P; | 0.8027 | >−1.0726 | 26.6 | 72.9 | 73.4 | 27.1 | 0.007233 | 0.000439 | −12.6853 |
| N_NEU_FLFS_Area; | | | | | | | | | |
| N_WBC_SS_P; | 0.802 | >−1.1483 | 25.6 | 74.3 | 74.4 | 25.7 | 0.007525 | 0.005929 | −15.5463 |
| N_WBC_FS_W; | | | | | | | | | |
| N_WBC_FS_P; | 0.8019 | >−1.2151 | 30.9 | 78.2 | 69.1 | 21.8 | 0.013986 | 9.747724 | −26.4096 |
| N_WBC_FS_CV; | | | | | | | | | |
| N_WBC_FL_CV; | 0.8019 | >−0.9149 | 25.9 | 72.7 | 74.1 | 27.3 | −4.1213 | 0.000439 | −0.98817 |
| N_NEU_FLSS_Area; | | | | | | | | | |
| N_WBC_SS_W; | 0.8015 | >−1.1835 | 25.6 | 75.3 | 74.4 | 24.7 | 0.003138 | 0.003743 | −7.76688 |
| N_NEU_FS_W; | | | | | | | | | |
| N_WBC_SS_W; | 0.8011 | >−1.1392 | 24.6 | 74.5 | 75.4 | 25.5 | 0.002793 | 0.000198 | −7.04498 |
| N_NEU_FLSS_Area; | | | | | | | | | |
| N_WBC_FS_P; | 0.8005 | >−1.1613 | 28.8 | 74.9 | 71.2 | 25.1 | 0.008411 | 0.007504 | −19.1726 |
| N_WBC_FS_W; | | | | | | | | | |
| N_WBC_FS_P; | 0.8005 | >−0.9853 | 26.6 | 72.5 | 73.4 | 27.5 | 0.008906 | 0.000312 | −15.892 |
| N_NEU_FLSS_Area; | | | | | | | | | |
| N_WBC_FLSS_Area; | 0.8005 | >−1.0461 | 26.6 | 73.7 | 73.4 | 26.3 | 0.0004 | −5.27736 | −2.13522 |
| N_NEU_FL_CV; | | | | | | | | | |
| N_WBC_FS_P; | 0.8005 | >−0.9539 | 26.9 | 73.1 | 73.1 | 26.9 | 0.009387 | 0.006638 | −17.3811 |
| N_NEU_FS_W; | | | | | | | | | |
| N_WBC_SSFS_Area; | 0.8002 | >−1.004 | 29.6 | 75.7 | 70.4 | 24.3 | −0.00054 | 0.001157 | −4.68241 |
| N_NEU_FLFS_Area; | | | | | | | | | |
| N_WBC_FS_W; | 0.8002 | >−1.1054 | 23.9 | 72.3 | 76.1 | 27.7 | 0.00587 | 0.007148 | −15.232 |
| N_NEU_SS_P; | | | | | | | | | |
| N_WBC_SSFS_Area; | 0.8002 | >−1.0045 | 30.9 | 75.1 | 69.1 | 24.9 | −0.00068 | 0.000751 | −3.04041 |
| N_NEU_FLSS_Area; | | | | | | | | | |
| N_WBC_FS_W; | 0.7989 | >−1.0754 | 24.9 | 73.5 | 75.1 | 26.5 | 0.011766 | −0.00029 | −9.80896 |
| N_WBC_SSFS_Area; | | | | | | | | | |
| N_WBC_FS_W; | 0.7989 | >−1.1133 | 28.5 | 75.5 | 71.5 | 24.5 | 0.003832 | 0.000467 | −8.19068 |
| N_NEU_FLFS_Area; | | | | | | | | | |
| N_WBC_SS_W; | 0.7985 | >−1.3253 | 29.1 | 79 | 70.9 | 21 | 0.003602 | 5.445765 | −8.46534 |
| N_NEU_FS_CV; | | | | | | | | | |
| N_WBC_FS_W; | 0.7981 | >−1.1765 | 28.4 | 74.3 | 71.6 | 25.7 | 0.018242 | −13.944 | −8.2599 |
| N_WBC_FS_CV; | | | | | | | | | |
| N_WBC_FS_W; | 0.7973 | >−1.0833 | 26.2 | 72.1 | 73.8 | 27.9 | 0.004219 | 0.00026 | −7.94057 |
| N_NEU_FLSS_Area; | | | | | | | | | |
| N_WBC_SS_W; | 0.7971 | >−1.1919 | 24.3 | 73.5 | 75.7 | 26.5 | 0.004205 | 0.004038 | −12.6356 |
| N_NEU_FS_P; | | | | | | | | | |
| N_WBC_FLSS_Area; | 0.7966 | >−1.0693 | 28.2 | 75.9 | 71.8 | 24.1 | 0.000747 | −0.00082 | −3.43689 |
| N_WBC_SSFS_Area; | | | | | | | | | |
| N_WBC_SS_P; | 0.7961 | >−1.2242 | 27.1 | 75.8 | 72.9 | 24.2 | 0.009303 | 3.947558 | −16.53 |
| N_WBC_SS_CV; | | | | | | | | | |
| N_WBC_FS_P; | 0.7959 | >−1.0209 | 28.5 | 72.7 | 71.5 | 27.3 | 0.011184 | 0.000404 | −19.3315 |
| N_WBC_FLFS_Area; | | | | | | | | | |
| N_WBC_FL_CV; | 0.7954 | >−1.0005 | 28.1 | 74.5 | 71.9 | 25.5 | −4.68136 | 0.000393 | −0.75575 |
| N_WBC_FLSS_Area; | | | | | | | | | |
| N_WBC_SS_W; | 0.7954 | >−1.1963 | 25.7 | 75.3 | 74.3 | 24.7 | 0.003297 | 0.000122 | −7.19749 |
| N_WBC_FLSS_Area; | | | | | | | | | |
| N_NEU_SS_P; | 0.7952 | >−1.1264 | 26.5 | 72.9 | 73.5 | 27.1 | 0.006898 | 0.000232 | −11.7334 |
| N_NEU_FLSS_Area; | | | | | | | | | |
| N_WBC_SS_W; | 0.7946 | >−1.2271 | 26.1 | 75.4 | 73.9 | 24.6 | 0.006597 | −3.94407 | −5.48612 |
| N_WBC_SS_CV; | | | | | | | | | |
| N_WBC_SS_W; | 0.7944 | >−1.1249 | 23.1 | 71.3 | 76.9 | 28.7 | 0.003606 | 0.000159 | −7.5412 |
| N_WBC_FLFS_Area; | | | | | | | | | |
| N_WBC_FS_CV; | 0.7943 | >−0.9181 | 25.5 | 72.7 | 74.5 | 27.3 | 16.34442 | −7.16444 | −7.66986 |
| N_NEU_FL_CV; | | | | | | | | | |
| N_WBC_FS_W; | 0.7941 | >−1.1848 | 26.4 | 75.3 | 73.6 | 24.7 | 0.004852 | 0.002487 | −8.78878 |
| N_NEU_SS_W; | | | | | | | | | |
| N_WBC_SS_P; | 0.7936 | >−1.1235 | 23.2 | 70.3 | 76.8 | 29.7 | 0.005976 | 0.002835 | −11.901 |
| N_WBC_SS_W; | | | | | | | | | |
| N_WBC_SS_CV; | 0.7921 | >−1.0679 | 20.6 | 70.7 | 79.4 | 29.3 | 3.924639 | 0.008805 | −16.1487 |
| N_NEU_SS_P; | | | | | | | | | |
| N_WBC_SSFS_Area; | 0.7917 | >−1.111 | 30.7 | 76.9 | 69.3 | 23.1 | −0.00022 | 0.010514 | −5.7044 |
| N_NEU_FS_W; | | | | | | | | | |
| N_WBC_SS_W; | 0.7915 | >−1.0771 | 21 | 69.9 | 79 | 30.1 | 0.002807 | 0.005791 | −11.7955 |
| N_NEU_SS_P; | | | | | | | | | |
| N_WBC_SS_P; | 0.7913 | >−1.1408 | 27.7 | 73.5 | 72.3 | 26.5 | 0.007071 | 0.000221 | −11.6417 |
| N_NEU_FLSS_Area; | | | | | | | | | |
| N_WBC_FLFS_Area; | 0.7909 | >−0.9252 | 24.2 | 70.9 | 75.8 | 29.1 | 0.000648 | −5.51574 | −2.90345 |
| N_NEU_FL_CV; | | | | | | | | | |
| N_WBC_FS_W; | 0.7906 | >−1.0549 | 24 | 72.3 | 76 | 27.7 | 0.005205 | 0.000183 | −8.48618 |
| N_WBC_FLSS_Area; | | | | | | | | | |
| N_NEU_SS_P; | 0.7901 | >−1.1423 | 25.2 | 71.7 | 74.8 | 28.3 | 0.009514 | 3.22598 | −15.6565 |
| N_NEU_SS_CV; | | | | | | | | | |
| N_NEU_SS_W; | 0.79 | >−1.0947 | 26.1 | 72.1 | 73.9 | 27.9 | 0.00205 | 0.000451 | −6.86989 |
| N_NEU_FLFS_Area; | | | | | | | | | |

TABLE 14-6
Efficacy of PCT (procalcitonin) of prior art and the parameters
of the DIFF channel for diagnosis of sepsis

| Infection | | | False | True | True | False |
|---|---|---|---|---|---|---|
| marker | | Determination | positive | positive | negative | negative |
| parameter | ROC_AUC | threshold | rate | rate | rate | rate |
| PCT | 0.787 | 0.64 | 37.3% | 81.0% | 62.7% | 19.0% |
| D_Neu_SS_W | 0.687 | 252.764 | 45.4% | 74.1% | 54.6% | 25.9% |
| D_Neu_FL_W | 0.791 | 213.465 | 22.8% | 68.0% | 77.2% | 32.0% |
| D_Neu_FS_W | 0.545 | 586.385 | 22.6% | 32.2% | 77.4% | 67.8% |

Form the comparison between Table 14-6 and Tables 14-1 to 14-5, it can be seen that the parameters of WNB channel have better diagnostic performance than the parameters of DIFF channel and PCT for diagnosis of sepsis.

TABLE 14-7
Illustration of the statistical methods and testing methods
used in this example by taking three parameters as examples

| Infection | | | | |
|---|---|---|---|---|
| marker | Positive sample | Negative sample | | |
| parameter | Mean ± SD | Mean ± SD | F value | P value |
| N_WBC_FL_W | 2088.31 ± 299.3 | 1702.5 ± 232.7 | 674.92 | <0.0001 |
| N_NEU_FL_W | 1501.8 ± 232.6 | 1282.91 ± 175.7 | 363.67 | <0.0001 |
| N_NEU_FLSS_Area | 14819.3240 ± 180.4941 | 12161.8716 ± 2235.5756 | 192.94 | <0.0001 |

As can be seen from Table 14-7, this parameter is analyzed by Welch test, and there is a significant statistical difference between the two groups (p<0.0001)

As can be seen from Tables 12 to 14, the infection marker parameters provided in the disclosure can be used to effectively determine whether a subject has sepsis. For the same reasons as example 1, the disclosure accidentally discovered through in-depth investigation that a more useful feature can be found from the WNB channel than from the DIFF channel to diagnose sepsis.

### Example 4 Monitoring of Severe Infection

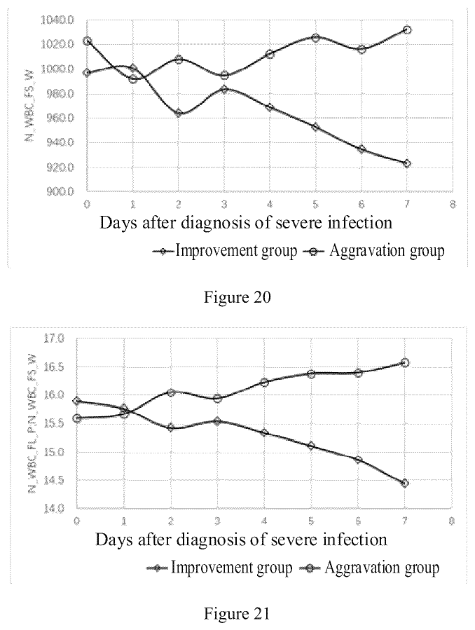

Blood samples from 50 patients with severe infection were subjected to consecutive blood routine test using the BC-6800 Plus blood cell analyzer produced by SHENZHEN MINDRAY BIO-MEDICAL ELECTRONICS CO., LTD. in accordance with the steps of example 1 of the disclosure, and the aforementioned method was adopted for monitoring the progression of severe infection based on the scattergram. 50 patients with severe infection were grouped according to their condition on the 7th day after the diagnosis of severe infection. If the degree of infection improved and the condition was stable on the 7th day after diagnosis, the patient was included in the improvement group (positive sample N=26). If the degree of infection did not improve significantly, the patient was still in the stage of severe infection or the patient died, the patient was included in the aggravation group (negative sample N=24). Table 15 shows the infection marker parameters used and their corresponding experimental data (the average values of the infection marker parameter values of the two groups of patients), FIG. 19 shows a dynamic trend change graph from monitoring with a single parameter N_WBC_FL_P as the infection marker parameter, FIG. 20 shows a dynamic trend change graph from monitoring with a single parameter N_WBC_FS_W as the infection marker parameter, and FIG. 21 shows a dynamic trend change graph from monitoring with a linear combination parameter of N_WBC_FL_P and N_WBC_FS_W (N_WBC_FL_P*0.003755+N_WBC_FS_W*0.009192) as the infection marker parameter, wherein the days after diagnosis of severe infection are taken as the horizontal axis and the average values of the infection marker parameter values of the two groups of patients are taken as the vertical axis.

TABLE 15
Different infection marker parameters and their corresponding experimental data

| | | | | Combination of |
|---|---|---|---|---|
| | X days after | | | N_WBC_FL_P and |
| Group | diagnosis | N_WBC_FL_P | N_WBC_FS_W | N_WBC_FS_W |
| Aggravation | 0 | 1789.87 | 996.92 | 15.88 |
| | 1 | 1747.14 | 1000.62 | 15.76 |
| | 2 | 1747.75 | 963.69 | 15.42 |
| | 3 | 1730.02 | 983.38 | 15.54 |
| | 4 | 1712.83 | 968.62 | 15.33 |
| | 5 | 1690.94 | 952.62 | 15.11 |
| | 6 | 1668.28 | 934.15 | 14.85 |
| | 7 | 1584.86 | 923.08 | 14.44 |
| Improvement | 0 | 1647.79 | 1022.67 | 15.59 |
| | 1 | 1741.44 | 992.00 | 15.66 |
| | 2 | 1804.87 | 1008.00 | 16.04 |
| | 3 | 1807.95 | 994.67 | 15.93 |
| | 4 | 1844.82 | 1012.00 | 16.23 |
| | 5 | 1851.54 | 1025.33 | 16.38 |
| | 6 | 1878.85 | 1016.00 | 16.39 |
| | 7 | 1887.26 | 1032.00 | 16.57 |

As can be seen from Table 15 and FIGS. 19-21, the infection marker parameters provided in the disclosure can be used to effectively monitor the progression of the infection status of patients with severe infection.

### Example 5 Monitoring of Sepsis Condition

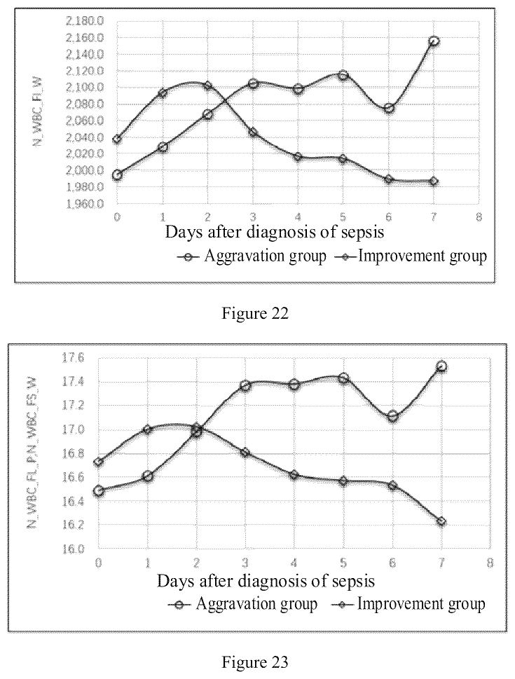

Blood samples from 76 patients with sepsis were subjected to consecutive blood routine test using the BC-6800 Plus blood cell analyzer produced by SHENZHEN MINDRAY BIO-MEDICAL ELECTRONICS CO., LTD. in accordance with the steps of example 1 of the disclosure, and the aforementioned method was adopted for monitoring the progression of sepsis condition based on the scattergram. 76 patients with sepsis were grouped according to their condition on the 7th day after the diagnosis of sepsis. If the degree of infection improved and the condition was stable on the 7th day after diagnosis, the patient was included in the improvement group (positive sample N=55). If the degree of infection did not improve significantly, the patient was still in the stage of severe infection or the patient died, the patient was included in the aggravation group (negative sample N=21). Table 16 shows the infection marker parameters used and their corresponding experimental data (median of values of the infection marker parameter for both groups of patients). FIG. 22 shows a dynamic trend change graph from monitoring with N_WBC_FL_W as the infection marker parameter, and FIG. 23 shows a dynamic trend change graph from monitoring with a linear combination of N_WBC_FL_P and N_WBC_FS_W (0.0040875*N_WBC_FL_P+0.00905881*N_WBC_FS_W) as the infection marker parameter, wherein the days after diagnosis of sepsis are taken as the horizontal axis and the median values of the infection marker parameter values of the two groups of patients are taken as the vertical axis.

TABLE 16
Different infection marker parameters and their corresponding experimental data

| | | | | | Combination of |
|---|---|---|---|---|---|
| | X days after | | | | N_WBC_FL_P and |
| Group | diagnosis | N_WBC_FL_W | N_WBC_FL_P | N_WBC_FS_W | N_WBC_FS_W |
| Aggravation | 0 | 1994.67 | 1704.71 | 1051.43 | 16.49269 |
| | 1 | 2028.19 | 1746.58 | 1045.33 | 16.60862 |
| | 2 | 2067.81 | 1795.99 | 1063.62 | 16.97623 |
| | 3 | 2104.38 | 1871.91 | 1072.76 | 17.36939 |
| | 4 | 2098.29 | 1849.92 | 1083.43 | 17.37612 |
| | 5 | 2115.05 | 1864.07 | 1083.43 | 17.43397 |
| | 6 | 2075.43 | 1815.19 | 1069.71 | 17.10994 |
| | 7 | 2156.19 | 1954.26 | 1052.95 | 17.52654 |
| Improvement | 0 | 2038.29 | 1772.98 | 1046.29 | 16.72516 |
| | 1 | 2093.14 | 1860.11 | 1037.14 | 16.99848 |
| | 2 | 2101.71 | 1864.18 | 1037.14 | 17.0151 |
| | 3 | 2046.86 | 1842.11 | 1024.57 | 16.81101 |
| | 4 | 2016.57 | 1816.56 | 1014.86 | 16.61857 |
| | 5 | 2013.71 | 1775.86 | 1027.43 | 16.56611 |
| | 6 | 1989.71 | 1814.60 | 1005.71 | 16.52773 |
| | 7 | 1987.49 | 1772.27 | 991.42 | 16.22523 |

As can be seen from Table 16 and FIGS. 22 and 23, the infection marker parameters provided in the disclosure can be used to effectively monitor the progression of sepsis of the subject.

### Example 6 Prognostic Analysis of Sepsis

270 blood samples were subjected to test using the BC-6800 Plus blood cell analyzer produced by SHENZHEN MINDRAY BIO-MEDICAL ELECTRONICS CO., LTD. in accordance with the steps of example 1 of the disclosure, and the aforementioned method was adopted for analysis of sepsis prognosis based on the scattergram. Among them, 68 positive samples died at 28 days, and 202 negative samples survived at 28 days. Table 17 shows the efficacy of using single leukocyte characteristic parameters as infection marker parameters for determining whether the sepsis prognosis is good in this example, and Tables 18 show the efficacy of using parameter combinations as infection marker parameters for determining whether the sepsis prognosis is good in this example, wherein infection marker parameters are calculated by the function Y=A×X1+B×X2+C based on the parameter combinations in the Table 18, where Y represents an infection marker parameter, X1 represents the first leukocyte parameter, X2 represents the second leukocyte parameter, and A, B, and C are constants.

TABLE 17
Efficacy of single parameters in determining whether the sepsis prognosis is good

| | | | False | True | True | False |
|---|---|---|---|---|---|---|
| Single | | Determination | positive | positive | negative | negative |
| parameter | ROC_AUC | threshold | rate % | rate % | rate % | rate % |
| N_WBC_FL_W | 0.7964 | >2128 | 21.3 | 67.6 | 78.7 | 32.4 |
| N_WBC_FS_W | 0.7371 | >1040 | 26.7 | 70.6 | 73.3 | 29.4 |
| N_WBC_FLSS_Area | 0.7118 | >14494.72 | 39.1 | 70.6 | 60.9 | 29.4 |
| N_WBC_FS_CV | 0.7073 | >0.7875 | 32.7 | 66.2 | 67.3 | 33.8 |
| N_WBC_FLFS_Area | 0.7033 | >10726.4 | 30.2 | 63.2 | 69.8 | 36.8 |

TABLE 18
Efficacy of two-parameter combination in determining whether the sepsis prognosis is good

| | | | False | True | True | False | | | |
|---|---|---|---|---|---|---|---|---|---|
| Parameter | | Determination | positive | positive | negative | negative | | | |
| combination | ROC_AUC | threshold | rate % | rate % | rate % | rate % | A | B | C |
| N_WBC_FL_P; | 0.814 | >−1.1218 | 21.8 | 73.5 | 78.2 | 26.5 | 0.004356 | 14.99812 | −20.9347 |
| N_WBC_FS_CV | | | | | | | | | |
| N_WBC_FL_W; | 0.8053 | >−0.9686 | 22.3 | 72.1 | 77.7 | 27.9 | 0.00528 | 0.004209 | −16.479 |
| N_WBC_FS_W | | | | | | | | | |
| N_WBC_SS_W; | 0.805 | >−1.1389 | 27.7 | 73.5 | 72.3 | 26.5 | 0.00118 | 0.005554 | −14.511 |
| N_WBC_FL_W | | | | | | | | | |
| N_WBC_FL_P; | 0.8048 | >−0.8947 | 22.3 | 73.5 | 77.7 | 26.5 | 0.007096 | 10.58775 | −26.3003 |
| N_WBC_FL_CV | | | | | | | | | |
| N_WBC_SS_P; | 0.8045 | >−1.2205 | 30.7 | 77.9 | 69.3 | 22.1 | 0.003126 | 0.005785 | −16.9482 |
| N_WBC_FL_W | | | | | | | | | |
| N_WBC_SS_CV; | 0.8041 | >−1.1164 | 27.2 | 75 | 72.8 | 25 | 1.368887 | 0.005772 | −14.882 |
| N_WBC_FL_W | | | | | | | | | |
| N_WBC_FL_W; | 0.8039 | >−0.8941 | 21.3 | 70.6 | 78.7 | 29.4 | 0.005627 | 3.401492 | −15.5168 |
| N_WBC_FS_CV | | | | | | | | | |
| N_WBC_FL_P; | 0.8023 | >−1.1298 | 22.3 | 70.6 | 77.7 | 29.4 | 0.003767 | 0.010494 | −18.9127 |
| N_WBC_FS_W | | | | | | | | | |
| N_WBC_FL_W; | 0.8007 | >−0.9302 | 19.8 | 72.1 | 80.2 | 27.9 | 0.006162 | 0.005279 | −20.8978 |
| N_WBC_FS_P | | | | | | | | | |
| N_WBC_FL_W; | 0.7974 | >−0.7068 | 18.3 | 66.2 | 81.7 | 33.8 | 0.005966 | 5.92E−05 | −14.1144 |
| N_WBC_SSFS_Area | | | | | | | | | |
| N_WBC_FL_W; | 0.7971 | >−0.6871 | 16.8 | 66.2 | 83.2 | 33.8 | 0.005826 | 3.85E−05 | −13.828 |
| N_WBC_FLSS_Area | | | | | | | | | |
| N_WBC_FL_P; | 0.7968 | >−0.819 | 20.8 | 67.6 | 79.2 | 32.4 | 0.00034 | 0.005898 | −14.0224 |
| N_WBC_FL_W | | | | | | | | | |
| N_WBC_FL_W; | 0.7966 | >−0.8818 | 21.3 | 69.1 | 78.7 | 30.9 | 0.006179 | −0.27734 | −13.6655 |
| N_WBC_FL_CV | | | | | | | | | |
| N_WBC_FL_W; | 0.796 | >−0.9117 | 22.3 | 69.1 | 77.7 | 30.9 | 0.006002 | 2.97E−05 | −13.9327 |
| N_WBC_FLFS_Area | | | | | | | | | |
| N_WBC_SS_W; | 0.7878 | >−1.3184 | 29.2 | 76.5 | 70.8 | 23.5 | 0.003046 | 0.003567 | −12.3755 |
| N_WBC_FL_P | | | | | | | | | |
| N_WBC_SS_CV; | 0.7831 | >−1.4189 | 33.7 | 80.9 | 66.3 | 19.1 | 5.00643 | 0.003887 | −14.6224 |
| N_WBC_FL_P | | | | | | | | | |
| N_WBC_FL_CV; | 0.7726 | >−0.9676 | 19.8 | 70.6 | 80.2 | 29.4 | −9.11892 | 23.2954 | −8.99711 |
| N_WBC_FS_CV | | | | | | | | | |
| N_WBC_FL_P; | 0.7715 | >−0.9061 | 21.8 | 64.7 | 78.2 | 35.3 | 0.003877 | 0.00045 | −12.5969 |
| N_WBC_SSFS_Area | | | | | | | | | |
| N_WBC_FL_P; | 0.7678 | >−1.1019 | 28.7 | 69.1 | 71.3 | 30.9 | 0.002907 | 0.000261 | −10.3655 |
| N_WBC_FLSS_Area | | | | | | | | | |
| N_WBC_FL_P; | 0.7614 | >−1.0232 | 28.2 | 69.1 | 71.8 | 30.9 | 0.002813 | 0.000424 | −10.8004 |
| N_WBC_FLFS_Area | | | | | | | | | |
| N_WBC_FL_CV; | 0.7601 | >−1.0712 | 23.3 | 72.1 | 76.7 | 27.9 | −5.01104 | 0.012921 | −8.75004 |
| N_WBC_FS_W | | | | | | | | | |
| N_WBC_FS_W; | 0.7519 | >−1.1929 | 28.7 | 75 | 71.3 | 25 | 0.006147 | 0.000169 | −10.0256 |
| N_WBC_FLSS_Area | | | | | | | | | |
| N_WBC_SS_W; | 0.7489 | >−1.1013 | 24.8 | 69.1 | 75.2 | 30.9 | 0.001372 | 0.006849 | −10.3323 |
| N_WBC_FS_W | | | | | | | | | |
| N_WBC_SS_CV; | 0.7438 | >−0.7441 | 16.3 | 64.7 | 83.7 | 35.3 | 1.575295 | 0.007685 | −11.0799 |
| N_WBC_FS_W | | | | | | | | | |
| N_WBC_SS_P; | 0.7429 | >−1.2495 | 31.2 | 76.5 | 68.8 | 23.5 | 0.002866 | 0.007794 | −12.6548 |
| N_WBC_FS_W | | | | | | | | | |
| N_WBC_FS_W; | 0.7424 | >−1.1434 | 29.2 | 73.5 | 70.8 | 26.5 | 0.006118 | 0.000277 | −10.4094 |
| N_WBC_FLFS_Area | | | | | | | | | |
| N_WBC_SS_P; | 0.7392 | >−0.9708 | 25.2 | 64.7 | 74.8 | 35.3 | 0.004672 | 9.426769 | −14.1881 |
| N_WBC_FS_CV | | | | | | | | | |
| N_WBC_FS_W; | 0.7373 | >−0.9328 | 23.3 | 69.1 | 76.7 | 30.9 | 0.009134 | −8.7E−06 | −10.4907 |
| N_WBC_SSFS_Area | | | | | | | | | |
| N_WBC_FS_P; | 0.7359 | >−1.1553 | 26.7 | 72.1 | 73.3 | 27.9 | 0.007686 | 11.646 | −20.4001 |
| N_WBC_FS_CV | | | | | | | | | |
| N_WBC_FS_W; | 0.7352 | >−1.1711 | 26.7 | 72.1 | 73.3 | 27.9 | 0.009929 | −1.38436 | −10.3076 |
| N_WBC_FS_CV | | | | | | | | | |
| N_WBC_FS_P; | 0.7351 | >−1.1683 | 26.7 | 72.1 | 73.3 | 27.9 | 0.000834 | 0.008873 | −11.4018 |
| N_WBC_FS_W | | | | | | | | | |
| N_WBC_SS_W; | 0.735 | >−1.2413 | 33.2 | 70.6 | 66.8 | 29.4 | 0.001351 | 0.000204 | −6.2658 |
| N_WBC_FLSS_Area | | | | | | | | | |
| N_WBC_SS_P; | 0.7331 | >−1.1552 | 34.7 | 67.6 | 65.3 | 32.4 | 0.005224 | 0.003046 | −13.0168 |
| N_WBC_FL_P | | | | | | | | | |
| N_WBC_SS_W; | 0.7327 | >−1.3064 | 33.7 | 69.1 | 66.3 | 30.9 | 0.001579 | 0.000349 | −7.27831 |
| N_WBC_FLFS_Area | | | | | | | | | |
| N_WBC_FS_P; | 0.731 | >−0.9242 | 20.3 | 58.8 | 79.7 | 41.2 | 0.006309 | 0.000294 | −13.8316 |
| N_WBC_FLSS_Area | | | | | | | | | |
| N_WBC_SS_P; | 0.7309 | >−1.1278 | 30.7 | 69.1 | 69.3 | 30.9 | 0.003756 | 0.000413 | −10.0642 |
| N_WBC_FLFS_Area | | | | | | | | | |
| N_WBC_FS_CV; | 0.7302 | >−1.1704 | 30.2 | 67.6 | 69.8 | 32.4 | 5.39972 | 0.000203 | −8.41737 |
| N_WBC_FLSS_Area | | | | | | | | | |

As can be seen from Tables 17 and 18, the infection marker parameters provided in the disclosure can be used to effectively determine the prognosis of sepsis in patients.

### Example 7 Identification of Bacterial Infection and Viral Infection

491 blood samples were subjected to test using the BC-6800 Plus blood cell analyzer produced by SHENZHEN MINDRAY BIO-MEDICAL ELECTRONICS CO., LTD. in accordance with the steps of example 1 of the disclosure, and the aforementioned method was adopted for determining infection type based on the scattergram. Among them, there were 237 bacterial infection samples (that is, positive samples) and 254 viral infection samples.

Inclusion criteria for these cases: adult ICU patients with or without acute infection. Exclusion criteria: pregnant people, myelosuppressed people on chemotherapy, people on immunosuppressant treatment, patients with hematologic diseases.

For the bacterial infection samples: there were suspicious or definite infection sites, and the laboratory bacterial culture results were positive, that is, all of (1)-(3) were satisfied
(1) Evidence of bacterial infection: (meeting any of the following 1-4)
1. There was a definite infection site
2. Inflammatory markers (WBC, CRP, PCT, etc.) were elevated
3. Microbial culture showed positive results
4. Imaging findings suggested infection
(2) The change of SOFA score from baseline <2
(3) The change of the clinically recognized organ failure index score <2

For the virus infection samples: there were suspicious or definite infection sites, and the virus antigen or antibody test was positive. For example, any of the following was met:
(1) Influenza A virus or influenza B virus antibody test was positive
(2) Epstein-Barr virus antibody test was positive
(3) Cytomegalovirus antibody test was positive.

Table 19 shows the efficacy of a single leukocyte characteristic parameter as an infection marker parameter for the identification of bacterial infection and viral infection in this example, and Table 20-1 show the efficacy of parameter combinations as infection marker parameters for the identification of bacterial infection and viral infection in this example, wherein infection marker parameters are calculated by the function Y=A×X1+B×X2+C based on the parameter combinations in the Table 20-1, where Y represents an infection marker parameter, X1 represents the first leukocyte parameter, X2 represents the second leukocyte parameter, and A, B, and C are constants.

TABLE 19
Efficacy of single parameter for the identification of bacterial infection and viral infection

| | | Determi- | False | True | True | False |
|---|---|---|---|---|---|---|
| | | nation | positive | positive | negative | negative |
| Single parameter | ROC_AUC | threshold | rate % | rate % | rate % | rate % |
| N_WBC_FS_P | 0.879 | >1318.3915 | 18.9 | 78 | 81.1 | 22 |
| N_WBC_FL_P | 0.8648 | >1450.1095 | 19.3 | 79.2 | 80.7 | 20.8 |
| N_WBC_FS_W | 0.8501 | >1008 | 15 | 73.7 | 85 | 26.3 |
| N_WBC_FL_W | 0.8442 | >1744 | 22.4 | 73.3 | 77.6 | 26.7 |
| N_WBC_FLFS_Area | 0.8176 | >9313.28 | 22 | 74.2 | 78 | 25.8 |
| N_WBC_FLSS_Area | 0.8046 | >11909.12 | 20.1 | 70.3 | 79.9 | 29.7 |
| N_WBC_SS_P | 0.7925 | >1137.061 | 27.6 | 70.8 | 72.4 | 29.2 |
| N_WBC_SS_W | 0.7297 | >1328 | 24.4 | 66.9 | 75.6 | 33.1 |

TABLE 20-1
Efficacy of two-parameter combination for the identification of bacterial infection and viral infection

| | | Determi- | False | True | True | False | | | |
|---|---|---|---|---|---|---|---|---|---|
| Parameter | | nation | positive | positive | negative | negative | | | |
| combination | ROC_AUC | threshold | rate % | rate % | rate % | rate % | A | B | C |
| N_WBC_FL_CV; | 0.9262 | >−0.4777 | 13.8 | 83.9 | 86.2 | 16.1 | −14.4674 | 0.020962 | −4.40612 |
| N_WBC_FS_W | | | | | | | | | |
| N_WBC_FS_P; | 0.9221 | >−0.9129 | 17.7 | 91.9 | 82.3 | 8.1 | 0.027475 | 0.000746 | −43.7208 |
| N_WBC_FLFS_Area | | | | | | | | | |
| N_WBC_FS_P; | 0.9213 | >−0.586 | 15.4 | 87.3 | 84.6 | 12.7 | 0.026493 | 0.000432 | −40.542 |
| N_WBC_FLSS_Area | | | | | | | | | |
| N_WBC_FL_P; | 0.9212 | >−0.6576 | 16.5 | 83.9 | 83.5 | 16.1 | 0.004847 | 0.012517 | −20.3103 |
| N_WBC_FS_W | | | | | | | | | |
| N_WBC_FS_P; | 0.9126 | >−0.826 | 16.5 | 86.9 | 83.5 | 13.1 | 0.020585 | 0.01052 | −38.2282 |
| N_WBC_FS_W | | | | | | | | | |
| N_WBC_FL_W; | 0.9124 | >−0.5809 | 13.8 | 80.5 | 86.2 | 19.5 | 0.004837 | 0.022143 | −38.1666 |
| N_WBC_FS_P | | | | | | | | | |
| N_WBC_FS_P; | 0.9123 | >−0.8642 | 16.9 | 86.9 | 83.1 | 13.1 | 0.028819 | 14.94444 | −49.906 |
| N_WBC_FS_CV | | | | | | | | | |
| N_WBC_FS_W; | 0.912 | >−0.8165 | 16.5 | 86.4 | 83.5 | 13.6 | 0.034981 | −32.9948 | −10.5109 |
| N_WBC_FS_CV | | | | | | | | | |
| N_WBC_FL_P; | 0.91 | >−0.857 | 21.3 | 86.4 | 78.7 | 13.6 | 0.004142 | 0.019317 | −32.1114 |
| N_WBC_FS_P | | | | | | | | | |
| N_WBC_FL_P; | 0.8978 | >−0.471 | 16.1 | 80.5 | 83.9 | 19.5 | 0.005905 | 0.000563 | −14.3319 |
| N_WBC_SSFS_Area | | | | | | | | | |
| N_WBC_FL_P; | 0.8977 | >−0.3316 | 15.4 | 79.2 | 84.6 | 20.8 | 0.004712 | 0.000517 | −12.2122 |
| N_WBC_FLFS_Area | | | | | | | | | |
| N_WBC_FL_P; | 0.8966 | >−0.5131 | 19.3 | 81.8 | 80.7 | 18.2 | 0.005587 | 13.07023 | −18.6806 |
| N_WBC_FS_CV | | | | | | | | | |
| N_WBC_FL_CV; | 0.8938 | >−0.3663 | 19.7 | 81.8 | 80.3 | 18.2 | −17.431 | 29.44575 | −2.08245 |
| N_WBC_FS_CV | | | | | | | | | |
| N_WBC_FL_P; | 0.8934 | >−0.4048 | 16.1 | 79.7 | 83.9 | 20.3 | 0.00481 | 0.000319 | −11.2908 |
| N_WBC_FLSS_Area | | | | | | | | | |
| N_WBC_FS_P; | 0.8917 | >−0.5796 | 20.1 | 85.2 | 79.9 | 14.8 | 0.026888 | 0.000383 | −39.3911 |
| N_WBC_SSFS_Area | | | | | | | | | |
| N_WBC_SS_CV; | 0.8917 | >−0.5622 | 18.5 | 83.9 | 81.5 | 16.1 | 3.332173 | 0.027948 | −41.2304 |
| N_WBC_FS_P | | | | | | | | | |
| N_WBC_SS_W; | 0.8903 | >−0.5033 | 18.5 | 83.5 | 81.5 | 16.5 | 0.001563 | 0.025515 | −36.1817 |
| N_WBC_FS_P | | | | | | | | | |
| N_WBC_FL_W; | 0.8878 | >−0.3344 | 15 | 75 | 85 | 25 | 0.004175 | 0.009635 | −17.3938 |
| N_WBC_FS_W | | | | | | | | | |
| N_WBC_FL_CV; | 0.8856 | >−0.2683 | 19.3 | 83.1 | 80.7 | 16.9 | −10.865 | 0.000898 | 4.246537 |
| N_WBC_FLFS_Area | | | | | | | | | |
| N_WBC_SS_W; | 0.8831 | >−0.6756 | 20.1 | 81.8 | 79.9 | 18.2 | 0.002389 | 0.00531 | −11.5122 |
| N_WBC_FL_P | | | | | | | | | |
| N_WBC_SS_P; | 0.8803 | >−0.5561 | 24.4 | 83.5 | 75.6 | 16.5 | 0.002481 | 0.024909 | −36.0762 |
| N_WBC_FS_P | | | | | | | | | |
| N_WBC_FL_CV; | 0.8797 | >−0.332 | 18.1 | 82.6 | 81.9 | 17.4 | −11.0552 | 0.000569 | 6.14118 |
| N_WBC_FLSS_Area | | | | | | | | | |
| N_WBC_FL_CV; | 0.8779 | >−0.3141 | 16.9 | 75.8 | 83.1 | 24.2 | −3.80262 | 0.025085 | −28.9403 |
| N_WBC_FS_P | | | | | | | | | |
| N_WBC_SS_P; | 0.8779 | >−0.5951 | 19.3 | 80.1 | 80.7 | 19.9 | 0.006051 | 0.004837 | −14.4706 |
| N_WBC_FL_P | | | | | | | | | |
| N_WBC_SS_CV; | 0.8778 | >−0.3032 | 15.4 | 75 | 84.6 | 25 | 3.755923 | 0.005742 | −13.3492 |
| N_WBC_FL_P | | | | | | | | | |
| N_WBC_FS_W; | 0.8692 | >−0.3773 | 16.9 | 75 | 83.1 | 25 | 0.011284 | 0.000364 | −15.0936 |
| N_WBC_FLFS_Area | | | | | | | | | |
| N_WBC_FL_P; | 0.8688 | >−0.5144 | 19.3 | 79.2 | 80.7 | 20.8 | 0.006311 | 2.686449 | −12.9168 |
| N_WBC_FL_CV | | | | | | | | | |
| N_WBC_FL_P; | 0.8686 | >−0.4786 | 19.3 | 78.4 | 80.7 | 21.6 | 0.003984 | 0.002326 | −10.326 |
| N_WBC_FL_W | | | | | | | | | |
| N_WBC_SS_CV; | 0.867 | >−0.3873 | 16.1 | 75.4 | 83.9 | 24.6 | −4.96287 | 0.019506 | −14.1191 |
| N_WBC_FS_W | | | | | | | | | |
| N_WBC_FS_W; | 0.8667 | >−0.4754 | 18.5 | 76.3 | 81.5 | 23.7 | 0.011898 | 0.000196 | −14.62 |
| N_WBC_FLSS_Area | | | | | | | | | |
| N_WBC_FL_W; | 0.8665 | >−0.3949 | 18.9 | 79.7 | 81.1 | 20.3 | 0.005734 | −6.28385 | −2.87391 |
| N_WBC_FL_CV | | | | | | | | | |
| N_WBC_SS_P; | 0.8656 | >−0.4623 | 15.7 | 77.1 | 84.3 | 22.9 | 0.003496 | 0.01293 | −17.3523 |
| N_WBC_FS_W | | | | | | | | | |
| N_WBC_FL_CV; | 0.8622 | >−0.242 | 20.9 | 80.1 | 79.1 | 19.9 | −14.9109 | 0.000997 | 8.341987 |
| N_WBC_SSFS_Area | | | | | | | | | |
| N_WBC_FL_W; | 0.8611 | >−0.2901 | 18.5 | 74.6 | 81.5 | 25.4 | 0.004714 | 0.000361 | −11.9305 |
| N_WBC_FLFS_Area | | | | | | | | | |
| N_WBC_FL_W; | 0.8593 | >−0.286 | 20.5 | 75.4 | 79.5 | 24.6 | 0.004911 | 0.000205 | −11.3282 |
| N_WBC_FLSS_Area | | | | | | | | | |
| N_WBC_SS_P; | 0.859 | >−0.4077 | 21.3 | 78.8 | 78.7 | 21.2 | 0.004866 | 0.005158 | −14.9084 |
| N_WBC_FL_W | | | | | | | | | |
| N_WBC_FS_W; | 0.855 | >−0.4641 | 18.5 | 77.1 | 81.5 | 22.9 | 0.018146 | −0.00029 | −15.9582 |
| N_WBC_SSFS_Area | | | | | | | | | |
| N_WBC_SS_W; | 0.8531 | >−0.4641 | 18.9 | 75.8 | 81.1 | 24.2 | −0.00198 | 0.018958 | −16.7417 |
| N_WBC_FS_W | | | | | | | | | |
| N_WBC_FL_W; | 0.852 | >−0.2934 | 20.5 | 75 | 79.5 | 25 | 0.0058 | 0.000207 | −12.3492 |
| N_WBC_SSFS_Area | | | | | | | | | |
| N_WBC_SS_P; | 0.8506 | >−0.3095 | 21.3 | 78 | 78.7 | 22 | 0.007109 | 0.000579 | −13.7963 |
| N_WBC_FLFS_Area | | | | | | | | | |
| N_WBC_SS_W; | 0.8506 | >−0.303 | 20.9 | 78.8 | 79.1 | 21.2 | 0.005068 | −12.0972 | 7.229139 |
| N_WBC_FL_CV | | | | | | | | | |
| N_WBC_FL_W; | 0.8492 | >−0.2192 | 18.5 | 72 | 81.5 | 28 | 0.005685 | 4.00197 | −13.3035 |
| N_WBC_FS_CV | | | | | | | | | |
| N_WBC_SS_W; | 0.8469 | >−0.2594 | 19.7 | 72.9 | 80.3 | 27.1 | 0.000601 | 0.005852 | −11.3572 |
| N_WBC_FL_W | | | | | | | | | |
| N_WBC_FLFS_Area; | 0.8455 | >−0.1293 | 17.7 | 72.9 | 82.3 | 27.1 | 0.001322 | −0.00076 | −5.62311 |
| N_WBC_SSFS_Area | | | | | | | | | |
| N_WBC_SS_CV | 0.8441 | >−0.3829 | 22 | 74.2 | 78 | 25.8 | −0.35825 | 0.006211 | −10.7371 |
| ;N_WBC_FL_W | | | | | | | | | |
| N_WBC_FLSS_Area; | 0.8433 | >−0.1584 | 18.5 | 74.6 | 81.5 | 25.4 | 0.000992 | −0.00103 | −2.51441 |
| N_WBC_SSFS_Area | | | | | | | | | |
| N_WBC_SS_P; | 0.8396 | >−0.369 | 22.8 | 79.2 | 77.2 | 20.8 | 0.00659 | 0.000332 | −11.6971 |
| N_WBC_FLSS_Area | | | | | | | | | |
| N_WBC_SS_P; | 0.8294 | >−0.2247 | 25.6 | 75.4 | 74.4 | 24.6 | 0.011086 | −7.92893 | −3.49517 |
| N_WBC_FL_CV | | | | | | | | | |
| N_WBC_SS_W; | 0.8212 | >−0.1606 | 18.1 | 72 | 81.9 | 28 | 0.001063 | 0.000676 | −7.97939 |
| N_WBC_FLFS_Area | | | | | | | | | |
| N_WBC_SS_CV; | 0.8201 | >−0.1602 | 20.9 | 73.3 | 79.1 | 26.7 | −0.71348 | 0.000778 | −6.62595 |
| N_WBC_FLFS_Area | | | | | | | | | |
| N_WBC_SS_P; | 0.8188 | >−0.2774 | 20.1 | 71.6 | 79.9 | 28.4 | 0.009336 | 8.311957 | −17.2544 |
| N_WBC_FS_CV | | | | | | | | | |
| N_WBC_FS_CV; | 0.8176 | >−0.1746 | 20.5 | 72.9 | 79.5 | 27.1 | 0.97859 | 0.00073 | −7.7712 |
| N_WBC_FLFS_Area | | | | | | | | | |
| N_WBC_FLFS_Area; | 0.8174 | >−0.1134 | 18.9 | 71.2 | 81.1 | 28.8 | 0.00072 | 2.32E−05 | −7.20822 |
| N_WBC_FLSS_Area | | | | | | | | | |
| N_WBC_SS_CV; | 0.8146 | >−0.1111 | 21.7 | 72.5 | 78.3 | 27.5 | 8.556574 | 13.7437 | 5.982061 |
| N_WBC_FL_CV | | | | | | | | | |
| N_WBC_SS_CV; | 0.8083 | >−0.0844 | 16.5 | 69.1 | 83.5 | 30.9 | −0.97662 | 0.000489 | −4.81488 |
| N_WBC_FLSS_Area | | | | | | | | | |
| N_WBC_SS_W; | 0.8079 | >−0.2443 | 20.9 | 72 | 79.1 | 28 | 0.000758 | 0.000423 | −6.23533 |
| N_WBC_FLSS_Area | | | | | | | | | |
| N_WBC_FS_CV; | 0.8039 | >−0.1561 | 17.3 | 69.5 | 82.7 | 30.5 | 2.472525 | 0.000427 | −7.13044 |
| N_WBC_FLSS_Area | | | | | | | | | |

TABLE 20-2
Efficacy of using PCT (procalcitonin) of prior art, and the parameters of the
DIFF channel for the identification of bacterial infection and viral infection

| Infection | | Determi- | False | True | True | False |
|---|---|---|---|---|---|---|
| marker | | nation | positive | positive | negative | negative |
| parameter | ROC_AUC | threshold | rate | rate | rate | rate |
| PCT | 0.851 | 0.554 | 7.9% | 67.3% | 92.1% | 32.7% |
| D_Neu_SS_W | 0.733 | 259.275 | 24.4% | 60.2% | 75.6% | 39.8% |
| D_Neu_FL_W | 0.836 | 206.183 | 20.1% | 75.0% | 79.9% | 25.0% |
| D_Neu_FS_W | 0.601 | 611.240 | 34.6% | 56.4% | 65.4% | 43.6% |

From the comparison between Table 20-2 and Tables 19 and 20-1, it can be seen that the parameters of WNB channel have similar diagnostic and therapeutic efficacy to or even better diagnostic and therapeutic efficacy than PCT in the identification of bacterial infections, and in addition, the parameters also have better diagnostic performance than the parameters of DIFF channel in the differential diagnosis of bacterial infections.

TABLE 20-3
Illustration of the statistical methods and testing methods
used in this example by taking three parameters as examples

| Infection | | | | |
|---|---|---|---|---|
| marker | Positive sample | Negative sample | | |
| parameter | Mean ± SD | Mean ± SD | F value | P value |
| N_WBC_FS_P | 1367.119 ± 65.7629 | 1293.4373 ± 33.4095 | −4.47 | <0.0001 |
| N_WBC_FL_P | 1728.4606 ± 340.3289 | 1333.4406 ± 167.3573 | 12.24 | <0.0001 |
| N_WBC_FS_W | 1084.3 ± 118.1 | 963.4 ± 49.4 | 3.13 | <0.0001 |

As can be seen from Table 20-3, this parameter is analyzed by Welch test, and there is a significant statistical difference between the two groups (p<0.0001)

As can be seen from Tables 19 and 20-1 and 20-2, the infection marker parameters provided in the disclosure can be used to effectively identify a bacterial infection and a viral infection. For the same reasons as example 1, the disclosure accidentally discovered through in-depth investigation that a more useful feature can be found from the WNB channel than from the DIFF channel to identify a bacterial infection and a viral infection.

### Example 8. Identification of an Infectious Inflammation and a Non-Infectious Inflammation

515 blood samples were subjected to test using the BC-6800 Plus blood cell analyzer produced by SHENZHEN MINDRAY BIO-MEDICAL ELECTRONICS CO., LTD. in accordance with the steps of example 1 of the disclosure, and the aforementioned method was adopted for identifying an infectious inflammation based on the scattergram. Among them, there were 399 infectious inflammation samples, that is, positive samples, and 116 non-infectious inflammation samples, that is, negative samples.

Inclusion criteria for these cases: adult ICU patients with acute inflammation or with suspected acute inflammation. Exclusion criteria: pregnant people, myelosuppressed people on chemotherapy, people on immunosuppressant treatment, patients with hematologic diseases.

For the infectious inflammation samples: there was evidence of bacterial and/or viral infection; and there was inflammation (meeting any of the following was sufficient)
1. Local inflammatory manifestations or systemic inflammatory response manifestations
2. Tissue damage: damage caused by physical or chemical factors such as high temperature, low temperature, radioactive substances and ultraviolet rays
3. Mechanical damage: damage caused by chemicals such as strong acids, alkalis, etc
4. Tissue necrosis: tissue necrosis and damage caused by ischemia or hypoxia
5. Allergy: abnormal state of the body's immune response, such as autoimmune diseases

For the non-infectious inflammation samples: inflammatory responses caused by physical, chemical and other factors, which met both (1) and (2):
(1) No evidence of bacterial infection
(2) Presence of inflammation (meeting any of the following was sufficient)
1. Local inflammatory manifestations or systemic inflammatory response manifestations
2. Tissue damage: damage caused by physical or chemical factors such as high temperature, low temperature, radioactive substances and ultraviolet rays
3. Mechanical damage: damage caused by chemicals such as strong acids, alkalis, etc
4. Tissue necrosis: tissue necrosis and damage caused by ischemia or hypoxia
5. Allergy: abnormal state of the body's immune response, such as autoimmune diseases

Table 21 shows the efficacy of using single leukocyte characteristic parameters as infection marker parameters for determining an infectious inflammation in this example, and Table 22-1 shows the efficacy of using parameter combinations as infection marker parameters for determining an infectious inflammation in this example, wherein infection marker parameters are calculated by the function Y=A×X1+B×X2+C based on the parameter combinations in the Table 22-1, where Y represents an infection marker parameter, X1 represents the first leukocyte parameter, X2 represents the second leukocyte parameter, and A, B, and C are constants.

TABLE 21
Efficacy of single parameters for identification of an infectious
inflammation and a non-infectious inflammation

| | | Determi- | False | True | True | False |
|---|---|---|---|---|---|---|
| | | nation | positive | positive | negative | negative |
| Single parameter | ROC_AUC | threshold | rate % | rate % | rate % | rate % |
| N_WBC_FL_W | 0.924 | >1808 | 14.7 | 85.6 | 85.3 | 14.4 |
| N_WBC_FL_P | 0.8735 | >1637.309 | 17.2 | 77.8 | 82.8 | 22.2 |
| N_WBC_SS_W | 0.8731 | >1328 | 17.2 | 78.5 | 82.8 | 21.5 |
| N_WBC_FS_W | 0.8692 | >944 | 19.8 | 81.1 | 80.2 | 18.9 |
| N_WBC_SS_P | 0.8359 | >1140.0115 | 22.4 | 76.5 | 77.6 | 23.5 |
| N_WBC_FS_P | 0.8 | >1279.999 | 23.3 | 71.7 | 76.7 | 28.3 |
| N_WBC_FS_CV | 0.783 | >0.7505 | 19 | 67.2 | 81 | 32.8 |
| N_WBC_SS_CV | 0.768 | >1.1525 | 20.7 | 69.4 | 79.3 | 30.6 |

TABLE 22-1
Efficacy of two-parameter combination for identification of an infectious inflammation and a non-infectious inflammation

| | | Determi- | False | True | True | False | | | |
|---|---|---|---|---|---|---|---|---|---|
| Parameter | | nation | positive | positive | negative | negative | | | |
| combination | ROC_AUC | threshold | rate % | rate % | rate % | rate % | A | B | C |
| N_WBC_FL_P; | 0.9431 | >0.7362 | 13.8 | 89.1 | 86.2 | 10.9 | 0.004296 | 0.009719 | −15.3144 |
| N_WBC_FS_W | | | | | | | | | |
| N_WBC_FL_P; | 0.9391 | >0.8824 | 8.6 | 86.1 | 91.4 | 13.9 | 0.004775 | 13.28345 | −16.658 |
| N_WBC_FS_CV | | | | | | | | | |
| N_WBC_SS_W; | 0.9371 | >0.8394 | 12.1 | 86.4 | 87.9 | 13.6 | 0.003752 | 0.004303 | −11.0935 |
| N_WBC_FL_P | | | | | | | | | |
| N_WBC_FL_W; | 0.9358 | >0.7516 | 12.1 | 86.9 | 87.9 | 13.1 | 0.005878 | 0.003336 | −13.0782 |
| N_WBC_FS_W | | | | | | | | | |
| N_WBC_SS_CV; | 0.9357 | >0.684 | 12.9 | 87.6 | 87.1 | 12.4 | 6.846993 | 0.004928 | −15.0248 |
| N_WBC_FL_P | | | | | | | | | |
| N_WBC_SS_W; | 0.9338 | >0.6326 | 13.8 | 87.9 | 86.2 | 12.1 | 0.001255 | 0.006015 | −11.836 |
| N_WBC_FL_W | | | | | | | | | |
| N_WBC_FL_W; | 0.9327 | >0.8146 | 14.7 | 87.6 | 85.3 | 12.4 | 0.006225 | 0.010676 | −24.2504 |
| N_WBC_FS_P | | | | | | | | | |
| N_WBC_SS_P; | 0.9316 | >0.7405 | 12.1 | 86.6 | 87.9 | 13.4 | 0.00378 | 0.005958 | −14.387 |
| N_WBC_FL_W | | | | | | | | | |
| N_WBC_SS_CV; | 0.9313 | >0.7055 | 12.9 | 86.4 | 87.1 | 13.6 | 1.800278 | 0.006288 | −12.7394 |
| N_WBC_FL_W | | | | | | | | | |
| N_WBC_FL_W; | 0.9265 | >0.8233 | 12.9 | 85.6 | 87.1 | 14.4 | 0.006519 | 1.067155 | −11.8387 |
| N_WBC_FS_CV | | | | | | | | | |
| N_WBC_FL_CV; | 0.9252 | >0.9496 | 13.8 | 86.6 | 86.2 | 13.4 | −7.82314 | 0.014351 | −3.67145 |
| N_WBC_FS_W | | | | | | | | | |
| N_WBC_FL_P; | 0.9239 | >0.6991 | 13.8 | 86.6 | 86.2 | 13.4 | 0.007019 | 9.282028 | −21.1036 |
| N_WBC_FL_CV | | | | | | | | | |
| N_WBC_FL_P; | 0.9234 | >1.1556 | 10.3 | 81.6 | 89.7 | 18.4 | 0.001168 | 0.005706 | −11.4278 |
| N_WBC_FL_W | | | | | | | | | |
| N_WBC_FL_W; | 0.9218 | >1.1866 | 9.5 | 81.6 | 90.5 | 18.4 | 0.006744 | −2.20766 | −8.89375 |
| N_WBC_FL_CV | | | | | | | | | |
| N_WBC_FL_CV; | 0.9171 | >1.1798 | 11.2 | 82.3 | 88.8 | 17.7 | −11.3778 | 24.17808 | −3.80884 |
| N_WBC_FS_CV | | | | | | | | | |
| N_WBC_SS_P; | 0.9092 | >0.8106 | 19 | 87.1 | 81 | 12.9 | 0.007061 | 0.003745 | −13.1725 |
| N_WBC_FL_P | | | | | | | | | |
| N_WBC_SS_W; | 0.9089 | >0.9408 | 12.1 | 83.8 | 87.9 | 16.2 | 0.005187 | −6.4277 | 1.452877 |
| N_WBC_FL_CV | | | | | | | | | |
| N_WBC_FL_P; | 0.893 | >1.158 | 15.5 | 80.8 | 84.5 | 19.2 | 0.003788 | 0.009871 | −17.74 |
| N_WBC_FS_P | | | | | | | | | |
| N_WBC_SS_W; | 0.8912 | >0.9503 | 16.4 | 82.6 | 83.6 | 17.4 | 0.003273 | 0.011863 | −18.6389 |
| N_WBC_FS_P | | | | | | | | | |
| N_WBC_SS_CV; | 0.8893 | >0.8556 | 19.8 | 83.3 | 80.2 | 16.7 | 6.270673 | 0.01678 | −27.8675 |
| N_WBC_FS_P | | | | | | | | | |
| N_WBC_SS_W; | 0.8887 | >0.9871 | 13.8 | 80.6 | 86.2 | 19.4 | 0.001864 | 0.006783 | −7.98118 |
| N_WBC_FS_W | | | | | | | | | |
| N_WBC_SS_P; | 0.8883 | >0.9893 | 17.2 | 80.6 | 82.8 | 19.4 | 0.005449 | 0.00697 | −11.9176 |
| N_WBC_FS_W | | | | | | | | | |
| N_WBC_FS_P; | 0.8876 | >0.8068 | 20.7 | 84.1 | 79.3 | 15.9 | 0.015639 | 11.89857 | −27.9203 |
| N_WBC_FS_CV | | | | | | | | | |
| N_WBC_FS_P; | 0.885 | >0.7639 | 22.4 | 84.6 | 77.6 | 15.4 | 0.009189 | 0.007896 | −18.3163 |
| N_WBC_FS_W | | | | | | | | | |
| N_WBC_FS_W; | 0.8831 | >1.025 | 15.5 | 78.3 | 84.5 | 21.7 | 0.015708 | −9.73115 | −6.73232 |
| N_WBC_FS_CV | | | | | | | | | |
| N_WBC_SS_P; | 0.8799 | >0.9096 | 19 | 81.1 | 81 | 18.9 | 0.00801 | 3.687035 | −12.4963 |
| N_WBC_SS_CV | | | | | | | | | |
| N_WBC_SS_W; | 0.8789 | >0.9949 | 16.4 | 79.8 | 83.6 | 20.2 | 0.004798 | −2.05387 | −3.0257 |
| N_WBC_SS_CV | | | | | | | | | |
| N_WBC_SS_CV; | 0.8757 | >1.0471 | 12.9 | 77.8 | 87.1 | 22.2 | 1.793437 | 0.008536 | −9.21974 |
| N_WBC_FS_W | | | | | | | | | |
| N_WBC_SS_P; | 0.8757 | >0.9404 | 19.8 | 79.3 | 80.2 | 20.7 | 0.005397 | 0.002244 | −8.19785 |
| N_WBC_SS_W | | | | | | | | | |
| N_WBC_SS_P; | 0.8749 | >1.0358 | 18.1 | 79.3 | 81.9 | 20.7 | 0.007789 | 7.440523 | −13.4705 |
| N_WBC_FS_CV | | | | | | | | | |
| N_WBC_SS_W; | 0.8731 | >0.9688 | 15.5 | 78.3 | 84.5 | 21.7 | 0.00313 | 3.715312 | −5.94219 |
| N_WBC_FS_CV | | | | | | | | | |
| N_WBC_SS_CV; | 0.8558 | >1.0558 | 18.1 | 73 | 81.9 | 27 | 9.076347 | −7.76849 | −0.54283 |
| N_WBC_FL_CV | | | | | | | | | |
| N_WBC_SS_P; | 0.8511 | >1.06 | 19.8 | 75.8 | 80.2 | 24.2 | 0.006814 | 0.007903 | −16.9119 |
| N_WBC_FS_P | | | | | | | | | |
| N_WBC_SS_P; | 0.8462 | >1.024 | 20.7 | 75.8 | 79.3 | 24.2 | 0.009651 | −2.20524 | −7.46833 |
| N_WBC_FL_CV | | | | | | | | | |
| N_WBC_FL_CV; | 0.8009 | >1.0003 | 27.6 | 75.8 | 72.4 | 24.2 | 0.463272 | 0.014822 | −18.427 |
| N_WBC_FS_P | | | | | | | | | |
| N_WBC_SS_CV; | 0.8004 | >1.1191 | 16.4 | 69.7 | 83.6 | 30.3 | 2.883613 | 7.0589 | −7.52639 |
| N_WBC_FS_CV | | | | | | | | | |

TABLE 22-2
Efficacy of using PCT (procalcitonin) of prior art, and the parameters of the DIFF channel
for identification of an infectious inflammation and a non-infectious inflammation

| | | Determi- | False | True | True | False |
|---|---|---|---|---|---|---|
| Infection marker | | nation | positive | positive | negative | negative |
| parameter | ROC_AUC | threshold | rate | rate | rate | rate |
| PCT | 0.855 | 0.44 | 32.1% | 89.6% | 67.9% | 10.4% |
| D_Neu_SS_W | 0.744 | 290.101 | 7.8% | 45.7% | 92.2% | 54.3% |
| D_Neu_FL_W | 0.836 | 220.534 | 14.7% | 67.3% | 85.3% | 32.7% |
| D_Neu_FS_W | 0.557 | 563.910 | 37.9% | 51.3% | 62.1% | 48.7% |

From the comparison between Table 22-2 and Table 21 and 22-1, it can be seen that the parameters of WNB channel have similar diagnostic and therapeutic efficacy to or even better diagnostic and therapeutic efficacy than PCT in the identification of an infectious inflammation and a non-infectious inflammation, and in addition, the parameters also have better diagnostic performance than the parameters of DIFF channel in the identification of an infectious inflammation and a non-infectious inflammation.

TABLE 22-3
Illustration of the statistical methods and testing methods
used in this example by taking three parameters as examples

| Infection | | | | |
|---|---|---|---|---|
| marker | Positive sample | Negative sample | | |
| parameter | Mean ± SD | Mean ± SD | F value | P value |
| N_WBC_FL_W | 2116.7 ± 287.1 | 1645.2 ± 173.2 | 21.82 | <0.0001 |
| N_WBC_FL_P | 1875.8059 ± 345.0117 | 1417.2917 ± 243.4785 | 12.76 | <0.0001 |
| N_WBC_SS_W | 1562.6 ± 328.1 | 1227.9 ± 141.4 | 15.88 | <0.0001 |

As can be seen from Table 22-3, this parameter is analyzed by Welch test, and there is a significant statistical difference between the two groups (p<0.0001)

As can be seen from Tables 21 and 22-1, 22-2, the infection marker parameters provided in the disclosure can be used to effectively identify an infectious inflammation and a non-infectious inflammation. For the same reasons as example 1, the disclosure accidentally discovered through in-depth investigation that a more useful feature can be found from the WNB channel than from the DIFF channel to identify an infectious inflammation and a non-infectious inflammation.

### Example 9 Evaluation of Therapeutic Effect on Sepsis

Blood samples of 28 patients receiving treatment on sepsis were subjected to blood routine test using the BC-6800 Plus blood cell analyzer produced by SHENZHEN MINDRAY BIO-MEDICAL ELECTRONICS CO., LTD. in accordance with the steps of example 1, and the aforementioned method was adopted for evaluation of therapeutic effect on sepsis based on the scattergram. Specifically, 28 patients diagnosed with sepsis were treated with antibiotics, and blood samples from the patients were subjected to blood routine test 5 days later, and the parameters in the following table were obtained. Based on the therapeutic effects over 5 days, the patients were divided into effective group and ineffective group and the patients with clinical significant improvement of symptoms were divided into the effective group, otherwise divided into the ineffective group. Among them, 11 patients belonged to the ineffective group and 17 patients belonged to the effective group.

Table 23 shows the use of a single leukocyte characteristic parameter as an infection marker parameter for determining the efficacy on sepsis in this embodiment. Where N_FL_PULWID_MEAN refers to the average pulse width of the side fluorescence signal of the particles in the leukocyte population of the WNB channel scattergram; N_FS_PULWID_MEAN refers to the average pulse width of the forward scatter signal of the particles in the leukocyte population of the WNB channel scattergram; N_SS_PULWID_MEAN refers to the average pulse width of the side scatter signal of the particles in the leukocyte population of the WNB channel scattergram; N_WBC_FL_R refers to the right boundary value of the side fluorescence intensity distribution in the leukocyte population of the WNB channel scattergram (shown in FIG. 6).

TABLE 23
Single parameters for determining the therapeutic effect on sepsis

| | | Determi- | False | True | True | False |
|---|---|---|---|---|---|---|
| | | nation | positive | positive | negative | negative |
| Single parameter | ROC_AUC | threshold | rate % | rate % | rate % | rate % |
| N_WBC_FL_P; | 0.8663 | >23.682 | 5.9 | 72.7 | 94.1 | 27.3 |
| N_FL_PULWID_MEAN; | 0.861 | >−0.166 | 23.5 | 90.9 | 76.5 | 9.1 |
| N_FS_PULWID_MEAN; | 0.8503 | >−0.0965 | 17.6 | 81.8 | 82.4 | 18.2 |
| N_WBC_FL_W; | 0.8476 | >−48 | 17.6 | 72.7 | 82.4 | 27.3 |
| N_WBC_FL_R; | 0.7861 | >−1.5 | 11.8 | 72.7 | 88.2 | 27.3 |
| N_WBC_FS_P; | 0.7754 | >12.171 | 23.5 | 72.7 | 76.5 | 27.3 |
| N_SS_PULWID_MEAN; | 0.754 | >0.015 | 17.6 | 63.6 | 82.4 | 36.4 |
| N_WBC_FS_W; | 0.7433 | >−48 | 35.3 | 90.9 | 64.7 | 9.1 |
| N_WBC_SS_P; | 0.7273 | >16.7385 | 17.6 | 63.6 | 82.4 | 36.4 |
| N_WBC_SS_W; | 0.6952 | >32.5 | 29.4 | 63.6 | 70.6 | 36.4 |
| N_WBC_FS_CV; | 0.6711 | >−0.0125 | 35.3 | 72.7 | 64.7 | 27.3 |
| N_WBC_FLSS_Area; | 0.6684 | >−235.52 | 35.3 | 72.7 | 64.7 | 27.3 |
| N_WBC_FLFS_Area; | 0.6658 | >−399.36 | 41.2 | 72.7 | 58.8 | 27.3 |
| N_WBC_FL_CV; | 0.6631 | >0.0135 | 41.2 | 72.7 | 58.8 | 27.3 |
| N_WBC_SSFS_Area; | 0.5989 | >337.92 | 29.4 | 63.6 | 70.6 | 36.4 |
| N_WBC_SS_CV; | 0.5775 | >0.092 | 17.6 | 45.5 | 82.4 | 54.5 |

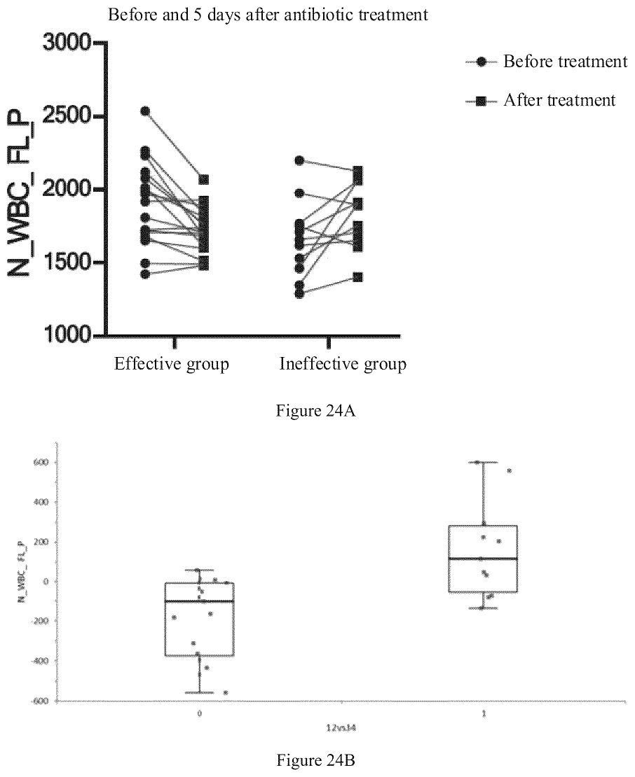

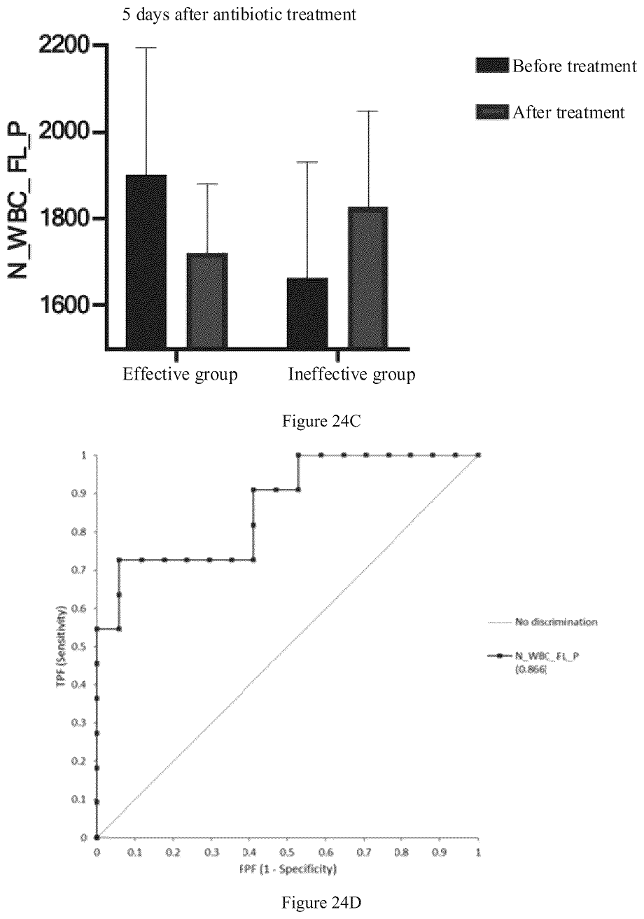

FIGS. 24A-24D visually show results of detection of efficacy on sepsis using N_WBC_FL_P as a single parameter.

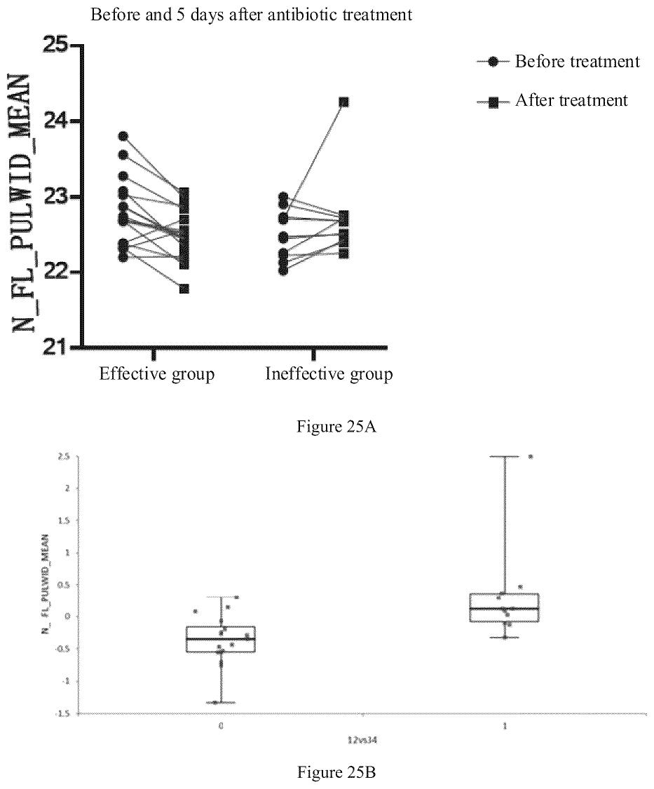

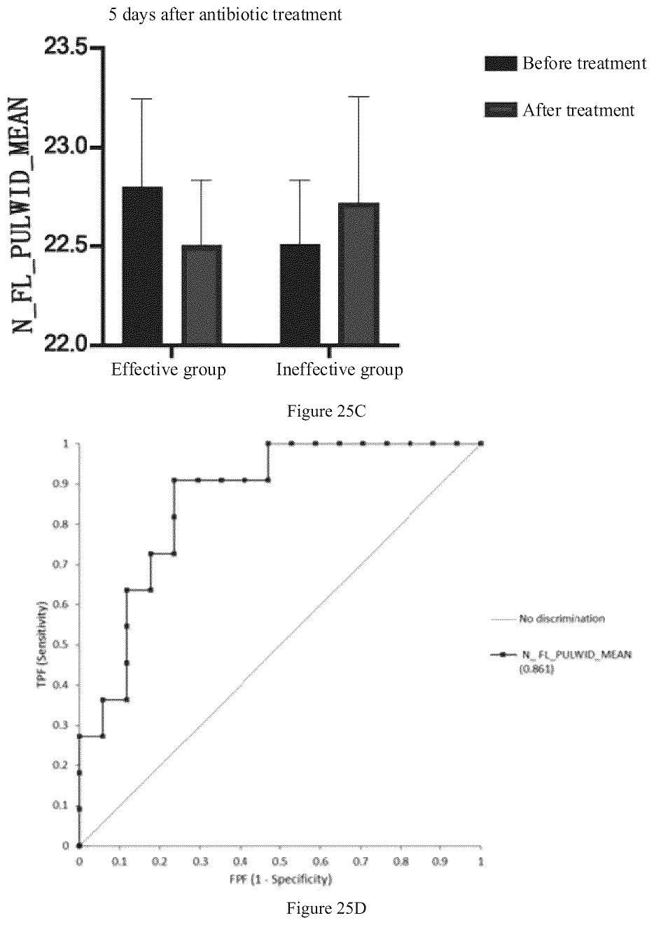

FIGS. 25A-25D visually show results of detection of efficacy on sepsis using N_FL_PULWID_MEAN as a single parameter.

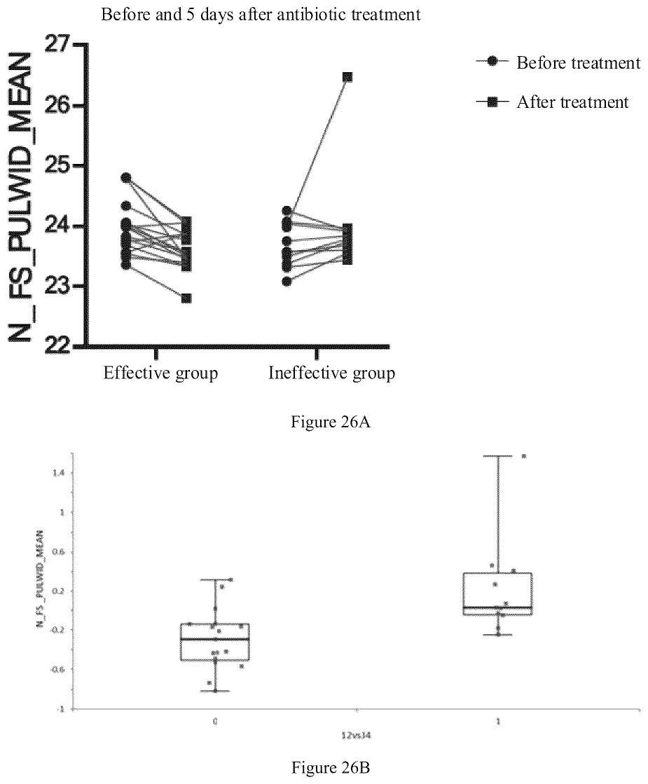

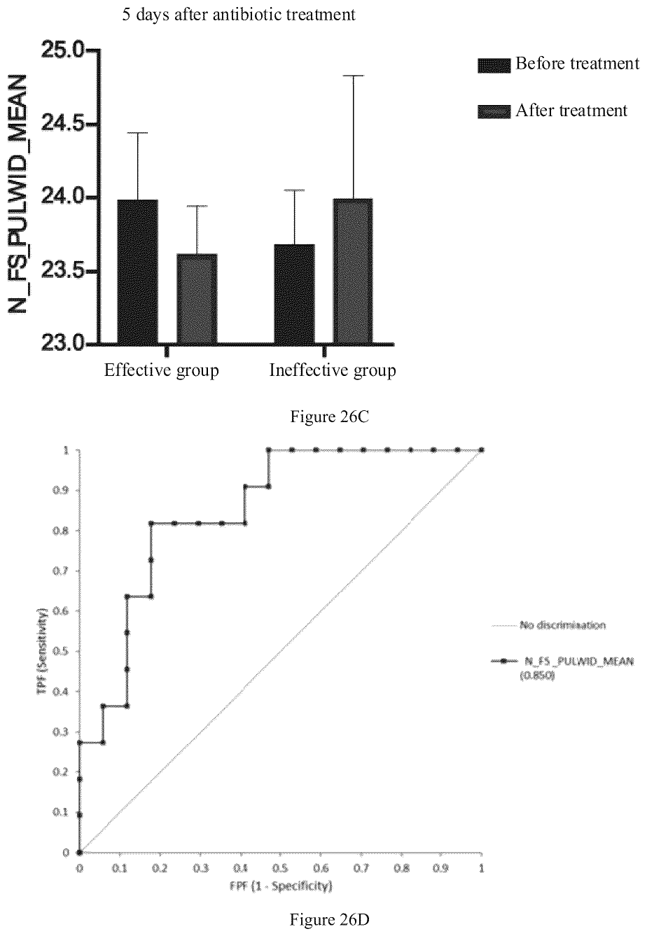

FIGS. 26A-26D visually show results of detection of efficacy on sepsis using N_FS_PULWID_MEAN as a single parameter.

Table 24 shows the use of the combination of the two parameters “N_WBC_FL_P” and “N_WBC_FS_W” as an infection marker parameter for determining the therapeutic effect on sepsis. The physical meaning of the two-parameter combination is to combine the center of gravity of the internal nucleic acid content of the WBC particles of the first detection channel with the distribution width of the volume of the WBC particles of the first detection channel.

The infection marker parameter was calculated from the two-parameter combination through the function

Y=0.0040875×N_WBC_FL_P+0.00905881×N_WBC_FS_W−16.60028217, where, Y represents the infection marker parameter.

TABLE 24

| Parameters for | | | | | | |
|---|---|---|---|---|---|---|
| evaluation of | | | False | True | True | False |
| therapeutic | | Diagnostic | positive | positive | negative | negative |
| effect on sepsis | ROC_AUC | threshold | rate | rate | rate | rate |
| Combination | 0.872 | −0.4451 | 17.6% | 90.9% | 82.4% | 9.1% |
| parameter | | | | | | |

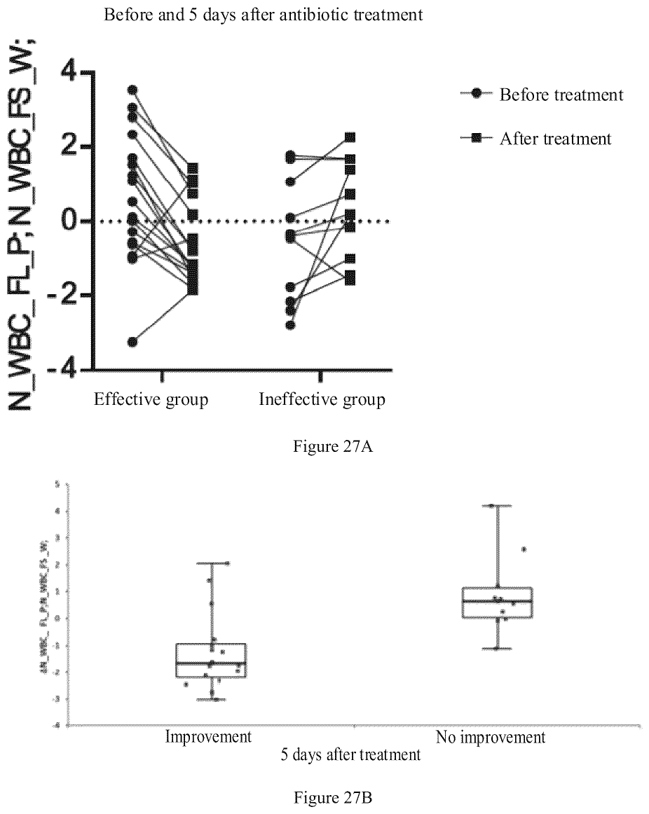

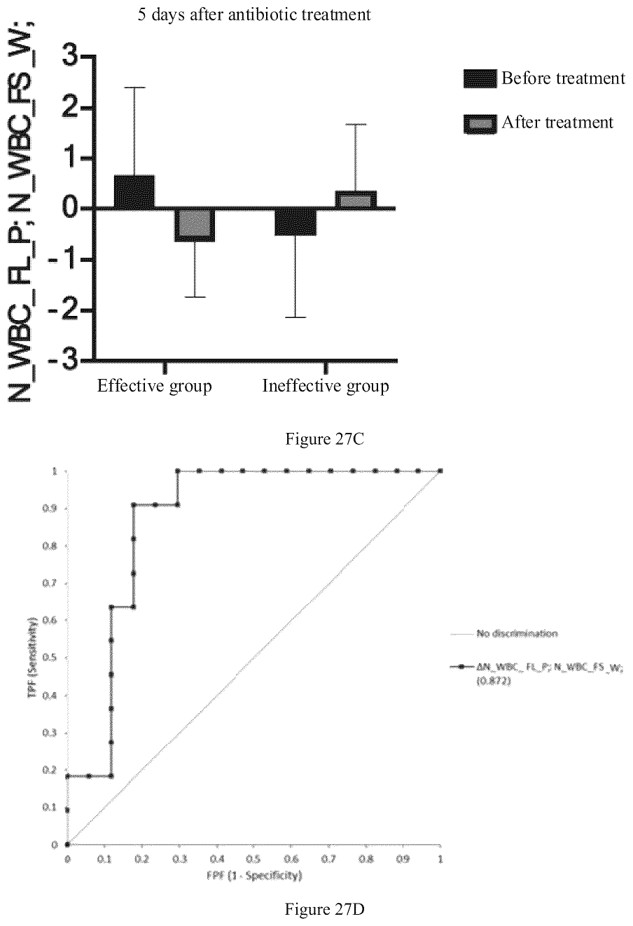

FIGS. 27A-27D visually show results of detection of efficacy on sepsis using a combination of the two parameters “N_WBC_FL_P” and “N_WBC_FS_W” as the infection marker parameter.

Table 25 shows the use of the combination of the two parameters “N_WBC_FL_W” and “N_WBC_FS_P” as an infection marker parameter for determining the therapeutic effect on sepsis. The physical meaning of the two-parameter combination is to combine the distribution width of the internal nucleic acid content of the WBC particles of the first detection channel with the center of gravity of the volume of the WBC particles of the first detection channel.

The infection marker parameter was obtained from the two-parameter combination through the function Y=0.00609253×N_WBC_FL_W+0.00587667×N_WBC_FS_P−20.07103538, where, Y represents the infection marker parameter.

TABLE 25

| Parameters for | | | | | | |
|---|---|---|---|---|---|---|
| evaluation of | | | False | True | True | False |
| therapeutic | | Diagnostic | positive | positive | negative | negative |
| effect on sepsis | ROC_AUC | threshold | rate | rate | rate | rate |
| Combination | 0.845 | −0.6059 | 23.5% | 90.9% | 76.5% | 9.1% |
| parameter | | | | | | |

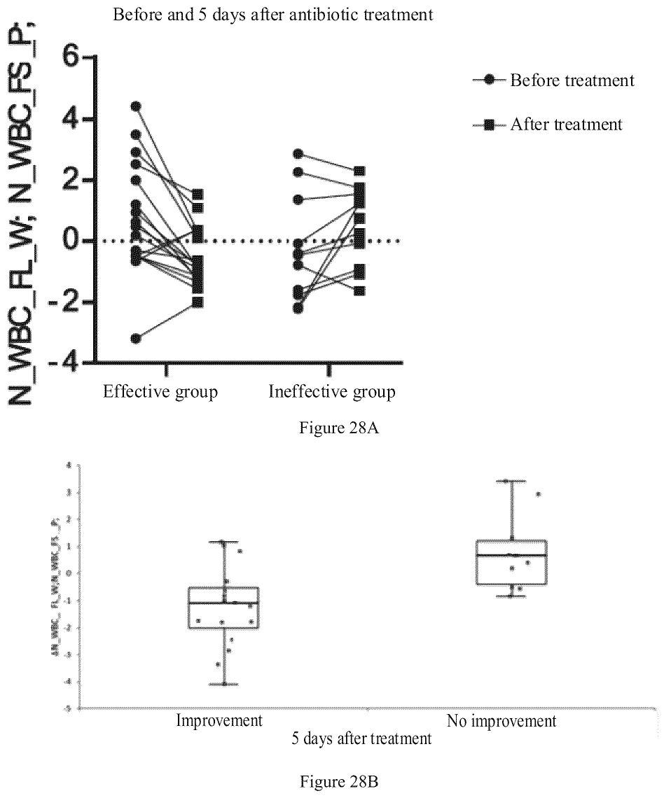

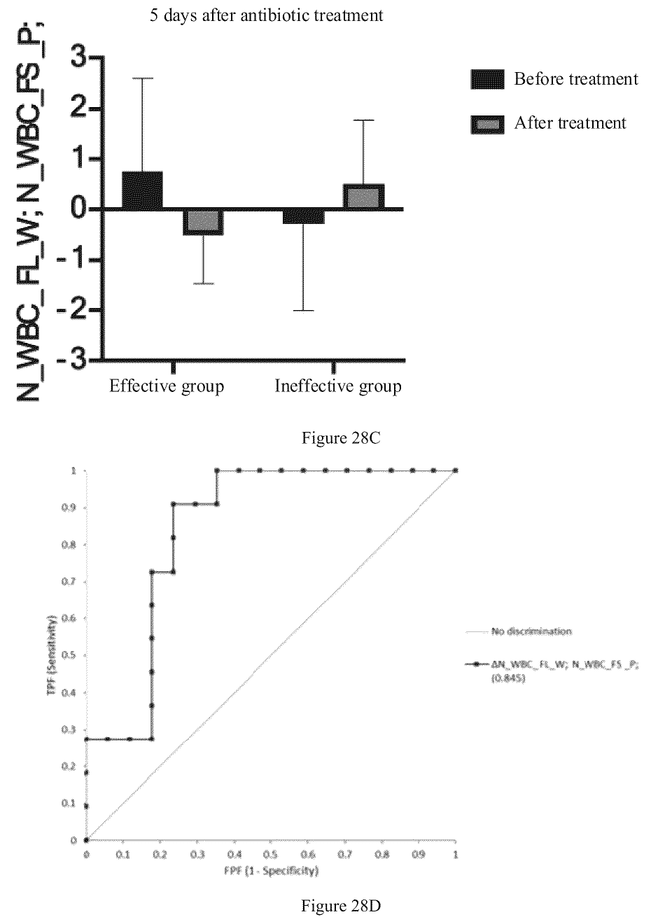

FIGS. 28A-28D visually show results of detection of efficacy on sepsis using a combination of the two parameters “N_WBC_FL_W” and “N_WBC_FS_P” as the infection marker parameter.

Table 26 shows the use of the combination of the two parameters “N_WBC_FL_P” and “N_WBC_FS_CV” as an infection marker parameter for determining the therapeutic effect on sepsis. The physical meaning of the two-parameter combination is to combine the central position of the internal nucleic acid content of the WBC particles of the first detection channel with the dispersion degree of the volume of the WBC particles of the first detection channel.

The infection marker parameter was obtained from the two-parameter combination through the function
Y=0.00462573×N_WBC_FL_P+12.43796108×N_WBC_FS_CV−18.03119401, where, Y represents the infection marker parameter.

TABLE 26

| Parameters for | | | | | | |
|---|---|---|---|---|---|---|
| evaluation of | | | False | True | True | False |
| therapeutic | | Diagnostic | positive | positive | negative | negative |
| effect on sepsis | ROC_AUC | threshold | rate | rate | rate | rate |
| Combination | 0.872 | −0.5031 | 17.6% | 90.9% | 82.4% | 9.1% |
| parameter | | | | | | |

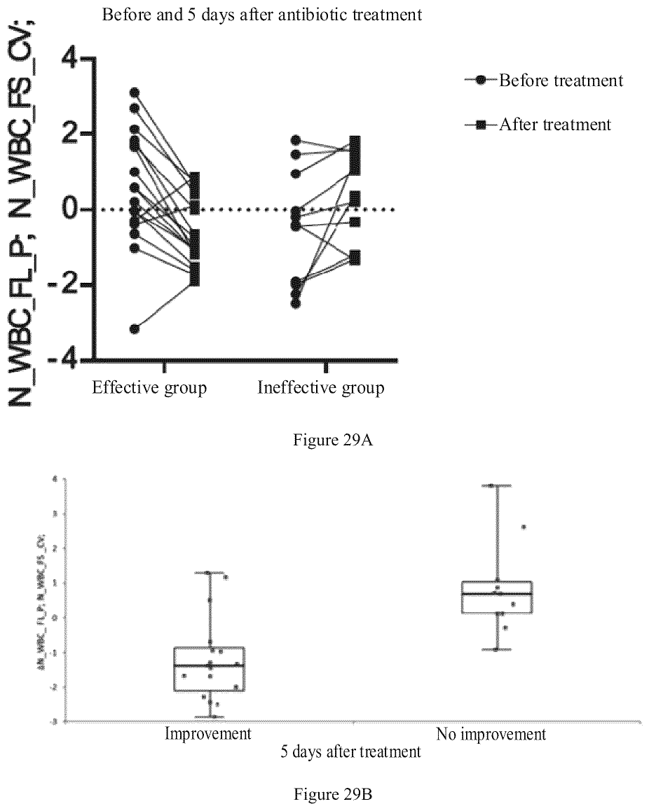

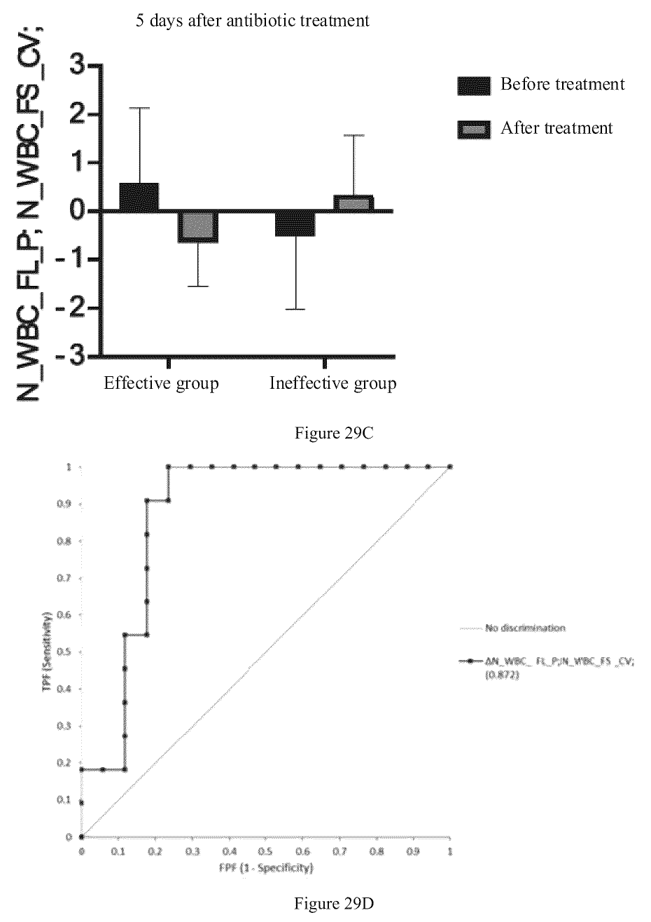

FIGS. 29A-29D visually show results of detection of efficacy on sepsis using a combination of the two parameters “N_WBC_FL_P” and “N_WBC_FS_CV” as the infection marker parameter.

Table 27 shows the combination of DIFF+WNB dual channel parameters “N_WBC_FL_W” and “D_Neu_FL_W” as an infection marker parameter for determining the therapeutic effect on sepsis. The physical meaning of the two-parameter combination is to combine the distribution width of the internal nucleic acid content of the WBC particles of the first detection channel and the distribution width of the internal nucleic acid content of the neutrophils of the second detection channel.

The infection marker parameter was obtained from the two-parameter combination through the function.

Y=0.00623272×N_WBC_FL_W+0.01806527× D_Neu_FL_W-16.84312131, where, Y represents the infection marker parameter.

TABLE 27

| Parameters for | | | | | | |
|---|---|---|---|---|---|---|
| evaluation of | | | False | True | True | False |
| therapeutic | | Diagnostic | positive | positive | negative | negative |
| effect on sepsis | ROC_AUC | threshold | rate | rate | rate | rate |
| Combination | 0.888 | −0.5564 | 17.6% | 81.8% | 82.4% | 18.2% |
| parameter | | | | | | |

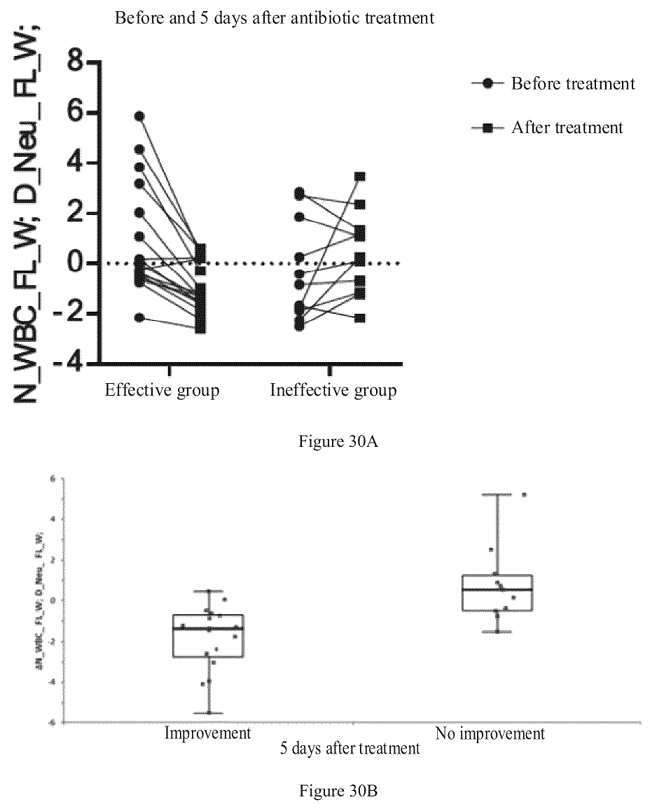

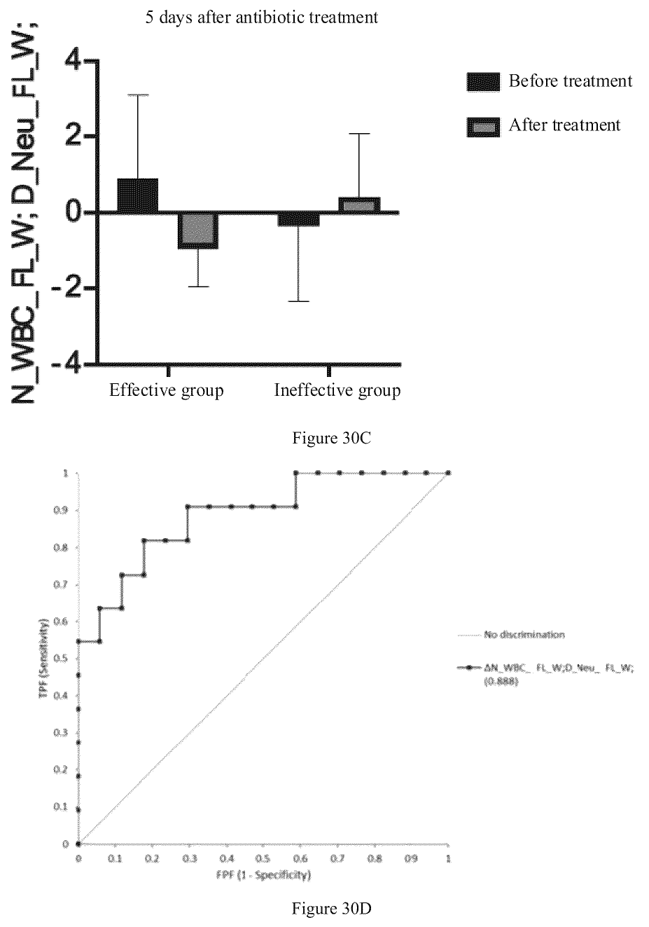

FIGS. 30A-30D visually show results of detection of efficacy on sepsis using a combination of the two parameters “N_WBC_FL_W” and “D_Neu_FL_W” as the infection marker parameter.

Table 28 shows the combination of DIFF+WNB dual channel parameters “N_WBC_FL W” and “D_Neu_FL_CV” as an infection marker parameter for determining the therapeutic effect on sepsis. The physical meaning of the two-parameter combination is to combine the distribution width of the internal nucleic acid content of the WBC particles of the first detection channel and the dispersion degree of the internal nucleic acid content of the neutrophils of the second detection channel.

The infection marker parameter was obtained from the two-parameter combination through the function
Y=0.00688519×N_WBC_FL_W+11.27099282×D_Neu_FL_CV-19.2998686, where, Y represents the infection marker parameter.

TABLE 28

| Parameters for | | | | | | |
|---|---|---|---|---|---|---|
| evaluation of | | | False | True | True | False |
| therapeutic | | Diagnostic | positive | positive | negative | negative |
| effect on sepsis | ROC_AUC | threshold | rate | rate | rate | rate |
| Combination | 0.850 | −0.042 | 11.8% | 72.7% | 88.2% | 27.3% |
| parameter | | | | | | |

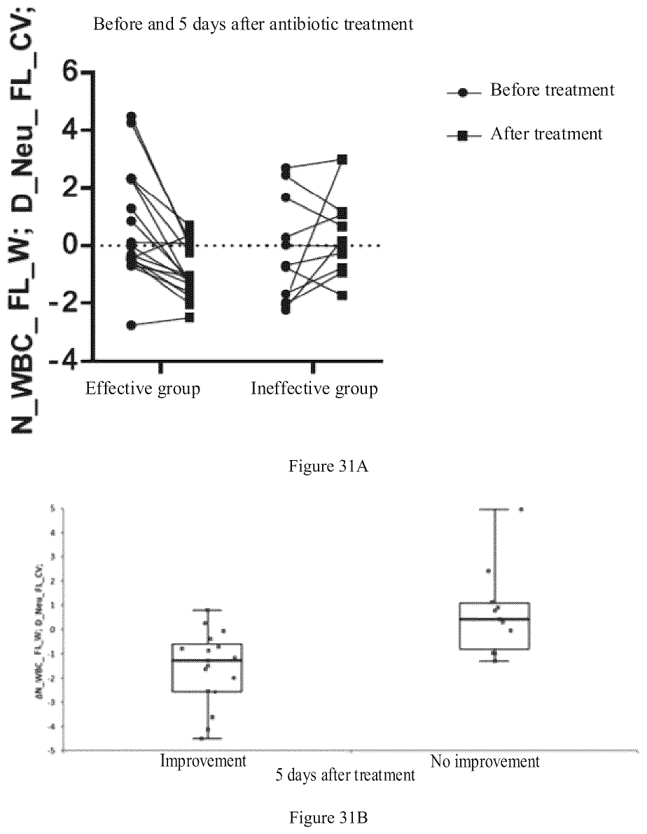

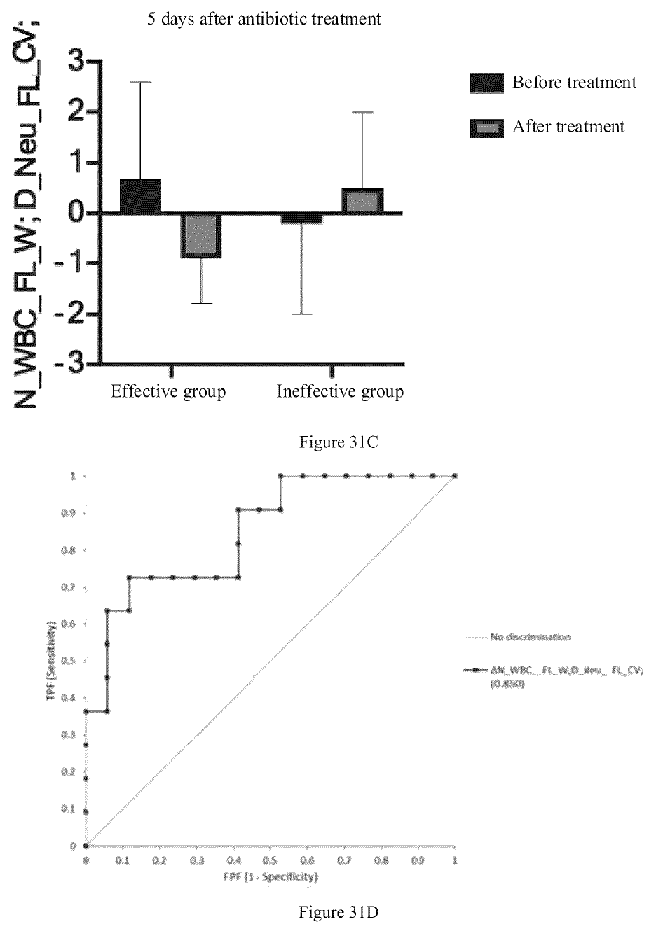

FIGS. 31A-31D visually show results of detection of efficacy on sepsis using a combination of the two parameters “N_WBC_FL_W” and “D_Neu_FL_CV” as the infection marker parameter.

### Example 10 Count Value Combined with Parameters for Diagnosis of Sepsis

1748 blood samples were subjected to blood routine test using the BC-6800 Plus blood cell analyzer produced by SHENZHEN MINDRAY BIO-MEDICAL ELECTRONICS CO., LTD. in accordance with the steps similar to example 3 of the disclosure, and the aforementioned method was adopted for diagnosis of sepsis based on the scattergram. Among them, there were 506 sepsis samples, that is, positive samples, and 1,242 non-sepsis samples, that is, negative samples.

Inclusion criteria for these 1748 cases: adult ICU patients with or without acute infection. Exclusion criteria: pregnant people, myelosuppressed people on chemotherapy, people on immunosuppressant treatment, patients with hematologic diseases.

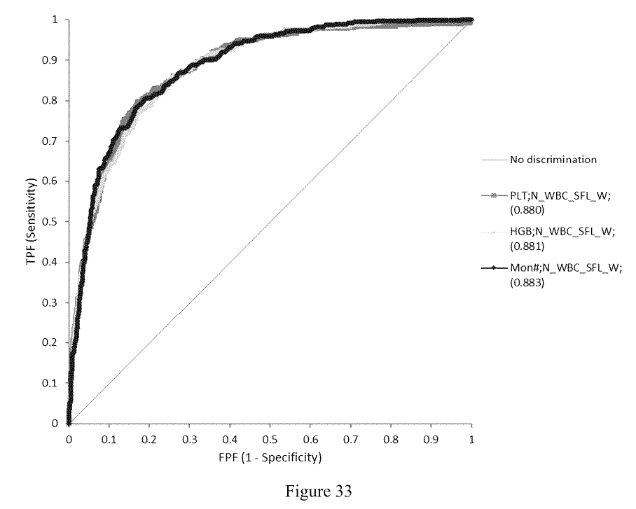

Table 29 shows the infection marker parameters used and their corresponding diagnostic efficacy, and FIG. 33 shows ROC curves corresponding to the infection marker parameters in Table 29. In Table 29:

$$Combination parameter 1 = - 0.61535116 * Mon + 0.00766353 * N\_WBC \_FL \_W - 15.04738706 ;$$

$$Combination parameter 2 = - 0.03077968 * HGB + 0.0893391 {8}^{*} N\_WBC \_FL \_W - 5.72270 \; 269 ;$$

$$Combination parameter 3 = - 0.00395999 * PLT + 0.00 \; 606333 * N\_WBC \_FL \_W - 11.55000862 .$$

TABLE 29
Efficacy of different infection marker parameters for diagnosis of sepsis

| Infection | | Determi- | False | True | True | False |
|---|---|---|---|---|---|---|
| marker | | nation | positive | positive | negative | negative |
| parameter | ROC_AUC | threshold | rate | rate | rate | rate |
| Combination | 0.8826 | >−0.9689 | 18.7% | 80.2% | 81.3% | 19.8% |
| parameter 1 | | | | | | |
| Combination | 0.8808 | >−0.8956 | 17.7% | 77.8% | 82.3% | 22.2% |
| parameter 2 | | | | | | |
| Combination | 0.8801 | >−0.9222 | 17.1% | 79.6% | 82.9% | 20.4% |
| parameter 3 | | | | | | |

From the comparison between Table 14-6 and Table 29, the combination parameter of monocyte counts, or hemoglobin values, or platelet counts combined with parameters of the WNB channel has better diagnostic performance in the diagnosis of sepsis than PCT or DIFF channel alone. It shows that the count values of leukocytes and platelets as well as the hemoglobin concentration of red blood cells in blood routine test can be used as the first leukocyte parameter, which is combined with the parameters of WNB channel to calculate the infection characteristic parameters for diagnosis of sepsis.

TABLE 30
Illustration of the statistical methods and testing methods
used in this example by taking three parameters as examples

| Infection | | | | |
|---|---|---|---|---|
| marker | Positive sample | Negative sample | | |
| parameter | Mean ± SD | Mean ± SD | F value | P value |
| Combination | 0.55 ± 1.87 | −2.36 ± 1.64 | −1017.29 | <0.0001 |
| parameter 1 | | | | |
| Combination | 0.35 ± 1.98 | −2.17 ± 1.40 | −1098.71 | <0.0001 |
| parameter 2 | | | | |
| Combination | 0.39 ± 1.92 | −2.18 ± 1.45 | −1093.70 | <0.0001 |
| parameter 3 | | | | |

As can be seen from Table 30, this parameter is analyzed by Welch test, and there is a significant statistical difference between the two groups (p<0.0001).

The features or combinations thereof mentioned above in the description, the drawings of the description, and claims can be combined with each other arbitrarily or used separately as long as they are meaningful within the scope of the disclosure and do not contradict each other. The advantages and features described with reference to the blood cell analyzer provided by the embodiment of the disclosure are applicable in a corresponding manner to the use of the blood cell analysis method and infection marker parameters provided by the embodiment of the disclosure, and vice versa.

The foregoing description merely relates to the embodiments of the disclosure, and is not intended to limit the scope of patent of the disclosure. All equivalent variations made by using the content of the specification and the accompanying drawings of the disclosure from the concept of the disclosure, or the direct/indirect applications of the contents in other related technical fields all fall within the scope of patent protection of the disclosure.

1. A method for indicating an infection status of a subject, comprising:
obtaining a blood sample to be tested from the subject;
preparing a test sample containing a part of the blood sample to be tested, a hemolytic agent, and a staining agent for identifying nucleated red blood cells;
passing particles in the test sample one by one through an optical detection region irradiated with light to obtain optical information generated by the particles in the test sample after being irradiated with light;
calculating at least one leukocyte characteristic parameter of at least one target particle population in the test sample from the optical information;
obtaining an infection marker parameter based on the at least one leukocyte characteristic parameter; and
indicating the infection status of the subject based on the infection marker parameter.

2. The method of claim 1, wherein the at least one target particle population is selected from one or more of leukocyte population, neutrophil population and lymphocyte population; or the at least one target particle population comprises leukocyte population or neutrophil population.

3. The method of claim 1, wherein calculating at least one leukocyte characteristic parameter of at least one target particle population in the test sample from the optical information comprises:
calculating a scatter or fluorescence signal intensity distribution center of gravity of the target particle population;
calculating a scatter or fluorescence signal intensity distribution width of the target particle population;
calculating a scatter or fluorescence signal intensity distribution coefficient of variation of the target particle population;
calculating an average value of scatter or fluorescence signal pulse widths of the target particle population;
calculating an area of a distribution region in a two-dimensional scattergram generated by two light intensities of the target particle population;
calculating a volume of a distribution region in a three-dimensional scattergram generated by three light intensities of the target particle population; or
calculating a boundary value of a scatter or fluorescence signal intensity distribution of the target particle population.

4. The method of claim 1, wherein the infection marker parameter is selected from one of the cell characteristic parameters or is obtained from a combination of a plurality of cell characteristic parameters of the cell characteristic parameters;
the one or more leukocyte characteristic parameters are selected from:
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, a side fluorescence intensity distribution coefficient of variation of the leukocyte population, an average value of side fluorescence signal pulse width, an average value of forward scatter signal pulse width, and an average value of side scatter signal pulse width and a right boundary value of side fluorescence intensity distribution of the leukocyte population;
an area of a distribution region of the leukocyte population in a two-dimensional scattergram generated by two light intensities selected from forward scatter intensity, side scatter intensity and side fluorescence intensity, a volume of a distribution region of the leukocyte population in a three-dimensional scattergram generated by forward scatter intensity, side scatter intensity and side fluorescence intensity;
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, and a side fluorescence intensity distribution coefficient of variation of the neutrophil population;
an area of a distribution region of the neutrophil population in a two-dimensional scattergram generated by two light intensities selected from forward scatter intensity, side scatter intensity and side fluorescence intensity, a volume of a distribution region of the neutrophil population in a three-dimensional scattergram generated by forward scatter intensity, side scatter intensity and side fluorescence intensity;
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, and a side fluorescence intensity distribution coefficient of variation of the lymphocyte population; and
an area of a distribution region of the lymphocyte population in a two-dimensional scattergram generated by two light intensities selected from forward scatter intensity, side scatter intensity and side fluorescence intensity, a volume of a distribution region of the lymphocyte population in a three-dimensional scattergram generated by forward scatter intensity, side scatter intensity and side fluorescence intensity.

5. The method of claim 1, wherein calculating from the optical information at least one leukocyte characteristic parameter of at least one target particle population in the test sample and obtaining an infection marker parameter based on the at least one leukocyte characteristic parameter comprises:
obtaining one or more of the following leukocyte characteristic parameters from the optical information and obtaining the infection marker parameter based on the one or more leukocyte characteristic parameters:
a forward scatter intensity distribution center of gravity, a side scatter intensity distribution center of gravity, a side fluorescence intensity distribution center of gravity, a forward scatter intensity distribution width, a side scatter intensity distribution width, a side fluorescence intensity distribution width, a forward scatter intensity distribution coefficient of variation, a side scatter intensity distribution coefficient of variation, a side fluorescence intensity distribution coefficient of variation of the leukocyte population, and an area of a distribution region of the leukocyte population in a two-dimensional scattergram generated by two light intensities selected from forward scatter intensity, side scatter intensity and side fluorescence intensity, a volume of a distribution region of the leukocyte population in a three-dimensional scattergram generated by forward scatter intensity, side scatter intensity and side fluorescence intensity.

6. The method of claim 1, wherein indicating the infection status of the subject based on the infection marker parameter comprises: performing on the subject an early prediction of sepsis, diagnosis of sepsis, an identification between common infection and severe infection, a monitoring of the infection status, an analysis of sepsis prognosis, an identification between bacterial infection and viral infection, an evaluation of therapeutic effect on sepsis, or an identification between non-infectious inflammation and infectious inflammation based on the infection marker parameter.

7. The method of claim 6, wherein indicating the infection status of the subject based on the infection marker parameter further comprises:
while performing on the subject an early prediction of sepsis, outputting prompt information indicating that the subject is likely to progress to sepsis within a certain period of time starting from when the blood sample to be tested is collected, when the infection marker parameter satisfies a first preset condition;
the certain period of time is not greater than 48 hours, or the certain period of time is within 24 hours; and
obtaining the side fluorescence intensity distribution width of leukocyte population or the side fluorescence intensity distribution width of neutrophil population from the optical information and determining the obtained distribution width as the infection marker parameter; or obtaining a combination of the side fluorescence intensity distribution center of gravity of leukocyte population and the forward scatter intensity distribution width of leukocyte population from the optical information, and calculating the infection marker parameter based on the combination.

8. The method of claim 6, wherein indicating the infection status of the subject based on the infection marker parameter comprises:
while performing on the subject a diagnosis of sepsis, outputting prompt information indicating that the subject has sepsis, when the infection marker parameter satisfies a second preset condition, and
obtaining the side fluorescence intensity distribution width of leukocyte population or the side fluorescence intensity distribution width of neutrophil population from the optical information and determining the obtained distribution width as the infection marker parameter; or obtaining from the optical information a combination of the side fluorescence intensity distribution center of gravity of leukocyte population and the forward scatter intensity distribution width of leukocyte population, and calculating the infection marker parameter based on the combination.

9. The method of claim 6, wherein indicating the infection status of the subject based on the infection marker parameter comprises:
while performing on the subject an identification of between a common infection and a severe infection, outputting prompt information indicating that the subject has a severe infection, when the infection marker parameter satisfies a third preset condition;
wherein, obtaining from the optical information a side fluorescence intensity distribution width of leukocyte population or an area of the distribution region of neutrophil population in the two-dimensional scattergram generated by the side scatter intensity and the side fluorescence intensity, and determining the obtained distribution width or area of the distribution region as the infection marker parameter; or obtaining from the optical information a combination of the side fluorescence intensity distribution center of gravity of the leukocyte population and the forward scatter intensity distribution width of the leukocyte population, and calculating the infection marker parameter based on the combination.

10. The method of claim 6, wherein the subject is an infected patient suffering from a severe infection or sepsis; and
indicating the infection status of the subject based on the infection marker parameter comprises:
performing on the subject a monitoring of the infection status, monitoring a progress in the infection status of the subject based on the infection marker parameter.

11. The method of claim 10, wherein monitoring the progress of the infection of the subject based on the infection marker parameter comprises:
obtaining multiple values of the infection marker parameter, which are obtained by multiple tests of a blood sample from the subject at different time points;
determining whether the infection status of the subject is improving or not according to a trend of change in the multiple values of the infection marker parameter obtained by the multiple tests, or, outputting prompt information indicating that the infection status of the subject is improving, when the multiple values of the infection marker parameter obtained by the multiple tests gradually tend to decrease.

12. The method of claim 10, wherein calculating from the optical information at least one leukocyte characteristic parameter of at least one target particle population in the test sample and obtaining an infection marker parameter based on the at least one leukocyte characteristic parameter comprises:
obtaining the side fluorescence intensity distribution width of leukocyte population from the optical information and determining the obtained distribution width as the infection marker parameter; or
obtaining from the optical information a combination of the side fluorescence intensity distribution center of gravity of leukocyte population and the forward scatter intensity distribution width of leukocyte population, and calculating the infection marker parameter based on the combination.

13. The method of claim 6, wherein indicating the infection status of the subject based on the infection marker parameter comprises:
while performing on the subject an analysis of sepsis prognosis, outputting prompt information indicating that the subject is in favorable sepsis prognosis, when the infection marker parameter satisfies a fourth preset condition.

14. The method of claim 6, wherein indicating the infection status of the subject based on the infection marker parameter and outputting prompt information indicating the infection status of the subject comprise:
while performing on the subject an identification between bacterial infection and viral infection, determining whether the subject has the bacterial infection or the viral infection based on the infection marker parameter.

15. The method of claim 6, wherein indicating the infection status of the subject based on the infection marker parameter and outputting prompt information indicating the infection status of the subject comprise:
while performing on the subject an identification between non-infectious inflammation and infectious inflammation, outputting prompt information indicating that the subject has an infectious inflammation, when the infection marker parameter satisfies a fifth preset condition.

16. The method of claim 6, wherein indicating the infection status of the subject based on the infection marker parameter and outputting prompt information indicating the infection status of the subject comprise:
while performing on the subject an evaluation of therapeutic effect on sepsis, evaluating a therapeutic effect on sepsis of the subject based on the infection marker parameter, when the subject is a patient with sepsis who is receiving medication.

17. The method of claim 1, wherein the method further comprises:
identifying nucleated red blood cells in the test sample based on the optical information to obtain a nucleated red blood cell count.

18. The method of claim 1, wherein the method further comprises:
obtaining a leukocyte count of the test sample based on the optical information before obtaining from the optical information the at least one leukocyte characteristic parameter of at least one target particle population in the test sample, and output a retest instruction to retest the blood sample of the subject when the leukocyte count is less than a preset threshold, wherein a measurement amount of the sample to be retested is greater than a measurement amount of the sample to be tested; and
obtaining at least another leukocyte characteristic parameter of at least another target particle population from the optical information obtained by the retest, and obtain an infection marker parameter for evaluating the infection status of the subject based on the at least another leukocyte characteristic parameter.

19. The method of claim 1, wherein the method further comprises:
skipping outputting a value of the infection marker parameter, or output a value of the infection marker parameter and simultaneously output prompt information indicating that the value of the infection marker parameter is unreliable, when a preset characteristic parameter of the target particle population satisfies a sixth preset condition; or
skipping outputting a value of the infection marker parameter, or output a value of the infection marker parameter and simultaneously output prompt information indicating that the value of the infection marker parameter is unreliable, when a total number of particles of the target particle population is less than a preset threshold or when the target particle population overlaps with another particle population.

20. The method of claim 1, wherein the method further comprises:
calculating a plurality of parameters of the at least one target particle population in the test sample from the optical information,
obtaining a plurality of sets of the infection marker parameters for evaluating the infection status of the subject from the plurality of parameters,
calculating a credibility of each set of the infection marker parameters of the plurality of sets of the infection marker parameters, select at least one set of the infection marker parameters from the plurality of sets of the infection marker parameters based on respective credibility of the plurality of sets of the infection marker parameters to obtain the infection marker parameter.

21. The method of claim 1, wherein the method further comprises:
determining based on the optical information whether the blood sample to be tested has an abnormality that affects the evaluation of the infection status;
obtaining from the optical information the at least one leukocyte characteristic parameter of at least one target particle population unaffected by the abnormality to obtain the infection marker parameter, when it is determined that the blood sample to be tested has the abnormality that affects the evaluation of the infection status.

22. A blood cell analyzer, comprising:
a sample aspiration device configured to aspirate a blood sample of a subject to be tested;
a sample preparation device configured to prepare a test sample containing a part of the blood sample to be tested, a hemolytic agent, and a staining agent for identifying nucleated red blood cells;
an optical detection device comprising a flow cell, a light source and an optical detector, the flow cell being configured to allow the test sample to pass therethrough, the light source being configured to irradiate with light the test sample passing through the flow cell, and the optical detector being configured to detect optical information generated by the test sample under irradiation when passing through the flow cell; and
a processor configured to:
calculate from the optical information at least one leukocyte characteristic parameter of at least one target particle population in the test sample;
obtain an infection marker parameter for evaluating an infection status of the subject based on the at least one leukocyte characteristic parameter; and
output the infection marker parameter.

23. A method of using an infection marker parameter in indicating an infection status of a subject, wherein the infection marker parameter is obtained by:
obtaining at least one leukocyte characteristic parameter of at least one target particle population obtained by flow cytometry detection on a test sample containing a blood sample to be tested from the subject, a hemolytic agent and a staining agent for identifying nucleated red blood cells; and
obtaining an infection marker parameter based on the at least one leukocyte characteristic parameter.
